(19) United States
(12) Patent Application Publication
(10) Pub. No.: US 2024/0065096 A1
(43) Pub. Date: Feb. 22, 2024

(54) COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Byoungkwan LEE, Suwon-si, Gyeonggi-do (KR); Sunwoong SHIN, Suwon-si, Gyeonggi-do (KR); Sangshin LEE, Suwon-si, Gyeonggi-do (KR); Yoonman LEE, Suwon-si, Gyeonggi-do (KR); Jin Sook KIM, Suwon-si, Gyeonggi-do (KR); Namheon LEE, Suwon-si, Gyeonggi-do (KR); Mijin LEE, Suwon-si, Gyeonggi-do (KR); Kipo JANG, Suwon-si, Gyeonggi-do (KR); Sung-Hyun JUNG, Suwon-si, Gyeonggi-do (KR); Ho Kuk JUNG, Suwon-si, Gyeonggi-do (KR); Bowon CHOI, Suwon-si, Gyeonggi-do (KR); Jinseok JANG, Suwon-si, Gyeonggi-do (KR)

(21) Appl. No.: 18/268,382

(22) PCT Filed: Dec. 29, 2021
(86) PCT No.: PCT/KR2021/020122

§ 371 Date: Jun. 20, 2023

(30) Foreign Application Priority Data
Dec. 30, 2020 (KR) 10-2020-0187969
Feb. 25, 2021 (KR) 10-2021-0025841
Dec. 28, 2021 (KR) 10-2021-0190023
Dec. 28, 2021 (KR) 10-2021-0190024

(51) Int. Cl. H10K 85/60; C07D 251/24; C09K 11/06; C07D 403/12; C07D 405/04; C07D 209/80; C07D 405/12; C07D 487/04

(52) U.S. Cl. CPC H10K 85/636; C07D 251/24; C09K 11/06; H10K 85/633; C07D 403/12; C07D 405/04; C07D 209/80; C07D 405/12; C07D 487/04; H10K 85/6572; H10K 85/615; H10K 50/11

(57) ABSTRACT

The present invention relates to a composition for an organic optoelectronic device, and an organic optoelectronic device and display device comprising same, the composition including a first compound represented by Chemical Formula 1 and a second compound represented by Chemical Formula 2, or a first compound represented by Chemical Formula 1 and a third compound represented by a combination of Chemical Formula 3 and Chemical Formula 4.

The details of Chemical Formula 1 to Chemical Formula 4 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of realizing an organic optoelectronic device having a high efficiency and long life-span.

Another embodiment provides an organic optoelectronic device including the composition for the organic optoelectronic device.

Another embodiment provides a display device including the organic optoelectronic device.

### Technical Solution

According to an embodiment, a composition for an organic optoelectronic device includes a first compound represented by Chemical Formula 1 and a second compound represented by Chemical Formula 2, or a first compound represented by Chemical Formula 1 and a third compound represented by a combination of Chemical Formula 3 and Chemical Formula 4.

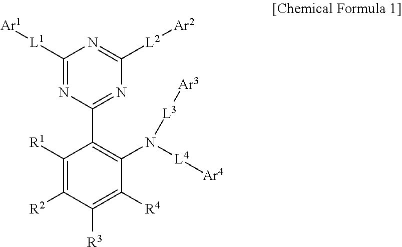

In Chemical Formula 1,
L¹ to L⁴ are each independently a single bond, a substituted or unsubstituted C6 to C30 arylene group, or a substituted or unsubstituted C2 to C30 heteroarylene group,
Ar¹ to Ar⁴ are each independently a substituted or unsubstituted C6 to C30 aryl group, or a substituted or unsubstituted C2 to C30 heterocyclic group,
R¹ to R⁴ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, and
R¹ to R⁴ are each independently present or adjacent groups are linked to each other to form a substituted or unsubstituted aromatic monocyclic ring, aromatic polycyclic ring, heteroaromatic monocyclic ring, or heteroaromatic polycyclic ring;

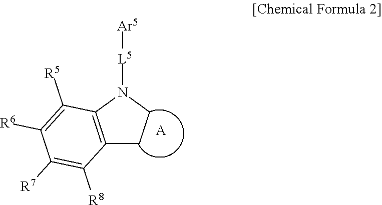

wherein, in Chemical Formula 2,
L⁵ is a single bond, or a substituted or unsubstituted C6 to C30 arylene group,
Ar⁵ is a substituted or unsubstituted C6 to C30 aryl group, or a substituted or unsubstituted C2 to C30 heterocyclic group, and
A is any one selected from the rings of Group I,

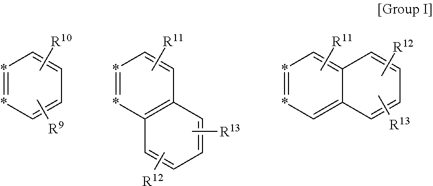

R⁵ to R¹³ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
at least one of R⁵ to R¹³ is a group represented by Chemical Formula a,

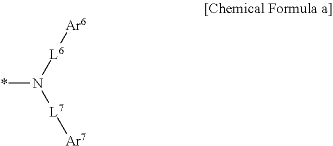

wherein, in Chemical Formula a,
L⁶ and L⁷ are each independently a single bond, or a substituted or unsubstituted C6 to C30 arylene group,
Ar⁶ and Ar⁷ are each independently a substituted or unsubstituted C6 to C30 aryl group, or a substituted or unsubstituted C2 to C30 heterocyclic group, and
* is a linking point,

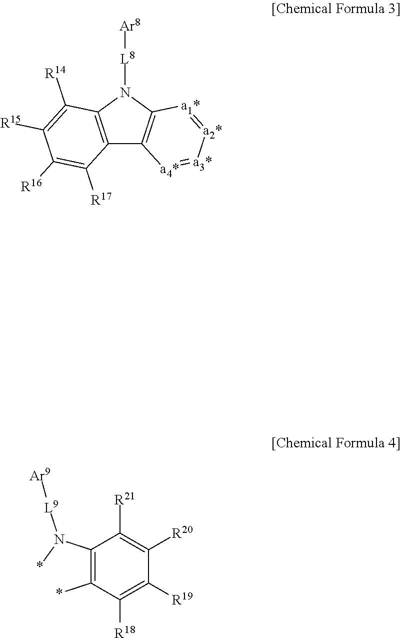

wherein, in Chemical Formula 3 and Chemical Formula 4,
adjacent two of a1* to a4* of Chemical Formula 3 are each independently a linking carbon (C) linked to * of Chemical Formula 4,
among a1* to a4* of Chemical Formula 3, the other two not linked to Chemical Formula 4 are CR^{a},
R^{a} and R¹⁴ to R²¹ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
L⁸ and L⁹ are each independently a single bond, a substituted or unsubstituted C6 to C30 arylene group, or a substituted or unsubstituted C2 to C30 heteroarylene group,
Ar⁸ and Ar⁹ are each independently a substituted or unsubstituted C6 to C30 aryl group, or a substituted or unsubstituted C2 to C30 heterocyclic group, and
* is a linking point.

According to another embodiment, an organic optoelectronic device includes an anode and a cathode facing each other, and at least one organic layer between the anode and the cathode, wherein the organic layer includes a light emitting layer and the light emitting layer includes the aforementioned composition for the organic optoelectronic device.

According to another embodiment, a display device including the organic optoelectronic device is provided.

### Advantageous Effects

High efficiency and long life-span organic optoelectronic devices may be implemented.

### DESCRIPTION OF THE DRAWINGS

FIGS. 1 to 4 are cross-sectional views illustrating an organic light emitting diode according to embodiments, respectively.

### DESCRIPTION OF SYMBOLS

100, 200, 300, 400: organic light emitting diode
105: organic layer
110: cathode
120: anode
130: light emitting layer
140: hole transport region
150: electron transport region

### MODE FOR INVENTION

Hereinafter, embodiments of the present invention are described in detail. However, these embodiments are exemplary, the present invention is not limited thereto and the present invention is defined by the scope of claims.

In the present specification, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples of the present invention, “substituted” refers to replacement of at least one hydrogen of a substituent or a compound by deuterium, a C1 to C20 alkyl group, a C6 to C30 aryl group, or a cyano group. In addition, in specific examples of the present invention, “substituted” refers to replacement of at least one hydrogen of a substituent or a compound by deuterium, a C1 to C5 alkyl group, a C6 to C18 aryl group, or a cyano group. In addition, in specific examples of the present invention, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group refers to a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group refers to a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a composition for an organic optoelectronic device according to an embodiment is described.

A composition for an organic optoelectronic device according to an embodiment includes a first compound represented by Chemical Formula 1 and a second compound represented by Chemical Formula 2, or a first compound represented by Chemical Formula 1 and a third compound represented by a combination of Chemical Formula 3 and Chemical Formula 4.

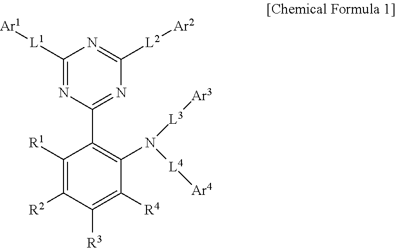

In Chemical Formula 1,
L¹ to L⁴ are each independently a single bond, a substituted or unsubstituted C6 to C30 arylene group, or a substituted or unsubstituted C2 to C30 heteroarylene group,
Ar¹ to Ar⁴ are each independently a substituted or unsubstituted C6 to C30 aryl group, or a substituted or unsubstituted C2 to C30 heterocyclic group,
R¹ to R⁴ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, and
R¹ to R⁴ are each independently present or adjacent groups are linked to each other to form a substituted or unsubstituted aromatic monocyclic ring, aromatic polycyclic ring, heteroaromatic monocyclic ring, or heteroaromatic polycyclic ring;

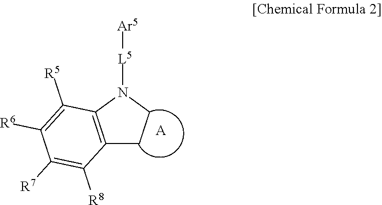

wherein, in Chemical Formula 2,
L⁵ is a single bond, or a substituted or unsubstituted C6 to C30 arylene group,
Ar⁵ is a substituted or unsubstituted C6 to C30 aryl group, or a substituted or unsubstituted C2 to C30 heterocyclic group, and
A is any one selected from the rings of Group I,

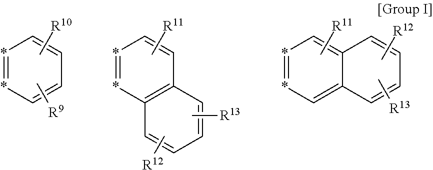

R⁵ to R¹³ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
at least one of R⁵ to R¹³ is a group represented by Chemical Formula a,

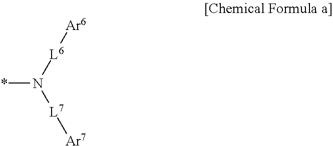

wherein, in Chemical Formula a,
L⁶ and L⁷ are each independently a single bond, or a substituted or unsubstituted C6 to C30 arylene group,
Ar⁶ and Ar⁷ are each independently a substituted or unsubstituted C6 to C30 aryl group, or a substituted or unsubstituted C2 to C30 heterocyclic group, and
* is a linking point,

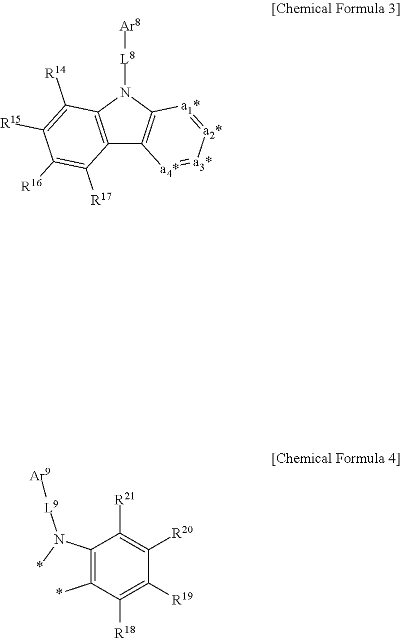

wherein, in Chemical Formula 3 and Chemical Formula 4,
adjacent two of a1* to a4* of Chemical Formula 3 are each independently a linking carbon (C) linked to * of Chemical Formula 4,
among a1* to a4* of Chemical Formula 3, the other two not linked to Chemical Formula 4 are CR^{a},
R^{a} and R¹⁴ to R²¹ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
L⁸ and L⁹ are each independently a single bond, a substituted or unsubstituted C6 to C30 arylene group, or a substituted or unsubstituted C2 to C30 heteroarylene group,
Ar⁸ and Ar⁹ are each independently a substituted or unsubstituted C6 to C30 aryl group, or a substituted or unsubstituted C2 to C30 heterocyclic group, and
* is a linking point.

The first compound represented by Chemical Formula 1 has a structure in which triazine and amine groups are linked by an ortho-linking group.

The first compound having such a structure has a bipolar characteristic by simultaneously including a triazine having an electronic characteristic and an amine group having a hole characteristic, and thus the LUMO energy level is shallowed.

By having a shallow LUMO energy level, a threshold voltage may be increased while maintaining a low driving voltage, so that the low grayscale phenomenon may be improved.

In particular, since the triazine and the amine group are linked through an ortho-linking group, a low deposition temperature compared to the molecular weight can be maintained, while a relatively high glass transition temperature (Tg) may be secured and deposition at a low temperature may be enabled, and thus thermal stability may be improved.

Meanwhile, the second compound has a structure in which carbazole or benzocarbazole is substituted with an amine group.

Since the second compound having such a structure has a high glass transition temperature and may be deposited at a relatively low temperature, it has excellent thermal stability.

The second compound has excellent hole transport characteristics, and is included together with the aforementioned first compound to increase the balance of holes and electrons, thereby greatly improving efficiency characteristics and life-span characteristics of a device to which the second compound is applied.

In addition, the third compound has an indolocarbazole structure.

The third compound having such a structure forms a flat planar structure in which an indole is fused to carbazole and has a shallow HOMO, and thus has relatively enhanced hole transport characteristics compared to carbazole and increases a balance of holes and electrons together with the first compound to greatly improve efficiency characteristics and life-span characteristics of the device to which they are applied.

According to an embodiment of the present invention, the first compound may be represented by any one of Chemical Formula 1-1 to Chemical Formula 1-4 according to the type of the linking group at the ortho position.

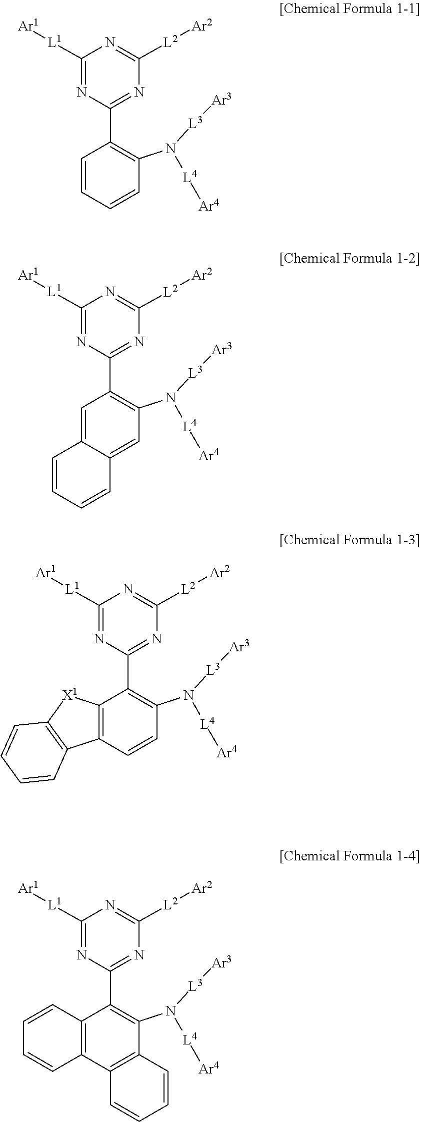

In Chemical Formula 1-1 to Chemical Formula 1-4, Ar¹ to Ar⁴ and L¹ to L⁴ are the same as described above, and X¹ is O or S.

For example, L¹ to L⁴ in Chemical Formula 1 may each independently be a single bond, a substituted or unsubstituted phenylene group, or a substituted or unsubstituted naphthylene group.

As a specific example, L¹ and L² of Chemical Formula 1 may each independently represent a single bond or a substituted or unsubstituted phenylene group.

As a specific example, L³ and L⁴ of Chemical Formula 1 may each independently be a single bond, a substituted or unsubstituted phenylene group, or a substituted or unsubstituted naphthylene group.

For example, Ar¹ and Ar² of Chemical Formula 1 may each independently be a substituted or unsubstituted phenyl group, a substituted or unsubstituted biphenyl group, a substituted or unsubstituted naphthyl group, a substituted or unsubstituted phenanthrenyl group, a substituted or unsubstituted It may be a triphenylene group, a substituted or unsubstituted chrysenyl group, a substituted or unsubstituted dibenzofuranyl group, a substituted or unsubstituted dibenzothiophenyl group, or a substituted or unsubstituted dibenzosilolyl group.

As a specific example, Ar¹ and Ar² of Chemical Formula 1 may each independently be a substituted or unsubstituted phenyl group, a substituted or unsubstituted biphenyl group, a substituted or unsubstituted naphthyl group, or a substituted or unsubstituted phenanthrenyl group.

For example, *-L¹-Ar¹ and *-L²-Ar² of Chemical Formula 1 may each independently be selected from the substituents of Group II.

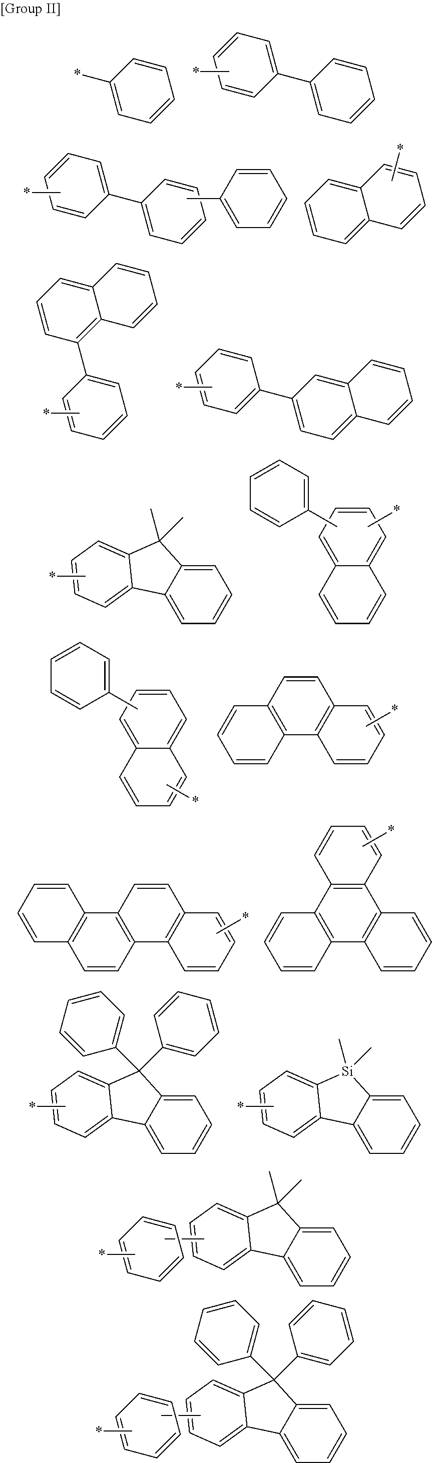

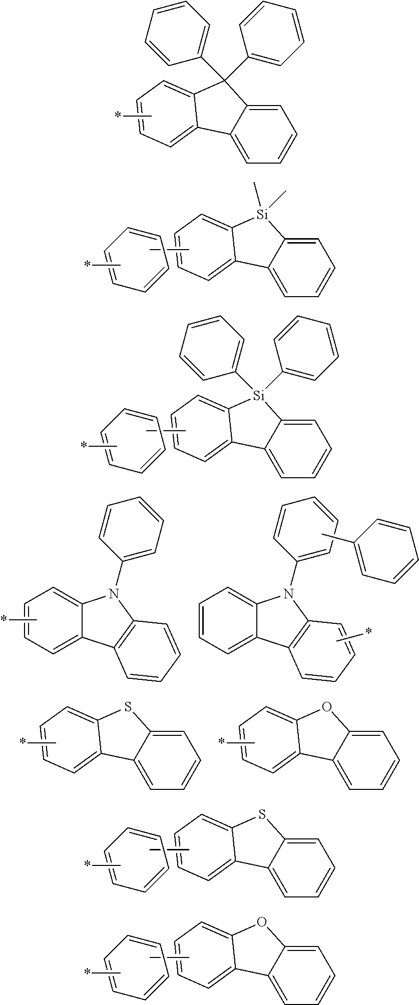

In Group II, * is a linking point.

For example, Ar³ and Ar⁴ of Chemical Formula 1 may each independently be a substituted or unsubstituted phenyl group, a substituted or unsubstituted biphenyl group, a substituted or unsubstituted naphthyl group, a substituted or unsubstituted fluorenyl group, a substituted or unsubstituted dibenzofuranyl group, a substituted or unsubstituted dibenzothiophenyl group, or a substituted or unsubstituted dibenzosilolyl group.

As a specific example, Ar³ and Ar⁴ of Chemical Formula 1 may each independently be a substituted or unsubstituted phenyl group, a substituted or unsubstituted biphenyl group, a substituted or unsubstituted naphthyl group, a substituted or unsubstituted fluorenyl group, or a substituted or unsubstituted dibenzosilolyl group.

For example, *-L³-Ar³ and *-L⁴-Ar⁴ of Chemical Formula 1 may each independently be selected from the substituents of Group III.

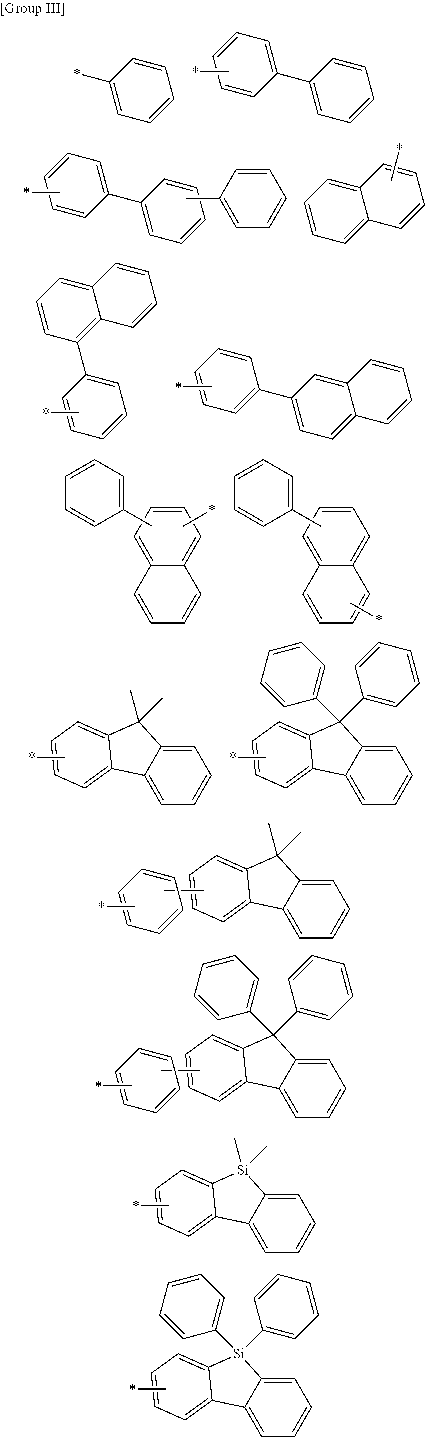

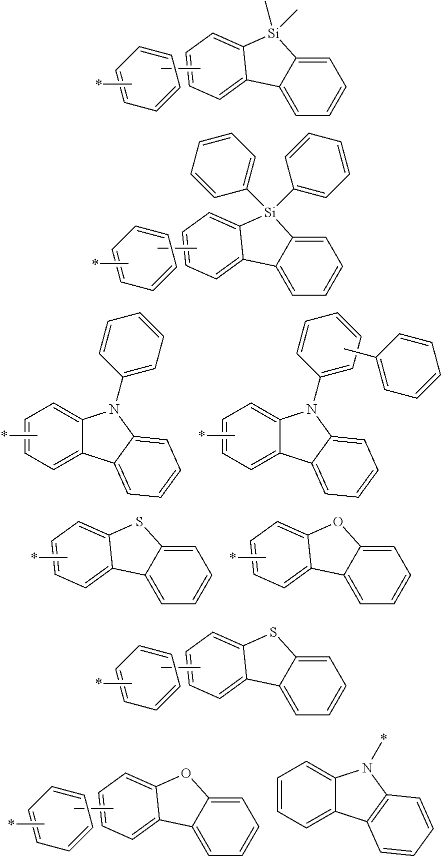

In Group III, * is a linking point.

For example, the first compound may be one selected from the compounds of Group 1, but is not limited thereto.

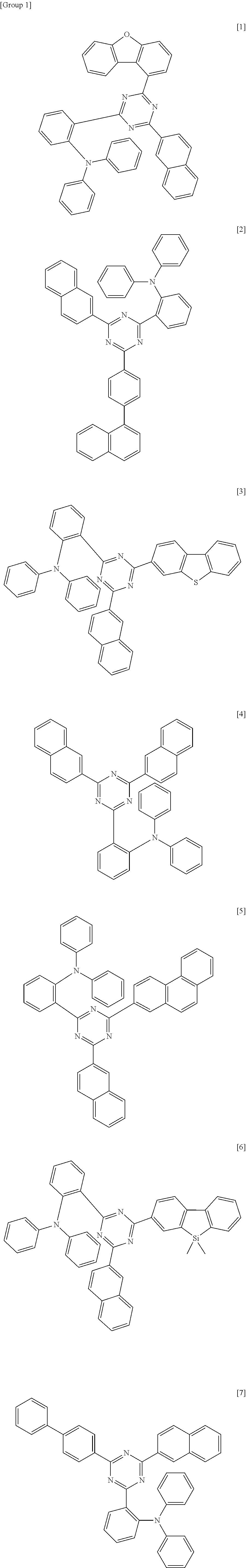

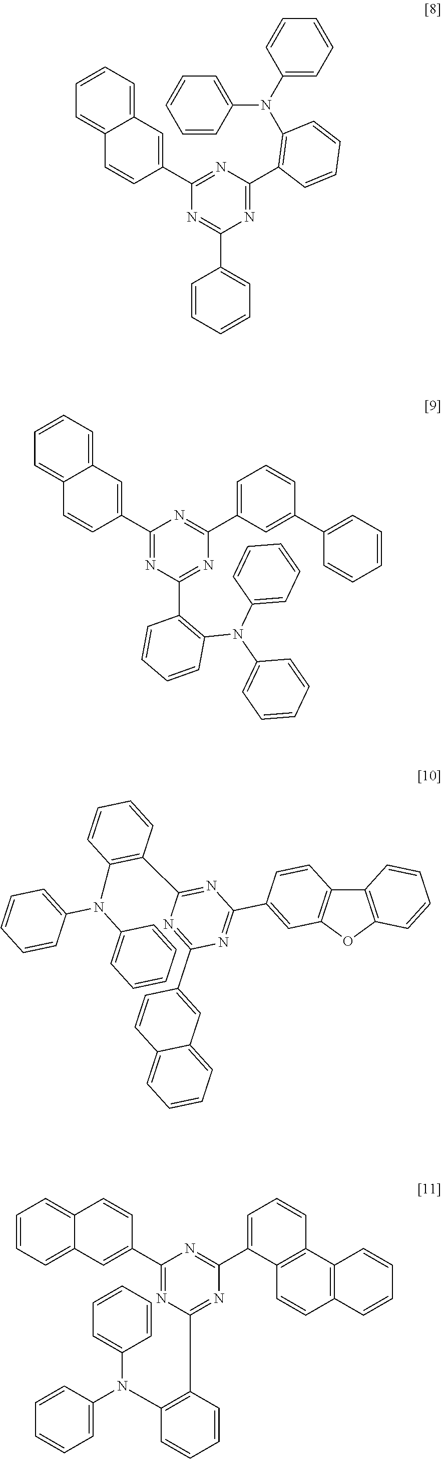 [SMILES: c1ccc(-c2nc(-c3ccc4ccccc4c3)nc(-c3ccccc3N(c3ccccc3)c3ccccc3)n2)cc1 ; c1ccc(-c2cccc(-c3nc(-c4ccc5ccccc5c4)nc(-c4ccccc4N(c4ccccc4)c4ccccc4)n3)c2)cc1 ; c1ccc(N(c2ccccc2)c2ccccc2-c2nc(-c3ccc4ccccc4c3)nc(-c3ccc4c(c3)oc3ccccc34)n2)cc1 ; c1ccc(N(c2ccccc2)c2ccccc2-c2nc(-c3ccc4ccccc4c3)nc(-c3cccc4c3ccc3ccccc34)n2)cc1]

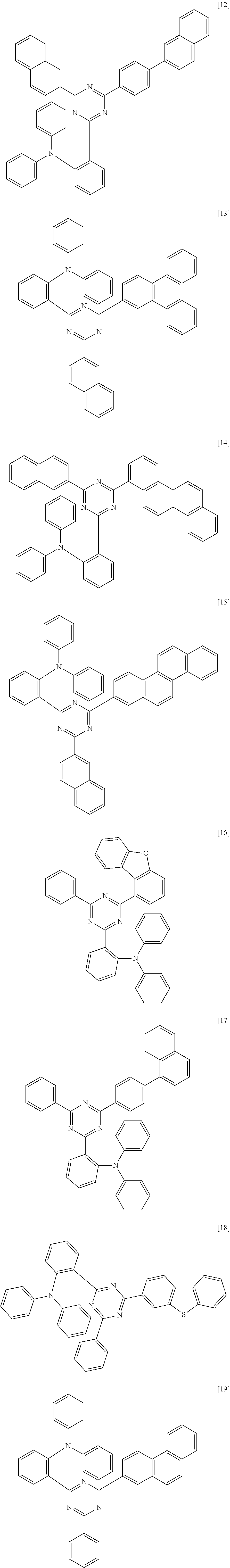

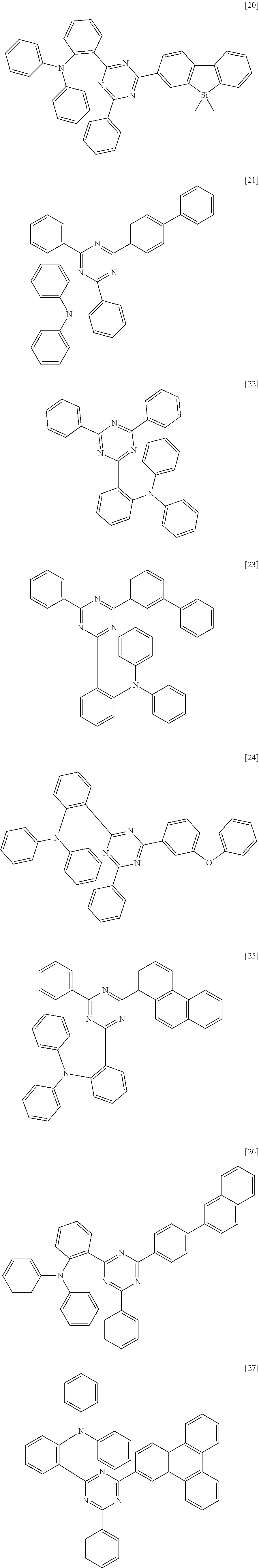

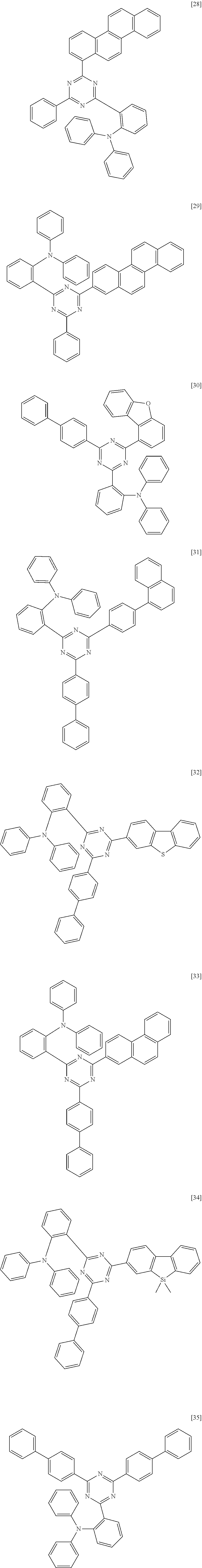

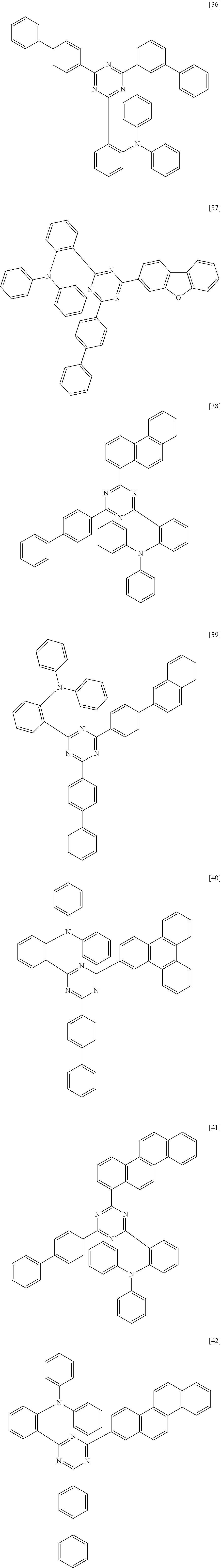

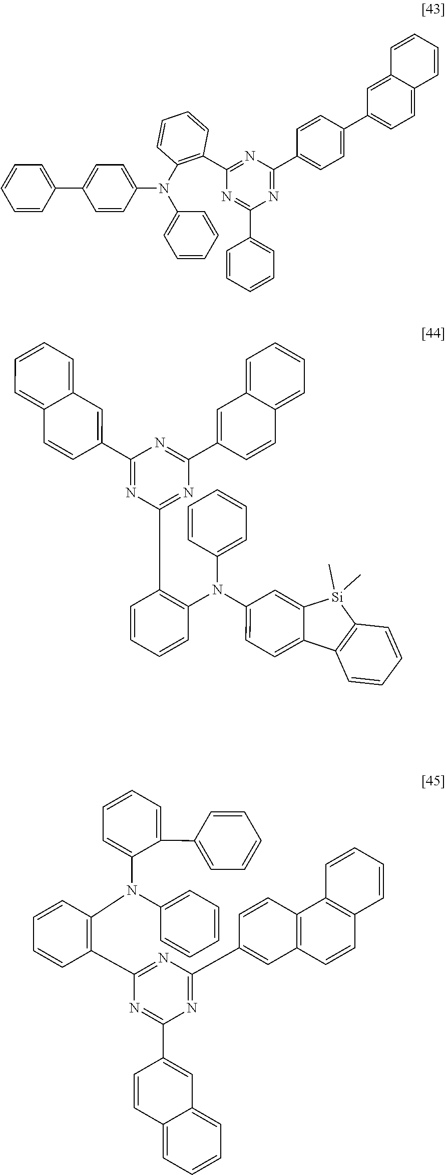 [SMILES: c1ccc(-c2ccc(N(c3ccccc3)c3ccccc3-c3nc(-c4ccccc4)nc(-c4ccc(-c5ccc6ccccc6c5)cc4)n3)cc2)cc1 ; C[Si]1(C)c2ccccc2-c2ccc(N(c3ccccc3)c3ccccc3-c3nc(-c4ccc5ccccc5c4)nc(-c4ccc5ccccc5c4)n3)cc21 ; c1ccc(-c2ccccc2N(c2ccccc2)c2ccccc2-c2nc(-c3ccc4ccccc4c3)nc(-c3ccc4c(ccc5ccccc54)c3)n2)cc1]

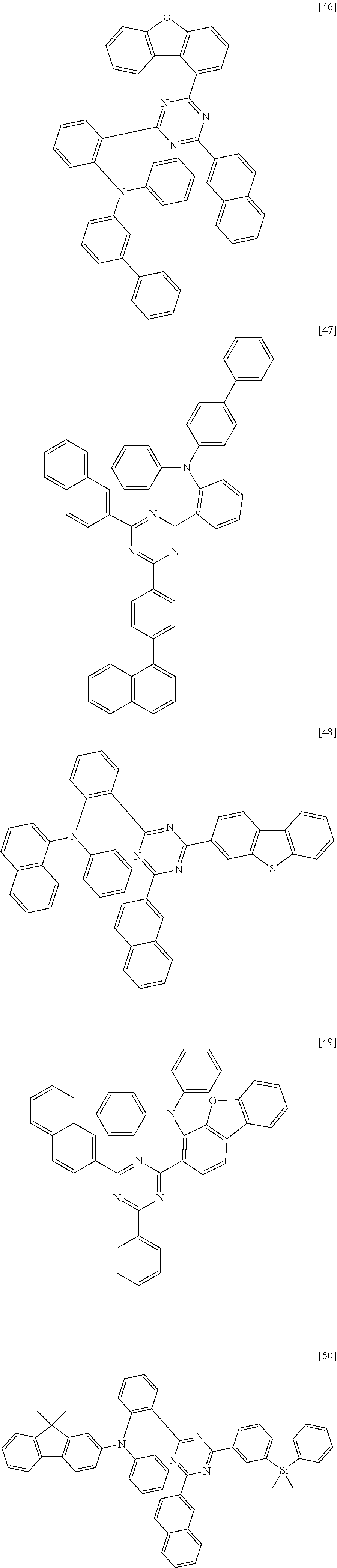 [SMILES: c1ccc(-c2cccc(N(c3ccccc3)c3ccccc3-c3nc(-c4ccc5ccccc5c4)nc(-c4cccc5oc6ccccc6c45)n3)c2)cc1 ; c1ccc(-c2ccc(N(c3ccccc3)c3ccccc3-c3nc(-c4ccc(-c5cccc6ccccc56)cc4)nc(-c4ccc5ccccc5c4)n3)cc2)cc1 ; c1ccc(N(c2ccccc2-c2nc(-c3ccc4ccccc4c3)nc(-c3ccc4c(c3)sc3ccccc34)n2)c2cccc3ccccc23)cc1 ; c1ccc(-c2nc(-c3ccc4ccccc4c3)nc(-c3ccc4c(oc5ccccc54)c3N(c3ccccc3)c3ccccc3)n2)cc1 ; CC1(C)c2ccccc2-c2ccc(N(c3ccccc3)c3ccccc3-c3nc(-c4ccc5c(c4)[Si](C)(C)c4ccccc4-5)nc(-c4ccc5ccccc5c4)n3)cc21]

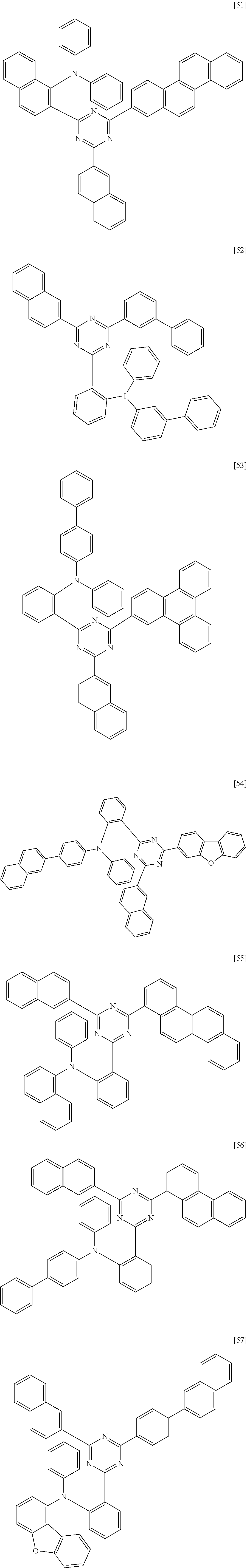

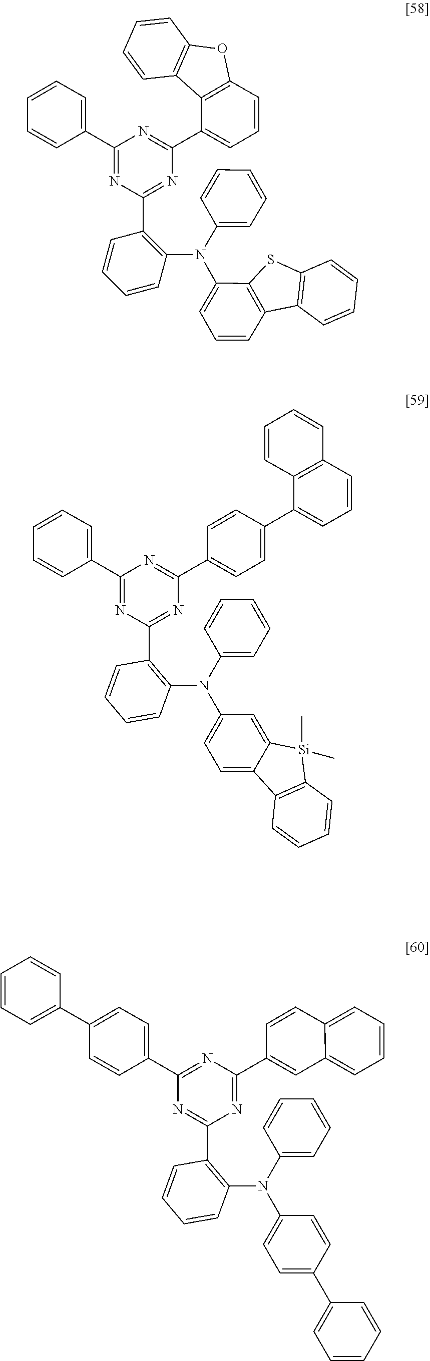 [SMILES: c1ccc(-c2nc(-c3ccccc3N(c3ccccc3)c3cccc4c3sc3ccccc34)nc(-c3cccc4oc5ccccc5c34)n2)cc1 ; C[Si]1(C)c2ccccc2-c2ccc(N(c3ccccc3)c3ccccc3-c3nc(-c4ccccc4)nc(-c4ccc(-c5cccc6ccccc56)cc4)n3)cc21 ; c1ccc(-c2ccc(-c3nc(-c4ccc5ccccc5c4)nc(-c4ccccc4N(c4ccccc4)c4ccc(-c5ccccc5)cc4)n3)cc2)cc1]

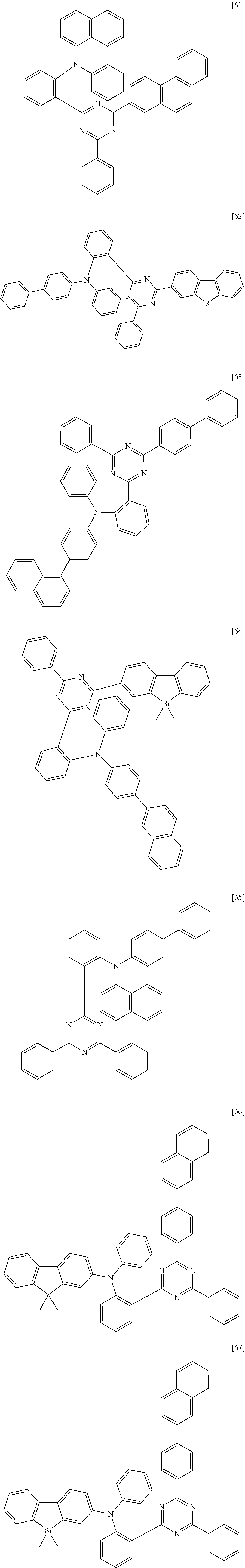

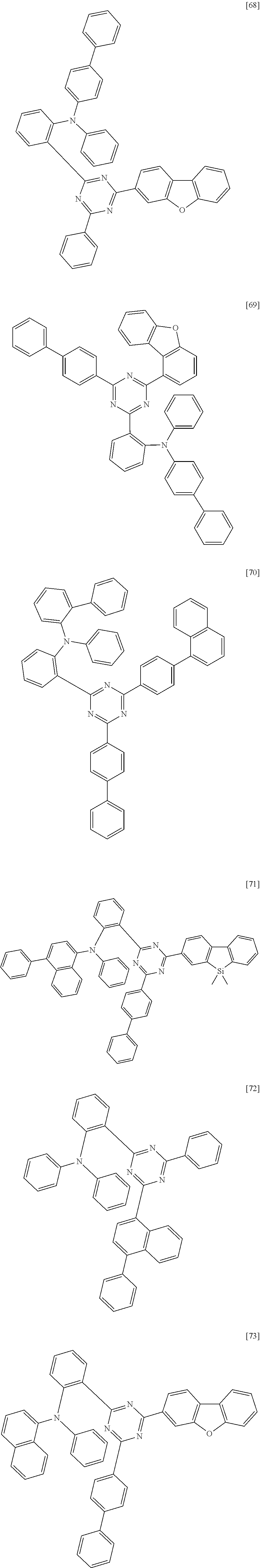

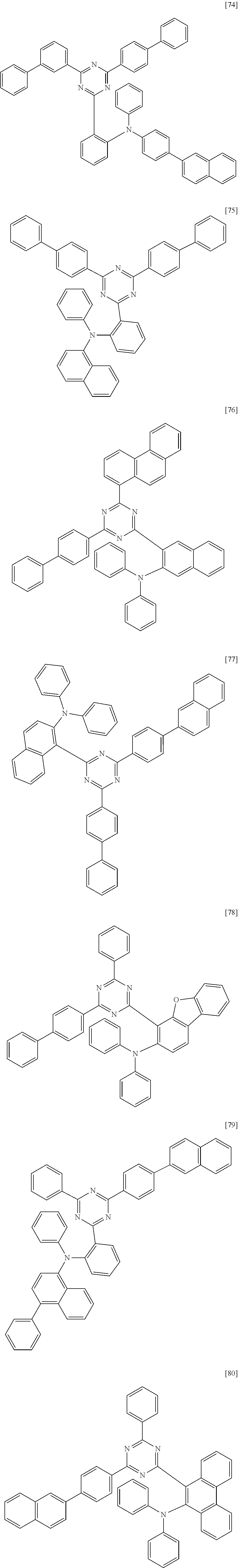

Meanwhile, the second compound may be represented by any one of Chemical Formula 2A-I to Chemical Formula 2D-I and Chemical Formula 2B-II to Chemical Formula 2D-II depending on the specific structure of ring A and the substitution direction of the amine group.

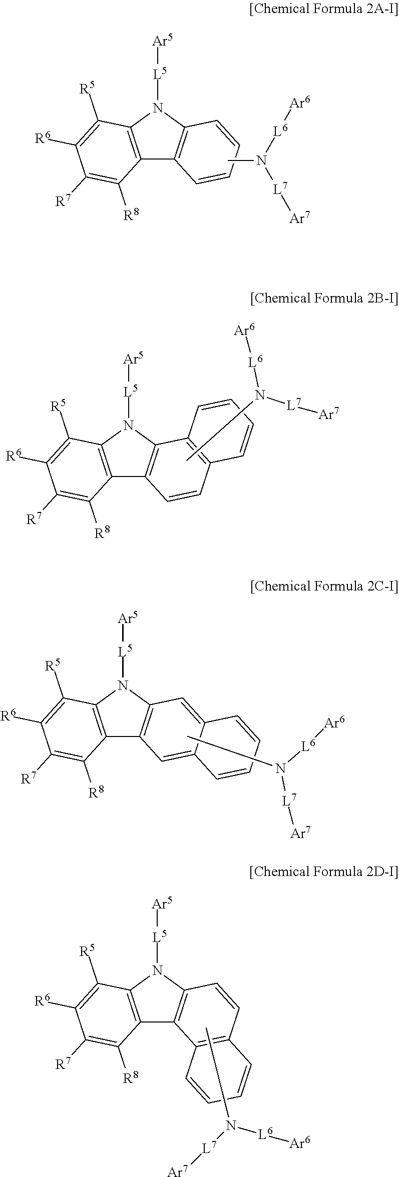

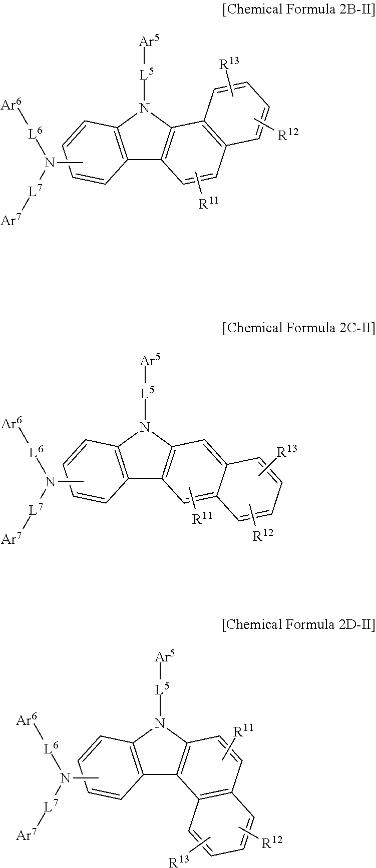

In Chemical Formula 2A-I to Chemical Formula 2D-I and Chemical Formula 2B-II to Chemical Formula 2D-II, L⁵ to L⁷ and Ar⁵ to Ar⁷ are the same as described above, and
R⁵ to R⁸ and R¹¹ to R¹³ are each independently hydrogen, deuterium, a substituted or unsubstituted C1 to C30 alkyl group, a substituted or unsubstituted C6 to C30 aryl group, or a substituted or unsubstituted C2 to C30 heterocyclic group.

For example, the second compound may be represented by any one of Chemical Formula 2A-I to Chemical Formula 2D-I, and

Chemical Formula 2A-I to Chemical Formula 2D-I may each be represented by any one of Chemical Formula 2A-I-1 to Chemical Formula 2A-I-4, Chemical Formula 2B-I-1 to Chemical Formula 2B-I-6, Chemical Formula 2C-I-1 to Chemical Formula 2C-I-6 and Chemical Formula 2D-I-1 to Chemical Formula 2D-I-6.

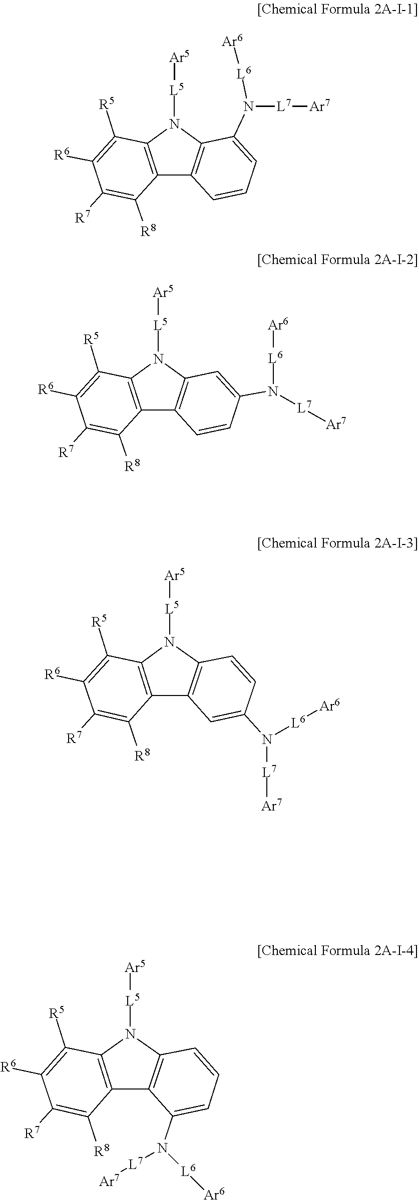

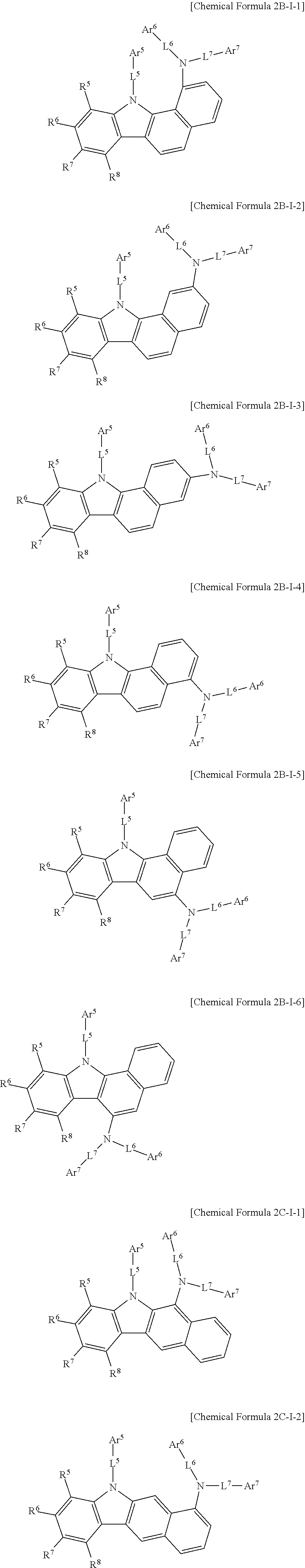

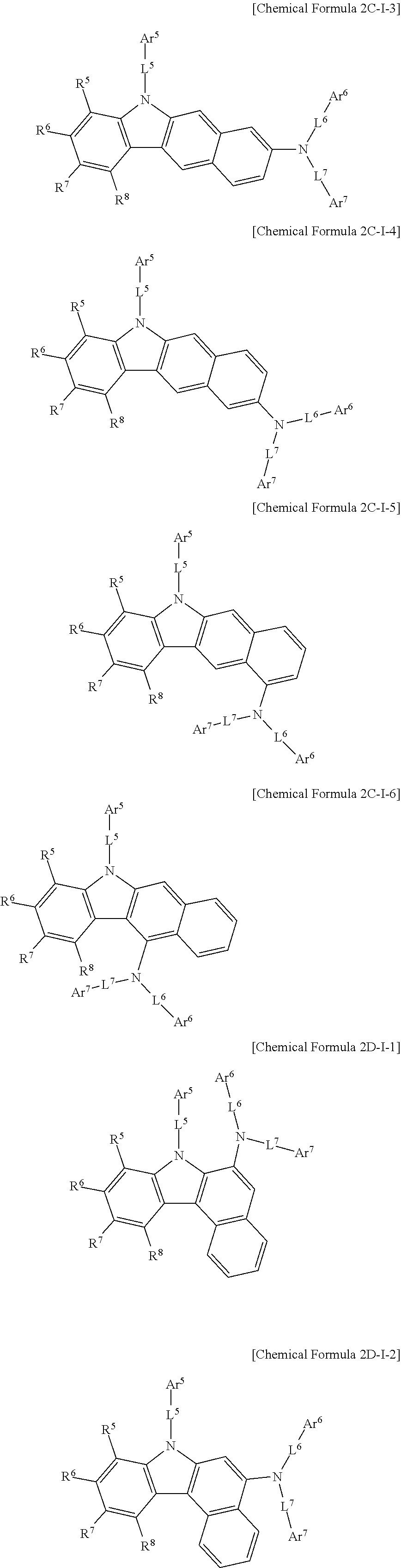

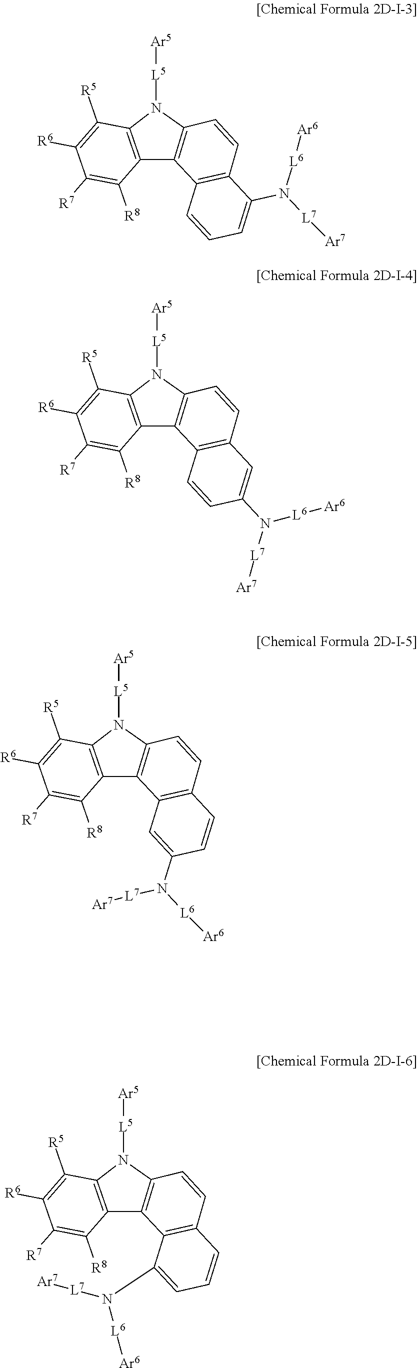

In Chemical Formula 2A-I-1 to Chemical Formula 2A-I-4, Chemical Formula 2B-I-1 to Chemical Formula 2B-I-6, Chemical Formula 2C-I-1 to Chemical Formula 2C-I-6, and Chemical Formula 2D-I-1 to Chemical Formula 2D-I-6, L⁵ to L⁷, Ar⁵ to Ar⁷, and R⁵ to R⁸ are the same as described above.

For example, L⁵ of Chemical Formula 2 may be a single bond.

For example, Ar⁵ of Chemical Formula 2 may be a substituted or unsubstituted phenyl group or a substituted or unsubstituted biphenyl group.

For example, L⁶ and L⁷ of Chemical Formula 2A-I to Chemical Formula 2D-I and Chemical Formula 2B-II to Chemical Formula 2D-II may each independently be a single bond or a substituted or unsubstituted C6 to C12 arylene group.

As a specific example, L⁶ and L⁷ of Chemical Formula 2A-I to Chemical Formula 2D-I and Chemical Formula 2B-II to Chemical Formula 2D-II may each independently be a single bond or a substituted or unsubstituted phenylene group.

For example, Ar⁶ and Ar⁷ of Chemical Formula 2 may each independently be a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted benzofuranfluorenyl group, or a substituted or unsubstituted benzothiophenefluorenyl group.

As a specific example, Ar⁶ and Ar⁷ of Chemical Formula 2 may each independently be a substituted or unsubstituted phenyl group, a substituted or unsubstituted biphenyl group, a substituted or unsubstituted naphthyl group, a substituted or unsubstituted dibenzofuranyl group, or a substituted or unsubstituted dibenzothiophenyl group.

For example, *-L⁶-Ar⁶ and *-L⁷-Ar⁷ of Chemical Formula 2A-I to Chemical Formula 2D-I and Chemical Formula 2B-II to Chemical Formula 2D-II may each independently be selected from substituents of Group IV.

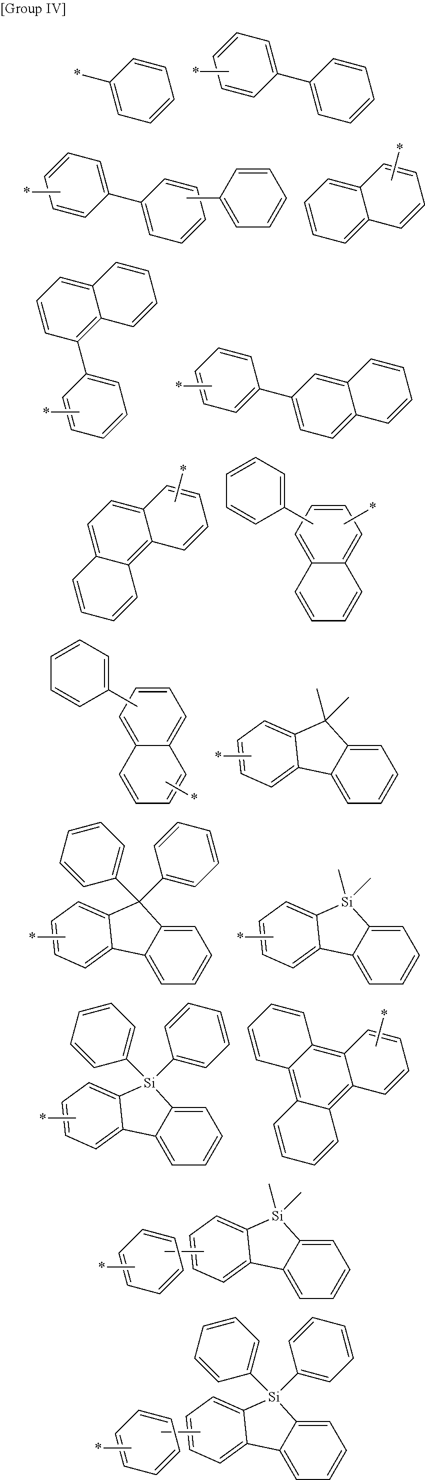

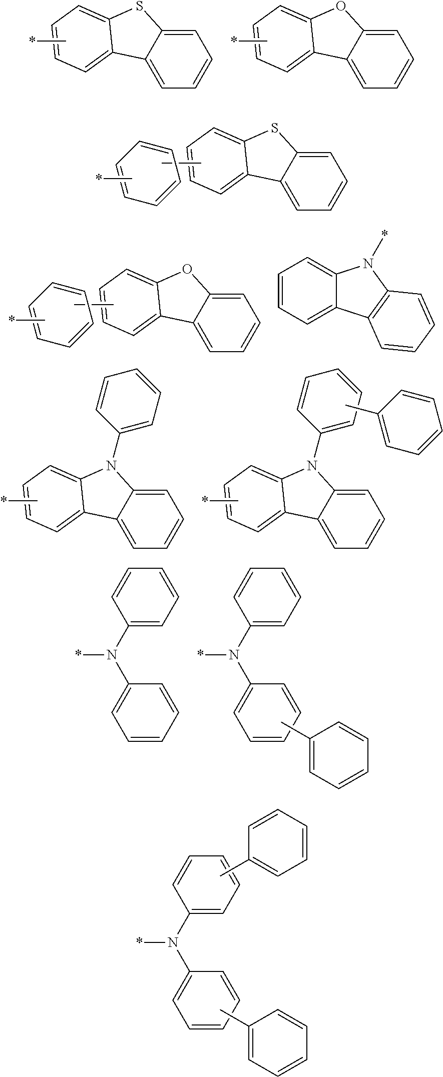

For example, R⁵ to R⁸ of Chemical Formula 2A-I to Chemical Formula 2D-I may each independently be hydrogen, deuterium, a substituted or unsubstituted C1 to C10 alkyl group, or a substituted or unsubstituted C6 to C12 aryl group.

As a specific example, R⁵ to R⁸ of Chemical Formula 2A-I to Chemical Formula 2D-I may each independently be hydrogen, deuterium, or a phenyl group.

For example, R¹¹ to R¹³ of Chemical Formula 2B-II to Chemical Formula 2D-II may each independently be hydrogen, deuterium, a substituted or unsubstituted C1 to C10 alkyl group, or a substituted or unsubstituted C6 to C12 aryl group.

As a specific example, R¹¹ to R¹³ of Chemical Formula 2B-II to Chemical Formula 2D-II may each independently be hydrogen, deuterium, or a phenyl group.

For example, the second compound may be one selected from the compounds of Group 2, but is not limited thereto.

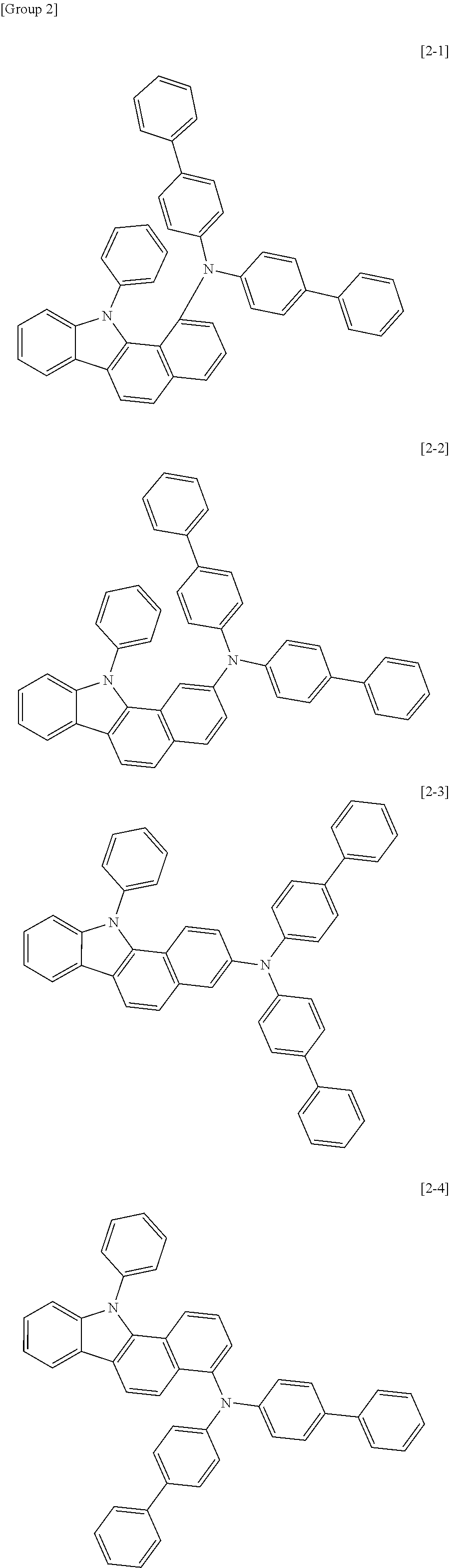 [SMILES: c1ccc(-c2ccc(N(c3ccc(-c4ccccc4)cc3)c3cccc4ccc5c6ccccc6n(-c6ccccc6)c5c34)cc2)cc1 ; c1ccc(-c2ccc(N(c3ccc(-c4ccccc4)cc3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1 ; c1ccc(-c2ccc(N(c3ccc(-c4ccccc4)cc3)c3cccc4c3ccc3c5ccccc5n(-c5ccccc5)c43)cc2)cc1]

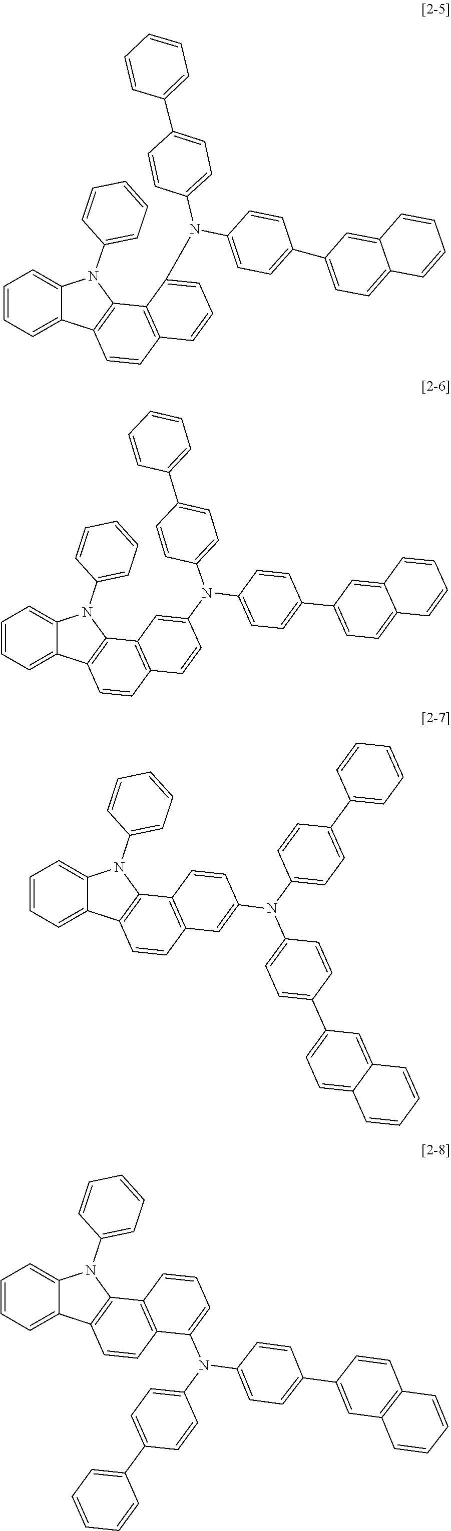 [SMILES: c1ccc(-c2ccc(N(c3ccc(-c4ccc5ccccc5c4)cc3)c3cccc4ccc5c6ccccc6n(-c6ccccc6)c5c34)cc2)cc1 ; c1ccc(-c2ccc(N(c3ccc(-c4ccc5ccccc5c4)cc3)c3ccc4ccc5c6ccccc6n(-c6ccccc6)c5c4c3)cc2)cc1 ; c1ccc(-c2ccc(N(c3ccc(-c4ccc5ccccc5c4)cc3)c3ccc4c(ccc5c6ccccc6n(-c6ccccc6)c45)c3)cc2)cc1 ; c1ccc(-c2ccc(N(c3ccc(-c4ccc5ccccc5c4)cc3)c3cccc4c3ccc3c5ccccc5n(-c5ccccc5)c43)cc2)cc1]

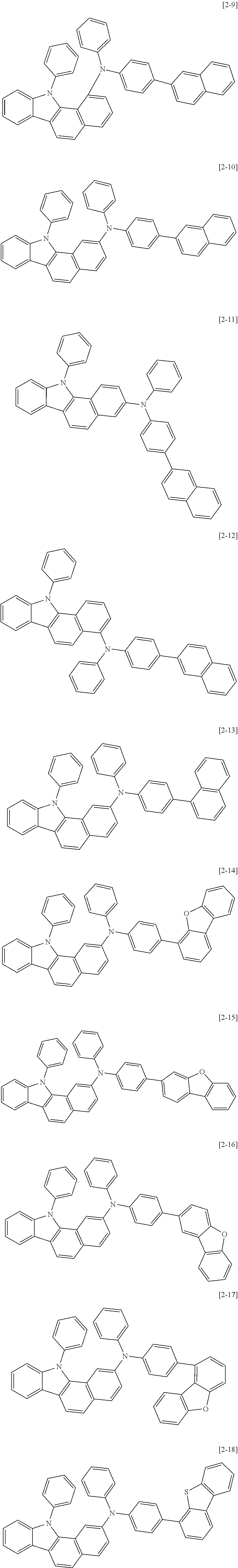

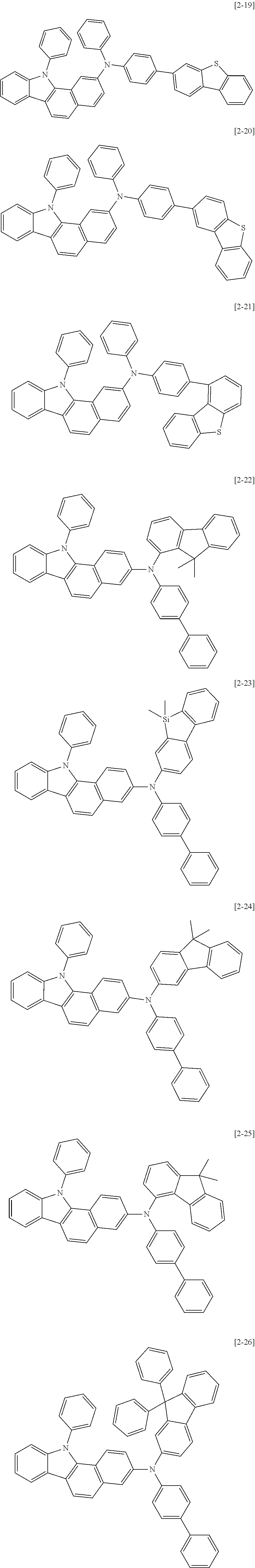

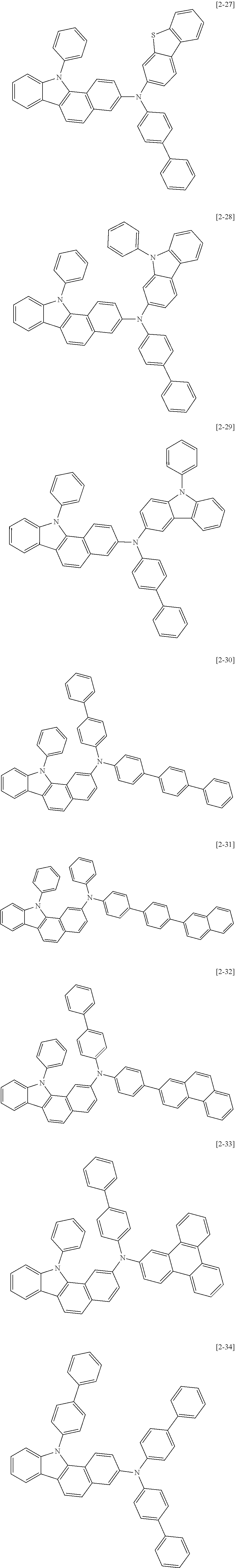

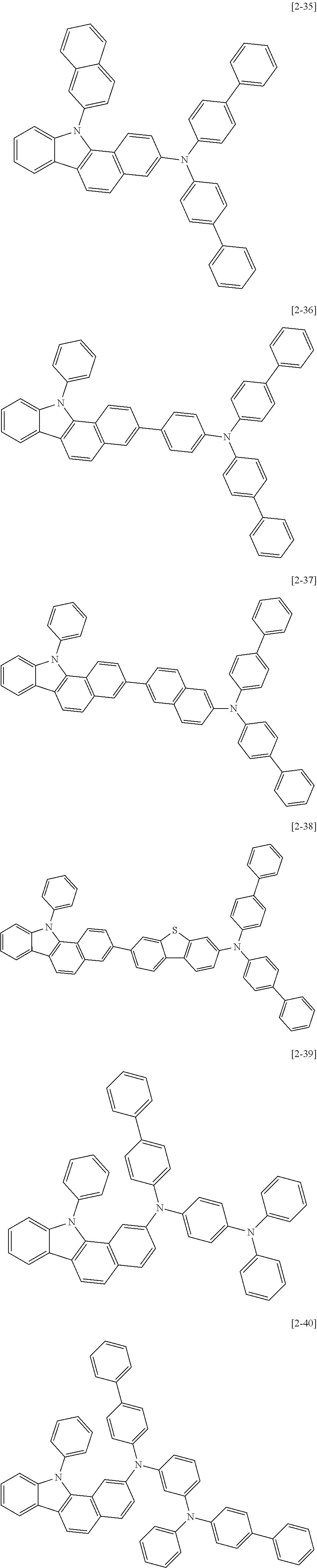

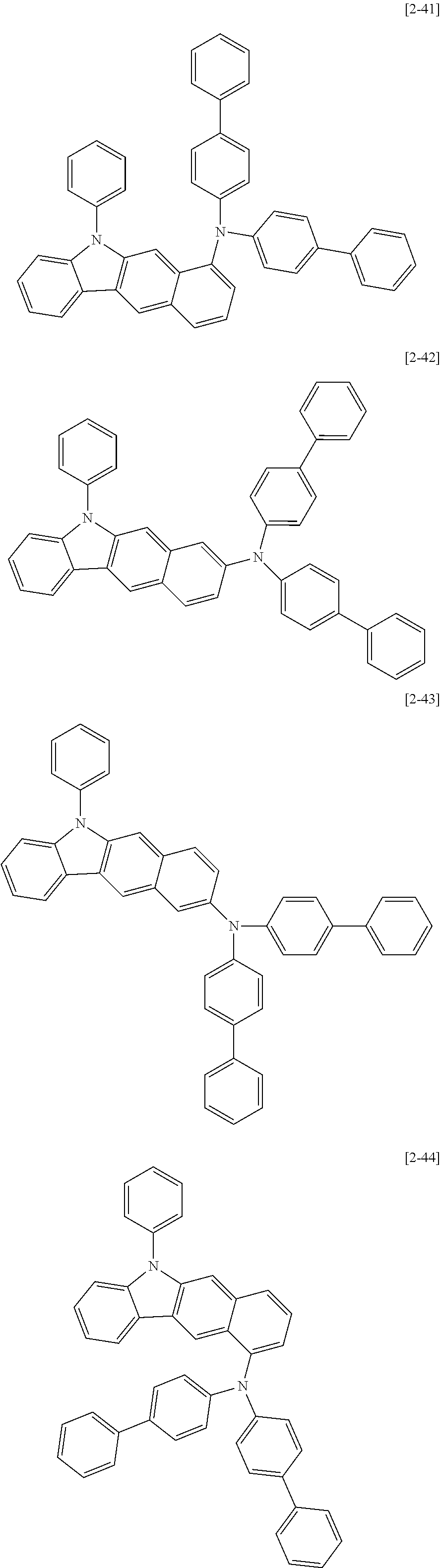 [SMILES: c1ccc(-c2ccc(N(c3ccc(-c4ccccc4)cc3)c3cccc4cc5c6ccccc6n(-c6ccccc6)c5cc34)cc2)cc1 ; c1ccc(-c2ccc(N(c3ccc(-c4ccccc4)cc3)c3ccc4cc5c6ccccc6n(-c6ccccc6)c5cc4c3)cc2)cc1 ; c1ccc(-c2ccc(N(c3ccc(-c4ccccc4)cc3)c3ccc4cc5c(cc4c3)c3ccccc3n5-c3ccccc3)cc2)cc1 ; c1ccc(-c2ccc(N(c3ccc(-c4ccccc4)cc3)c3cccc4cc5c(cc34)c3ccccc3n5-c3ccccc3)cc2)cc1]

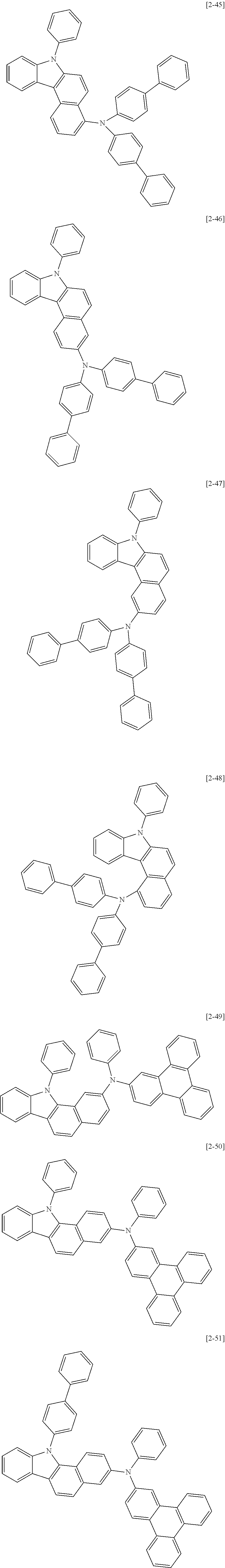

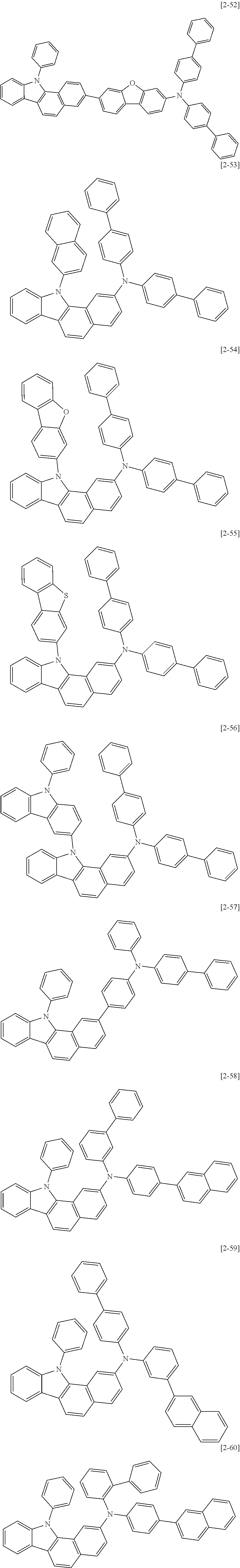

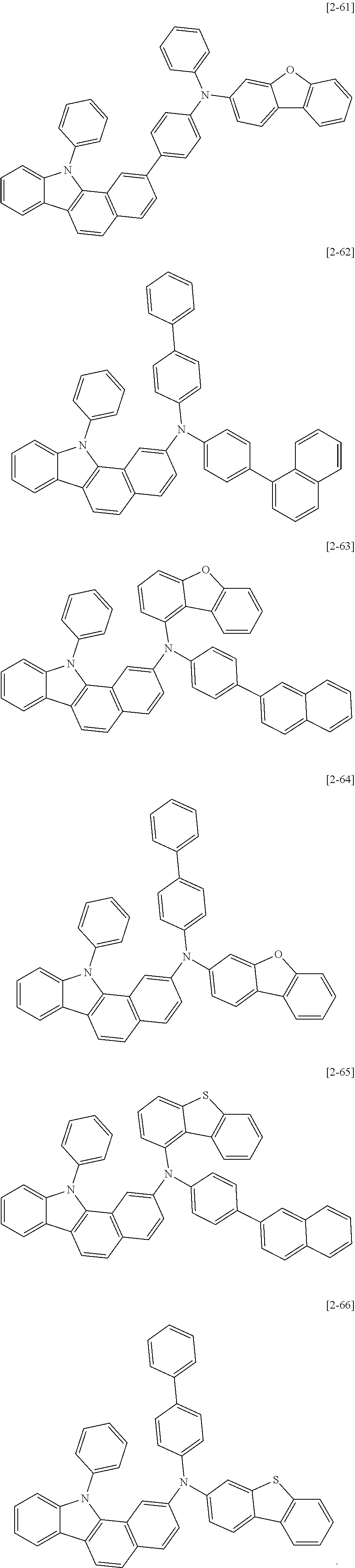

Meanwhile, the third compound may be represented by any one of Chemical Formula 3A to Chemical Formula 3E according to the fusion position of Chemical Formula 3 and Chemical Formula 4.

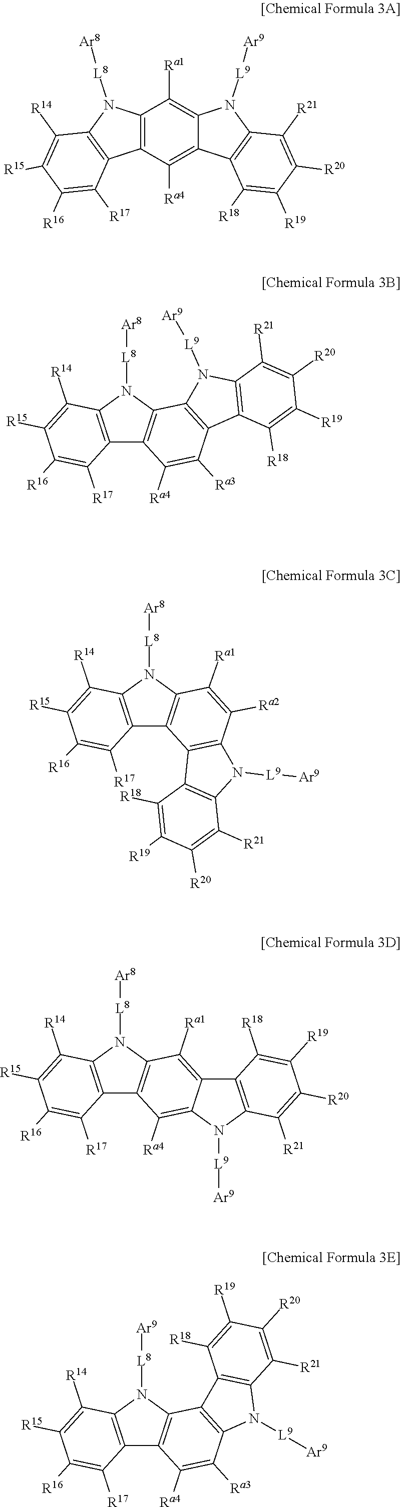

In Chemical Formula 3A to Chemical Formula 3E,
L⁸, L⁹, Ar⁸, Ar⁹, R^{a1} to R^{a4}, and R¹⁴ to R²¹ are the same as described above.

In an embodiment, the third compound may be represented by Chemical Formula 3C.

In particular, the third compound represented by Chemical Formula 3C has a shallow HOMO energy level, so that hole injection characteristics may be maximized.

For example, L⁸ and L⁹ of Chemical Formulas 3 and 4 may each independently be a single bond, a substituted or unsubstituted phenylene group, or a substituted or unsubstituted pyridinylene group.

For example, Ar⁸ and Ar⁹ of Chemical Formulas 3 and 4 may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted chrysenyl group, a substituted or unsubstituted carbazolyl group, a substituted or unsubstituted dibenzofuranyl group, a substituted or unsubstituted dibenzothiophenyl group, a substituted or unsubstituted pyridinyl group, a substituted or unsubstituted pyrimidinyl group, or a substituted or unsubstituted triazinyl group.

As a specific example, Ar⁸ and Ar⁹ of Chemical Formulas 3 and 4 may each independently be a substituted or unsubstituted phenyl group, a substituted or unsubstituted biphenyl group or a substituted or unsubstituted naphthyl group.

For example, R^{a1} to R^{a4} and R¹⁴ to R²¹ of Chemical Formula 3A to Chemical Formula 3E may each independently be hydrogen, deuterium, a substituted or unsubstituted C6 to C12 aryl group, or a substituted or unsubstituted C2 to C12 heterocyclic group.

As a specific example, R^{a1} to R^{a4} and R¹⁴ to R²¹ of Chemical Formula 3A to Chemical Formula 3E may each independently be hydrogen, deuterium, a substituted or unsubstituted phenyl group, a substituted or unsubstituted naphthyl group, a substituted or unsubstituted carbazolyl group, a substituted or unsubstituted dibenzofuranyl group, or a substituted or unsubstituted dibenzothiophenyl group.

For example, R^{a1} to R^{a4} and R¹⁴ to R²¹ of Chemical Formula 3A to Chemical Formula 3E may each independently be hydrogen or a phenyl group.

For example, *-L⁸-Ar⁸ and *-L⁹-Ar⁹ of Chemical Formulas 3 and 4 may each independently be selected from the substituents of Group V.

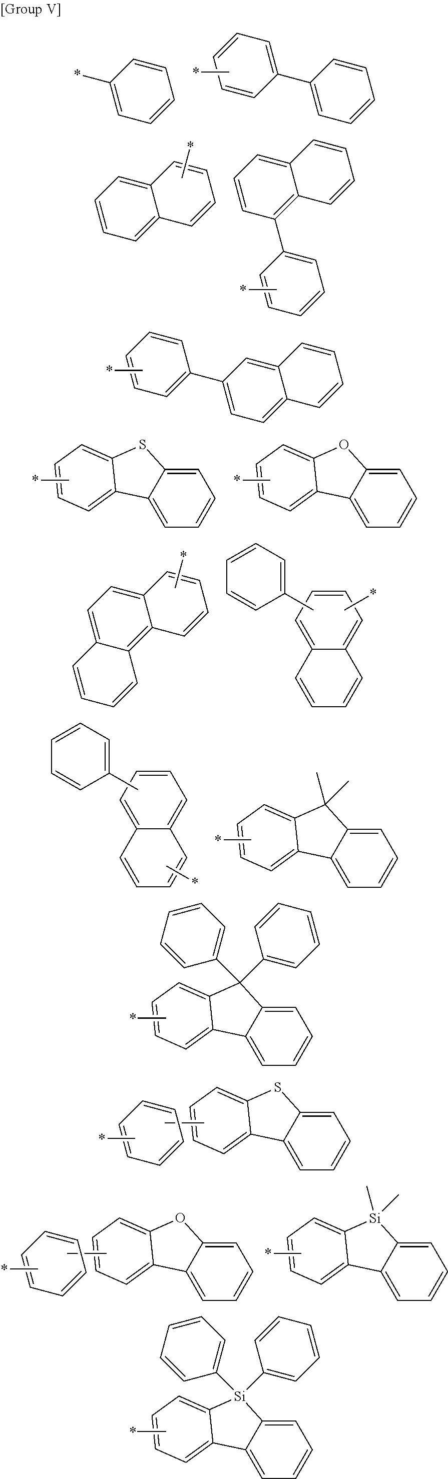

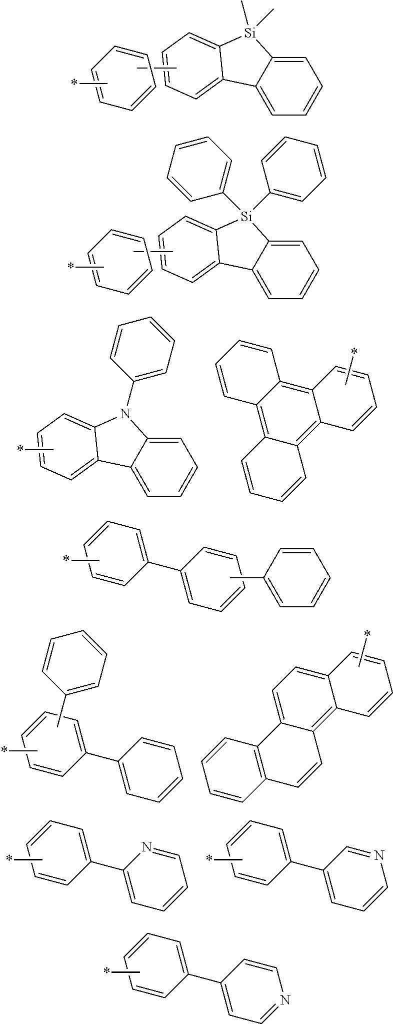

In Group V, * is a linking point.

For example, the third compound may be one selected from the compounds of Group 3, but is not limited thereto.

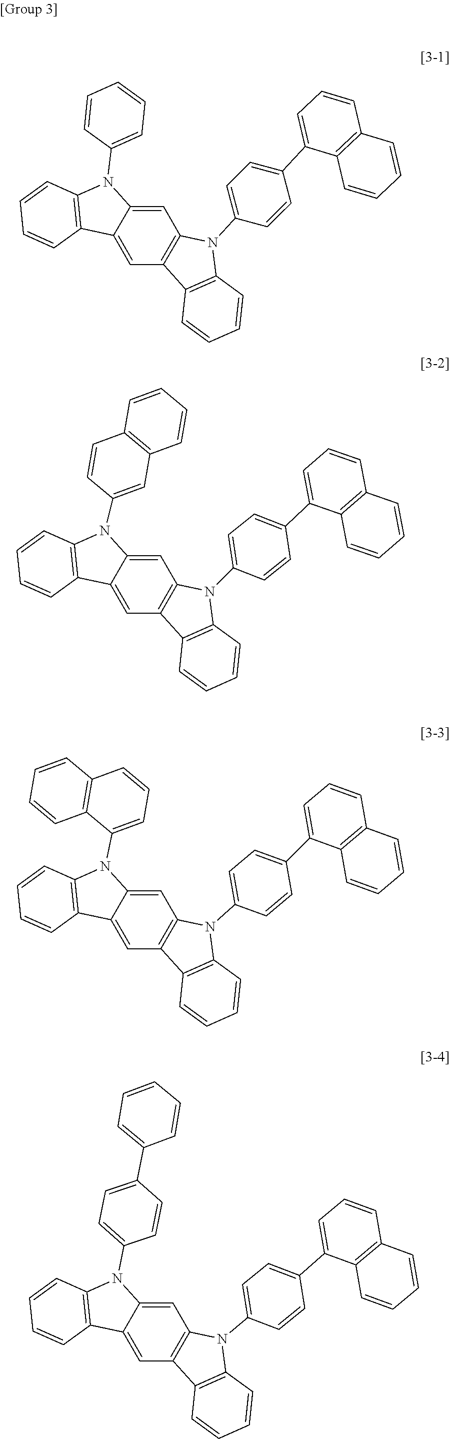 [SMILES: c1ccc(-n2c3ccccc3c3cc4c5ccccc5n(-c5ccc(-c6cccc7ccccc67)cc5)c4cc32)cc1 ; c1ccc2cc(-n3c4ccccc4c4cc5c6ccccc6n(-c6ccc(-c7cccc8ccccc78)cc6)c5cc43)ccc2c1 ; c1ccc2c(-c3ccc(-n4c5ccccc5c5cc6c7ccccc7n(-c7cccc8ccccc78)c6cc54)cc3)cccc2c1 ; c1ccc(-c2ccc(-n3c4ccccc4c4cc5c6ccccc6n(-c6ccc(-c7cccc8ccccc78)cc6)c5cc43)cc2)cc1]

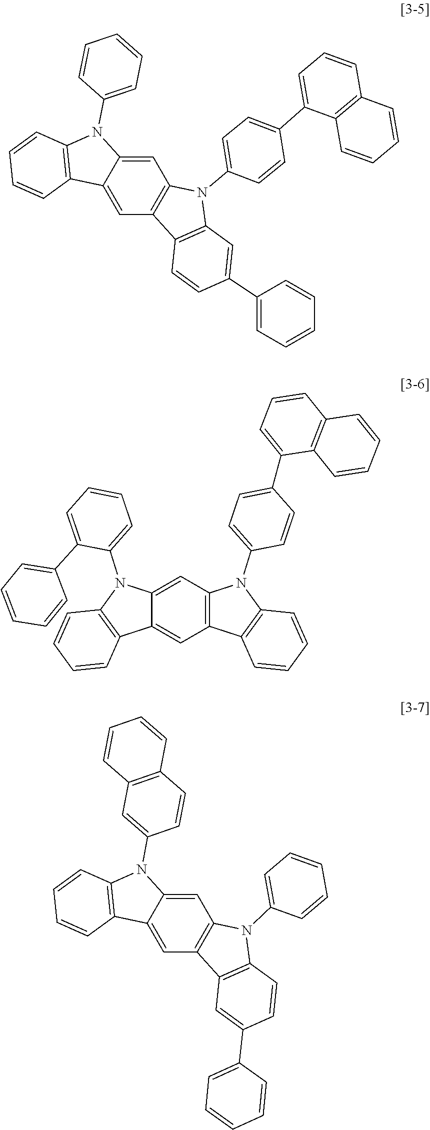 [SMILES: c1ccc(-c2ccc3c4cc5c6ccccc6n(-c6ccccc6)c5cc4n(-c4ccc(-c5cccc6ccccc56)cc4)c3c2)cc1 ; c1ccc(-c2ccccc2-n2c3ccccc3c3cc4c5ccccc5n(-c5ccc(-c6cccc7ccccc67)cc5)c4cc32)cc1 ; c1ccc(-c2ccc3c(c2)c2cc4c5ccccc5n(-c5ccc6ccccc6c5)c4cc2n3-c2ccccc2)cc1]

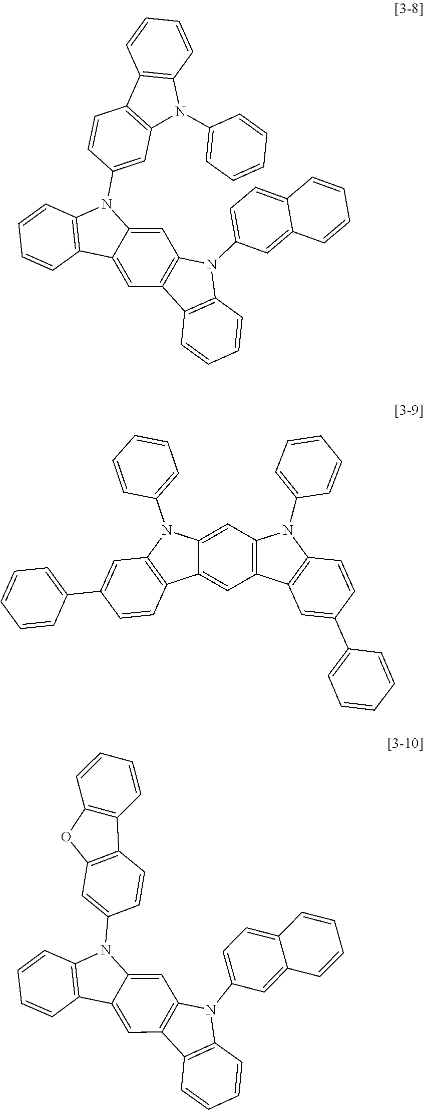 [SMILES: c1ccc(-n2c3ccccc3c3ccc(-n4c5ccccc5c5cc6c7ccccc7n(-c7ccc8ccccc8c7)c6cc54)cc32)cc1 ; c1ccc(-c2ccc3c(c2)c2cc4c5ccc(-c6ccccc6)cc5n(-c5ccccc5)c4cc2n3-c2ccccc2)cc1 ; c1ccc2cc(-n3c4ccccc4c4cc5c6ccccc6n(-c6ccc7c(c6)oc6ccccc67)c5cc43)ccc2c1]

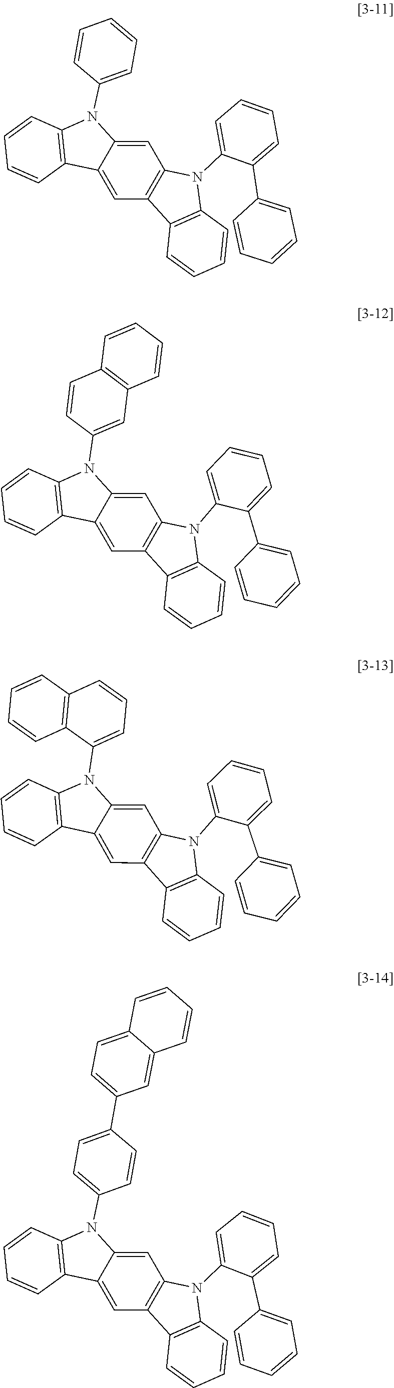 [SMILES: c1ccc(-c2ccccc2-n2c3ccccc3c3cc4c5ccccc5n(-c5ccccc5)c4cc32)cc1 ; c1ccc(-c2ccccc2-n2c3ccccc3c3cc4c5ccccc5n(-c5ccc6ccccc6c5)c4cc32)cc1 ; c1ccc(-c2ccccc2-n2c3ccccc3c3cc4c5ccccc5n(-c5cccc6ccccc56)c4cc32)cc1 ; c1ccc(-c2ccccc2-n2c3ccccc3c3cc4c5ccccc5n(-c5ccc(-c6ccc7ccccc7c6)cc5)c4cc32)cc1]

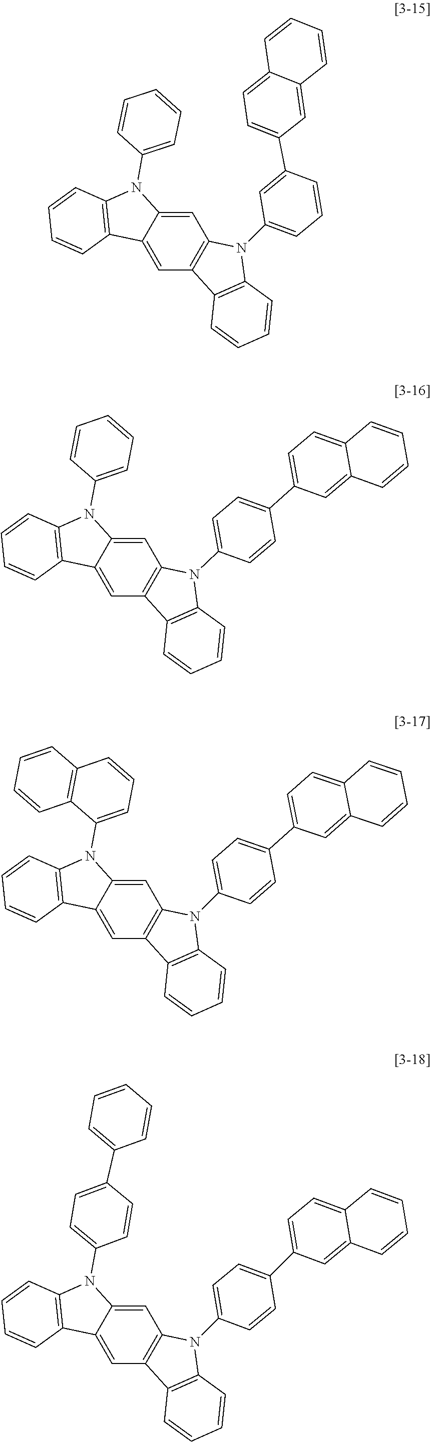 [SMILES: c1ccc(-n2c3ccccc3c3cc4c5ccccc5n(-c5cccc(-c6ccc7ccccc7c6)c5)c4cc32)cc1 ; c1ccc(-n2c3ccccc3c3cc4c5ccccc5n(-c5ccc(-c6ccc7ccccc7c6)cc5)c4cc32)cc1 ; c1ccc2cc(-c3ccc(-n4c5ccccc5c5cc6c7ccccc7n(-c7cccc8ccccc78)c6cc54)cc3)ccc2c1 ; c1ccc(-c2ccc(-n3c4ccccc4c4cc5c6ccccc6n(-c6ccc(-c7ccc8ccccc8c7)cc6)c5cc43)cc2)cc1]

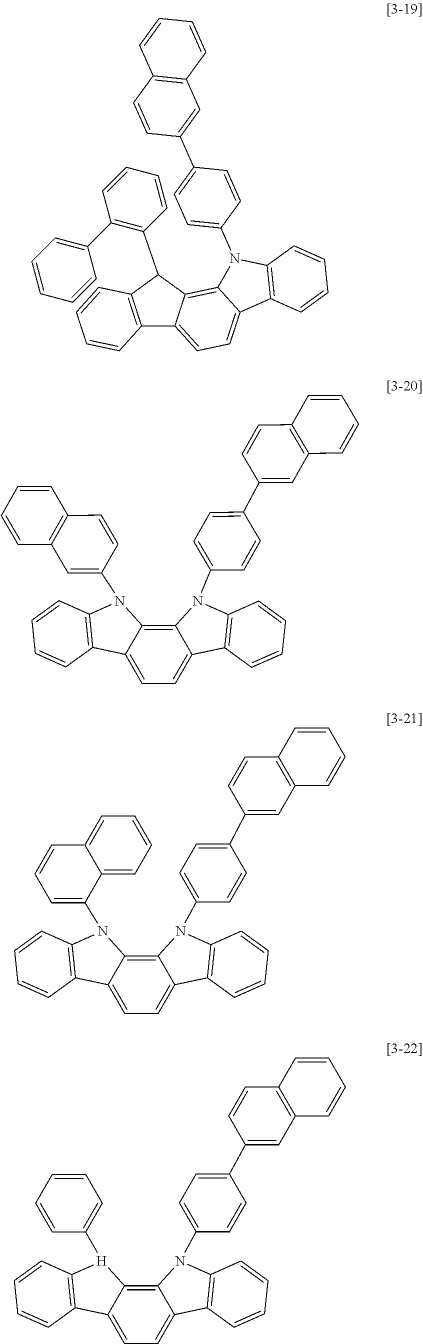

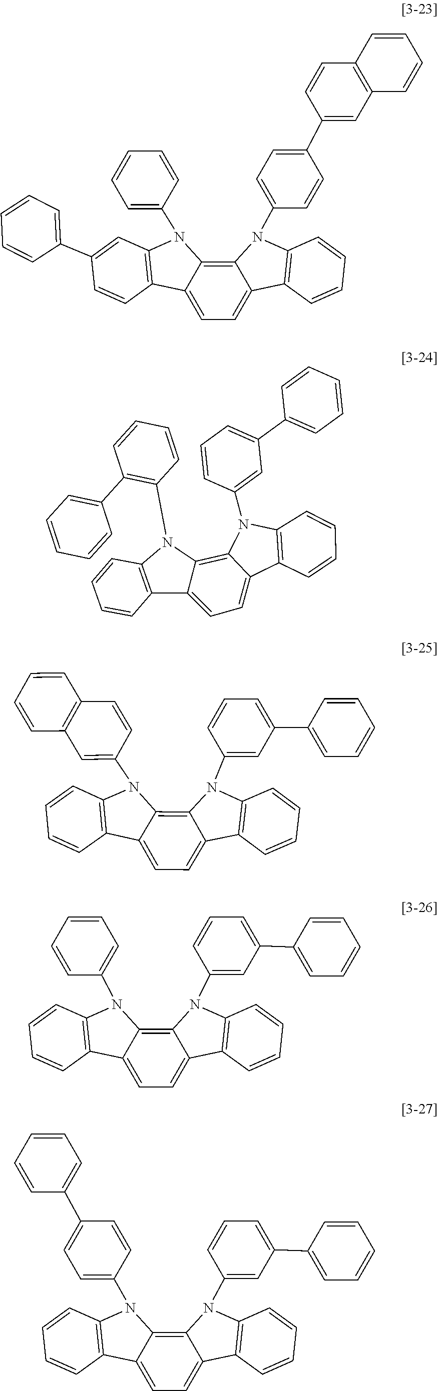 [SMILES: c1ccc(-c2ccc3c4ccc5c6ccccc6n(-c6ccc(-c7ccc8ccccc8c7)cc6)c5c4n(-c4ccccc4)c3c2)cc1 ; c1ccc(-c2cccc(-n3c4ccccc4c4ccc5c6ccccc6n(-c6ccccc6-c6ccccc6)c5c43)c2)cc1 ; c1ccc(-c2cccc(-n3c4ccccc4c4ccc5c6ccccc6n(-c6ccc7ccccc7c6)c5c43)c2)cc1 ; c1ccc(-c2cccc(-n3c4ccccc4c4ccc5c6ccccc6n(-c6ccccc6)c5c43)c2)cc1 ; c1ccc(-c2ccc(-n3c4ccccc4c4ccc5c6ccccc6n(-c6cccc(-c7ccccc7)c6)c5c43)cc2)cc1]

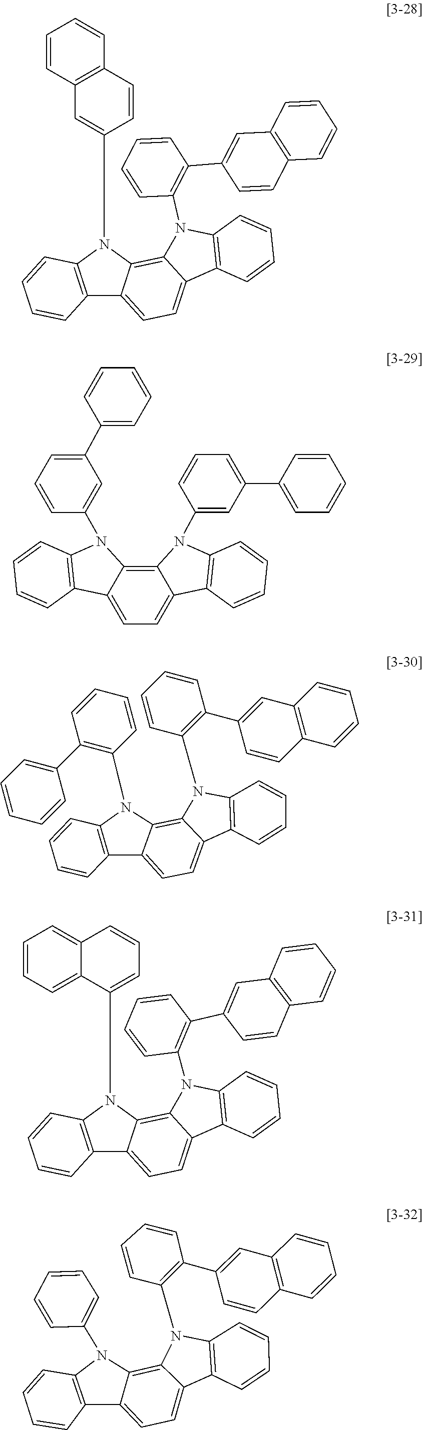 [SMILES: c1ccc(-n2c3ccccc3c3ccc4c5ccccc5n(-c5ccc6ccccc6c5)c4c32)c(-c2ccc3ccccc3c2)c1 ; c1ccc(-c2cccc(-n3c4ccccc4c4ccc5c6ccccc6n(-c6cccc(-c7ccccc7)c6)c5c43)c2)cc1 ; c1ccc(-c2ccccc2-n2c3ccccc3c3ccc4c5ccccc5n(-c5ccccc5-c5ccc6ccccc6c5)c4c32)cc1 ; c1ccc(-n2c3ccccc3c3ccc4c5ccccc5n(-c5cccc6ccccc56)c4c32)c(-c2ccc3ccccc3c2)c1 ; c1ccc(-n2c3ccccc3c3ccc4c5ccccc5n(-c5ccccc5-c5ccc6ccccc6c5)c4c32)cc1]

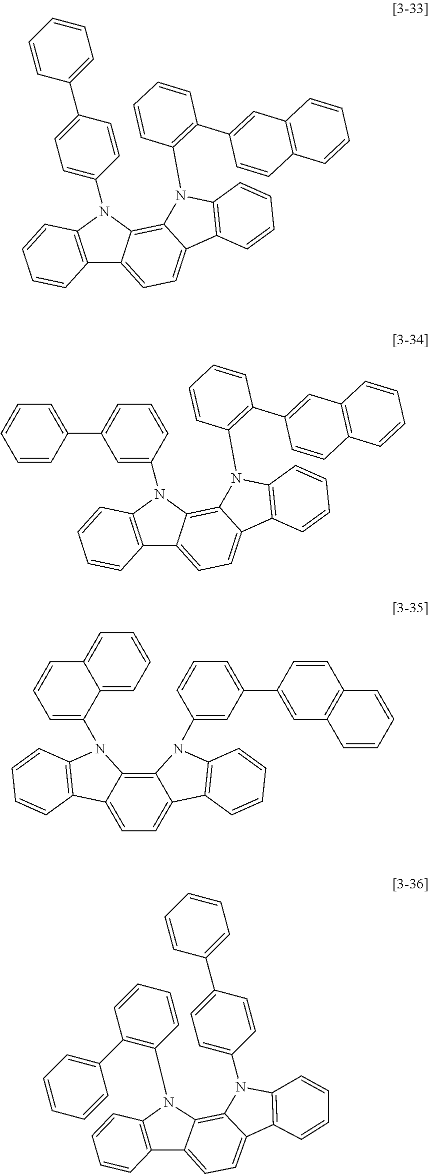 [SMILES: c1ccc(-c2ccc(-n3c4ccccc4c4ccc5c6ccccc6n(-c6ccccc6-c6ccc7ccccc7c6)c5c43)cc2)cc1 ; c1ccc(-c2cccc(-n3c4ccccc4c4ccc5c6ccccc6n(-c6ccccc6-c6ccc7ccccc7c6)c5c43)c2)cc1 ; c1cc(-c2ccc3ccccc3c2)cc(-n2c3ccccc3c3ccc4c5ccccc5n(-c5cccc6ccccc56)c4c32)c1 ; c1ccc(-c2ccc(-n3c4ccccc4c4ccc5c6ccccc6n(-c6ccccc6-c6ccccc6)c5c43)cc2)cc1]

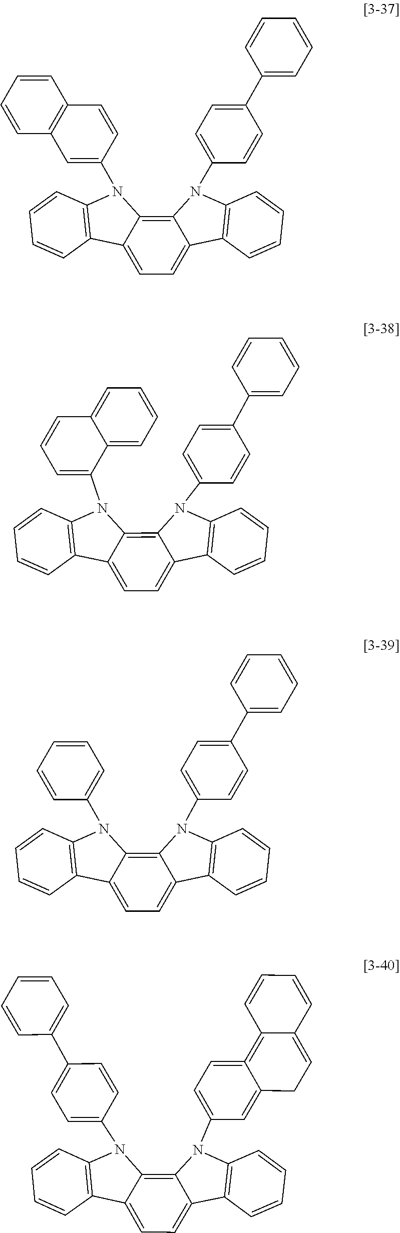

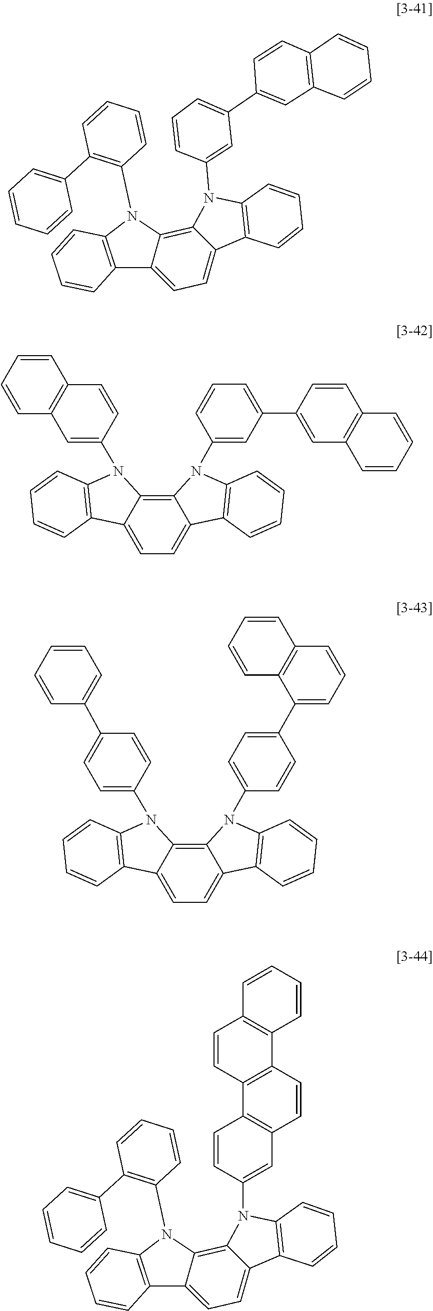 [SMILES: c1ccc(-c2ccccc2-n2c3ccccc3c3ccc4c5ccccc5n(-c5cccc(-c6ccc7ccccc7c6)c5)c4c32)cc1 ; c1cc(-c2ccc3ccccc3c2)cc(-n2c3ccccc3c3ccc4c5ccccc5n(-c5ccc6ccccc6c5)c4c32)c1 ; c1ccc(-c2ccc(-n3c4ccccc4c4ccc5c6ccccc6n(-c6ccc(-c7cccc8ccccc78)cc6)c5c43)cc2)cc1 ; c1ccc(-c2ccccc2-n2c3ccccc3c3ccc4c5ccccc5n(-c5ccc6c(ccc7c8ccccc8ccc67)c5)c4c32)cc1]

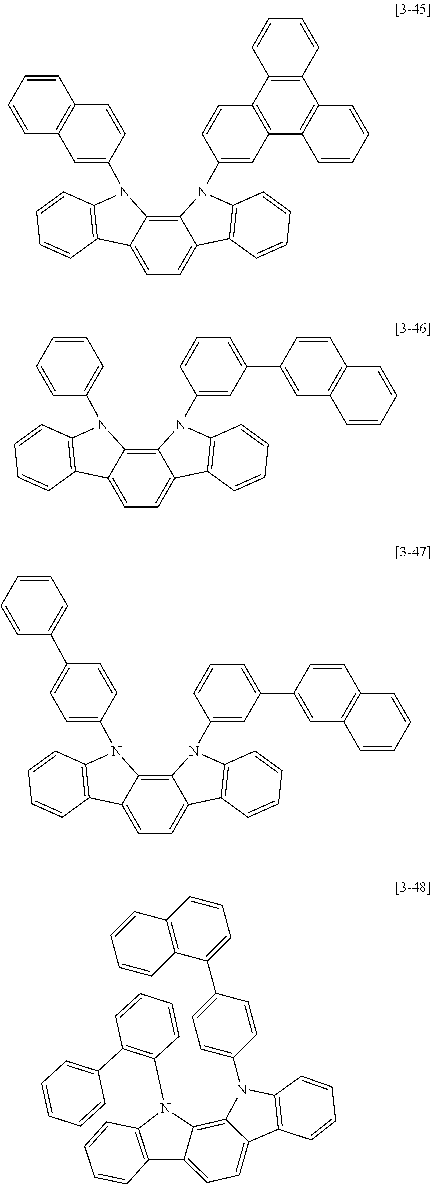 [SMILES: c1ccc2cc(-n3c4ccccc4c4ccc5c6ccccc6n(-c6ccc7c8ccccc8c8ccccc8c7c6)c5c43)ccc2c1 ; c1ccc(-n2c3ccccc3c3ccc4c5ccccc5n(-c5cccc(-c6ccc7ccccc7c6)c5)c4c32)cc1 ; c1ccc(-c2ccc(-n3c4ccccc4c4ccc5c6ccccc6n(-c6cccc(-c7ccc8ccccc8c7)c6)c5c43)cc2)cc1 ; c1ccc(-c2ccccc2-n2c3ccccc3c3ccc4c5ccccc5n(-c5ccc(-c6cccc7ccccc67)cc5)c4c32)cc1]

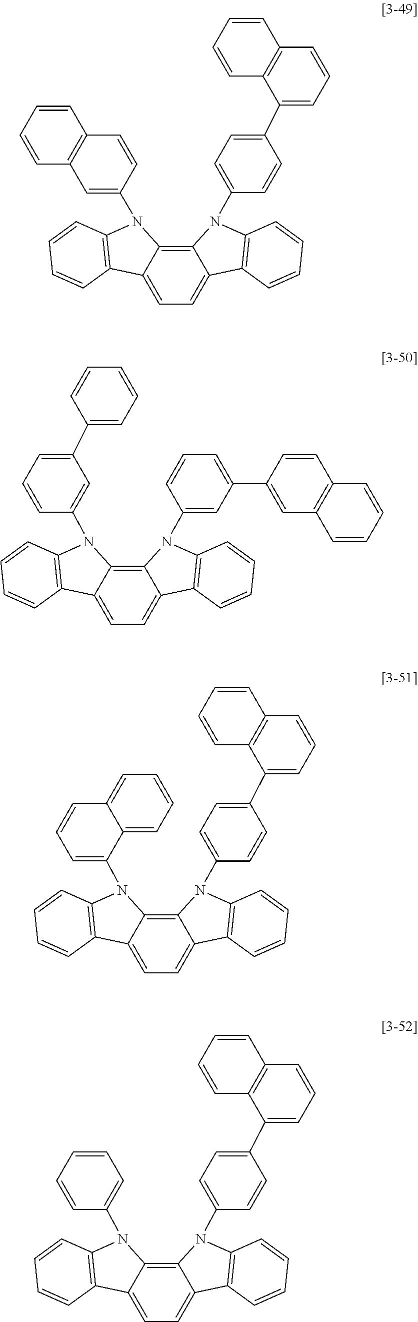 [SMILES: c1ccc2cc(-n3c4ccccc4c4ccc5c6ccccc6n(-c6ccc(-c7cccc8ccccc78)cc6)c5c43)ccc2c1 ; c1ccc(-c2cccc(-n3c4ccccc4c4ccc5c6ccccc6n(-c6cccc(-c7ccc8ccccc8c7)c6)c5c43)c2)cc1 ; c1ccc2c(-c3ccc(-n4c5ccccc5c5ccc6c7ccccc7n(-c7cccc8ccccc78)c6c54)cc3)cccc2c1 ; c1ccc(-n2c3ccccc3c3ccc4c5ccccc5n(-c5ccc(-c6cccc7ccccc67)cc5)c4c32)cc1]

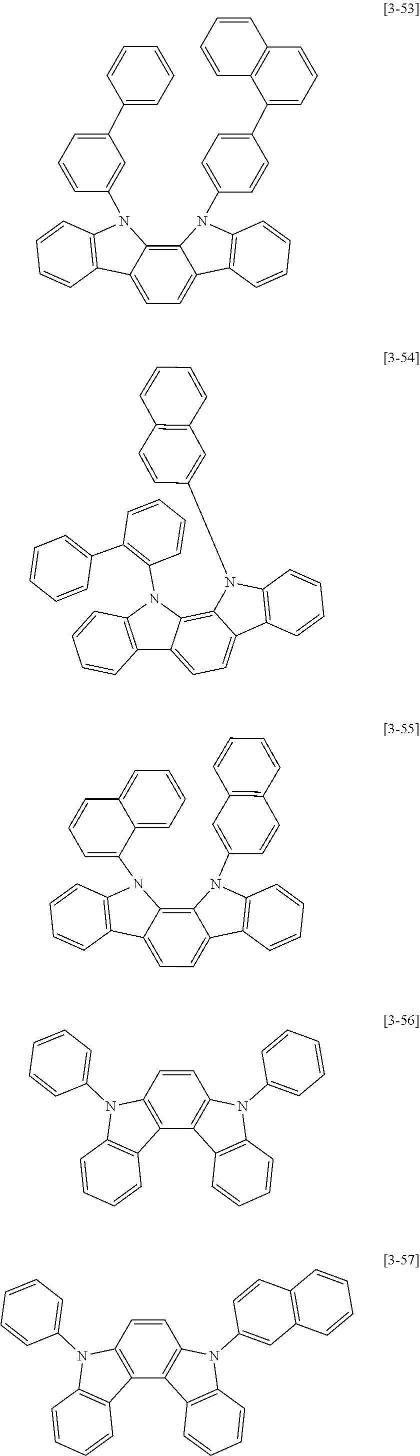 [SMILES: c1ccc(-c2cccc(-n3c4ccccc4c4ccc5c6ccccc6n(-c6ccc(-c7cccc8ccccc78)cc6)c5c43)c2)cc1 ; c1ccc(-c2ccccc2-n2c3ccccc3c3ccc4c5ccccc5n(-c5ccc6ccccc6c5)c4c32)cc1 ; c1ccc2cc(-n3c4ccccc4c4ccc5c6ccccc6n(-c6cccc7ccccc67)c5c43)ccc2c1 ; c1ccc(-n2c3ccccc3c3c4c5ccccc5n(-c5ccccc5)c4ccc32)cc1 ; c1ccc(-n2c3ccccc3c3c4c5ccccc5n(-c5ccc6ccccc6c5)c4ccc32)cc1]

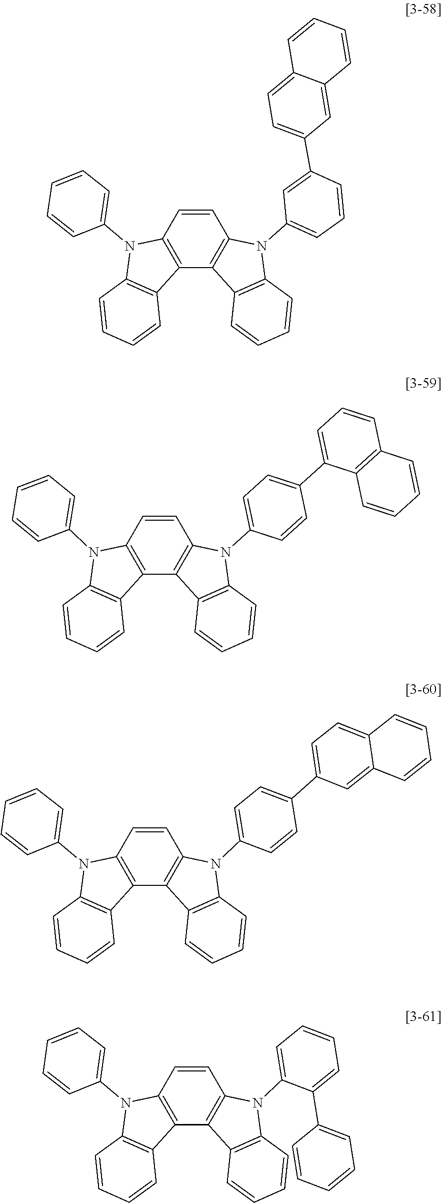 [SMILES: c1ccc(-n2c3ccccc3c3c4c5ccccc5n(-c5cccc(-c6ccc7ccccc7c6)c5)c4ccc32)cc1 ; c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-c2ccccc2-n2c3ccccc3c3c4c5ccccc5n(-c5ccccc5)c4ccc32)cc1]

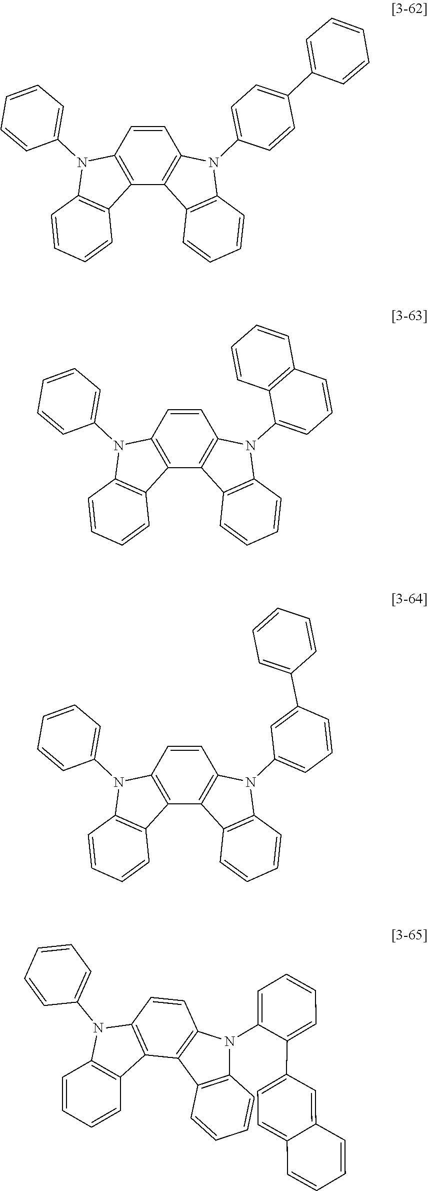 [SMILES: c1ccc(-c2ccc(-n3c4ccccc4c4c5c6ccccc6n(-c6ccccc6)c5ccc43)cc2)cc1 ; c1ccc(-n2c3ccccc3c3c4c5ccccc5n(-c5cccc6ccccc56)c4ccc32)cc1 ; c1ccc(-c2cccc(-n3c4ccccc4c4c5c6ccccc6n(-c6ccccc6)c5ccc43)c2)cc1 ; c1ccc(-n2c3ccccc3c3c4c5ccccc5n(-c5ccccc5-c5ccc6ccccc6c5)c4ccc32)cc1]

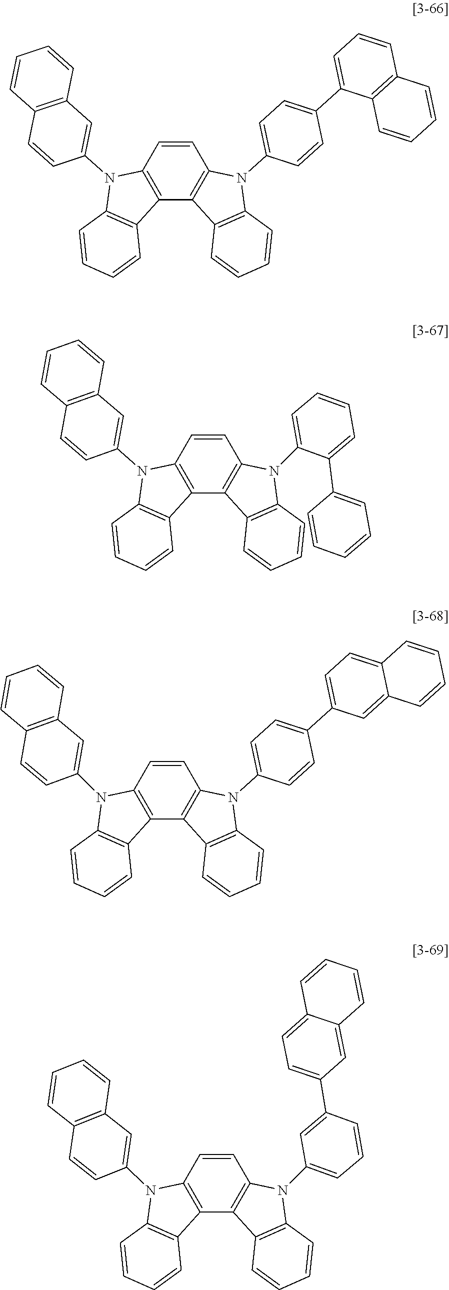

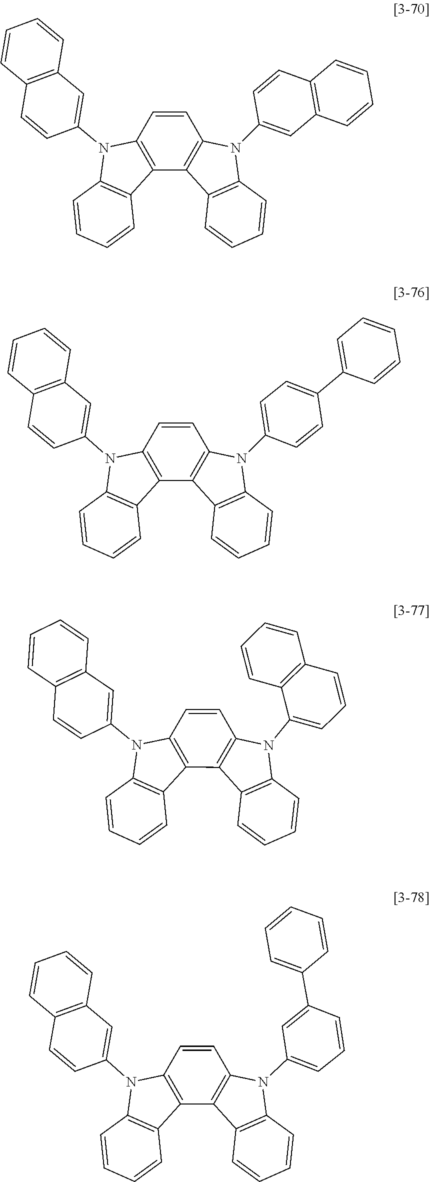 [SMILES: c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc2cc(-n3c4ccccc4c4c5c6ccccc6n(-c6cccc7ccccc67)c5ccc43)ccc2c1 ; c1ccc(-c2cccc(-n3c4ccccc4c4c5c6ccccc6n(-c6ccc7ccccc7c6)c5ccc43)c2)cc1]

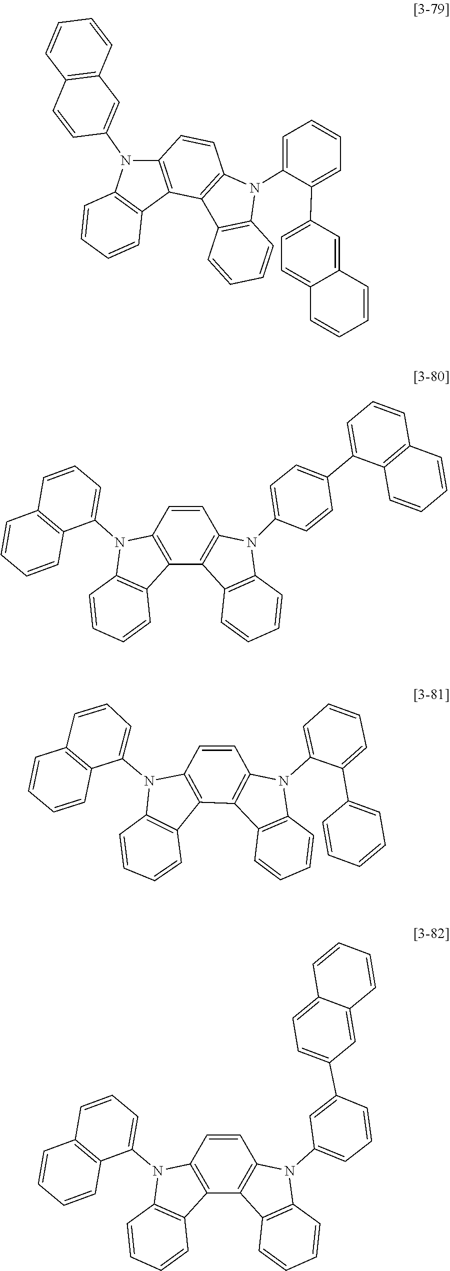 [SMILES: c1ccc(-n2c3ccccc3c3c4c5ccccc5n(-c5ccc6ccccc6c5)c4ccc32)c(-c2ccc3ccccc3c2)c1 ; c1ccc2c(-c3ccc(-n4c5ccccc5c5c6c7ccccc7n(-c7cccc8ccccc78)c6ccc54)cc3)cccc2c1 ; c1ccc(-c2ccccc2-n2c3ccccc3c3c4c5ccccc5n(-c5cccc6ccccc56)c4ccc32)cc1 ; c1cc(-c2ccc3ccccc3c2)cc(-n2c3ccccc3c3c4c5ccccc5n(-c5cccc6ccccc56)c4ccc32)c1]

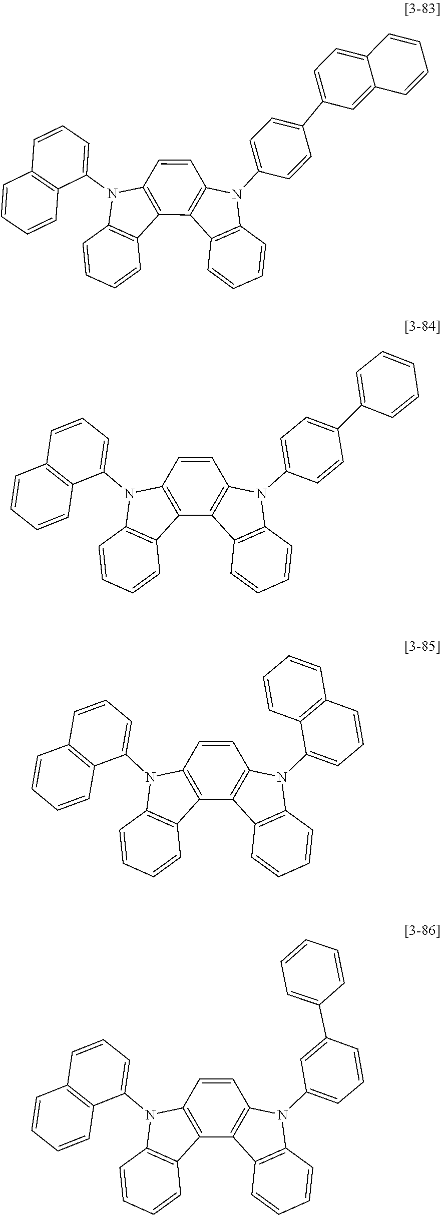 [SMILES: c1ccc2cc(-c3ccc(-n4c5ccccc5c5c6c7ccccc7n(-c7cccc8ccccc78)c6ccc54)cc3)ccc2c1 ; c1ccc(-c2ccc(-n3c4ccccc4c4c5c6ccccc6n(-c6cccc7ccccc67)c5ccc43)cc2)cc1 ; c1ccc2c(-n3c4ccccc4c4c5c6ccccc6n(-c6cccc7ccccc67)c5ccc43)cccc2c1 ; c1ccc(-c2cccc(-n3c4ccccc4c4c5c6ccccc6n(-c6cccc7ccccc67)c5ccc43)c2)cc1]

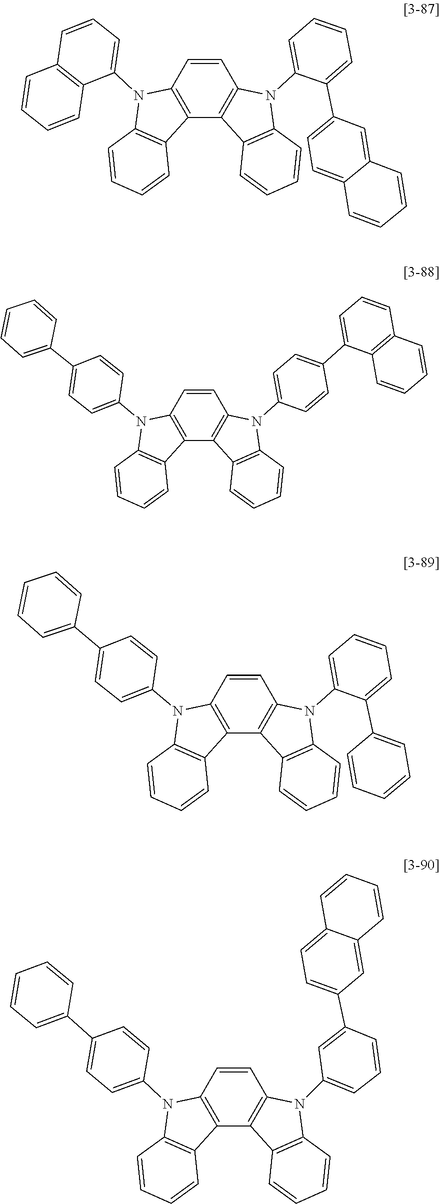 [SMILES: c1ccc(-n2c3ccccc3c3c4c5ccccc5n(-c5cccc6ccccc56)c4ccc32)c(-c2ccc3ccccc3c2)c1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccccc6-c6ccccc6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

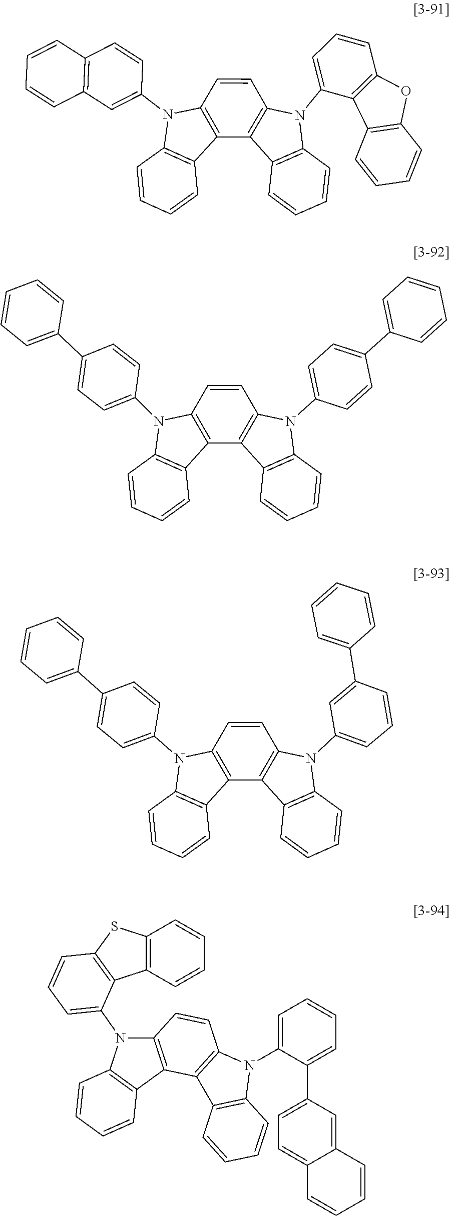 [SMILES: c1ccc2cc(-n3c4ccccc4c4c5c6ccccc6n(-c6cccc7oc8ccccc8c67)c5ccc43)ccc2c1 ; c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-n2c3ccccc3c3c4c5ccccc5n(-c5cccc6sc7ccccc7c56)c4ccc32)c(-c2ccc3ccccc3c2)c1]

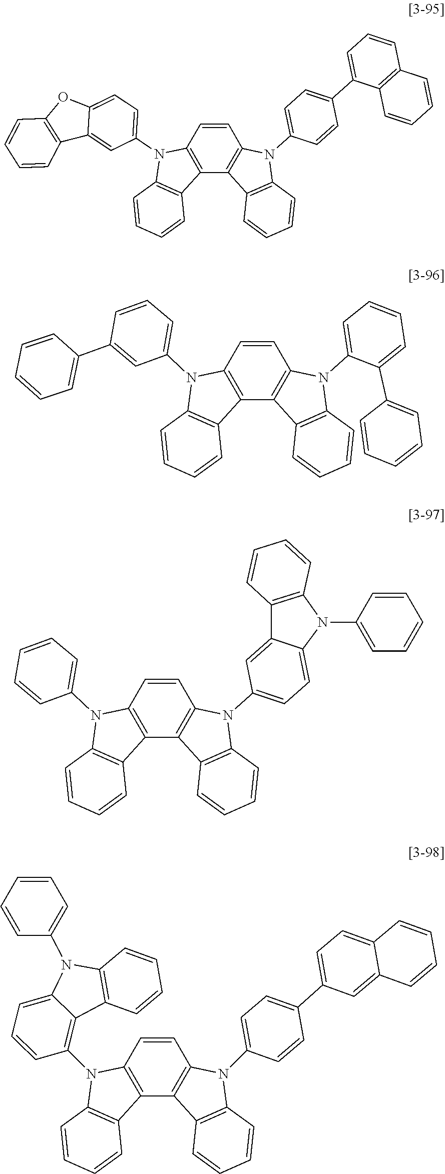 [SMILES: c1ccc2c(-c3ccc(-n4c5ccccc5c5c6c7ccccc7n(-c7ccc8oc9ccccc9c8c7)c6ccc54)cc3)cccc2c1 ; c1ccc(-c2cccc(-n3c4ccccc4c4c5c6ccccc6n(-c6ccccc6-c6ccccc6)c5ccc43)c2)cc1 ; c1ccc(-n2c3ccccc3c3cc(-n4c5ccccc5c5c6c7ccccc7n(-c7ccccc7)c6ccc54)ccc32)cc1 ; c1ccc(-n2c3ccccc3c3c(-n4c5ccccc5c5c6c7ccccc7n(-c7ccc(-c8ccc9ccccc9c8)cc7)c6ccc54)cccc32)cc1]

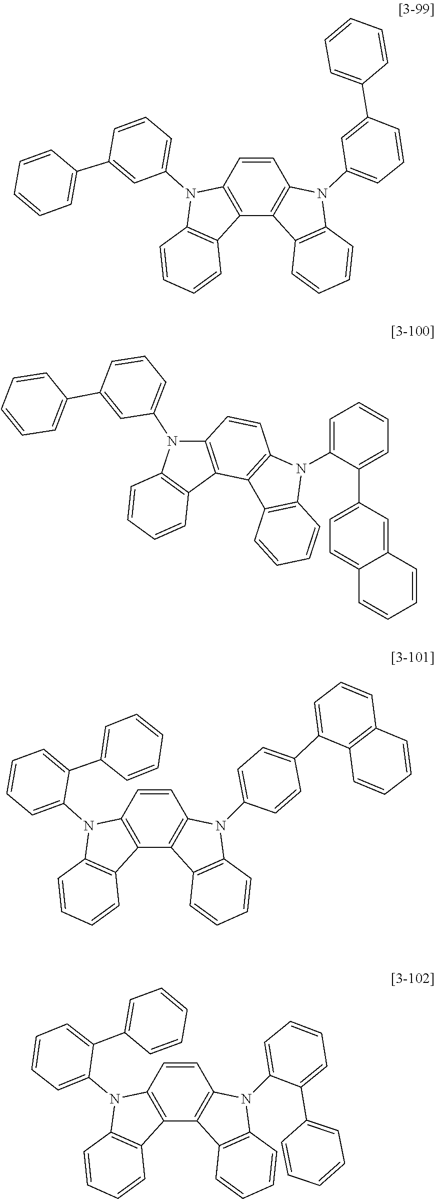 [SMILES: c1ccc(-c2cccc(-n3c4ccccc4c4c5c6ccccc6n(-c6cccc(-c7ccccc7)c6)c5ccc43)c2)cc1 ; c1ccc(-c2cccc(-n3c4ccccc4c4c5c6ccccc6n(-c6ccccc6-c6ccc7ccccc7c6)c5ccc43)c2)cc1 ; c1ccc(-c2ccccc2-n2c3ccccc3c3c4c5ccccc5n(-c5ccc(-c6cccc7ccccc67)cc5)c4ccc32)cc1 ; c1ccc(-c2ccccc2-n2c3ccccc3c3c4c5ccccc5n(-c5ccccc5-c5ccccc5)c4ccc32)cc1]

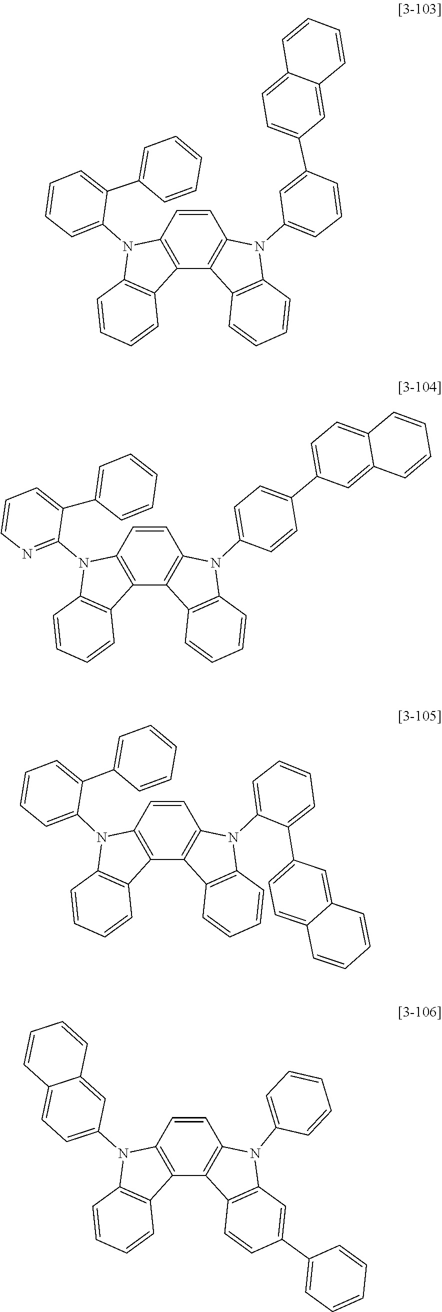 [SMILES: c1ccc(-c2ccccc2-n2c3ccccc3c3c4c5ccccc5n(-c5cccc(-c6ccc7ccccc7c6)c5)c4ccc32)cc1 ; c1ccc(-c2cccnc2-n2c3ccccc3c3c4c5ccccc5n(-c5ccc(-c6ccc7ccccc7c6)cc5)c4ccc32)cc1 ; c1ccc(-c2ccccc2-n2c3ccccc3c3c4c5ccccc5n(-c5ccccc5-c5ccc6ccccc6c5)c4ccc32)cc1 ; c1ccc(-c2ccc3c4c5c6ccccc6n(-c6ccc7ccccc7c6)c5ccc4n(-c4ccccc4)c3c2)cc1]

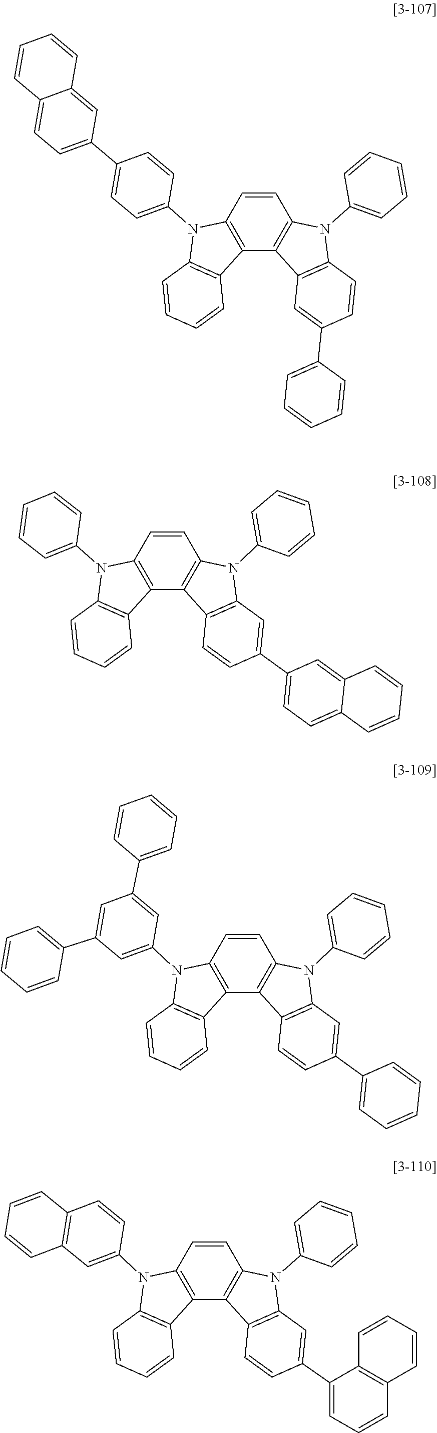 [SMILES: c1ccc(-c2ccc3c(c2)c2c4c5ccccc5n(-c5ccc(-c6ccc7ccccc7c6)cc5)c4ccc2n3-c2ccccc2)cc1 ; c1ccc(-n2c3ccccc3c3c4c5ccc(-c6ccc7ccccc7c6)cc5n(-c5ccccc5)c4ccc32)cc1 ; c1ccc(-c2cc(-c3ccccc3)cc(-n3c4ccccc4c4c5c6ccc(-c7ccccc7)cc6n(-c6ccccc6)c5ccc43)c2)cc1 ; c1ccc(-n2c3cc(-c4cccc5ccccc45)ccc3c3c4c5ccccc5n(-c5ccc6ccccc6c5)c4ccc32)cc1]

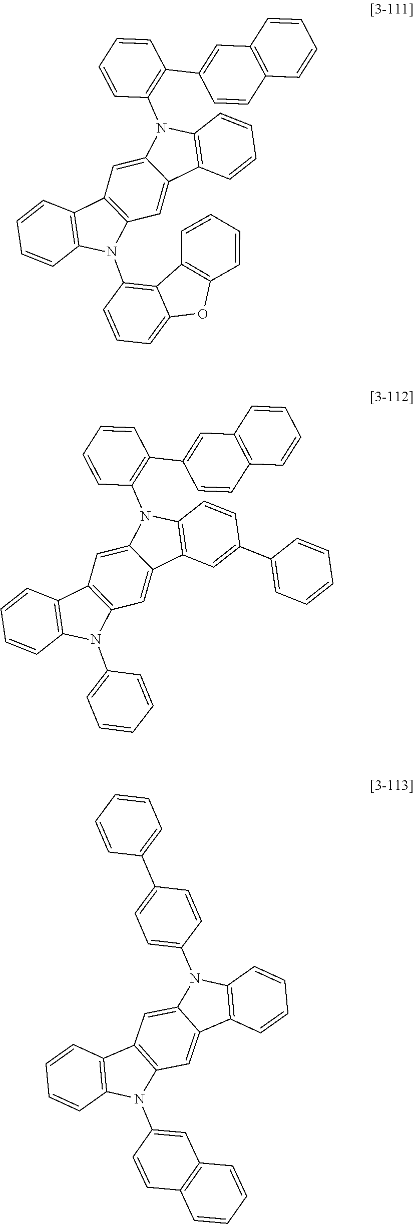 [SMILES: c1ccc(-n2c3ccccc3c3cc4c(cc32)c2ccccc2n4-c2cccc3oc4ccccc4c23)c(-c2ccc3ccccc3c2)c1 ; c1ccc(-c2ccc3c(c2)c2cc4c(cc2n3-c2ccccc2-c2ccc3ccccc3c2)c2ccccc2n4-c2ccccc2)cc1 ; c1ccc(-c2ccc(-n3c4ccccc4c4cc5c(cc43)c3ccccc3n5-c3ccc4ccccc4c3)cc2)cc1]

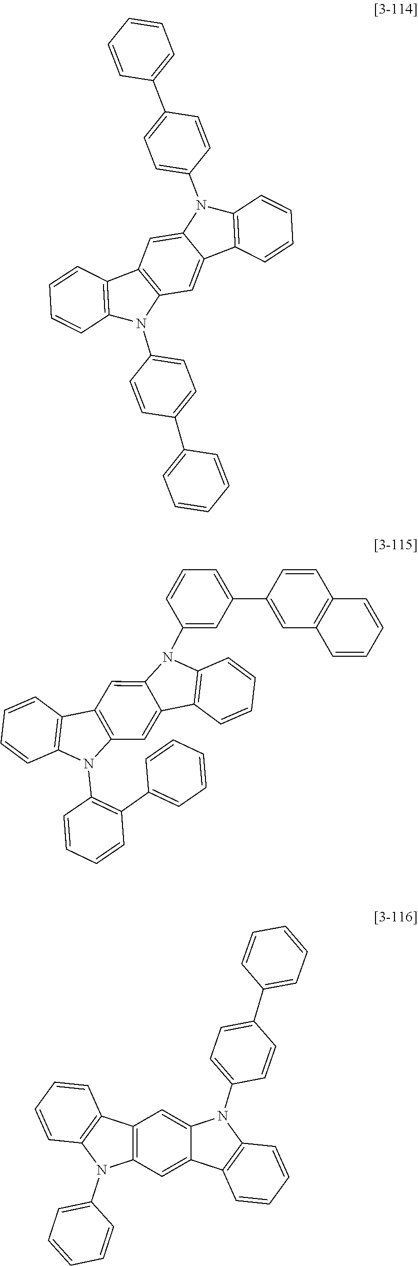 [SMILES: c1ccc(-c2ccc(-n3c4ccccc4c4cc5c(cc43)c3ccccc3n5-c3ccc(-c4ccccc4)cc3)cc2)cc1 ; c1ccc(-c2ccccc2-n2c3ccccc3c3cc4c(cc32)c2ccccc2n4-c2cccc(-c3ccc4ccccc4c3)c2)cc1 ; c1ccc(-c2ccc(-n3c4ccccc4c4cc5c(cc43)c3ccccc3n5-c3ccccc3)cc2)cc1]

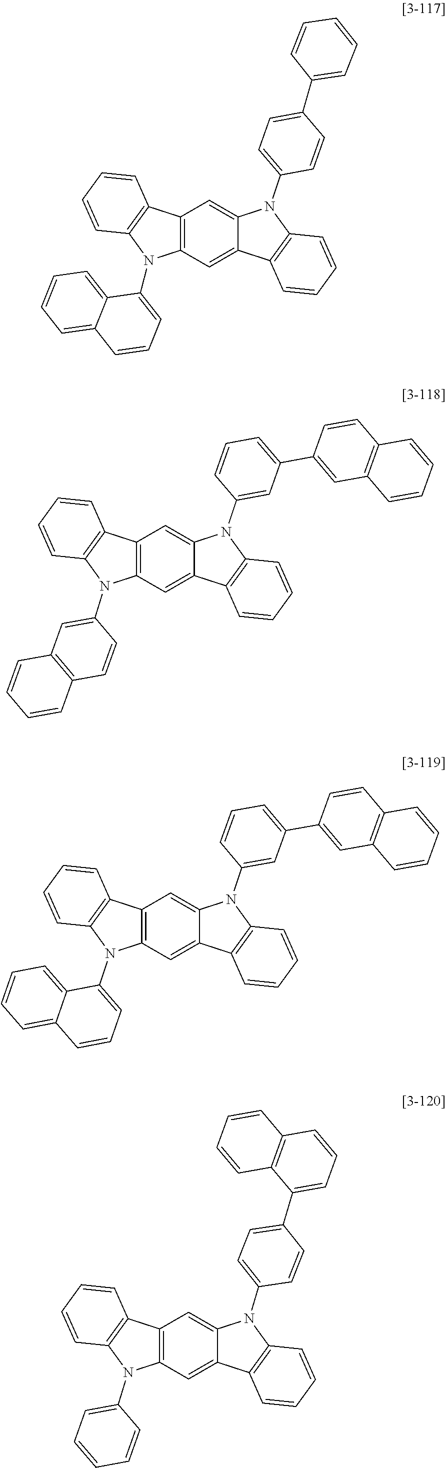 [SMILES: c1ccc(-c2ccc(-n3c4ccccc4c4cc5c(cc43)c3ccccc3n5-c3cccc4ccccc34)cc2)cc1 ; c1cc(-c2ccc3ccccc3c2)cc(-n2c3ccccc3c3cc4c(cc32)c2ccccc2n4-c2ccc3ccccc3c2)c1 ; c1cc(-c2ccc3ccccc3c2)cc(-n2c3ccccc3c3cc4c(cc32)c2ccccc2n4-c2cccc3ccccc23)c1 ; c1ccc(-n2c3ccccc3c3cc4c(cc32)c2ccccc2n4-c2ccc(-c3cccc4ccccc34)cc2)cc1]

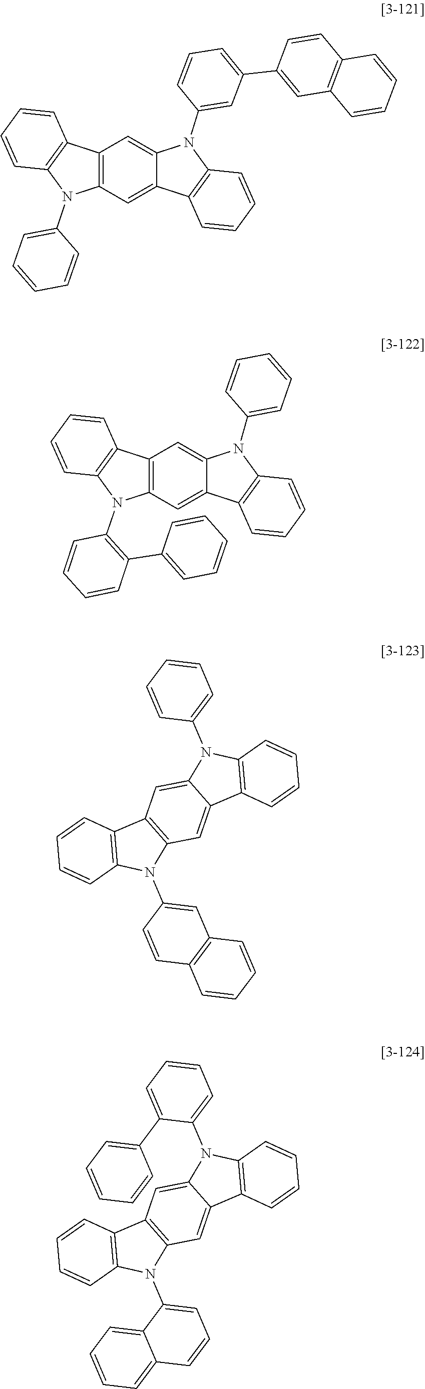 [SMILES: c1ccc(-n2c3ccccc3c3cc4c(cc32)c2ccccc2n4-c2cccc(-c3ccc4ccccc4c3)c2)cc1 ; c1ccc(-c2ccccc2-n2c3ccccc3c3cc4c(cc32)c2ccccc2n4-c2ccccc2)cc1 ; c1ccc(-n2c3ccccc3c3cc4c(cc32)c2ccccc2n4-c2ccc3ccccc3c2)cc1 ; c1ccc(-c2ccccc2-n2c3ccccc3c3cc4c(cc32)c2ccccc2n4-c2cccc3ccccc23)cc1]

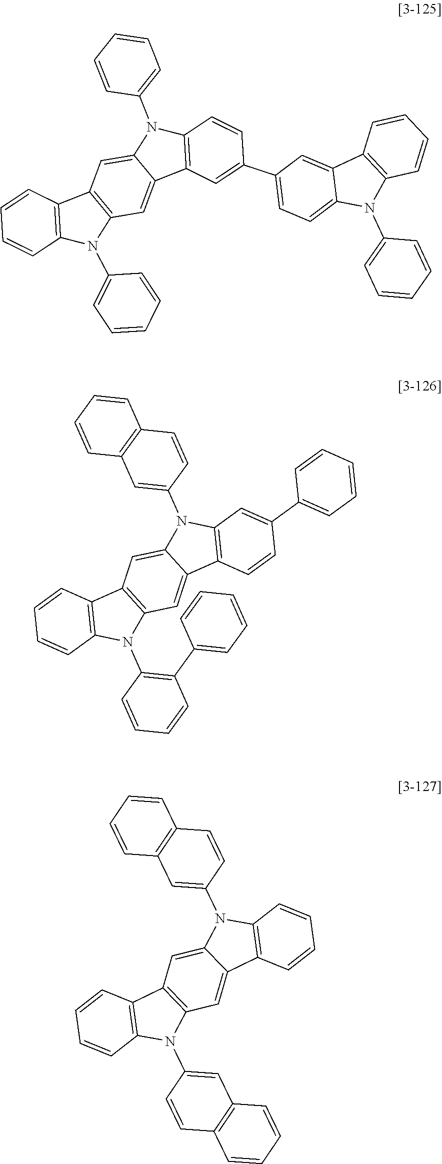 [SMILES: c1ccc(-n2c3ccccc3c3cc(-c4ccc5c(c4)c4cc6c(cc4n5-c4ccccc4)c4ccccc4n6-c4ccccc4)ccc32)cc1 ; c1ccc(-c2ccc3c4cc5c(cc4n(-c4ccc6ccccc6c4)c3c2)c2ccccc2n5-c2ccccc2-c2ccccc2)cc1 ; c1ccc2cc(-n3c4ccccc4c4cc5c(cc43)c3ccccc3n5-c3ccc4ccccc4c3)ccc2c1]

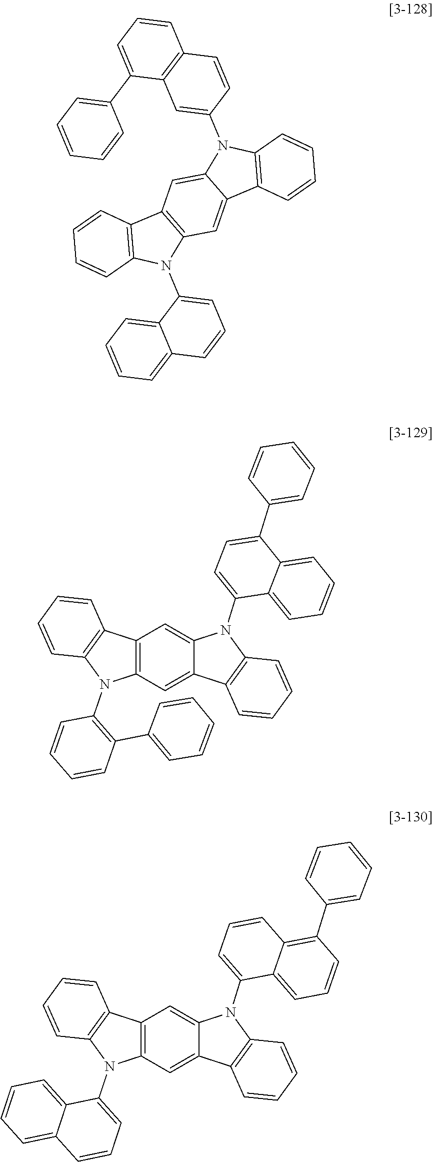 [SMILES: c1ccc(-c2cccc3ccc(-n4c5ccccc5c5cc6c(cc54)c4ccccc4n6-c4cccc5ccccc45)cc23)cc1 ; c1ccc(-c2ccccc2-n2c3ccccc3c3cc4c(cc32)c2ccccc2n4-c2ccc(-c3ccccc3)c3ccccc23)cc1 ; c1ccc(-c2cccc3c(-n4c5ccccc5c5cc6c(cc54)c4ccccc4n6-c4cccc5ccccc45)cccc23)cc1]

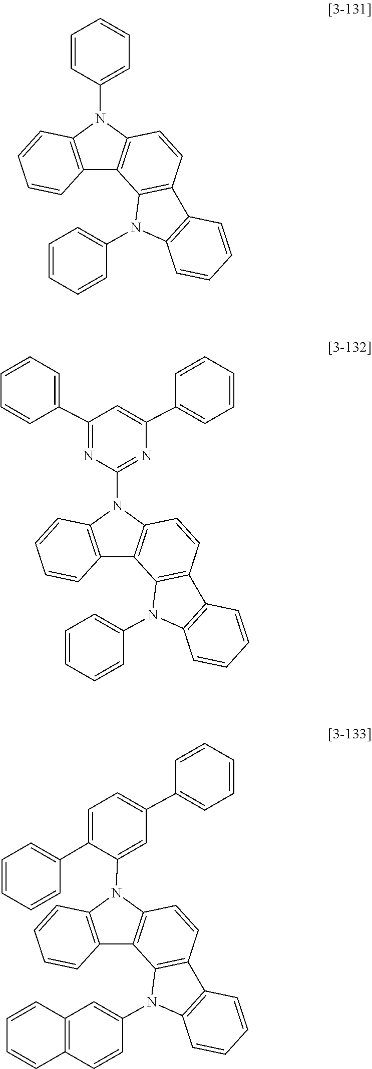 [SMILES: c1ccc(-n2c3ccccc3c3c2ccc2c4ccccc4n(-c4ccccc4)c23)cc1 ; c1ccc(-c2cc(-c3ccccc3)nc(-n3c4ccccc4c4c3ccc3c5ccccc5n(-c5ccccc5)c34)n2)cc1 ; c1ccc(-c2ccc(-c3ccccc3)c(-n3c4ccccc4c4c3ccc3c5ccccc5n(-c5ccc6ccccc6c5)c34)c2)cc1]

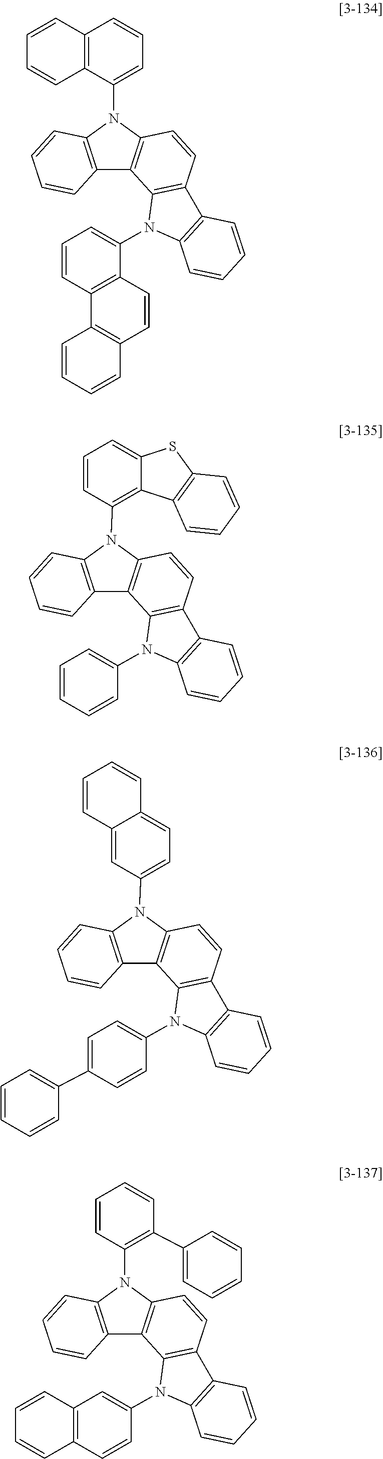 [SMILES: c1ccc2c(-n3c4ccccc4c4c3ccc3c5ccccc5n(-c5cccc6c5ccc5ccccc56)c34)cccc2c1 ; c1ccc(-n2c3ccccc3c3ccc4c(c5ccccc5n4-c4cccc5sc6ccccc6c45)c32)cc1 ; c1ccc(-c2ccc(-n3c4ccccc4c4ccc5c(c6ccccc6n5-c5ccc6ccccc6c5)c43)cc2)cc1 ; c1ccc(-c2ccccc2-n2c3ccccc3c3c2ccc2c4ccccc4n(-c4ccc5ccccc5c4)c23)cc1]

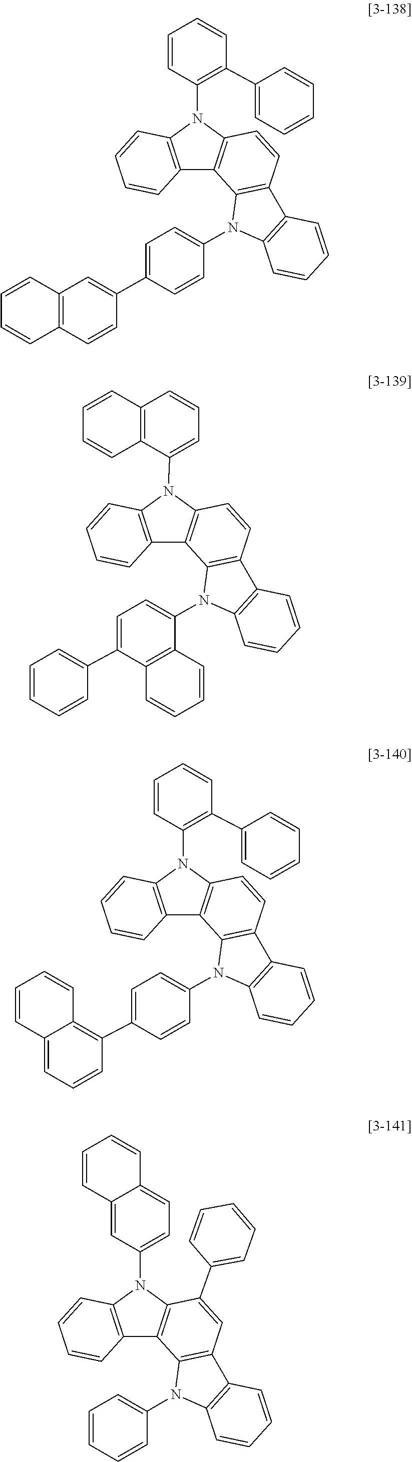 [SMILES: c1ccc(-c2ccccc2-n2c3ccccc3c3c2ccc2c4ccccc4n(-c4ccc(-c5ccc6ccccc6c5)cc4)c23)cc1 ; c1ccc(-c2ccc(-n3c4ccccc4c4ccc5c(c6ccccc6n5-c5cccc6ccccc56)c43)c3ccccc23)cc1 ; c1ccc(-c2ccccc2-n2c3ccccc3c3c2ccc2c4ccccc4n(-c4ccc(-c5cccc6ccccc56)cc4)c23)cc1 ; c1ccc(-c2cc3c4ccccc4n(-c4ccccc4)c3c3c4ccccc4n(-c4ccc5ccccc5c4)c23)cc1]

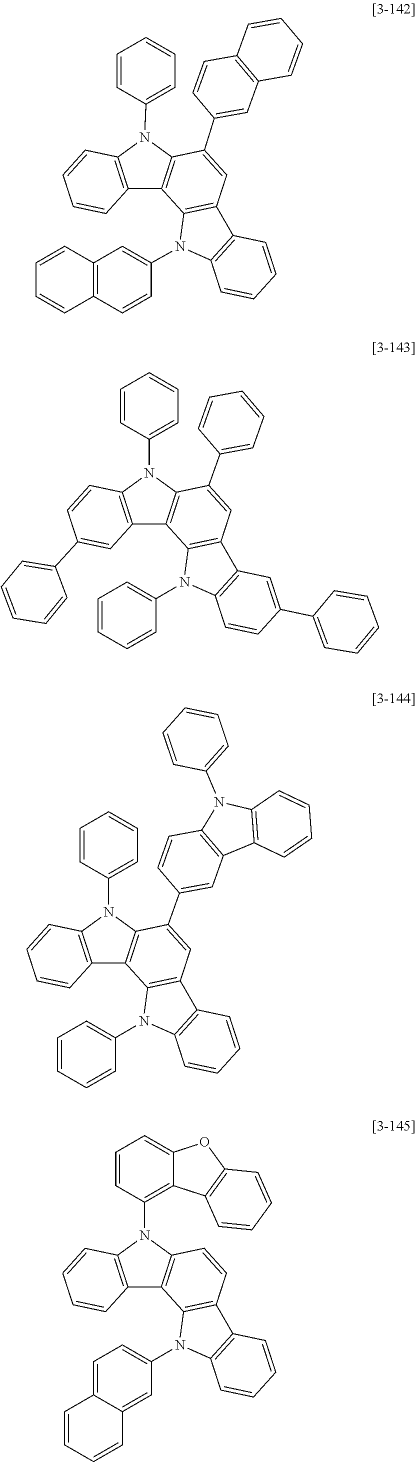 [SMILES: c1ccc(-n2c3ccccc3c3c2c(-c2ccc4ccccc4c2)cc2c4ccccc4n(-c4ccc5ccccc5c4)c23)cc1 ; c1ccc(-c2ccc3c(c2)c2c(c(-c4ccccc4)cc4c5cc(-c6ccccc6)ccc5n(-c5ccccc5)c42)n3-c2ccccc2)cc1 ; c1ccc(-n2c3ccccc3c3cc(-c4cc5c6ccccc6n(-c6ccccc6)c5c5c6ccccc6n(-c6ccccc6)c45)ccc32)cc1 ; c1ccc2cc(-n3c4ccccc4c4ccc5c(c6ccccc6n5-c5cccc6oc7ccccc7c56)c43)ccc2c1]

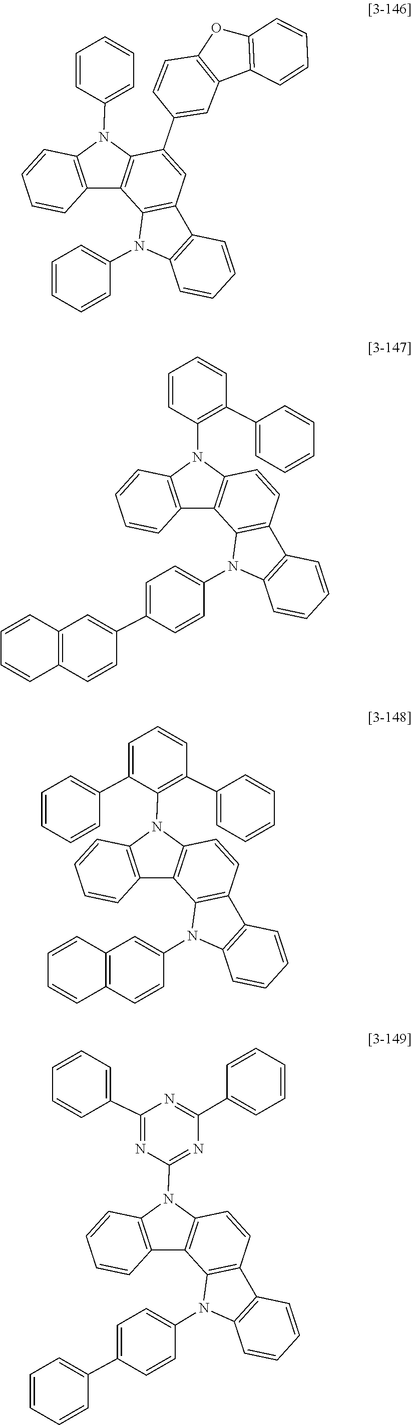 [SMILES: c1ccc(-n2c3ccccc3c3c2c(-c2ccc4oc5ccccc5c4c2)cc2c4ccccc4n(-c4ccccc4)c23)cc1 ; c1ccc(-c2ccccc2-n2c3ccccc3c3c2ccc2c4ccccc4n(-c4ccc(-c5ccc6ccccc6c5)cc4)c23)cc1 ; c1ccc(-c2cccc(-c3ccccc3)c2-n2c3ccccc3c3c2ccc2c4ccccc4n(-c4ccc5ccccc5c4)c23)cc1 ; c1ccc(-c2ccc(-n3c4ccccc4c4ccc5c(c6ccccc6n5-c5nc(-c6ccccc6)nc(-c6ccccc6)n5)c43)cc2)cc1]

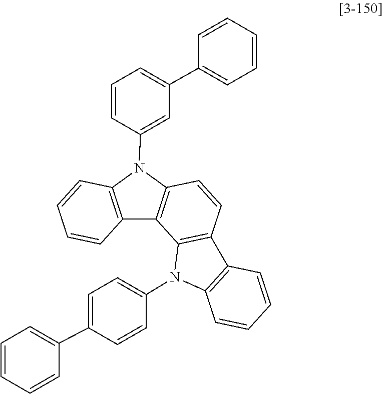 [SMILES: c1ccc(-c2ccc(-n3c4ccccc4c4ccc5c(c6ccccc6n5-c5cccc(-c6ccccc6)c5)c43)cc2)cc1]

The composition for an organic optoelectronic device according to the most specific embodiment of the present invention may include the first compound represented by any one of Chemical Formula 1-1 to Chemical Formula 1-4 and the second compound represented by Chemical Formula 2B-I-2.

Alternatively, the composition for an organic optoelectronic device according to the most specific embodiment of the present invention may include the first compound represented by any one of Chemical Formula 1-1 to Chemical Formula 1-4 and the third compound represented by any one of Chemical Formula 3A, Chemical Formula 3C and Chemical Formula 3E.

The first compound and the second compound, or the first compound and the third compound may be included in a weight ratio of, for example, about 1:99 to about 99:1. By being included in the above ranges, the efficiency and life-span may be improved by matching an appropriate weight ratio using the electron transport ability of the first compound and the hole transport ability of the second compound or the third compound to implement bipolar characteristics. Within these ranges, they may be included in a weight ratio of, for example, about 90:10 to 10:90, about 90:10 to 20:80, about 90:10 to 30:70, about 90:10 to 40:60, or about 90:10 to 50:50. For example, they may be included in a weight ratio of 60:40 to 50:50, for example, 50:50.

In an embodiment of the present invention, the first compound, the second compound, and the third compound may each be included as a host of the light emitting layer, for example, a phosphorescent host.

Hereinafter, an organic optoelectronic device to which the aforementioned composition for an organic optoelectronic device is applied will be described.

The organic optoelectronic device may be any device to convert electrical energy into photoenergy and vice versa without particular limitation, and may be, for example an organic photoelectric device, an organic light emitting diode, an organic solar cell, and an organic photo-conductor drum.

Herein, an organic light emitting diode as one example of an organic optoelectronic device is described referring to drawings.

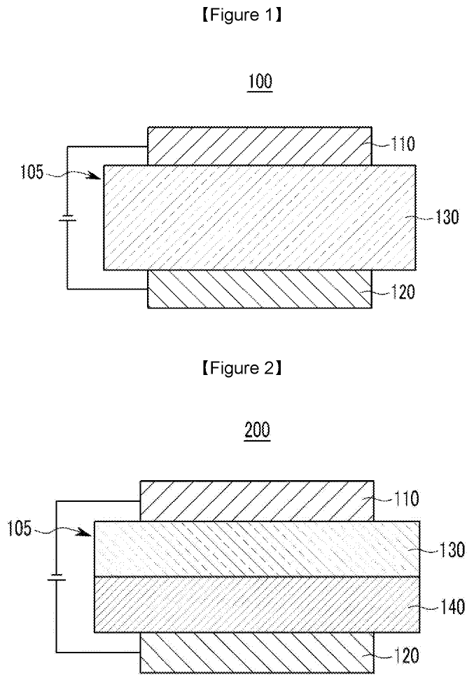

FIGS. 1 to 4 are cross-sectional views illustrating organic light emitting diodes according to embodiments.

Referring to FIG. 1, an organic light emitting diode 100 according to an embodiment includes an anode 120 and a cathode 110, facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example, a metal such as nickel, platinum, vanadium, chromium, copper, zinc, gold, and the like or an alloy thereof, a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, LiF/Al, and BaF₂/Ca, but is not limited thereto.

The organic layer 105 may include the aforementioned composition for an organic optoelectronic device.

The organic layer 105 may include a light emitting layer 130 and the light emitting layer 130 may include the aforementioned composition for an organic optoelectronic device.

The light emitting layer 130 may include, for example, the aforementioned composition for an organic optoelectronic device as a phosphorescent host.

The light emitting layer may further include one or more compounds in addition to the aforementioned host.

The light emitting layer may further include a dopant. The dopant may be, for example, a phosphorescent dopant, for example a phosphorescent dopant of red, green or blue, and may be, for example, a red phosphorescent dopant.

The composition for an organic optoelectronic device further including a dopant may be, for example, a red light emitting composition.

The dopant is a material mixed with the compound or composition for an organic optoelectronic device in a small amount to cause light emission and may be generally a material such as a metal complex that emits light by multiple excitation into a triplet or more. The dopant may be, for example, an inorganic, organic, or organic-inorganic compound, and may include one or two or more types.

An example of the dopant may be a phosphorescent dopant, and examples of the phosphorescent dopant may include an organometallic compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

L¹⁰MX² [Chemical Formula Z]

In Chemical Formula Z, M is a metal, L¹⁰ and X² are the same or different and are a ligand to form a complex compound with M.

The M may be for example Ir, Pt, Os, Ti, Zr, Hf, Eu, Tb, Tm, Fe, Co, Ni, Ru, Rh, Pd, or a combination thereof and L¹⁰ and X² may be for example a bidentate ligand.

The organic layer may further include a charge transport region in addition to the light emitting layer.

The charge transport region may be, for example, the hole transport region 140.

Referring to FIG. 2, the organic light emitting diode 200 further includes a hole transport region 140 in addition to the light emitting layer 130. The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130 and a hole transport auxiliary layer between the light emitting layer 130 and the hole transport layer, and at least one of compounds of Group A may be included in at least one of the hole transport layer and the hole transport auxiliary layer.

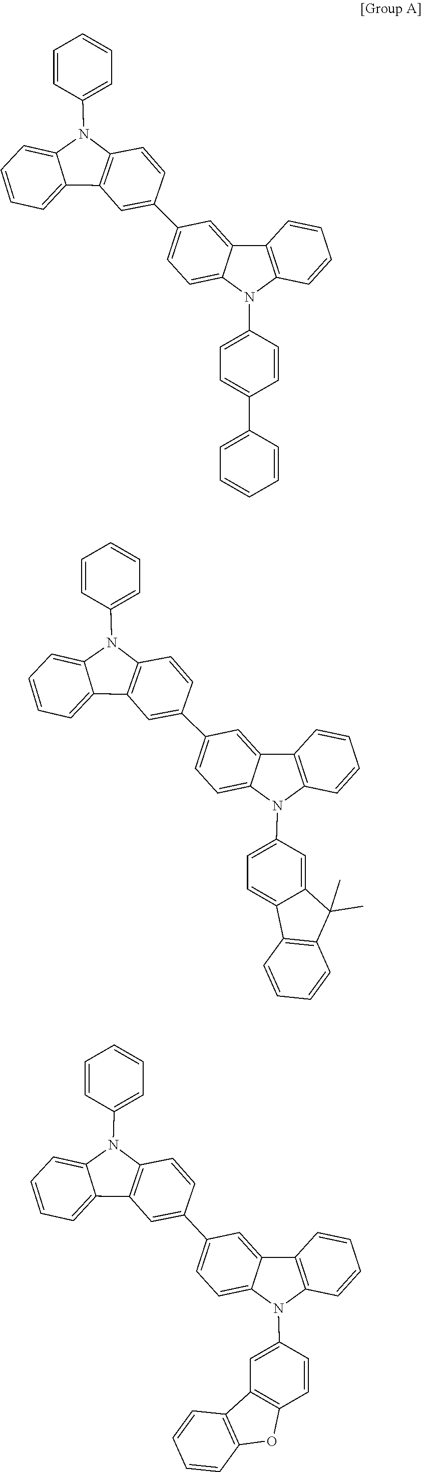 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

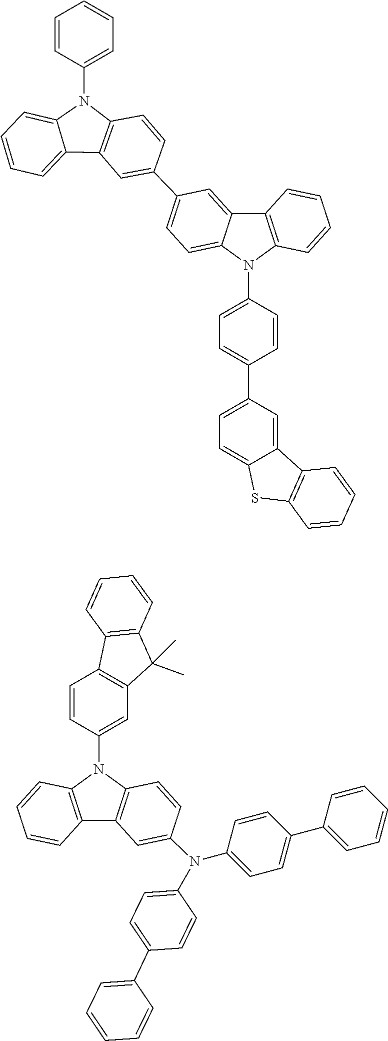 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

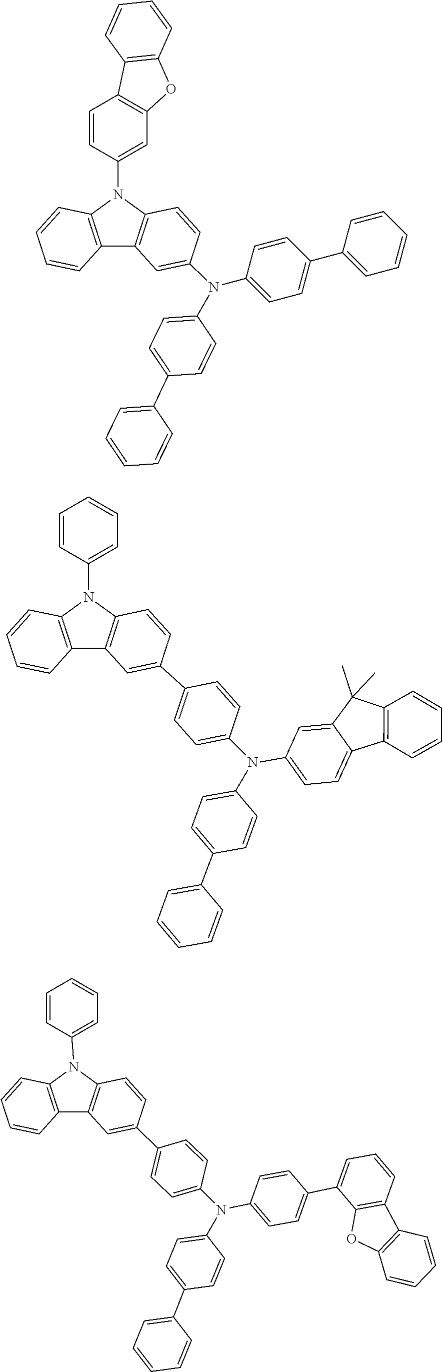 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

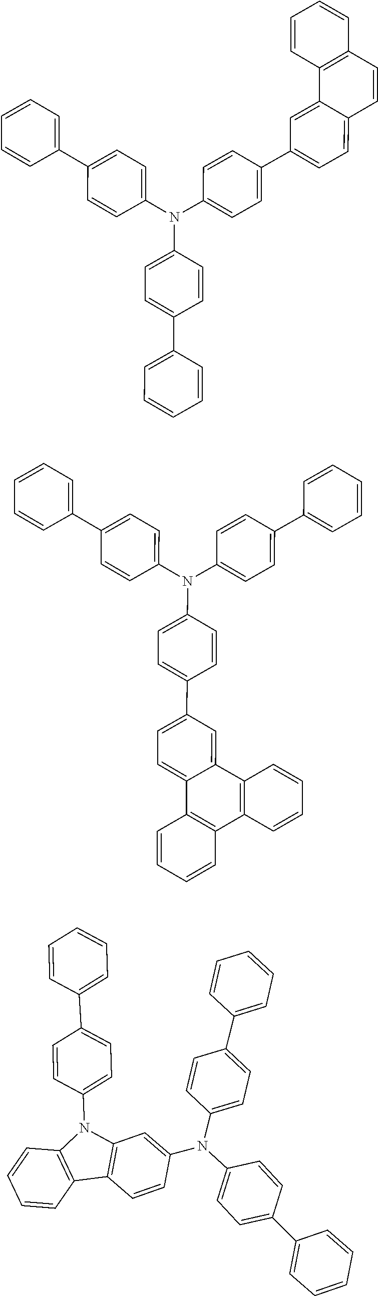 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

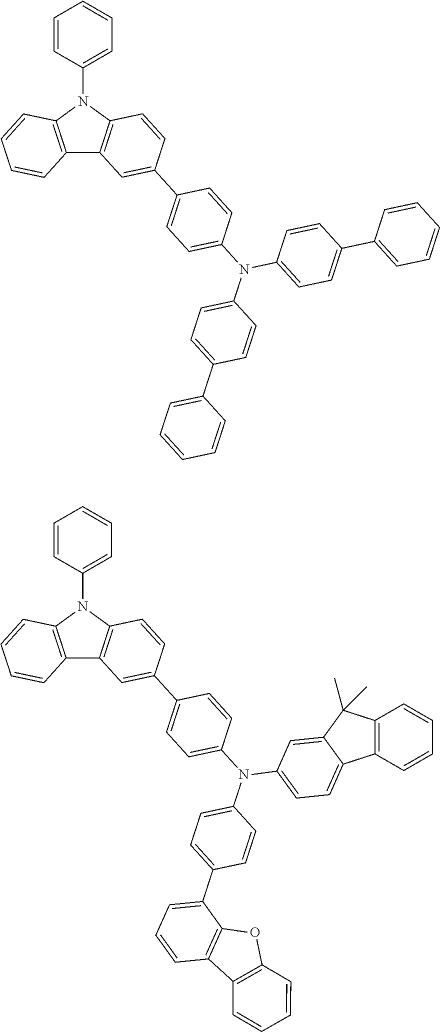 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

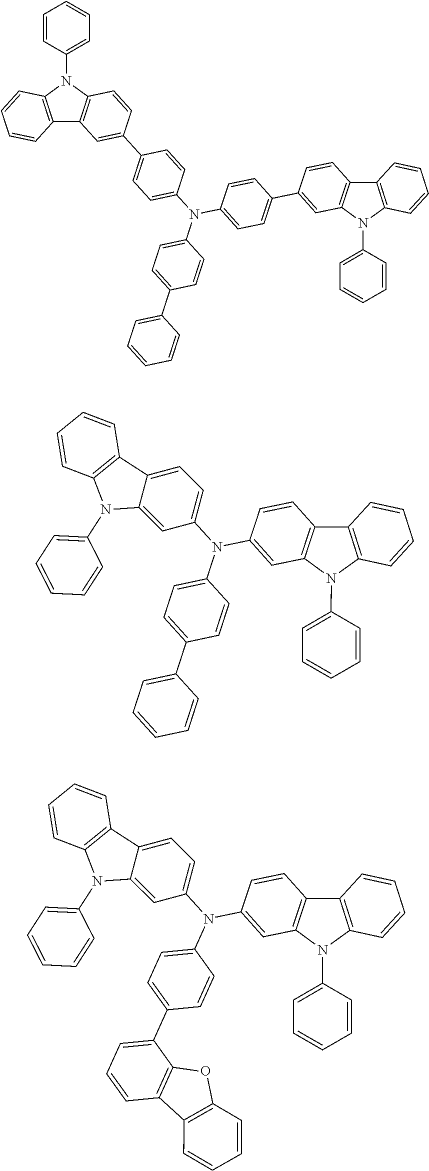 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1 ; c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

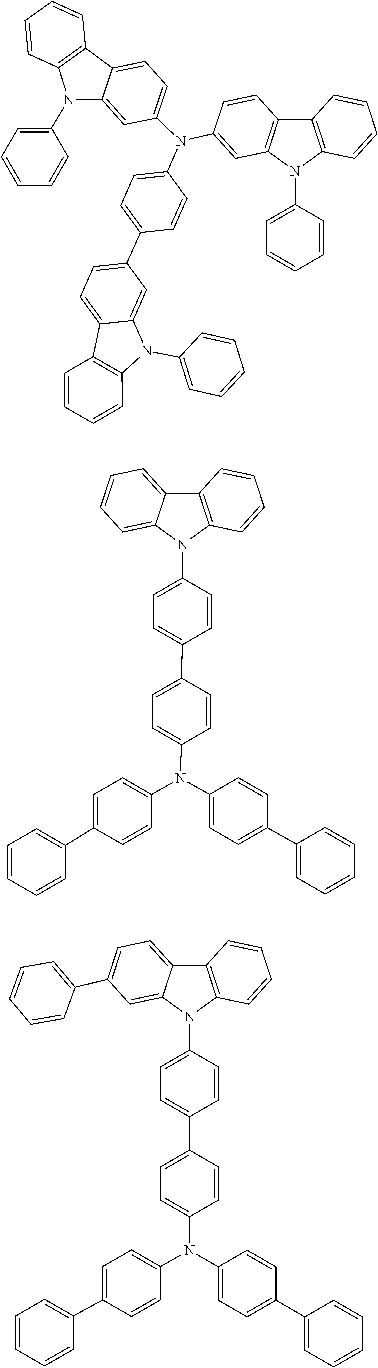 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

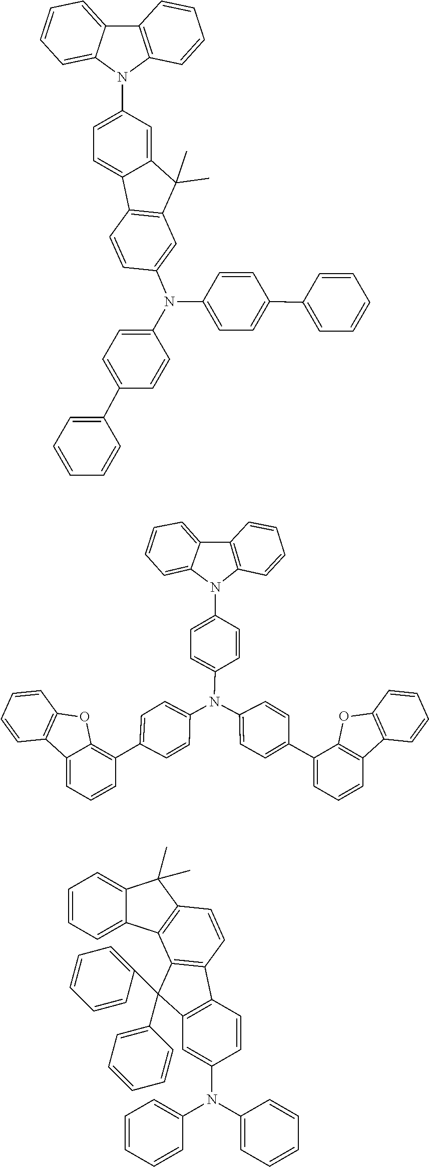 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

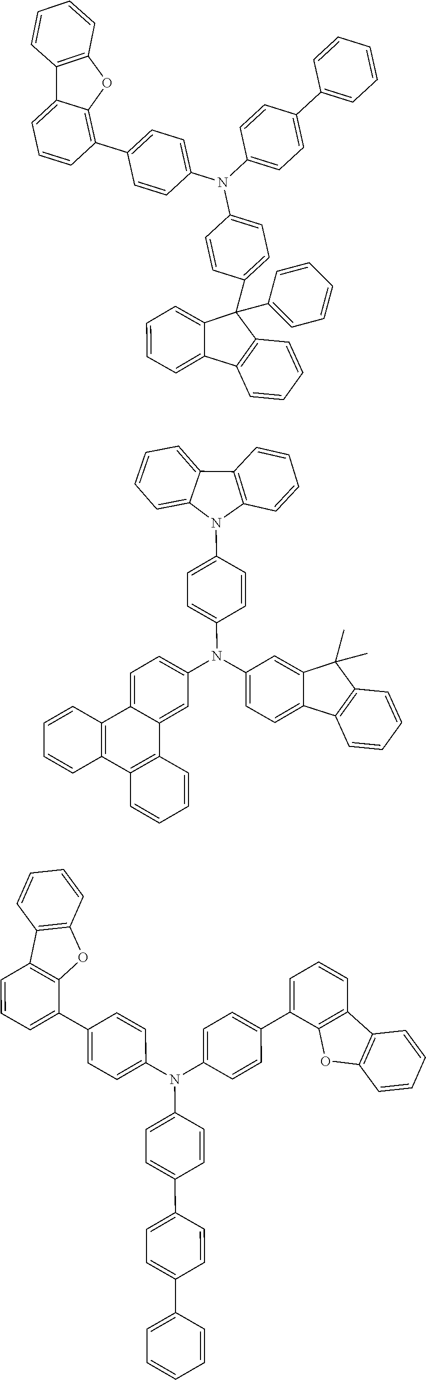 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

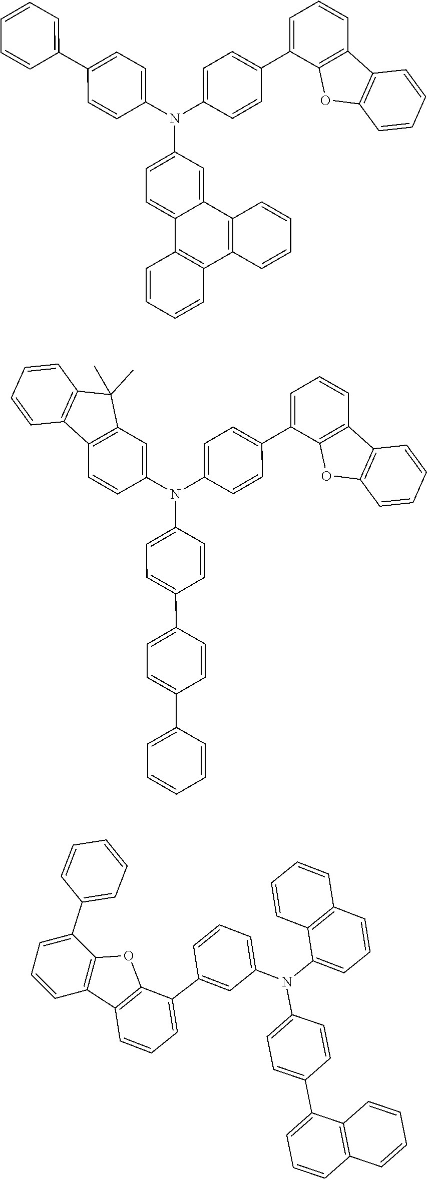 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

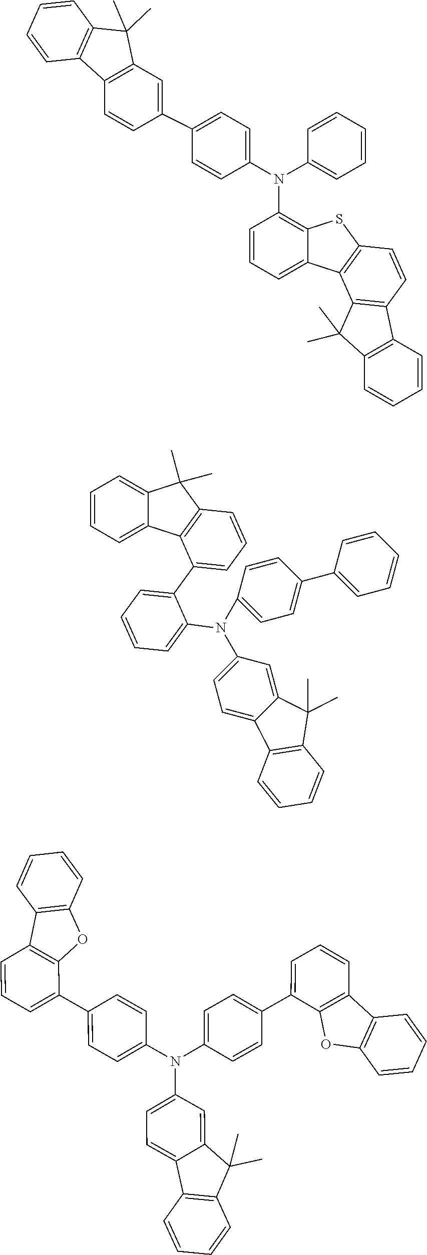 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

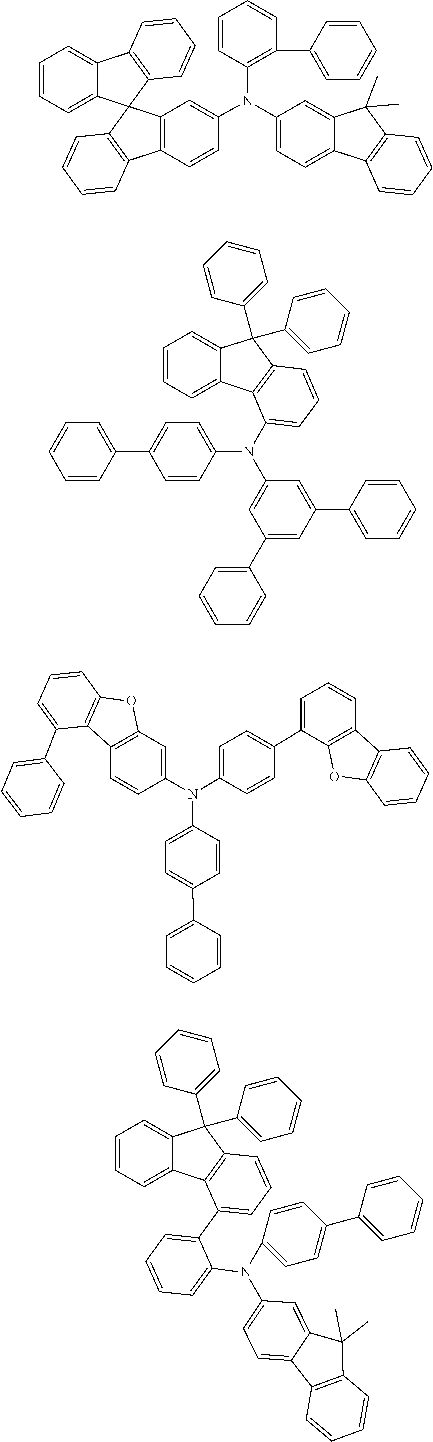 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

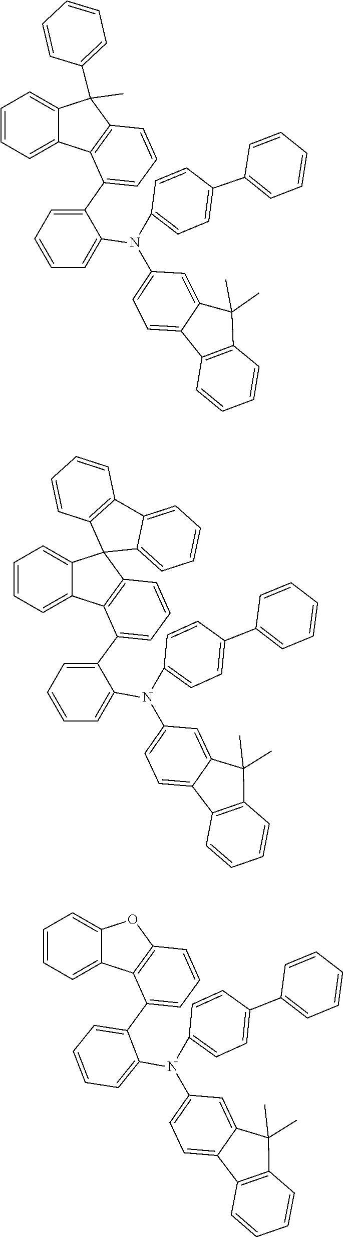 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

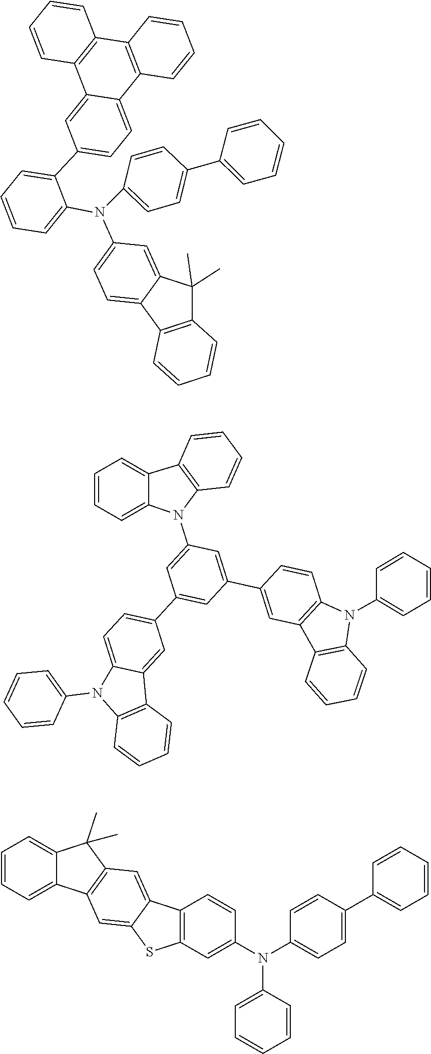 [SMILES: CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1 ; CC1(C)c2ccccc2-c2cc3sc4cc(N(c5ccccc5)c5ccc(-c6ccccc6)cc5)ccc4c3cc21]

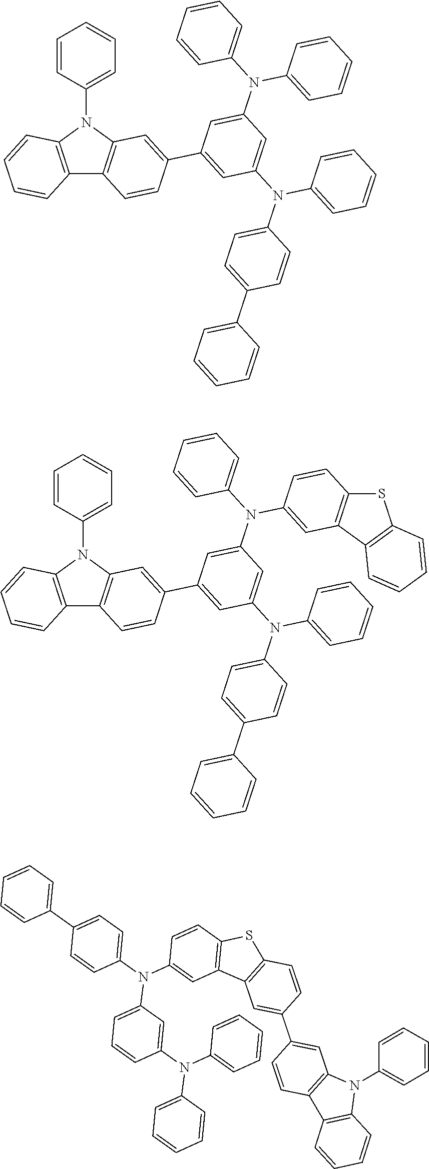 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

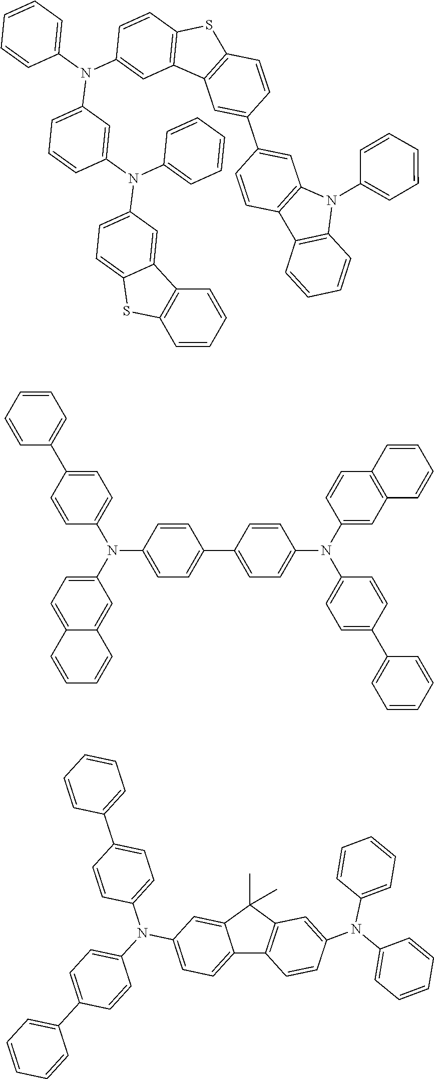 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

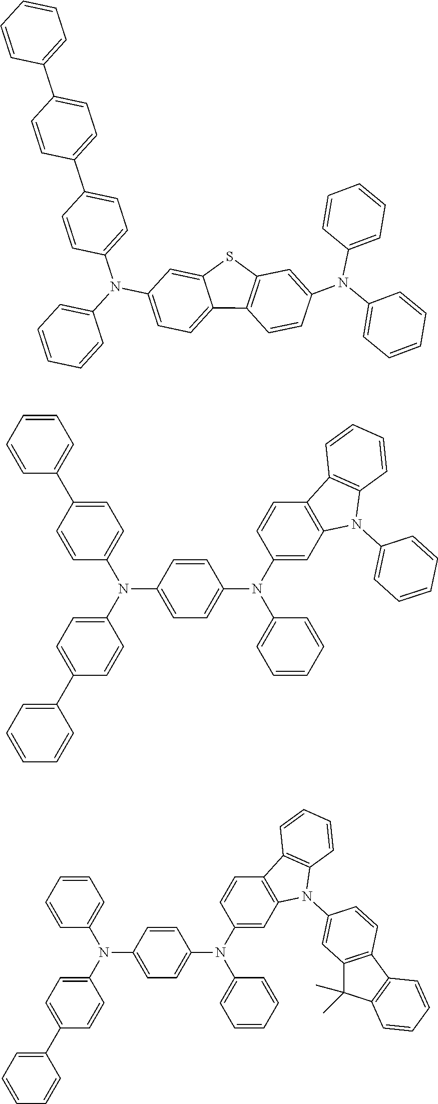 [SMILES: c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

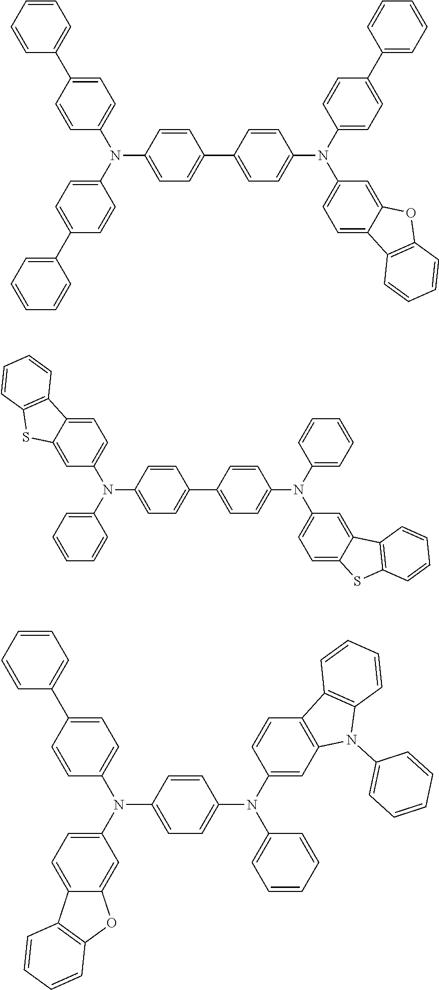 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

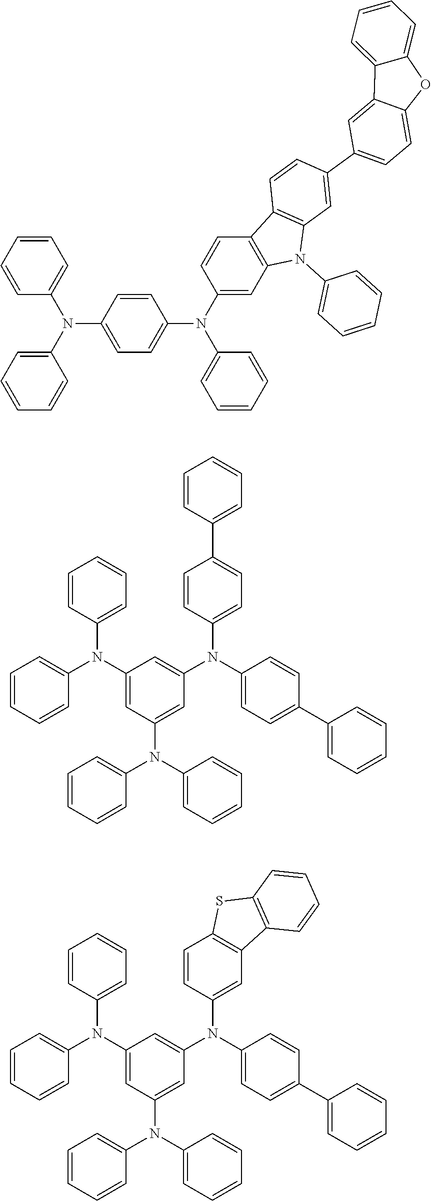 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

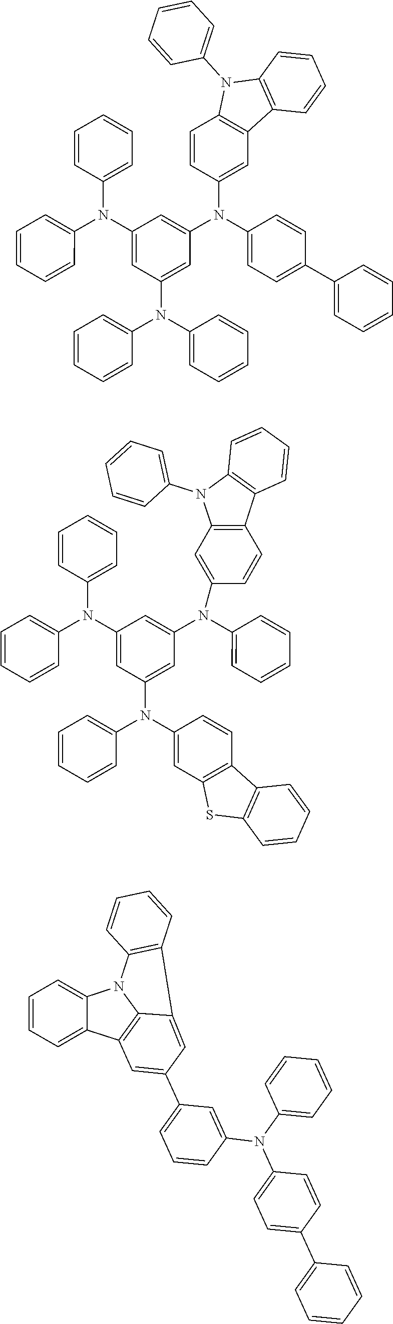 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

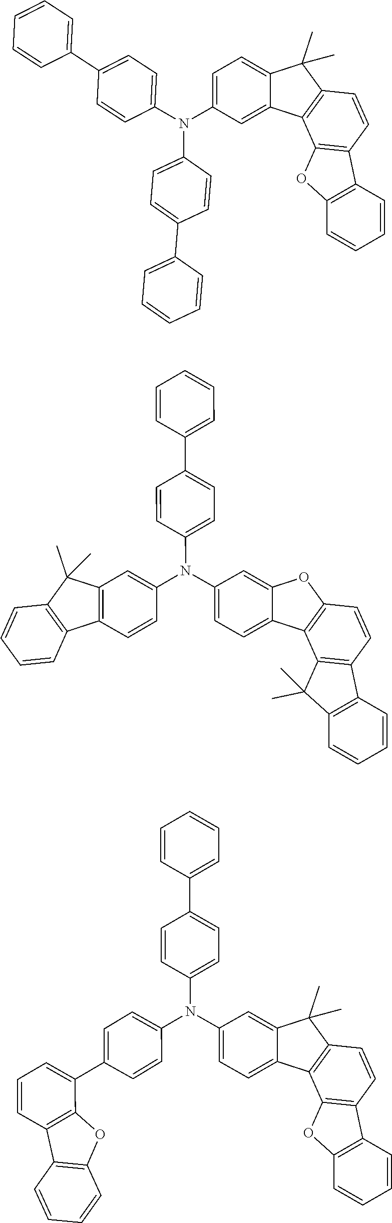 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21]

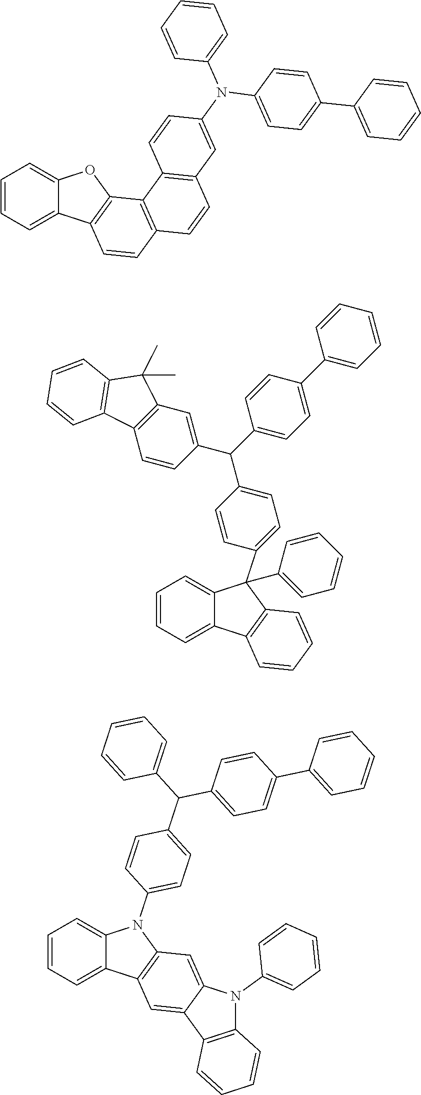

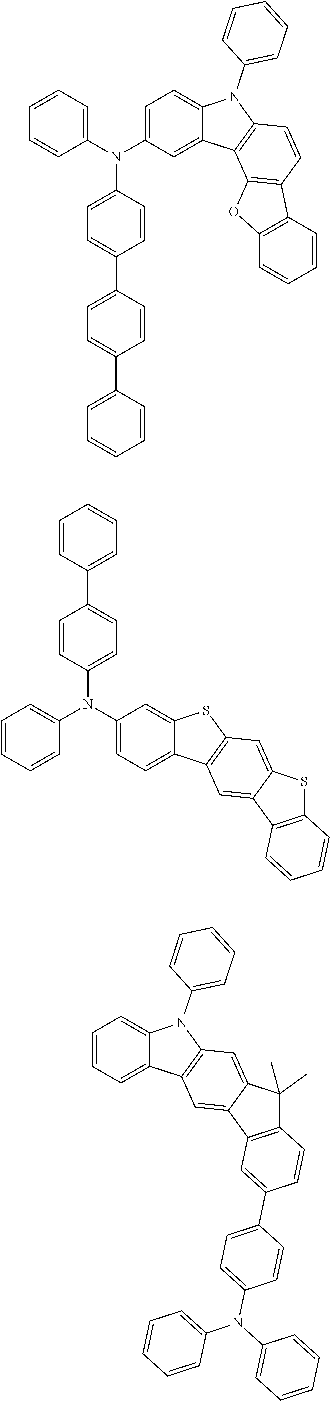 [SMILES: c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

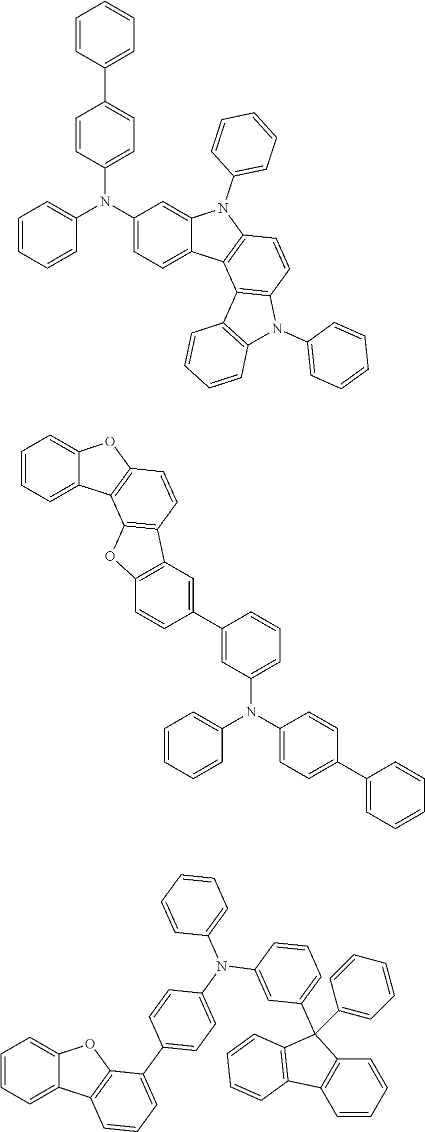 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

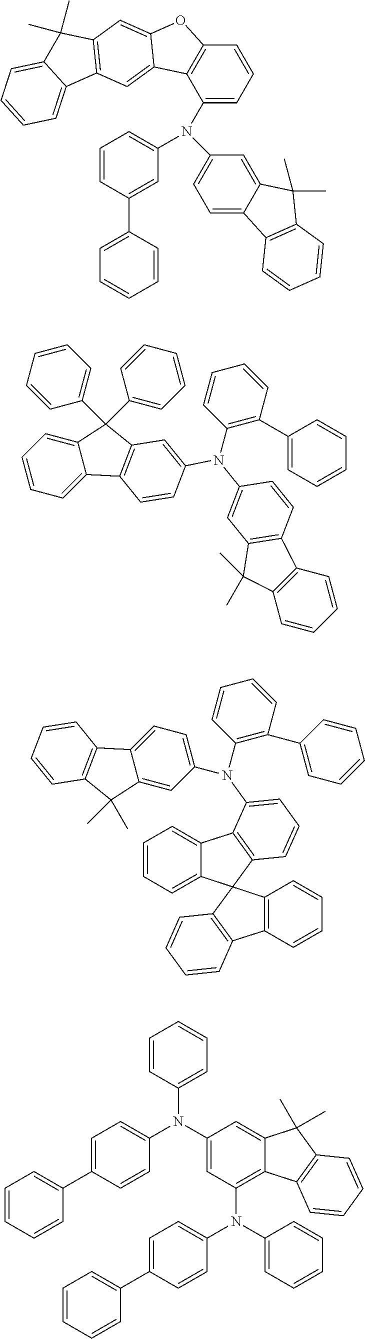 [SMILES: CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21 ; CC1(C)c2ccccc2-c2c(N(c3ccccc3)c3ccc(-c4ccccc4)cc3)cc(N(c3ccccc3)c3ccc(-c4ccccc4)cc3)cc21]

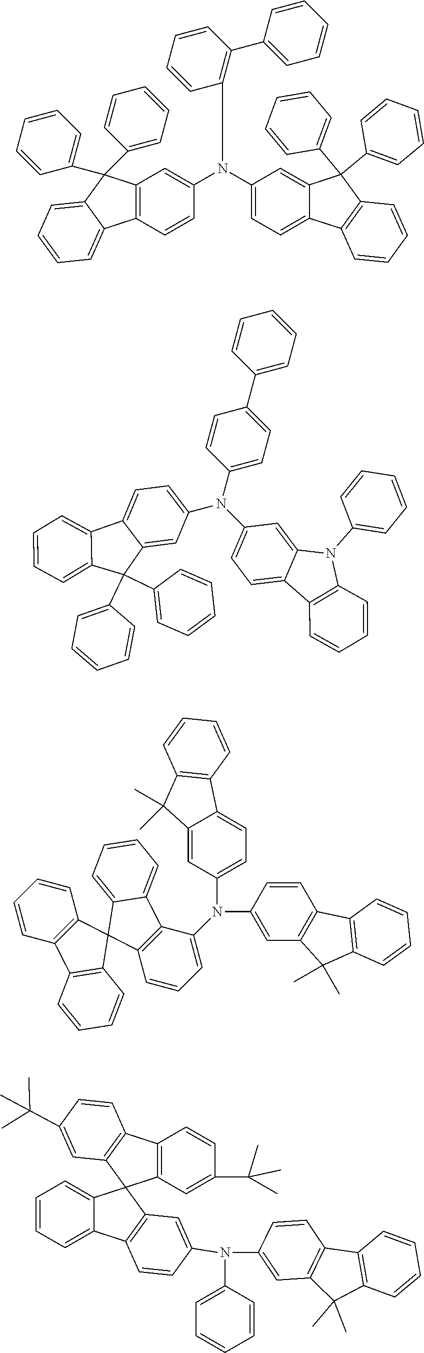 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

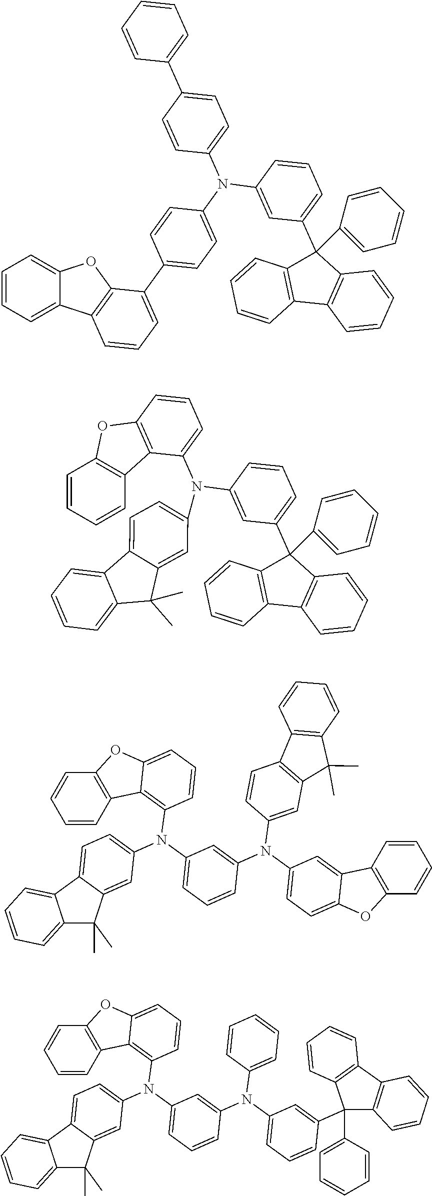 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21 ; CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

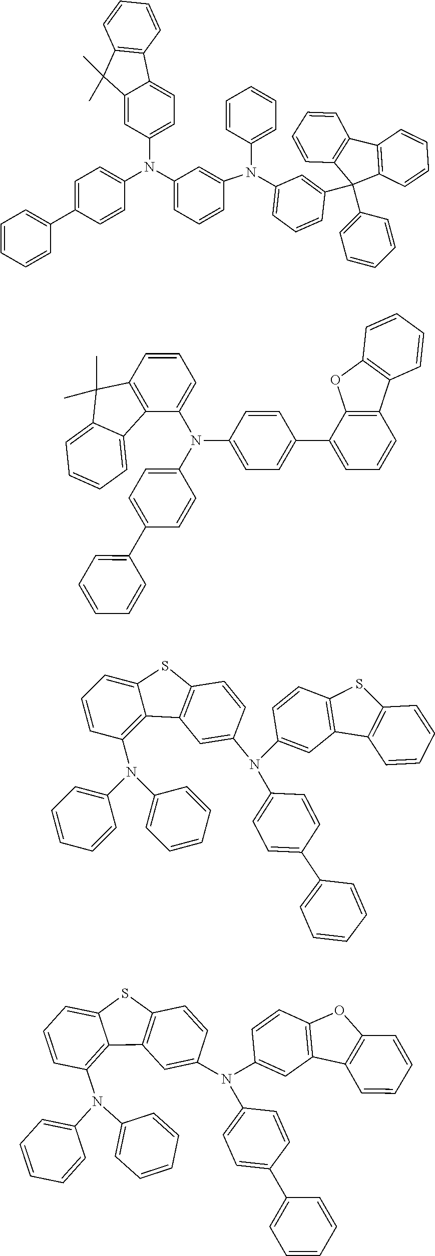 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

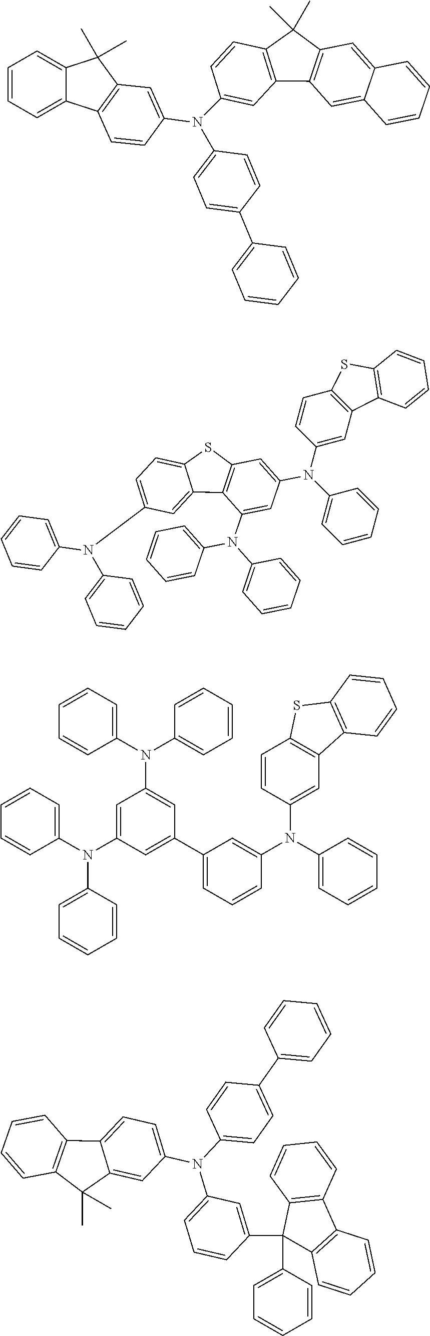 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, known compounds disclosed in U.S. Pat. No. 5,061,569A, JP1993-009471A, WO1995-009147A1, JP1995-126615A, JP1998-095973A, and the like and compounds similar thereto may be used in addition to the compound.

Also, the charge transport region may be, for example, the electron transport region 150.

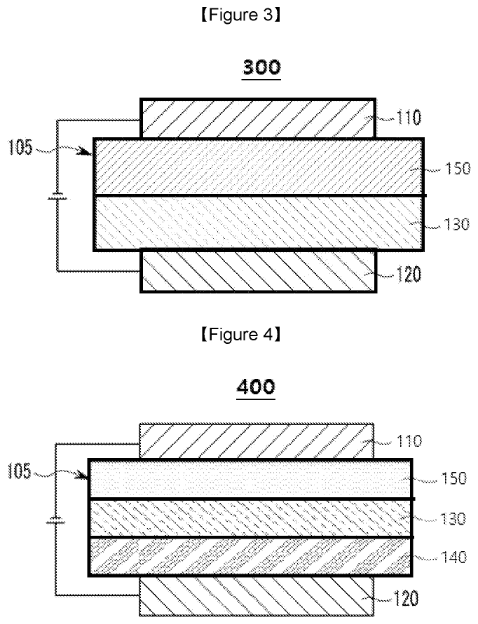

Referring to FIG. 3, the organic light emitting diode 300 further includes an electron transport region 150 in addition to the light emitting layer 130. The electron transport region 150 may further increase electron injection and/or electron mobility between the cathode 110 and the light emitting layer 130 and block holes.

Specifically, the electron transport region 150 may include an electron transport layer between the cathode 110 and the light emitting layer 130, and an electron transport auxiliary layer between the light emitting layer 130 and the electron transport layer and at least one of the compounds of Group B may be included in at least one of the electron transport layer and the electron transport auxiliary layer.

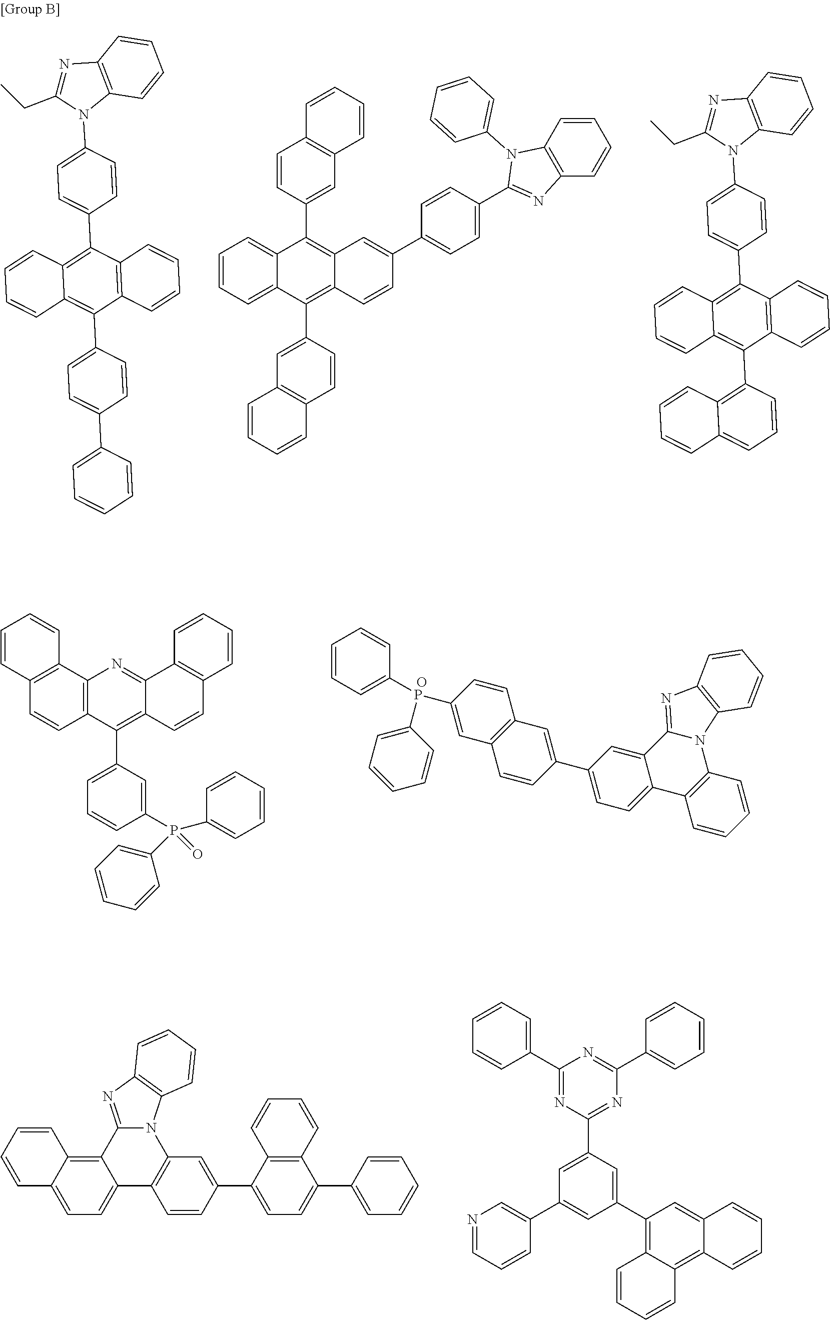

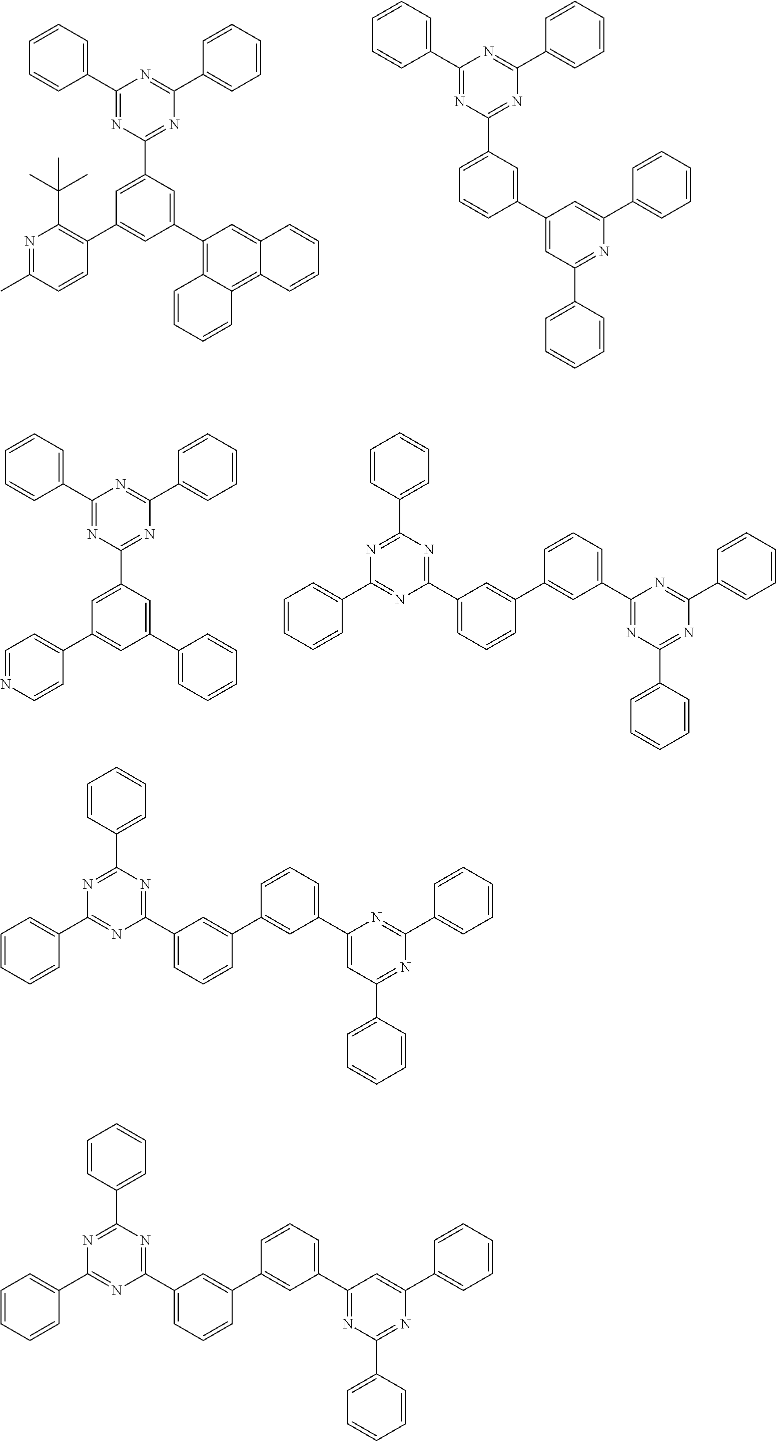 [SMILES: Cc1ccc(-c2cc(-c3nc(-c4ccccc4)nc(-c4ccccc4)n3)cc(-c3cc4ccccc4c4ccccc34)c2)c(C(C)(C)C)n1 ; c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

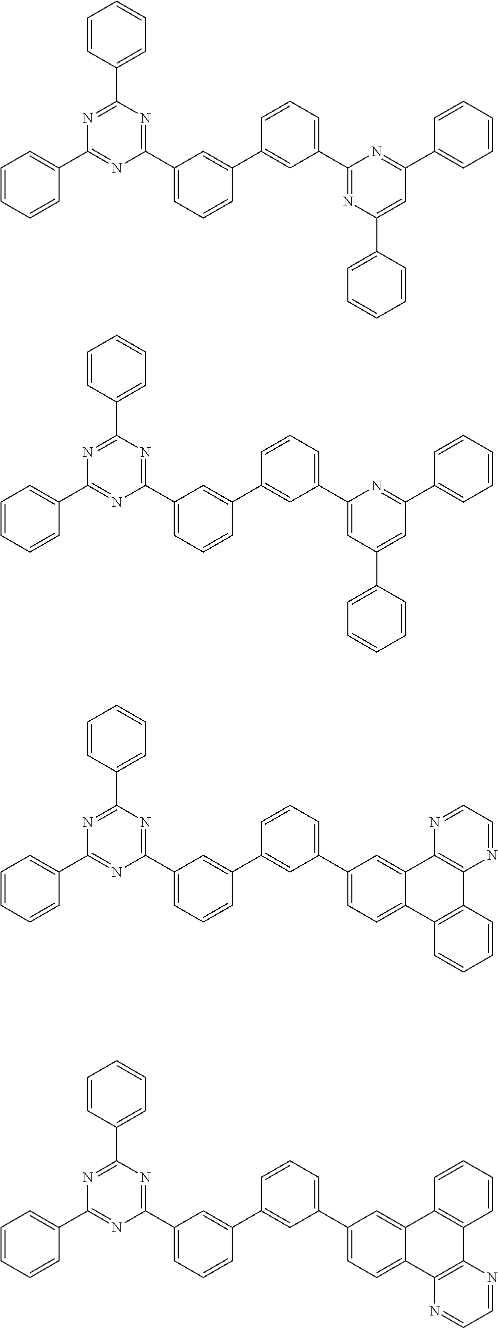 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

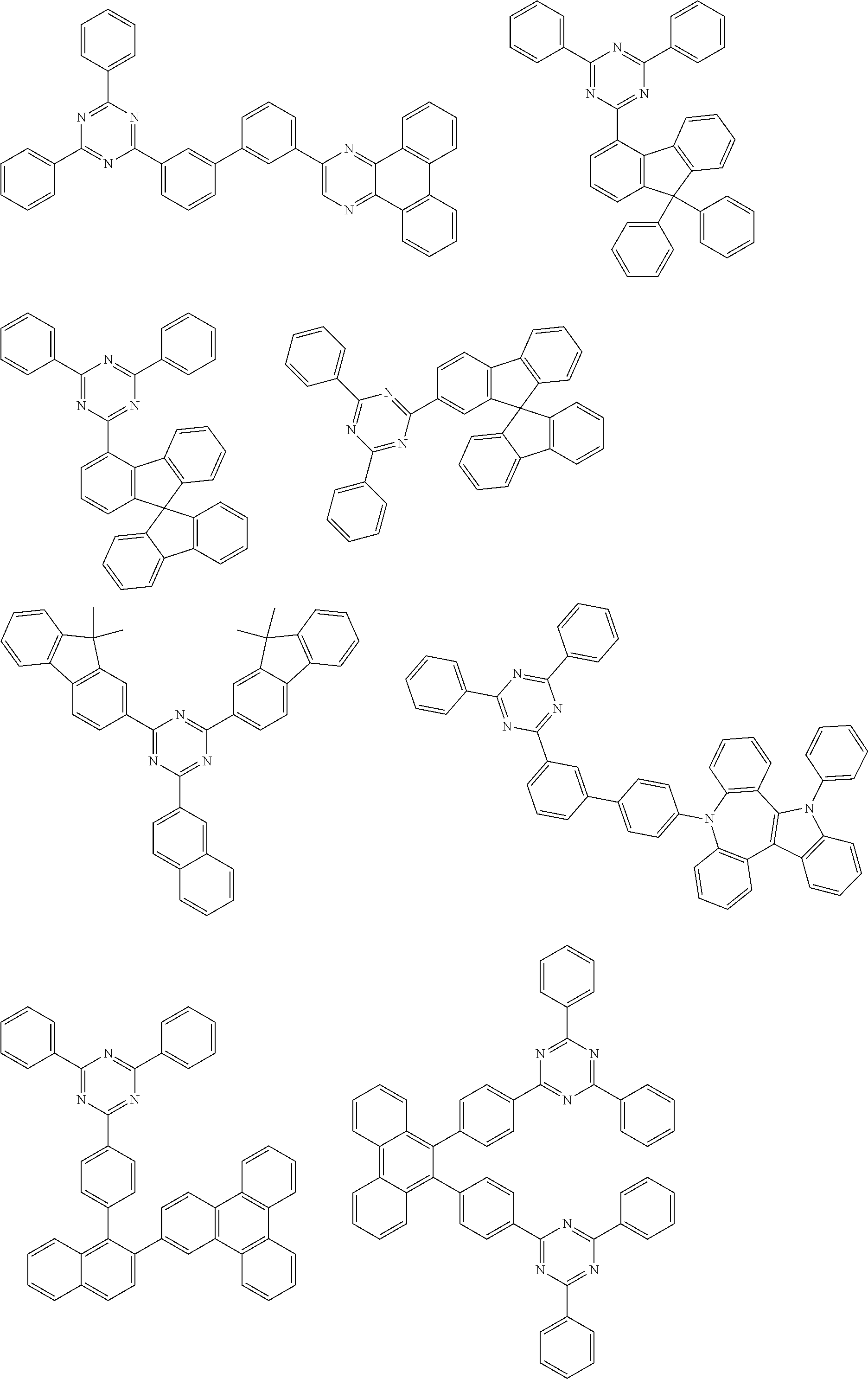

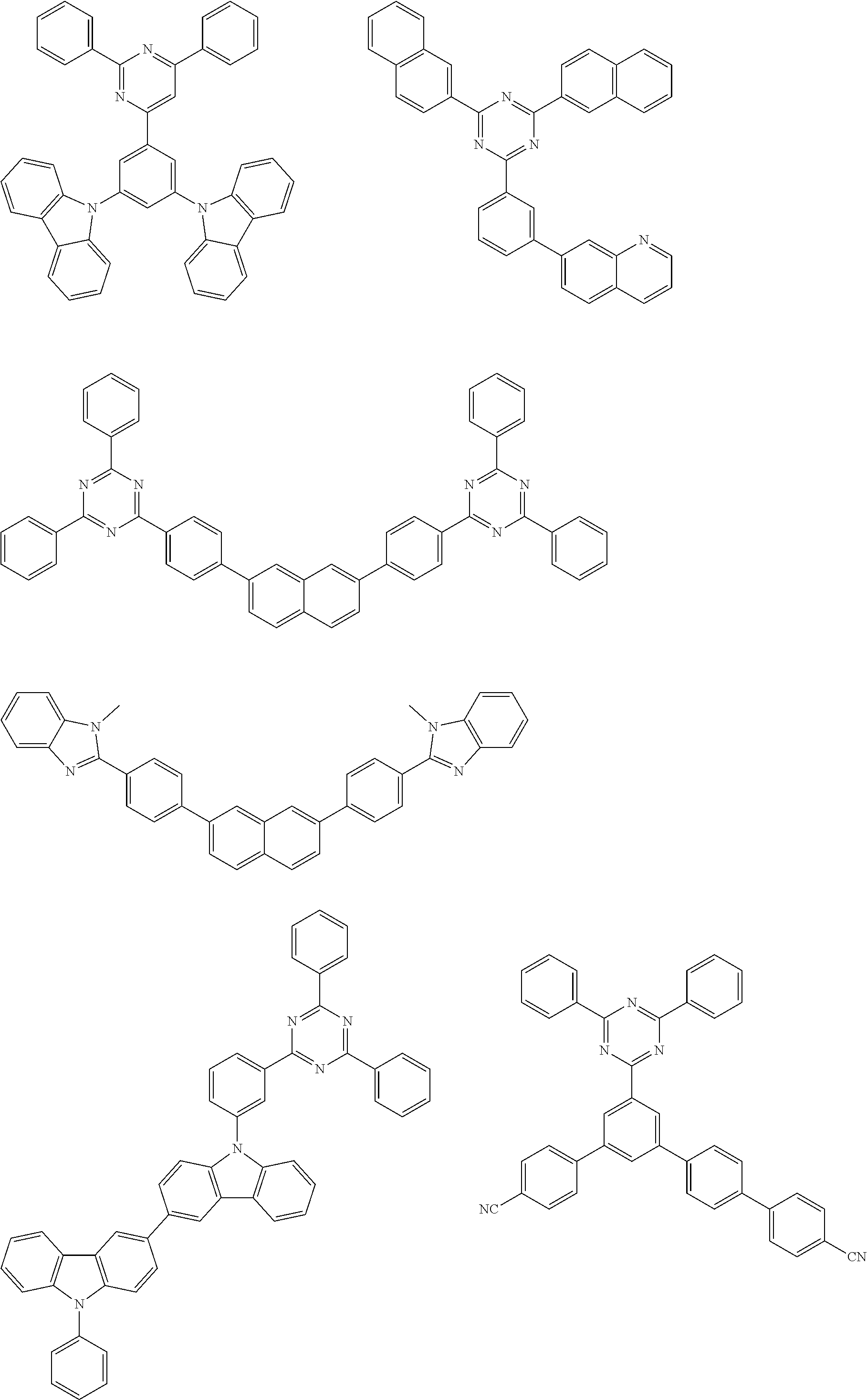

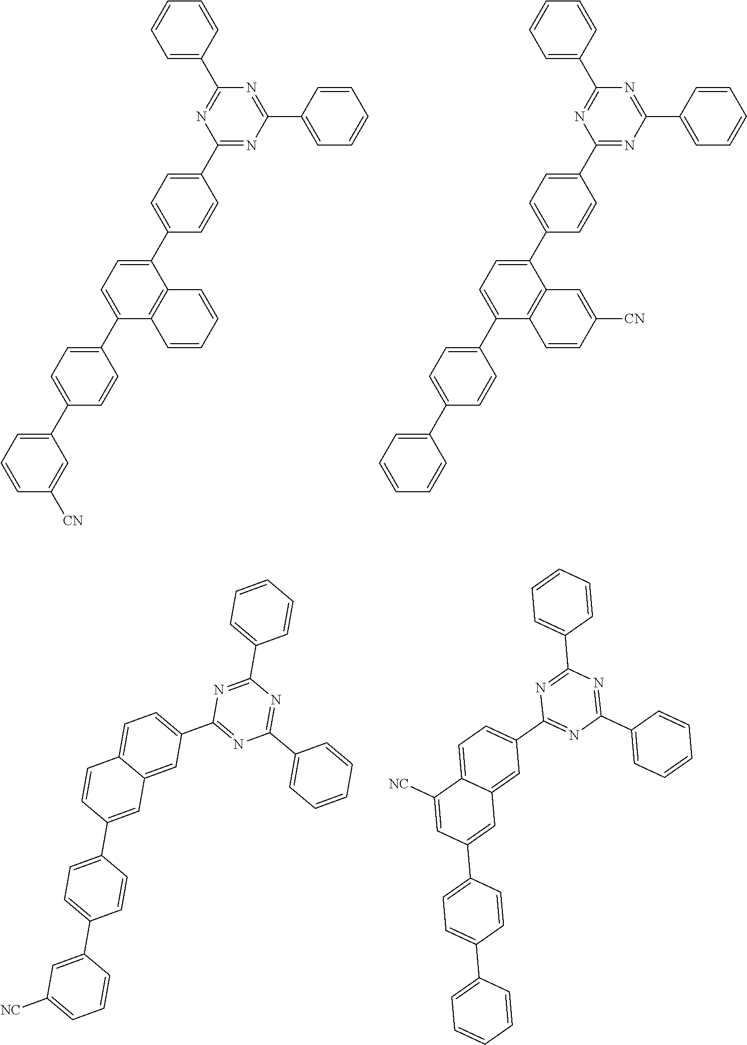 [SMILES: N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

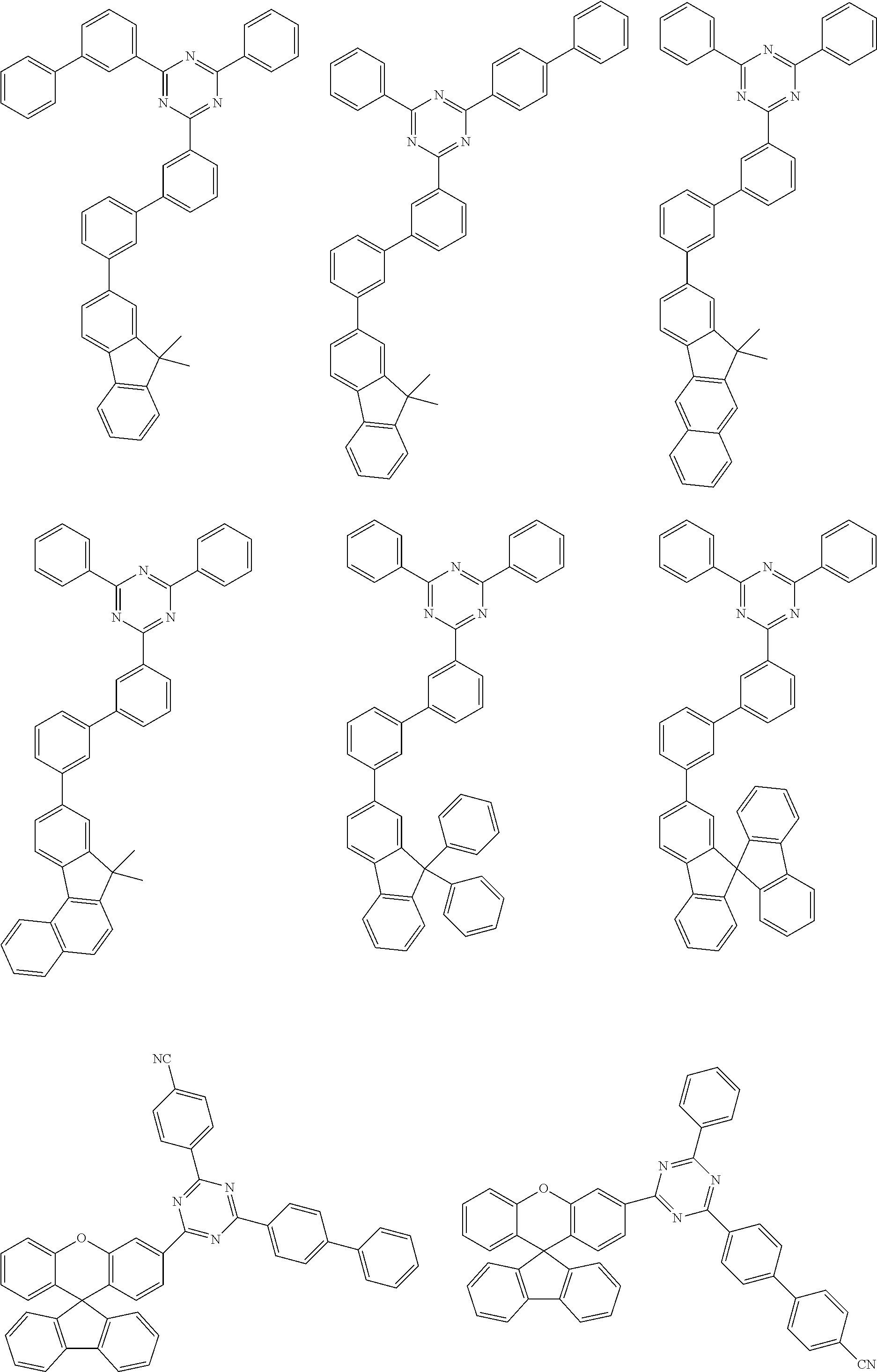

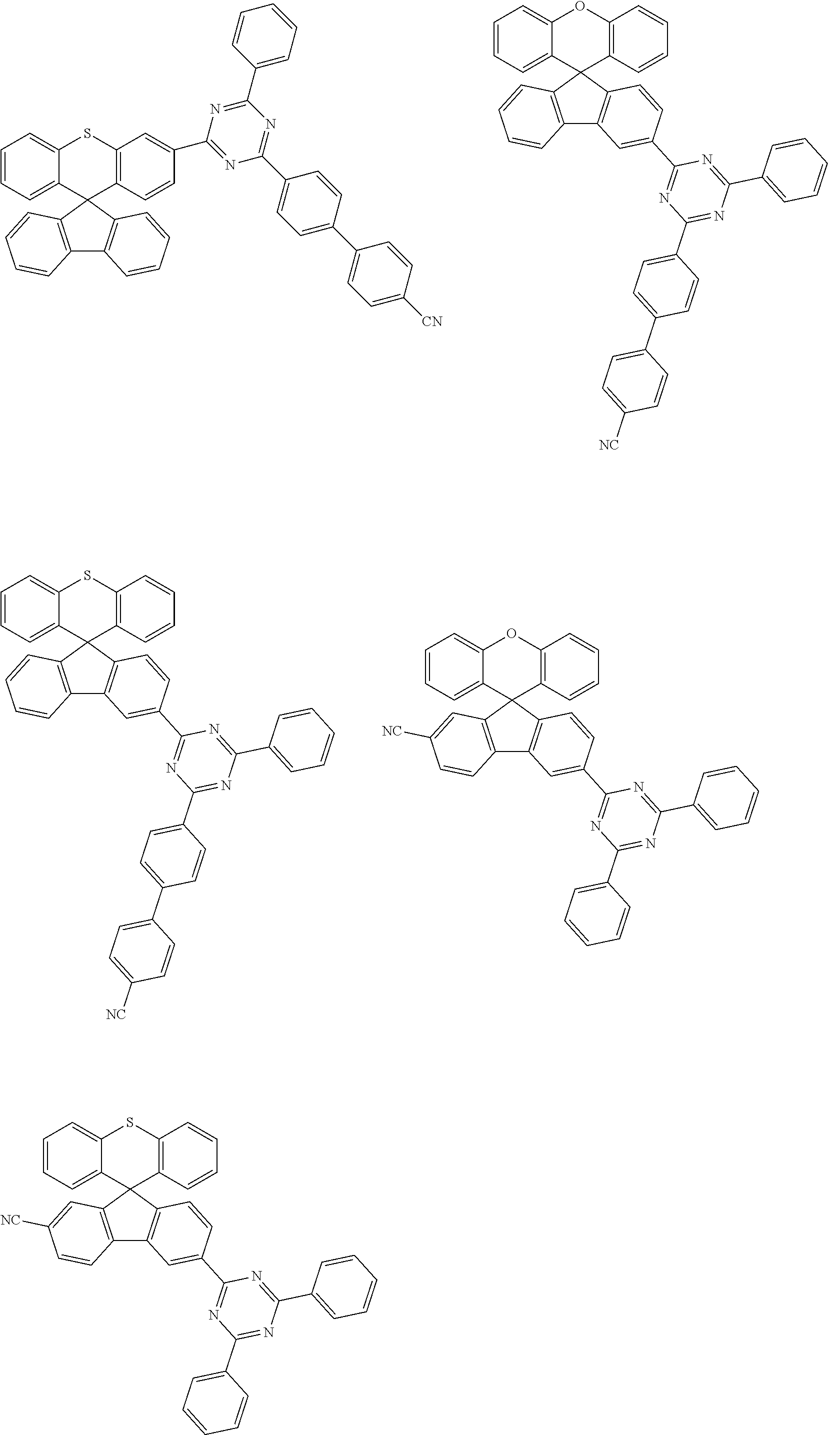 [SMILES: N#Cc1ccc(-c2ccc(-c3nc(-c4ccccc4)nc(-c4ccc5c(c4)Sc4ccccc4C54c5ccccc5-c5ccccc54)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1 ; N#Cc1ccc2c(c1)C1(c3ccccc3Oc3ccccc31)c1ccc(-c3nc(-c4ccccc4)nc(-c4ccccc4)n3)cc1-2 ; N#Cc1ccc2c(c1)C1(c3ccccc3Sc3ccccc31)c1ccc(-c3nc(-c4ccccc4)nc(-c4ccccc4)n3)cc1-2]

An embodiment of the present invention may provide an organic light emitting diode including the light emitting layer 130 as the organic layer 105 as shown in FIG. 1.

Another embodiment of the present invention may provide an organic light emitting diode including a hole transport region 140 in addition to the light emitting layer 130 as the organic layer 105 as shown in FIG. 2.

Another embodiment of the present invention may provide an organic light emitting diode including an electron transport region 150 in addition to the light emitting layer 130 as the organic layer 105 as shown in FIG. 3.

Another embodiment of the present invention may provide an organic light emitting diode including a hole transport region 140 and an electron transport region 150 in addition to the emission layer 130 as the organic layer 105 as shown in FIG. 4.

Another embodiment of the present invention may provide an organic light emitting diode further including an electron injection layer (not shown), a hole injection layer (not shown), etc. in addition to the light emitting layer 130 as the organic layer 105 in each of FIGS. 1 to 4.

The organic light emitting diodes 100, 200, 300, and 400 may be produced by forming an anode and a cathode on a substrate, forming an organic layer using a dry coating method such as evaporation, sputtering, plasma plating, and ion plating or a solution process, and forming a cathode or an anode thereon.

The organic light emitting diode may be applied to an organic light emitting display device.

### DETAILED DESCRIPTION OF THE EMBODIMENTS

Hereinafter, the embodiments are illustrated in more detail with reference to examples. However, these examples are exemplary, and the present scope is not limited thereto.

Hereinafter, starting materials and reactants used in Examples and Synthesis Examples were purchased from Sigma-Aldrich Co. Ltd., TCI Inc., Tokyo chemical industry or P&H tech as far as there in no particular comment or were synthesized by known methods.

### (Preparation of Compounds for Organic Optoelectronic Devices)

The compounds presented as a more specific example of the compound of the present invention were synthesized through the following steps.

### Synthesis of First Compound

### Synthesis Example 1: Synthesis of Compound 39

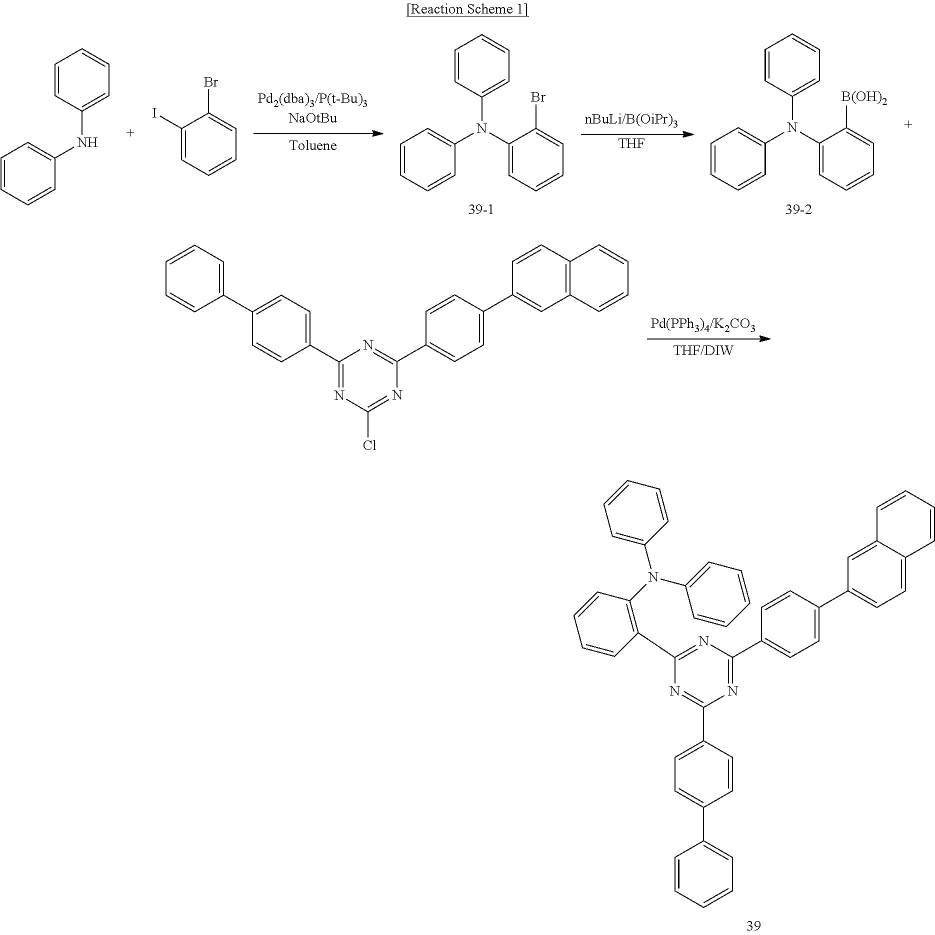

a) Synthesis of Intermediate 39-1

Diphenylamine (30.0 g, 177 mmol), 1-bromo-2-iodobenzene (50.2 g, 177 mmol), Pd₂(dba)₃ (8.1 g, 9 mmol), and NaO(t-Bu) (25.6 g, 266 mmol) were dissolved in 800 mL of toluene, and a P(t-Bu)₃ solution (5.4 g, 27 mmol) was slowly added thereto in a dropwise fashion and then, stirred under reflux at 130° C. for 12 hours. When a reaction was completed, the resultant was treated through column chromatography (n-hexane:dichloromethane), obtaining 38.3 g (66.7%) of Intermediate 39-1.

b) Synthesis of Intermediate 39-2

Intermediate 39-1 (38.3 g, 118 mmol) and triisopropyl borate (26.7 g, 142 mmol) were dissolved in 250 mL of anhydrous THE and then, stirred at −78° C. After 30 minutes, 2.5 M n-butyllithium solution (56.7 mL, 142 mmol) was slowly added thereto in a dropwise fashion and then, stirred for 12 hours. When a reaction was completed, the resultant was twice extracted with distilled water and dichloromethane, and an organic solvent of an organic layer therefrom was concentrated by using a rotary evaporator. The concentrated organic layer was slurry-stirred/purified with n-hexane, obtaining 29.9 g (87.6%) of Intermediate 39-2.

c) Synthesis of Compound 39

Intermediate 39-2 (10.0 g, 35 mmol), 2-([1,1′-biphenyl]-4-yl)-4-chloro-6-(4-(naphthalen-2-yl)phenyl)-1,3,5-triazine (16.3 g, 35 mmol), Pd(PPh₃)₄ (2.0 g, 2 mmol), and K₂CO₃ (14.3 g, 104 mmol) were dissolved in 200 mL of a mixed solution of tetrahydrofuran:distilled water=2:1 and then, stirred under reflux at 80° C. for 12 hours. When a reaction was completed, the resultant was recrystallized and purified with a mixed solution of dichloromethane:n-hexane, obtaining 16.7 g (71.0%) of Compound 39.

LC/MS calculated for: C49H34N4 Exact Mass: 678.28 found for 679.36 [M+H]

### Synthesis Example 2: Synthesis of Compound 43

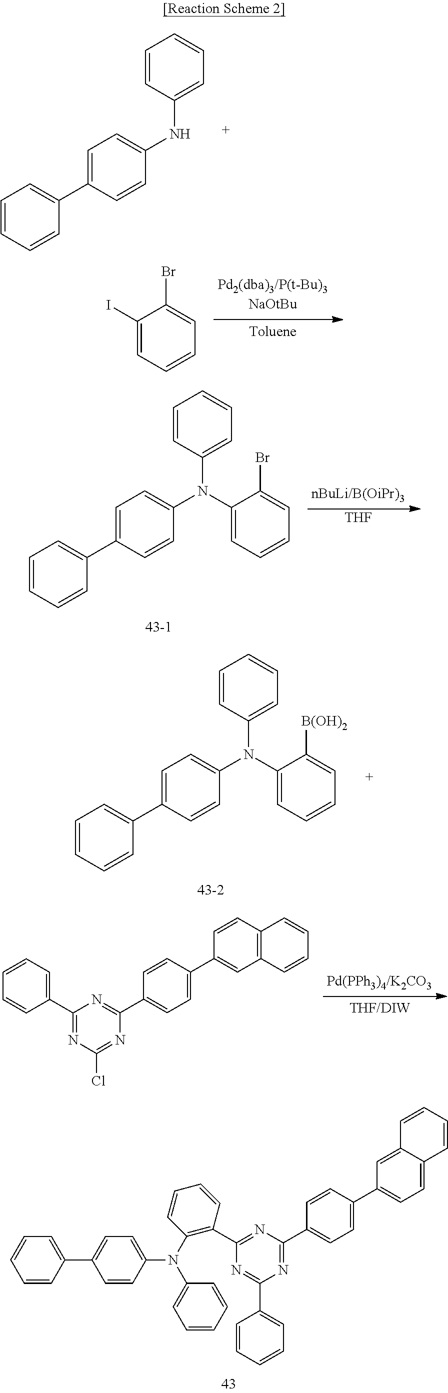

a) Synthesis of Intermediate 43-1

Intermediate 43-1 was synthesized in the same manner as the method of synthesizing/purifying Intermediate 39-1 of Synthesis Example 1 except that N-phenyl-[1,1′-biphenyl]-4-amine and 1-bromo-2-iodobenzene were used as a starting material.

b) Synthesis of Intermediate 43-2

Intermediate 43-2 was synthesized in the same manner as the method of synthesizing/purifying Intermediate 39-2 of Synthesis Example 1 except that Intermediate 43-1 was used as a starting material.

c) Synthesis of Compound 43

Compound 43 was synthesized in the same manner as the method of synthesizing/purifying Compound 39 of Synthesis Example 1 except that Intermediate 43-2 and 2-chloro-4-(4-(naphthalen-2-yl)phenyl)-6-phenyl-1,3,5-triazine were used as a starting material.

LC/MS calculated for: C49H34N4 Exact Mass: 678.28 found for 679.19 [M+H]

### Synthesis Example 3: Synthesis of Compound 66

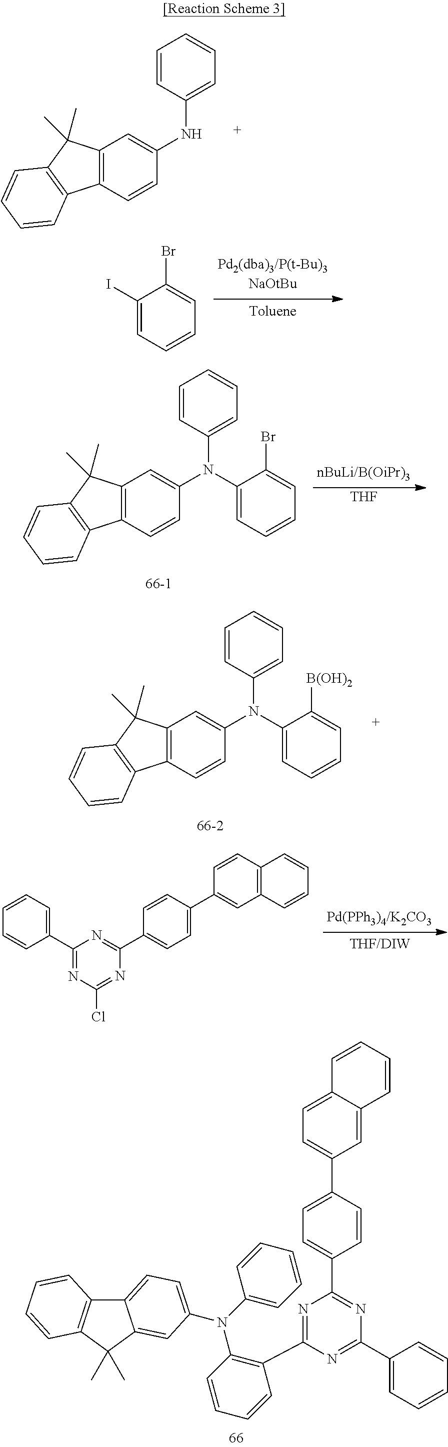

a) Synthesis of Intermediate 66-1

Intermediate 66-1 was synthesized in the same manner as the method of synthesizing/purifying Intermediate 39-1 of Synthesis Example 1 except that 9,9-dimethyl-N-phenyl-9H-fluoren-2-amine and 1-bromo-2-iodobenzene were used as a starting material.

b) Synthesis of Intermediate 66-2

Intermediate 66-2 was synthesized in the same manner as the method of synthesizing/purifying Intermediate 39-2 of Synthesis Example 1 except that Intermediate 66-1 was used as a starting material.

c) Synthesis of Compound 66

Compound 66 was synthesized in the same manner as the method of synthesizing/purifying Compound 39 of Synthesis Example 1 except that Intermediate 66-2 and 2-chloro-4-(4-(naphthalen-2-yl)phenyl)-6-phenyl-1,3,5-triazine were used as a starting material.

LC/MS calculated for: C52H38N4 Exact Mass: 718.31 found for 719.43 [M+H]

### Synthesis Example 4: Synthesis of Compound 67

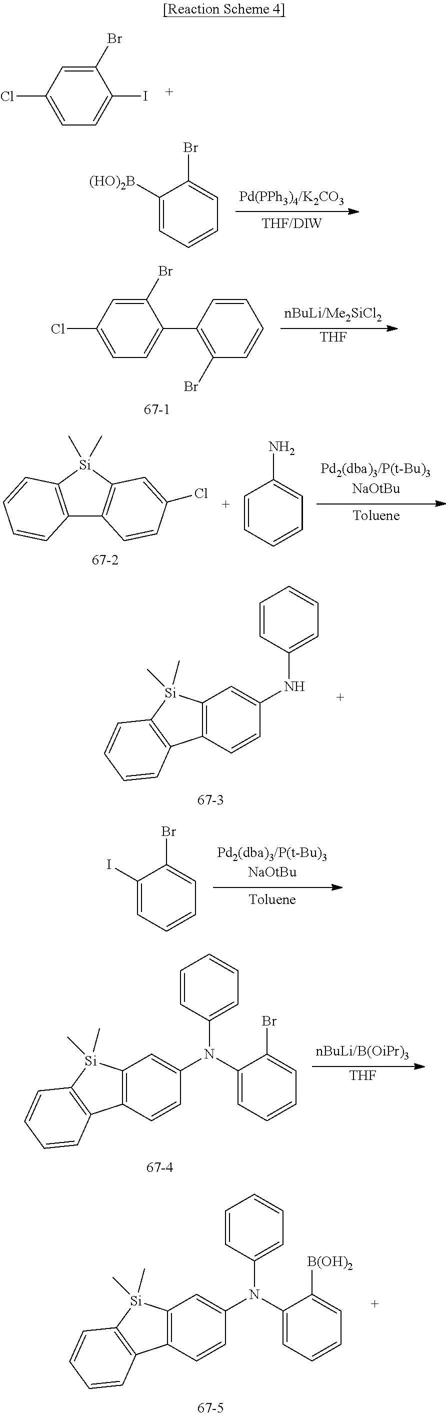

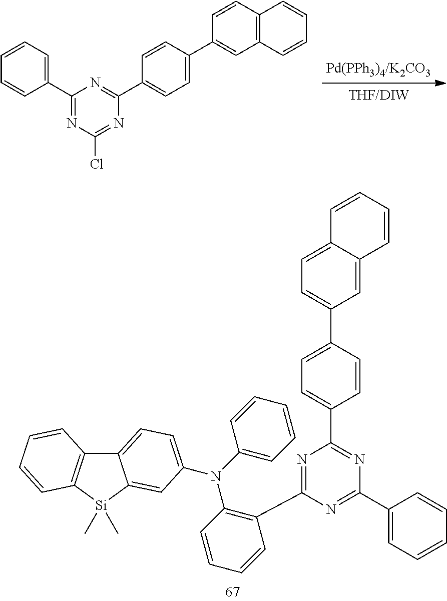 [SMILES: Clc1nc(-c2ccccc2)nc(-c2ccc(-c3ccc4ccccc4c3)cc2)n1 ; C[Si]1(C)c2ccccc2-c2ccc(N(c3ccccc3)c3ccccc3-c3nc(-c4ccccc4)nc(-c4ccc(-c5ccc6ccccc6c5)cc4)n3)cc21]

a) Synthesis of Intermediate 67-1

Intermediate 67-1 was synthesized in the same manner as the method of synthesizing Compound 39 of Synthesis Example 1 except that 2-bromo-4-chloro-1-iodobenzene and (2-bromophenyl)boronic acid were used as a starting material and then, purified through silica gel column chromatography.

b) Synthesis of Intermediate 67-2

Intermediate 67-1 (70.7 g, 204 mmol) was dissolved in 600 mL of anhydrous THE and then, stirred at −78° C. After 30 minutes, a 1.6 M n-butyllithium solution (370.0 mL, 592 mmol) was slowly added thereto in a dropwise fashion. After 30 minutes, dichlorodimethylsilane (92.2 g, 714 mmol) was slowly added thereto in a dropwise fashion and then, stirred for 12 hours. When a reaction was completed, the resultant was twice extracted with ethyl acetate and distilled water, and an organic layer therefrom was purified through silica gel column chromatography, obtaining 34.1 g (68.3%) of Intermediate 67-2.

c) Synthesis of Intermediate 67-3

Intermediate 67-3 was synthesized in the same synthesis method of synthesizing/purifying Intermediate 39-1 of Synthesis Example 1 except that Intermediate 67-2 and aniline were used as a starting material.

d) Synthesis of Intermediate 67-4

Intermediate 67-4 was synthesized in the same synthesis method of synthesizing/purifying Intermediate 39-1 of Synthesis Example 1 except that Intermediate 67-3 and 1-bromo-2-iodobenzene were used as a starting material.

e) Synthesis of Intermediate 67-5

Intermediate 67-5 was synthesized in the same synthesis method of synthesizing/purifying Intermediate 39-2 of Synthesis Example 1 except that Intermediate 67-4 was used as a starting material.

f) Synthesis of Compound 67

Compound 67 was synthesized in the same synthesis method of synthesizing/purifying Compound 39 of Synthesis Example 1 except that Intermediate 67-5 and 2-chloro-4-(4-(naphthalen-2-yl)phenyl)-6-phenyl-1,3,5-triazine were used as a starting material for the synthesis/purification.

LC/MS calculated for: C51H38N4Si Exact Mass: 734.29 found for 735.19 [M+H]

### Synthesis Example 5: Synthesis of Compound 76

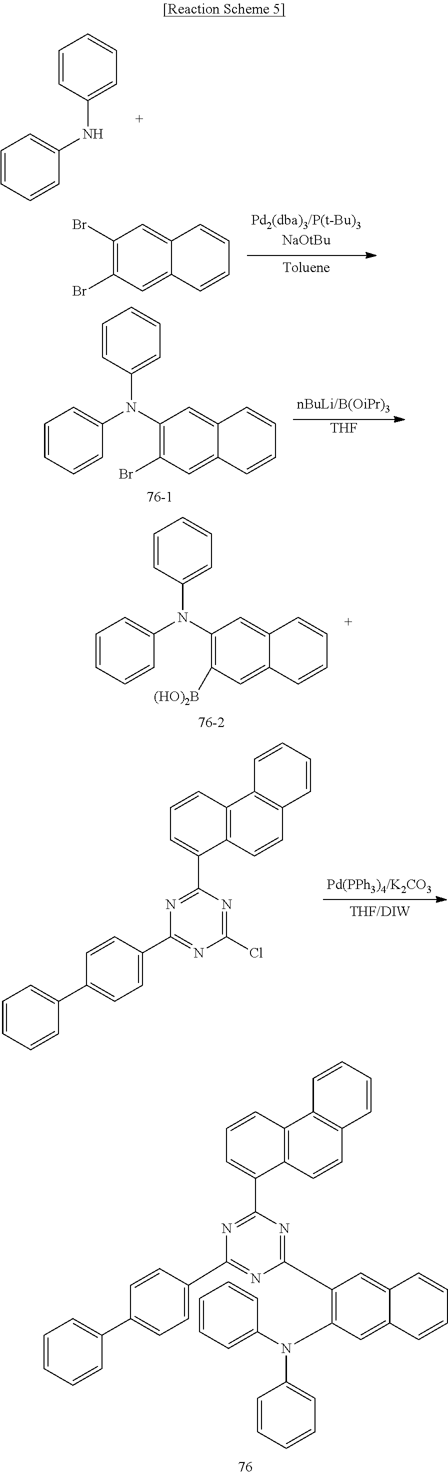

a) Synthesis of Intermediate 76-1

Intermediate 76-1 was synthesized in the same synthesis method of synthesizing/purifying Intermediate 39-1 of Synthesis Example 1 except that diphenylamine and 2,3-dibromonaphthalene were used as a starting material.

b) Synthesis of Intermediate 76-2

Intermediate 76-2 was synthesized in the same synthesis method of synthesizing/purifying Intermediate 39-2 of Synthesis Example 1 except that Intermediate 76-1 was used as a starting material.

c) Synthesis of Compound 76

Compound 76 was synthesized in the same synthesis method of synthesizing/purifying Compound 39 of Synthesis Example 1 except that Intermediate 76-2 and 2-([1,1′-biphenyl]-4-yl)-4-chloro-6-(phenanthren-1-yl)-1,3,5-triazine were used as a starting material.

LC/MS calculated for: C51H34N4 Exact Mass: 702.28 found for 703.33 [M+H]

### Synthesis Example 6: Synthesis of Compound 78

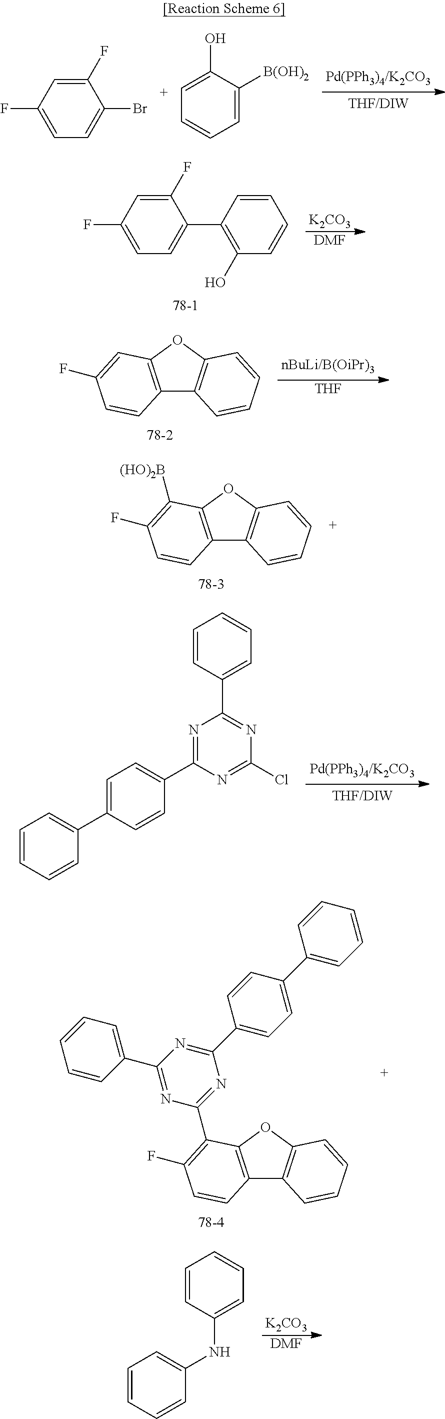

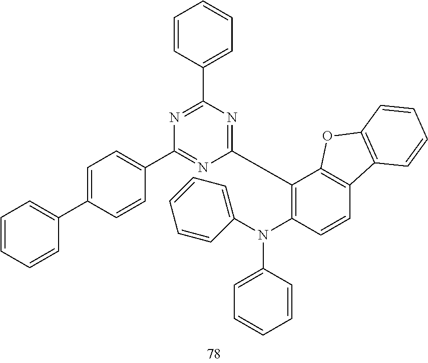 [SMILES: c1ccc(-c2ccc(-c3nc(-c4ccccc4)nc(-c4c(N(c5ccccc5)c5ccccc5)ccc5c4oc4ccccc45)n3)cc2)cc1]

a) Synthesis of Intermediate 78-1

Intermediate 78-1 was synthesized in the same synthesis method of synthesizing/purifying Compound 39 of Synthesis Example 1 except that 1-bromo-2,4-difluorobenzene and 2-hydroxyphenylboronic acid were used as a starting material.

b) Synthesis of Intermediate 78-2

Intermediate 78-1 (20.0 g, 97 mmol) and K₂CO₃ (40.2 g, 291 mmol) were dissolved in 100 mL of N,N-dimethylformamide and then, stirred under reflux at 140° C. for 12 hours. When a reaction was completed, the resultant was purified through column chromatography with a mixed solution of n-hexane:dichloromethane, obtaining 15.9 g (88.2%) of Intermediate 78-2.

c) Synthesis of Intermediate 78-3

Intermediate 78-2 (15.9 g, 85 mmol) was dissolved in 150 mL of anhydrous THE and then, stirred at −78° C. When sufficiently cooled, a 2.5 M n-butyllithium solution (41 mL, 103 mmol) was slowly added thereto in a dropwise fashion and then, stirred while maintained at −78° C. After 1 hour, triisopropyl borate (19.3 g, 103 mmol) was slowly added thereto in a dropwise fashion and then, stirred for 12 hours, while the temperature was slowly increased to room temperature. When a reaction was completed, an organic layer was twice extracted therefrom with ethyl acetate and distilled water and concentrated with a rotary evaporator and then, slurry-purified with n-hexane, obtaining 18.1 g (92.3%) of Intermediate 78-3.

d) Synthesis of Intermediate 78-4

Intermediate 78-4 was synthesized in the same synthesis method of synthesizing/purifying Compound 39 of Synthesis Example 1 except that Intermediate 78-3 and 2-([1,1′-biphenyl]-4-yl)-4-chloro-6-phenyl-1,3,5-triazine were used as a starting material.

e) Synthesis of Compound 78

Compound 78 was synthesized in the same synthesis method of synthesizing/purifying Intermediate 78-2 of Synthesis Example 6 except that Intermediate 78-4 and diphenylamine were used as a starting material.

LC/MS calculated for: C45H30N4O Exact Mass: 642.24 found for 643.66 [M+H]

### Synthesis Example 7: Synthesis of Compound 79

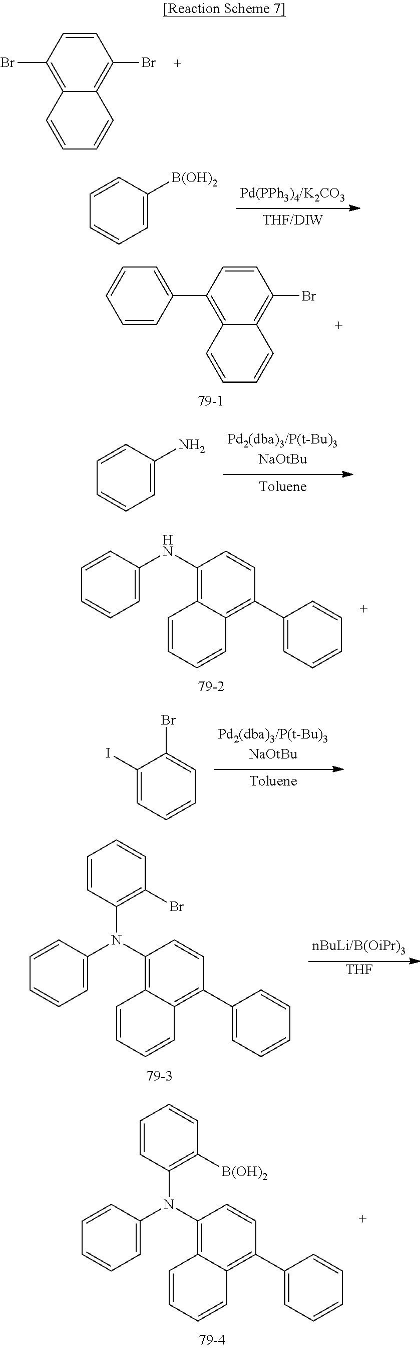

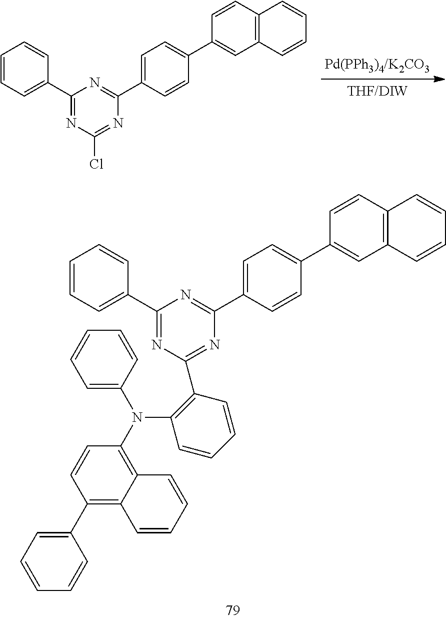 [SMILES: Clc1nc(-c2ccccc2)nc(-c2ccc(-c3ccc4ccccc4c3)cc2)n1 ; c1ccc(-c2nc(-c3ccc(-c4ccc5ccccc5c4)cc3)nc(-c3ccccc3N(c3ccccc3)c3ccc(-c4ccccc4)c4ccccc34)n2)cc1]

a) Synthesis of Intermediate 79-1

Intermediate 79-1 was synthesized in the same synthesis method of Compound 39 of Synthesis Example 1 except that 1-bromo-2,4-difluorobenzene and 2-hydroxyphenylboronic acid were used as a starting material and then, purified through column chromatography.

b) Synthesis of Intermediate 79-2

Intermediate 79-2 was synthesized in the same synthesis method of synthesizing/purifying Intermediate 39-1 of Synthesis Example 1 except that Intermediate 79-1 and aniline were used as a starting material.

c) Synthesis of Intermediate 79-3

Intermediate 79-3 was synthesized in the same synthesis method of synthesizing/purifying Intermediate 39-1 of Synthesis Example 1 except that Intermediate 79-2 and 1-bromo-2-iodobenzene were used as a starting material.

d) Synthesis of Intermediate 79-4

Intermediate 79-4 was synthesized in the same synthesis method of synthesizing/purifying Intermediate 39-2 of Synthesis Example 1 except that Intermediate 79-3 was used as a starting material.

e) Synthesis of Compound 79

Compound 79 was synthesized in the same synthesis method of synthesizing/purifying Compound 39 of Synthesis Example 1 except that Intermediate 79-4 and 2-chloro-4-(4-(naphthalen-2-yl)phenyl)-6-phenyl-1,3,5-triazine were used as a starting material.

LC/MS calculated for: C53H36N4 Exact Mass: 728.29 found for 729.28 [M+H]

### Synthesis Example 8: Synthesis of Compound 80

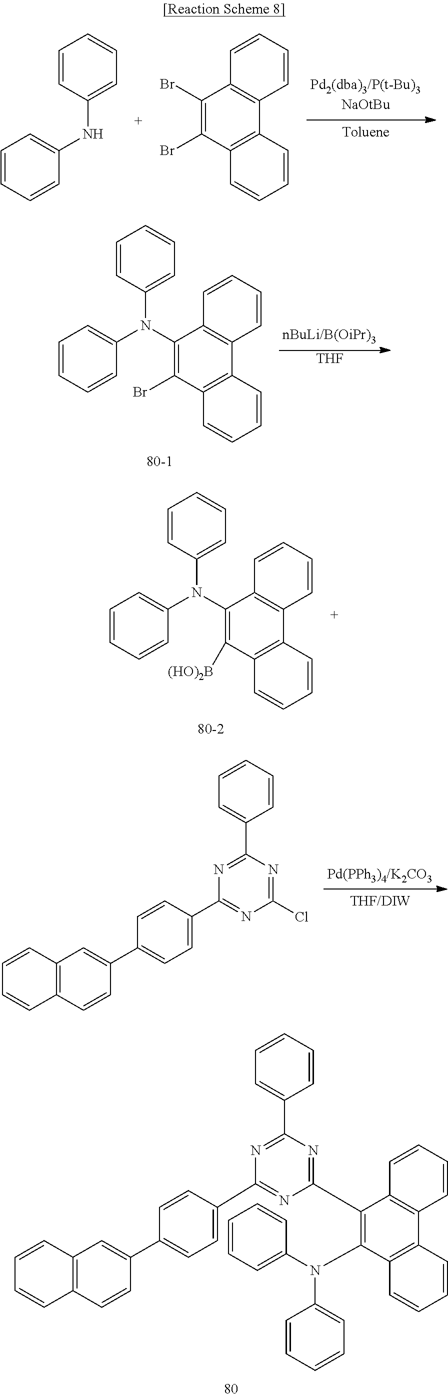

a) Synthesis of Intermediate 80-1

Intermediate 80-1 was synthesized in the same synthesis method of synthesizing/purifying Intermediate 39-1 of Synthesis Example 1 except that diphenylamine and 9,10-dibromophenanthrene were used as a starting material.

b) Synthesis of Intermediate 80-2

Intermediate 80-2 was synthesized in the same synthesis method of synthesizing/purifying Intermediate 39-2 of Synthesis Example 1 except that Intermediate 80-1 was used as a starting material.

c) Synthesis of Compound 80

Compound 80 was synthesized in the same synthesis method of synthesizing/purifying Compound 39 of Synthesis Example 1 except that Intermediate 80-2 and 2-chloro-4-(4-(naphthalen-2-yl)phenyl)-6-phenyl-1,3,5-triazine were used as a starting material.

LC/MS calculated for: C51H34N4 Exact Mass: 702.28 found for 703.34 [M+H]

### Comparative Synthesis Example 1: Synthesis of Compound A1

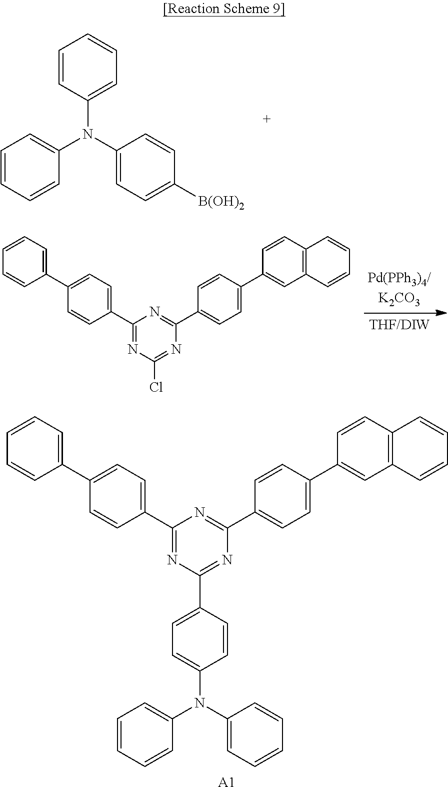

a) Synthesis of Compound A1

Compound A1 was synthesized in the same synthesis method of synthesizing/purifying Compound 39 of Synthesis Example 1 except that (4-(diphenylamino)phenyl)boronic acid and 2-([1,1′-biphenyl]-4-yl)-4-chloro-6-(4-(naphthalen-2-yl)phenyl)-1,3,5-triazine were used as a starting material.

LC/MS calculated for: C49H34N4 Exact Mass: 678.28 found for 679.23 [M+H]

### Comparative Synthesis Example 2: Synthesis of Compound A2

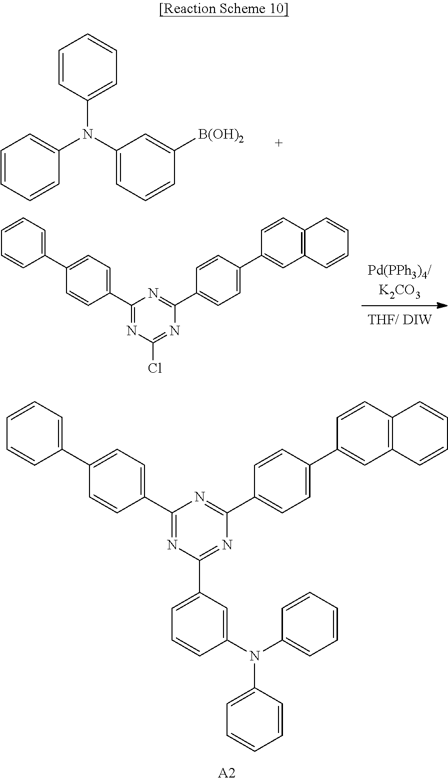

a) Synthesis of Compound A2

Compound A2 was synthesized in the same synthesis method of synthesizing/purifying Compound 39 of Synthesis Example 1 except that (3-(diphenylamino)phenyl)boronic acid and 2-([1,1′-biphenyl]-4-yl)-4-chloro-6-(4-(naphthalen-2-yl)phenyl)-1,3,5-triazine were used as a starting material. LC/MS calculated for: C49H34N4 Exact Mass: 678.28 found for 679.37 [M+H]

### Comparative Synthesis Example 3: Synthesis of Compound A3

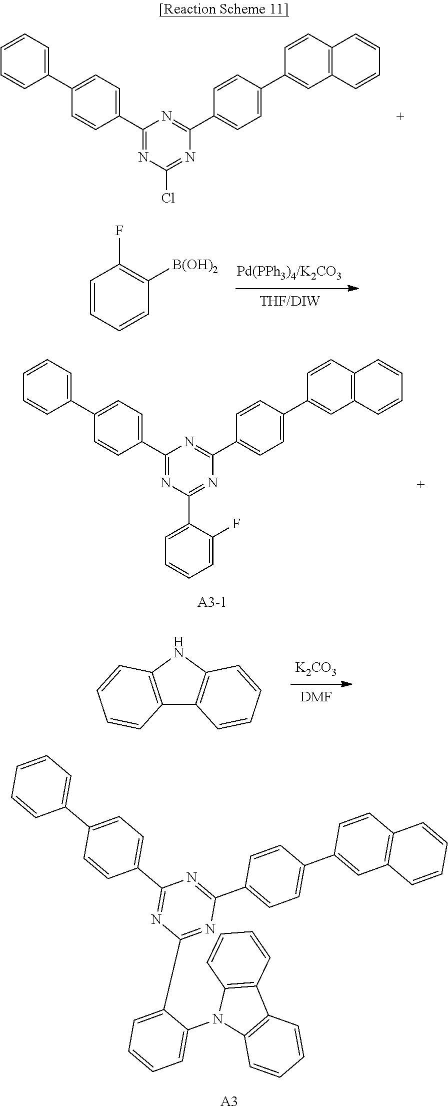

a) Synthesis of Intermediate A3-1

Intermediate A3-1 was synthesized in the same synthesis method of synthesizing/purifying Compound 39 of Synthesis Example 1 except that 2-([1,1′-biphenyl]-4-yl)-4-chloro-6-(4-(naphthalen-2-yl)phenyl)-1,3,5-triazine and 2-fluorophenylboronic acid were used as a starting material.

b) Synthesis of Compound A3

Intermediate A3-1 (10.0 g, 19 mmol), carbazole (3.2 g, 19 mmol), and K₂CO₃ (7.8 g, 57 mmol) were dissolved in 100 mL of N,N-dimethylformamide and then, stirred under reflux at 140° C. for 12 hours. When a reaction was completed, the resultant was recrystallized with toluene, obtaining 11.0 g (85.9%) of Compound A3.

LC/MS calculated for: C49H32N4 Exact Mass: 676.26 found for 677.41 [M+H]

### Synthesis of Second Compound

### Synthesis Example 9: Synthesis of Compound 2-2

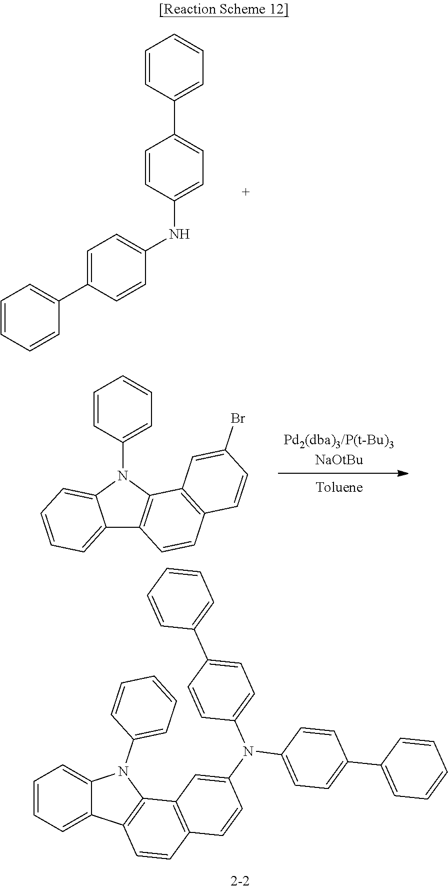

a) Synthesis of Compound 2-2

Compound 2-2 was synthesized in the same synthesis method of synthesizing/purifying Intermediate 39-1 of Synthesis Example 1 except that di([1,1′-biphenyl]-4-yl)amine and 2-bromo-11-phenyl-11H-benzo[a]carbazole were used as a starting material.

### Synthesis Example 10: Synthesis of Compound 2-6

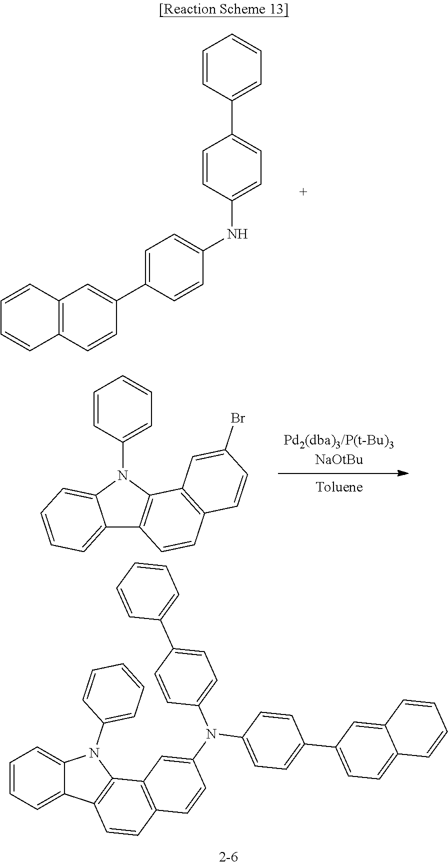

a) Synthesis of Compound 2-6

Compound 2-6 was synthesized in the same synthesis method of synthesizing/purifying Intermediate 39-1 of Synthesis Example 1 except that N-(4-(naphthalen-2-yl)phenyl)-[1,1′-biphenyl]-4-amine and 2-bromo-11-phenyl-11H-benzo[a]carbazole were used as a starting material.

### Synthesis Example 11: Synthesis of Compound 2-63

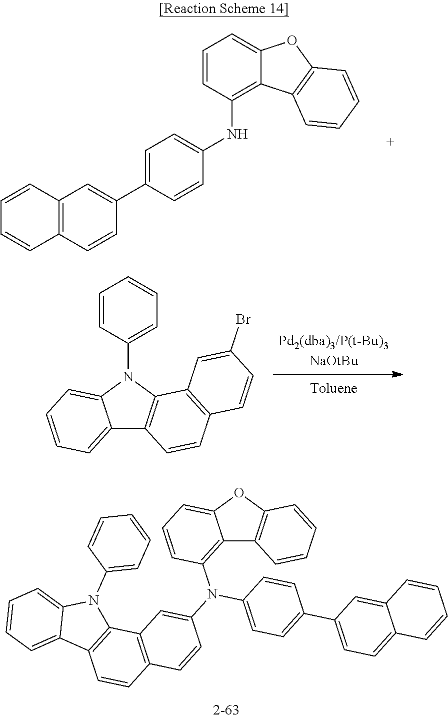

a) Synthesis of Compound 2-63

Compound 2-63 was synthesized in the same synthesis method of synthesizing/purifying Intermediate 39-1 of Synthesis Example 1 except that N-(4-(naphthalen-2-yl)phenyl)dibenzo[b,d]furan-1-amine and 2-bromo-11-phenyl-11H-benzo[a]carbazole were used as a starting material.

### Synthesis of Third Compound

### Synthesis Example 12: Synthesis of Compound 3-16

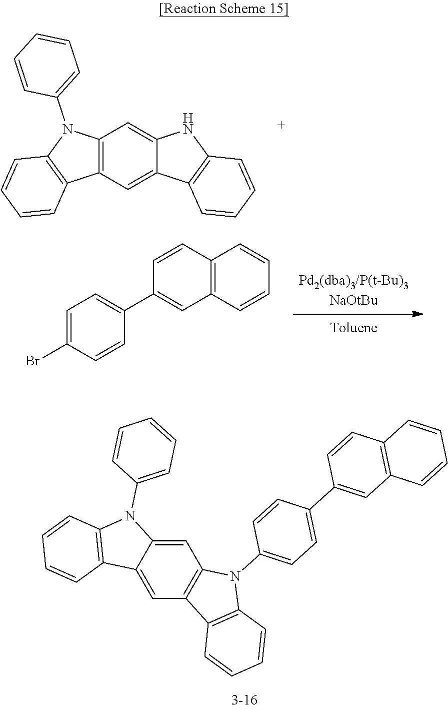

Compound 3-16 was synthesized in the same synthesis method of synthesizing/purifying Intermediate 39-1 of Synthesis Example 1 except that 5-phenyl-5,7-dihydroindolo[2,3-b]carbazole and 2-(4-bromophenyl)naphthalene were used as a starting material.

LC/MS calculated for: C40H26N2 Exact Mass: 534.21 found for 535.69 [M+H]

### Synthesis Example 13: Synthesis of Compound 3-59

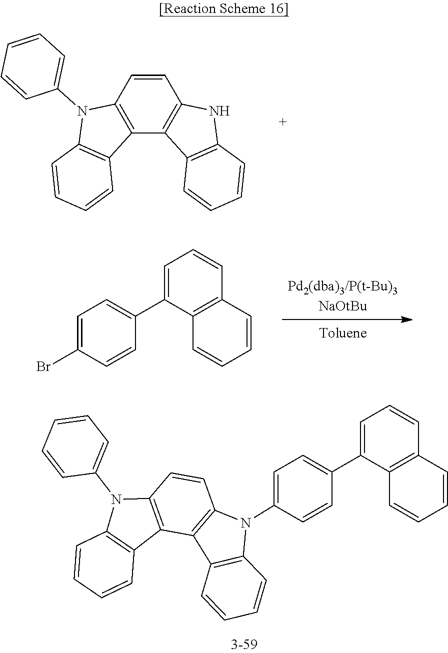

Compound 3-59 was synthesized in the same synthesis method of synthesizing/purifying Intermediate 39-1 of Synthesis Example 1 except that 5-phenyl-5,8-dihydroindolo[2,3-c]carbazole and 1-(4-bromophenyl)naphthalene were used as a starting material.

LC/MS calculated for: C40H26N2 Exact Mass: 534.21 found for 535.01 [M+H]

### Synthesis Example 14: Synthesis of Compound 3-60

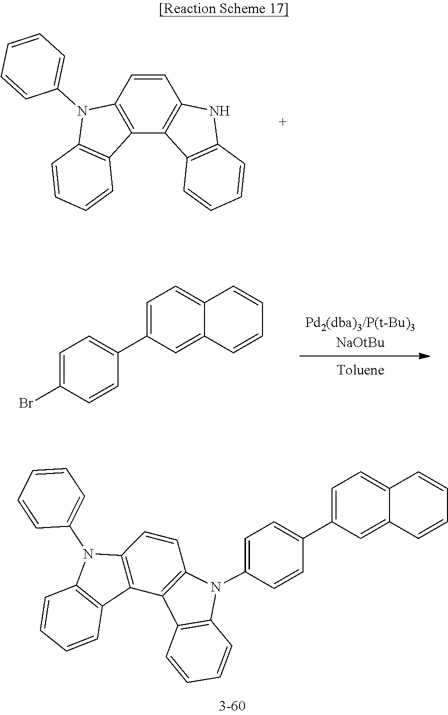 [SMILES: c1ccc(-n2c3ccccc3c3c4c(ccc32)[nH]c2ccccc24)cc1 ; Brc1ccc(-c2ccc3ccccc3c2)cc1 ; c1ccc(-n2c3ccccc3c3c4c5ccccc5n(-c5ccc(-c6ccc7ccccc7c6)cc5)c4ccc32)cc1]

Compound 3-60 was synthesized in the same synthesis method of synthesizing/purifying Intermediate 39-1 of Synthesis Example 1 except that 5-phenyl-5,8-dihydroindolo[2,3-c]carbazole and 2-(4-bromophenyl)naphthalene were used as a starting material.

LC/MS calculated for: C40H26N2 Exact Mass: 534.21 found for 535.12 [M+H]

### Synthesis Example 15: Synthesis of Compound 3-76

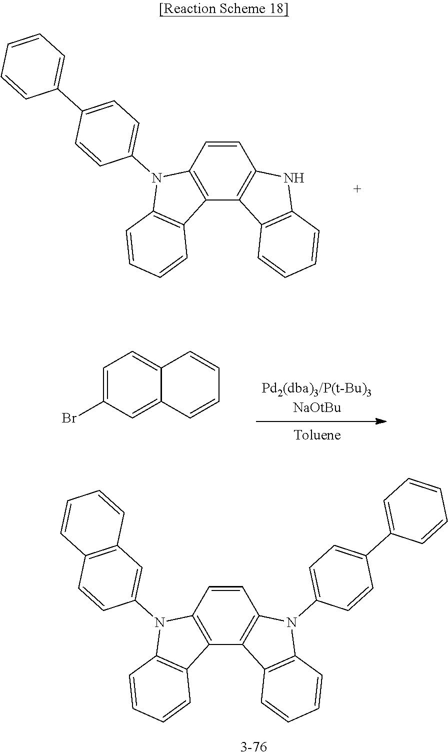 [SMILES: c1ccc(-c2ccc(-n3c4ccccc4c4c5c(ccc43)[nH]c3ccccc35)cc2)cc1 ; Brc1ccc2ccccc2c1 ; c1ccc(-c2ccc(-n3c4ccccc4c4c5c6ccccc6n(-c6ccc7ccccc7c6)c5ccc43)cc2)cc1]

Compound 3-76 was synthesized in the same synthesis method of synthesizing/purifying Intermediate 39-1 of Synthesis Example 1 except that 5-([1,1′-biphenyl]-4-yl)-5,8-dihydroindolo[2,3-c]carbazole and 2-bromon aphthalene were used as a starting material.

LC/MS calculated for: C40H26N2 Exact Mass: 534.21 found for 535.44 [M+H]

### Synthesis Example 16: Synthesis of Compound 3-106

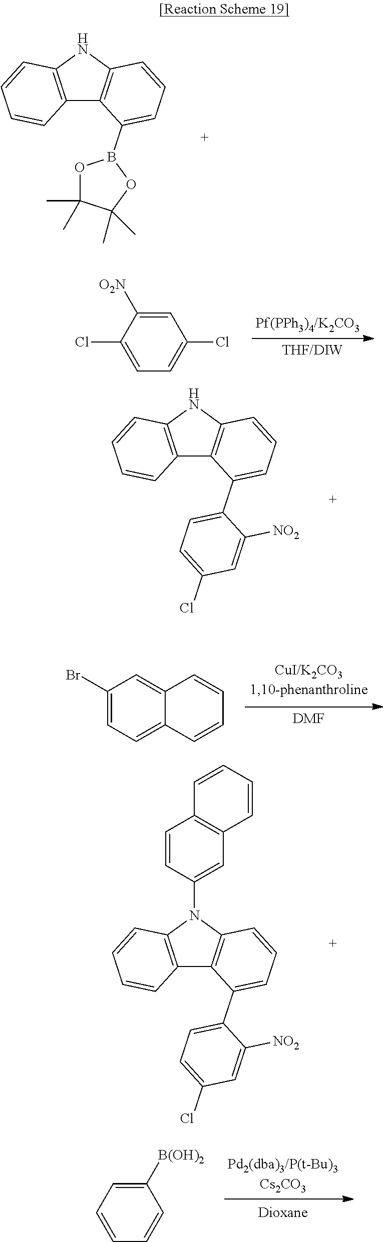

a) Synthesis of Intermediate 3-106-1

Intermediate 3-106-1 was synthesized in the same synthesis method of Compound 39 of Synthesis Example 1 except that 4-(4,4,5,5-tetramethyl-1,3,2-dioxaborolan-2-yl)-9H-carbazole and 1,4-dichloro-2-nitrobenzene were used as a starting material and then, purified through column chromatography.

b) Synthesis of Intermediate 3-106-2

Intermediate 3-106-1 (30.0 g, 93 mmol), 2-bromonaphthalene (23.1 g, 112 mmol), copper iodide (3.5 g, 19 mmol), 1,10-phenanthroline (2.0 g, 11 mmol), and K₂CO₃ (19.3 g, 139 mmol) were dissolved in 400 mL of N,N-dimethylformamide and then, stirred under reflux at 140° C. for 12 hours. When a reaction was completed, the resultant was recrystallized with toluene, obtaining 28.0 g (67.2%) of Intermediate 3-106-2.

c) Synthesis of Intermediate 3-106-3

Intermediate 3-106-2 (28.0 g, 62 mmol), phenylboronic acid (9.1 g, 75 mmol), Pd₂(dba)₃ (1.7 g, 1.9 mmol), a tri tert-butylphosphine solution (1.1 g, 6 mmol), and Cs₂CO₃ (40.6 g, 125 mmol) were dissolved in 200 mL of dioxane and then, stirred under reflux at 110° C. for 12 hours. When a reaction was completed, the resultant was recrystallized with toluene, obtaining 27.0 g (88.2%) of Intermediate 3-106-3.

d) Synthesis of Intermediate 3-106-4

Intermediate 3-106-3 (27.0 g, 55 mmol) and triphenylphosphine (43.3 g, 165 mmol) were dissolved in 250 mL of 1,2-dichlorobenzene and then, stirred under reflux at 180° C. for 12 hours. When a reaction was completed, the resultant was recrystallized with toluene, obtaining 16.4 g (65.0%) of Intermediate 3-106-4.

e) Synthesis of Compound 3-106

Compound 3-106 was synthesized in the same synthesis method of synthesizing/purifying Intermediate 39-1 of Synthesis Example 1 except that Intermediate 3-106-4 and bromobenzene were used as a starting material.

LC/MS calculated for: C40H26N2 Exact Mass: 534.21 found for 535.27 [M+H]

### Synthesis Example 17: Synthesis of Compound 3-147

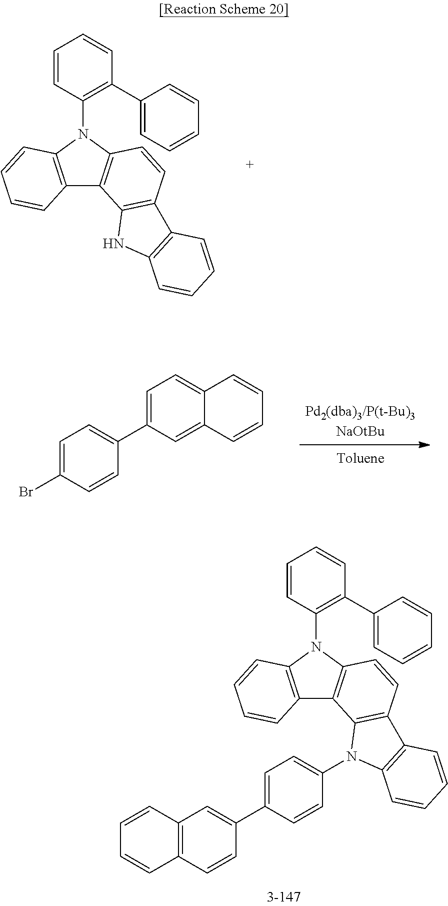 [SMILES: c1ccc(-c2ccccc2-n2c3ccccc3c3c4[nH]c5ccccc5c4ccc32)cc1 ; Brc1ccc(-c2ccc3ccccc3c2)cc1 ; c1ccc(-c2ccccc2-n2c3ccccc3c3c2ccc2c4ccccc4n(-c4ccc(-c5ccc6ccccc6c5)cc4)c23)cc1]

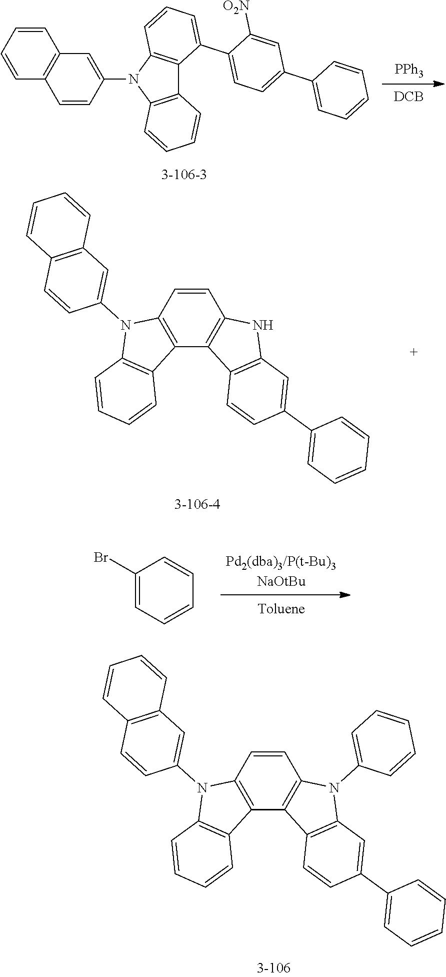

Compound 3-147 was synthesized in the same synthesis method of synthesizing/purifying Intermediate 39-1 of Synthesis Example 1 except that 5-([1,1′-biphenyl]-2-yl)-5,12-dihydroindolo[3,2-a]carbazole and 2-(4-bromophenyl)naphthalene were used as a starting material.

LC/MS calculated for: C46H30N2 Exact Mass: 610.24 found for 611.25 [M+H]

### (Manufacture of Organic Light Emitting Diode)

### Example 1

The glass substrate coated with ITO (Indium tin oxide) at a thickness of 1,500 Å was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (commercially available from Novaled) was vacuum-deposited on the ITO substrate to form a 100 Å-thick hole injection layer, and then Compound A was deposited to be 1300 Å-thick to form a hole transport layer. Compound B was deposited on the hole transport layer to a 700 Å-thick hole transport auxiliary layer. On the hole transport auxiliary layer, a 400 Å-thick light emitting layer was formed by simultaneously vacuum-depositing Compound 39 obtained in Synthesis Example 1 and Compound 2-2 obtained in Synthesis Example 9 as a host and doping 2 wt % of [Ir(piq)₂acac] as a dopant. Compound 39 and Compound 2-2 were used in a weight ratio of 5:5. Subsequently, on the light emitting layer, Compound C was deposited to a thickness of 50 Å to form an electron transport auxiliary layer, and a 300 Å-thick electron transport layer was formed by simultaneously vacuum-depositing Compound D and LiQ in a weight ratio of 1:1. On the electron transport layer, LiQ and Al were sequentially vacuum-deposited to be 15 Å thick and 1200 Å thick, manufacturing an organic light emitting diode having the following structure.

A structure of ITO/Compound A (3% NDP-9 doping, 100 Å)/Compound A (1300 Å) /Compound B (700 Å)/EML [Compound 39 (50%): Compound 2-2 (50%): [Ir(piq)₂acac] (2 wt %)] (400 Å)/Compound C (50 Å)/Compound D: Liq (300 Å)/LiQ (15 Å)/Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-di([1,1′-biphenyl]-4-yl)-7,7-dimethyl-7H-fluoreno[4,3-b]benzofuran-10-amine

Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinoline

### Examples 2 to 56 and Comparative Examples 1 to 6

Diodes of Examples 2 to 56 and Comparative Examples 1 to 6 were manufactured in the same manner as in Example 1, except that the host was changed as shown in Table 1 and Table 2.

### Evaluation

The characteristics of the organic light emitting diodes according to Examples 1 to 56 and Comparative Examples 1 to 6 were evaluated, and the results are shown in Tables 1 and 2. Specific measurement methods are as follows.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000 Å), while the voltage of the organic light emitting diodes was increased from 0 V to 10 V.

(3) Measurement of Luminous Efficiency

The luminous efficiency (cd/A) of the same current density (10 mA/cm²) was calculated using the luminance and current density measured from (1) and (2) above.

(4) T90 Measurement of Life-Span

The results were obtained by measuring a time when current efficiency (cd/A) was decreased down to 97%, while luminance (cd/m²) was maintained to be 6,000 cd/m².

(5) Measurement of Driving Voltage

The driving voltage and threshold voltage of each diode were measured at 15 mA/cm² using a current-voltmeter (Keithley 2400), and the results were obtained.

(6) Calculation of Driving Voltage Ratio (%)

The relative comparison values with the measured driving voltage values of Comparative Example 3 are shown in Table 1.

The relative comparison values with the measured values of the driving voltage of Comparative Example 6 are shown in Table 2.

(7) Calculation of Luminous Efficiency Ratio (%)

The relative comparison values with the luminous efficiency measurement values of Comparative Example 3 are shown in Table 1.

The relative comparison values with the luminous efficiency measurement values of Comparative Example 6 are shown in Table 2.

(8) Calculation of Life-span Ratio (%)

The relative comparison value with the T90(h) life-span measurement value of Comparative Example 3 are shown in Table 1.

The relative comparison value with the T90(h) life-span measurement value of Comparative Example 6 are shown in Table 2.

TABLE 1

| | | | Driving | Luminous | T90 |
|---|---|---|---|---|---|
| | | | voltage | efficiency | life-span |
| | First | Second | ratio | ratio | ratio |
| | host | host | (%) | (%) | (%) |
| Ex. 1 | 39 | 2-2 | 92 | 107 | 110 |
| Ex. 2 | 39 | 2-6 | 92 | 107 | 140 |
| Ex. 3 | 39 | 2-63 | 93 | 108 | 135 |
| Ex. 4 | 43 | 2-2 | 92 | 104 | 180 |
| Ex. 5 | 43 | 2-6 | 92 | 104 | 210 |
| Ex. 6 | 43 | 2-63 | 93 | 106 | 200 |
| Ex. 7 | 66 | 2-2 | 91 | 102 | 130 |
| Ex. 8 | 66 | 2-6 | 91 | 103 | 140 |
| Ex. 9 | 66 | 2-63 | 93 | 104 | 130 |
| Ex. 10 | 67 | 2-2 | 91 | 102 | 120 |
| Ex. 11 | 67 | 2-6 | 91 | 103 | 140 |
| Ex. 12 | 67 | 2-63 | 93 | 104 | 130 |
| Ex. 13 | 76 | 2-2 | 94 | 106 | 105 |
| Ex. 14 | 76 | 2-6 | 94 | 106 | 110 |
| Ex. 15 | 76 | 2-63 | 94 | 107 | 105 |
| Ex. 16 | 78 | 2-2 | 93 | 104 | 130 |
| Ex. 17 | 78 | 2-6 | 93 | 105 | 140 |
| Ex. 18 | 78 | 2-63 | 94 | 106 | 130 |
| Ex. 19 | 79 | 2-2 | 97 | 102 | 110 |
| Ex. 20 | 79 | 2-6 | 96 | 103 | 120 |
| Ex. 21 | 79 | 2-63 | 97 | 105 | 110 |
| Ex. 22 | 80 | 2-2 | 92 | 106 | 110 |
| Ex. 23 | 80 | 2-6 | 92 | 106 | 120 |
| Ex. 24 | 80 | 2-63 | 93 | 107 | 110 |
| Comp. Ex. 1 | A1 | 2-6 | 110 | 80 | 1 |
| Comp. Ex. 2 | A2 | 2-6 | 108 | 100 | 15 |
| Comp. Ex. 3 | A3 | 2-6 | 100 | 100 | 100 |

Referring to Table 1, the compounds according to the present invention exhibited an increased threshold voltage and significantly improved life-span compared to the comparative compounds.

TABLE 2

| | | | Driving | Luminous | T90 |
|---|---|---|---|---|---|
| | | | voltage | efficiency | life-span |
| | First | Second | ratio | ratio | ratio |
| | host | host | (%) | (%) | (%) |
| Ex. 25 | 39 | 3-16 | 94 | 110 | 110 |
| Ex. 26 | 39 | 3-59 | 90 | 112 | 120 |
| Ex. 27 | 39 | 3-60 | 90 | 111 | 140 |
| Ex. 28 | 39 | 3-76 | 90 | 111 | 140 |
| Ex. 29 | 39 | 3-106 | 91 | 112 | 145 |
| Ex. 30 | 39 | 3-147 | 94 | 110 | 110 |
| Ex. 31 | 43 | 3-16 | 94 | 110 | 115 |
| Ex. 32 | 43 | 3-59 | 90 | 111 | 140 |
| Ex. 33 | 43 | 3-60 | 91 | 110 | 150 |
| Ex. 34 | 43 | 3-76 | 90 | 110 | 150 |
| Ex. 35 | 43 | 3-106 | 91 | 111 | 170 |
| Ex. 36 | 43 | 3-147 | 94 | 110 | 110 |
| Ex. 37 | 66 | 3-59 | 90 | 108 | 120 |
| Ex. 38 | 66 | 3-60 | 91 | 108 | 140 |
| Ex. 39 | 66 | 3-76 | 91 | 108 | 140 |
| Ex. 40 | 66 | 3-106 | 91 | 109 | 150 |
| Ex. 41 | 67 | 3-59 | 92 | 107 | 120 |
| Ex. 42 | 67 | 3-60 | 92 | 108 | 140 |
| Ex. 43 | 67 | 3-76 | 92 | 108 | 140 |
| Ex. 44 | 67 | 3-106 | 92 | 109 | 150 |
| Ex. 45 | 76 | 3-16 | 93 | 106 | 110 |
| Ex. 46 | 76 | 3-60 | 93 | 107 | 130 |
| Ex. 47 | 76 | 3-76 | 92 | 107 | 130 |
| Ex. 48 | 76 | 3-106 | 92 | 107 | 140 |
| Ex. 49 | 78 | 3-60 | 93 | 108 | 130 |
| Ex. 50 | 78 | 3-76 | 93 | 108 | 130 |
| Ex. 51 | 78 | 3-106 | 93 | 108 | 125 |
| Ex. 52 | 78 | 3-147 | 96 | 107 | 110 |
| Ex. 53 | 79 | 3-60 | 94 | 107 | 110 |
| Ex. 54 | 79 | 3-106 | 94 | 108 | 110 |
| Ex. 55 | 80 | 3-60 | 91 | 110 | 120 |
| Ex. 56 | 80 | 3-106 | 91 | 111 | 125 |
| Comp. Ex. 4 | A1 | 3-60 | 110 | 80 | 1 |
| Comp. Ex. 5 | A2 | 3-60 | 103 | 96 | 5 |
| Comp. Ex. 6 | A3 | 3-60 | 100 | 100 | 100 |

Referring to Table 2, the compounds according to the present invention exhibited significantly improved driving voltage, luminous efficiency, and life-span compared to the comparative compounds.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, the composition comprising:
a first compound represented by Chemical Formula 1 and a second compound represented by Chemical Formula 2, or
a first compound represented by Chemical Formula 1 and a third compound represented by a combination of Chemical Formula 3 and Chemical Formula 4: 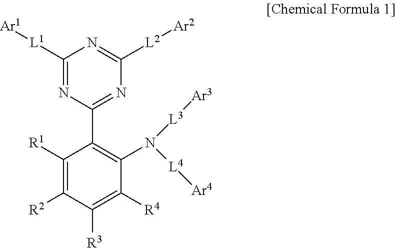
wherein, in Chemical Formula 1,
L¹ to L⁴ are each independently a single bond, a substituted or unsubstituted C6 to C30 arylene group, or a substituted or unsubstituted C2 to C30 heteroarylene group,
Ar¹ to Ar⁴ are each independently a substituted or unsubstituted C6 to C30 aryl group, or a substituted or unsubstituted C2 to C30 heterocyclic group,
R¹ to R⁴ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, and
R¹ to R⁴ are separately present or adjacent groups are linked to each other to form a substituted or unsubstituted aromatic monocyclic ring, aromatic polycyclic ring, heteroaromatic monocyclic ring, or heteroaromatic polycyclic ring; 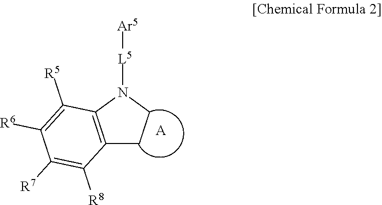
wherein, in Chemical Formula 2,
L⁵ is a single bond, or a substituted or unsubstituted C6 to C30 arylene group,
Ar⁵ is a substituted or unsubstituted C6 to C30 aryl group, or a substituted or unsubstituted C2 to C30 heterocyclic group, and
A is a ring of Group I, in which * is a linking carbon, 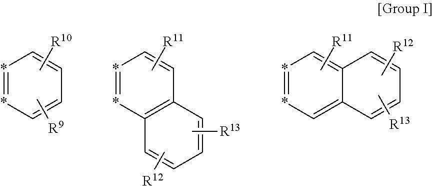
R⁵ to R¹³ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
at least one of R⁵ to R¹³ is a substituted amine group represented by Chemical Formula a, 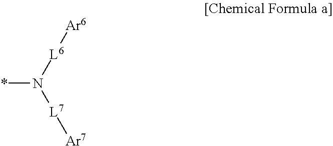
wherein, in Chemical Formula a,
L⁶ and L⁷ are each independently a single bond, or a substituted or unsubstituted C6 to C30 arylene group,
Ar⁶ and Ar⁷ are each independently a substituted or unsubstituted C6 to C30 aryl group, or a substituted or unsubstituted C2 to C30 heterocyclic group, and
* is a linking point; 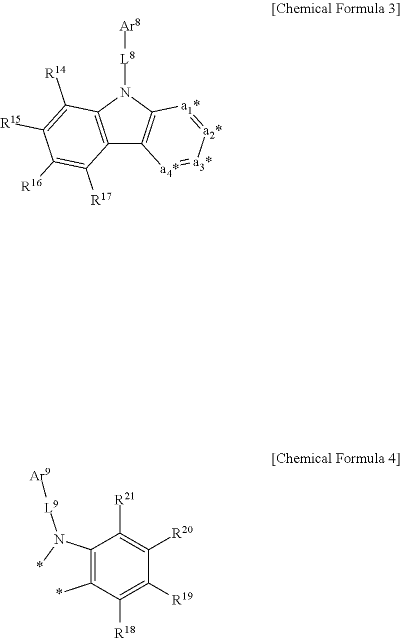
wherein, in Chemical Formula 3 and Chemical Formula 4,
adjacent two of a1* to a4* of Chemical Formula 3 are each independently a linking carbon (C) linked a * of Chemical Formula 4,
the remaining two of a1* to a4* of Chemical Formula 3, not linked at * of Chemical Formula 4 are CR^{a},
R^{a} and R¹⁴ to R²¹ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
L⁸ and L⁹ are each independently a single bond, a substituted or unsubstituted C6 to C30 arylene group, or a substituted or unsubstituted C2 to C30 heteroarylene group, and
Ar⁸ and Ar⁹ are each independently a substituted or unsubstituted C6 to C30 aryl group, or a substituted or unsubstituted C2 to C30 heterocyclic group.

2. The composition for an organic optoelectronic device of claim 1, wherein:
the first compound is represented by one of Chemical Formula 1-1 to Chemical Formula 1-4: 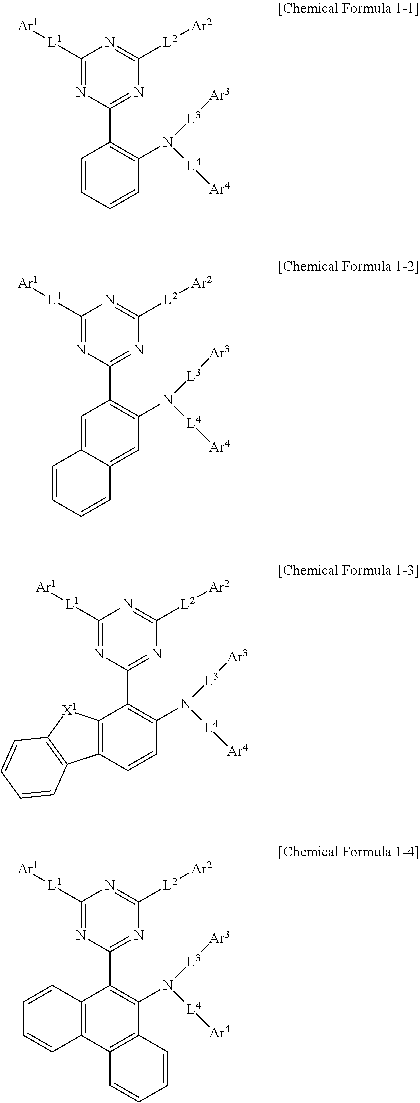
in Chemical Formula 1-1 to Chemical Formula 1-4,
Ar¹ to Ar⁴ and L¹ to L⁴ are defined the same as those of Chemical Formula 1, and
X¹ is O or S.

3. The composition for an organic optoelectronic device of claim 1, wherein L¹ to L⁴ of Chemical Formula 1 are each independently a single bond, a substituted or unsubstituted phenylene group, or a substituted or unsubstituted naphthylene group.

4. The composition for an organic optoelectronic device of claim 1, wherein Ar¹ and Ar² of Chemical Formula 1 are each independently a substituted or unsubstituted phenyl group, a substituted or unsubstituted biphenyl group, a substituted or unsubstituted naphthyl group, a substituted or unsubstituted phenanthrenyl group, a substituted or unsubstituted triphenylene group, a substituted or unsubstituted chrysenyl group, a substituted or unsubstituted dibenzofuranyl group, a substituted or unsubstituted dibenzothiophenyl group, or a substituted or unsubstituted dibenzosilolyl group.

5. The composition for an organic optoelectronic device of claim 1, wherein:
moieties *-L¹-Ar¹ and *-L²-Ar² of Chemical Formula 1 are each independently a moiety of Group II: 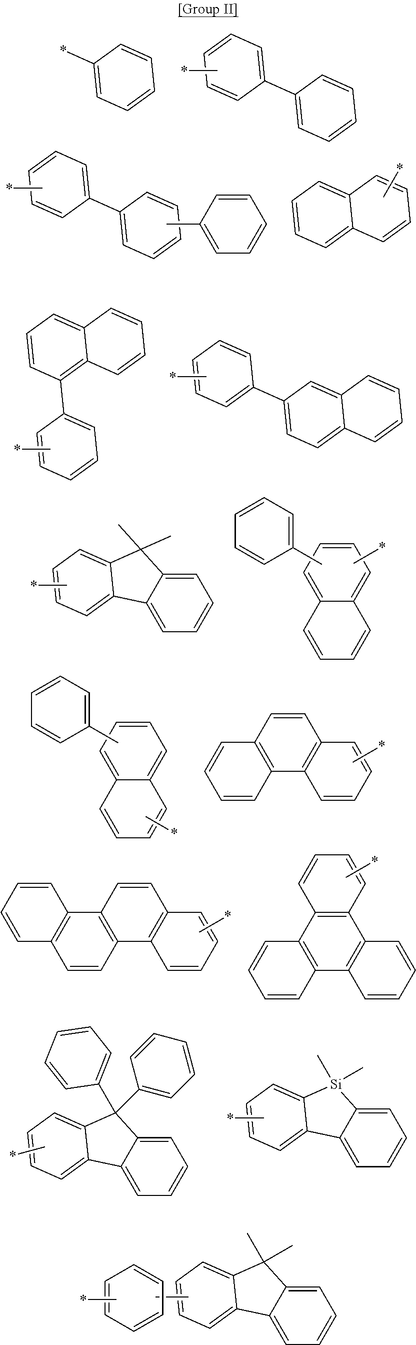 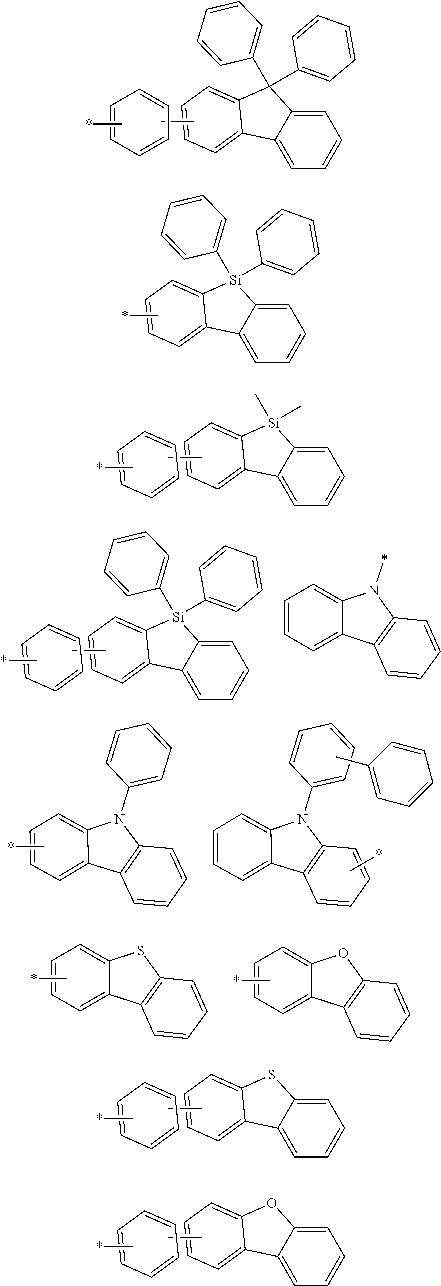
in Group I, * is a linking point.

6. The composition for an organic optoelectronic device of claim 1, wherein:
moieties *-L³-Ar³ and *-L⁴-Ar⁴ of Chemical Formula 1 are each independently a moiety of Group III: 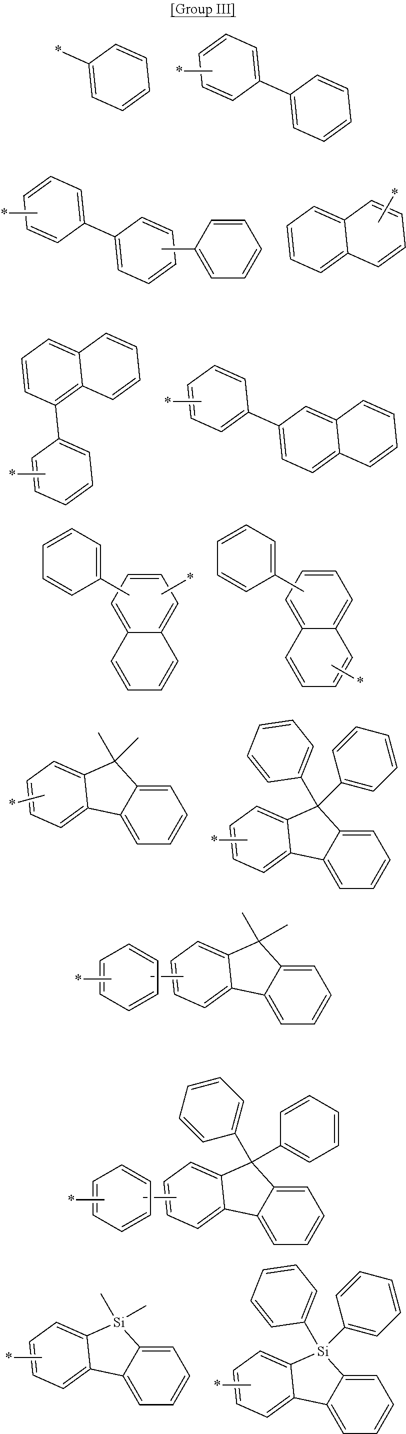 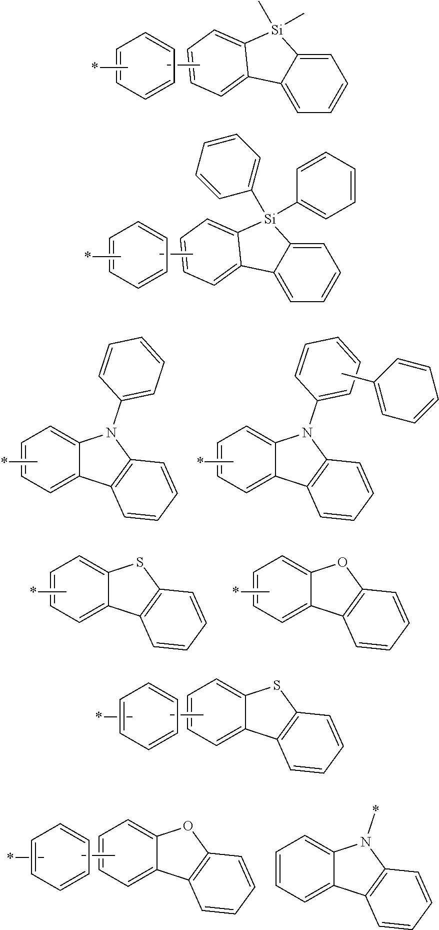
in Group III, * is a linking point.

7. The composition for an organic optoelectronic device of claim 1, wherein the first compound is a compound of Group 1:

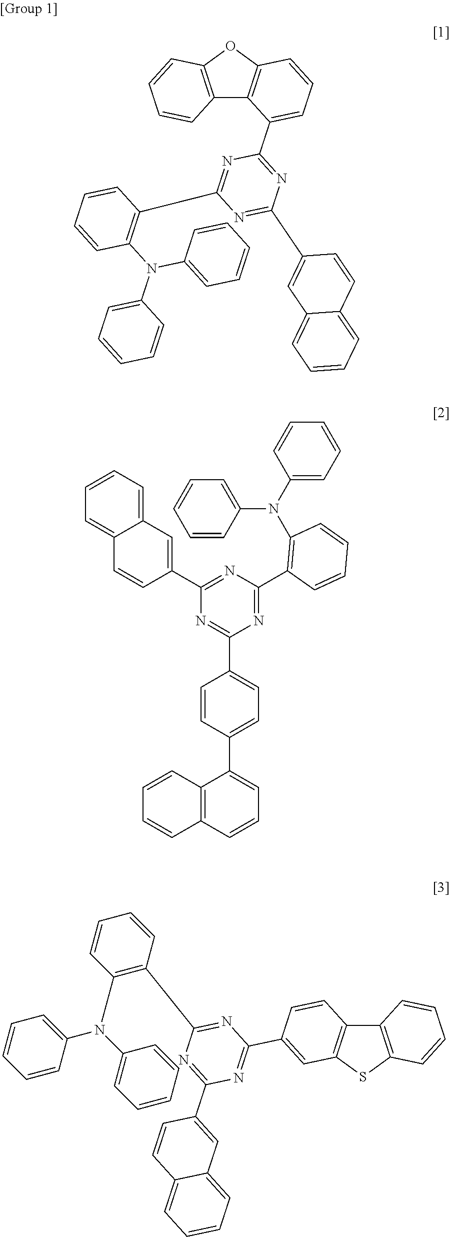 [SMILES: c1ccc(N(c2ccccc2)c2ccccc2-c2nc(-c3ccc4ccccc4c3)nc(-c3cccc4oc5ccccc5c34)n2)cc1 ; c1ccc(N(c2ccccc2)c2ccccc2-c2nc(-c3ccc(-c4cccc5ccccc45)cc3)nc(-c3ccc4ccccc4c3)n2)cc1 ; c1ccc(N(c2ccccc2)c2ccccc2-c2nc(-c3ccc4ccccc4c3)nc(-c3ccc4c(c3)sc3ccccc34)n2)cc1]

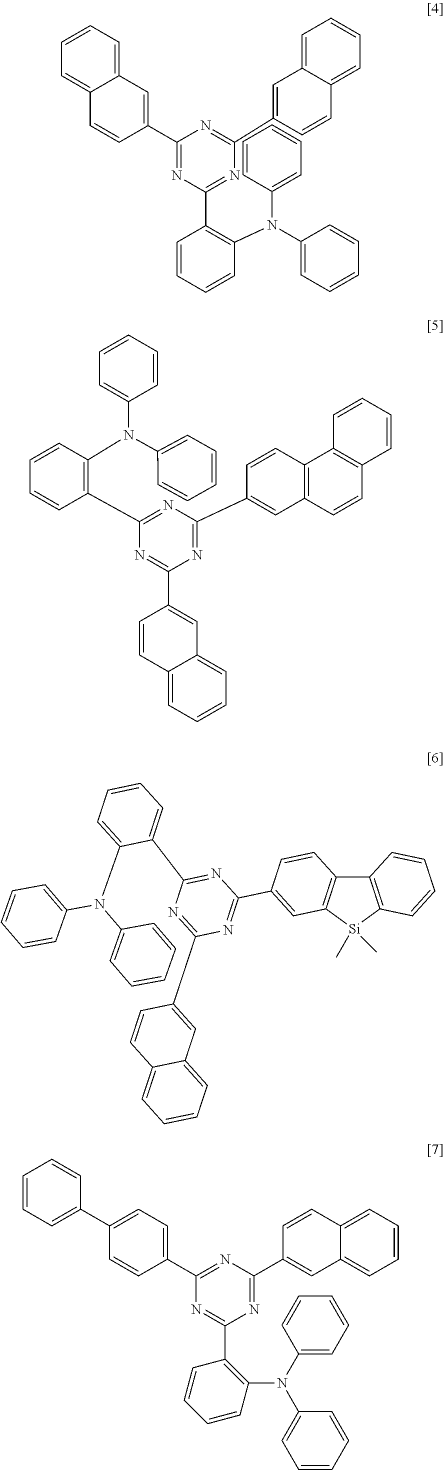 [SMILES: c1ccc(N(c2ccccc2)c2ccccc2-c2nc(-c3ccc4ccccc4c3)nc(-c3ccc4ccccc4c3)n2)cc1 ; c1ccc(N(c2ccccc2)c2ccccc2-c2nc(-c3ccc4ccccc4c3)nc(-c3ccc4c(ccc5ccccc54)c3)n2)cc1 ; C[Si]1(C)c2ccccc2-c2ccc(-c3nc(-c4ccc5ccccc5c4)nc(-c4ccccc4N(c4ccccc4)c4ccccc4)n3)cc21 ; c1ccc(-c2ccc(-c3nc(-c4ccc5ccccc5c4)nc(-c4ccccc4N(c4ccccc4)c4ccccc4)n3)cc2)cc1]

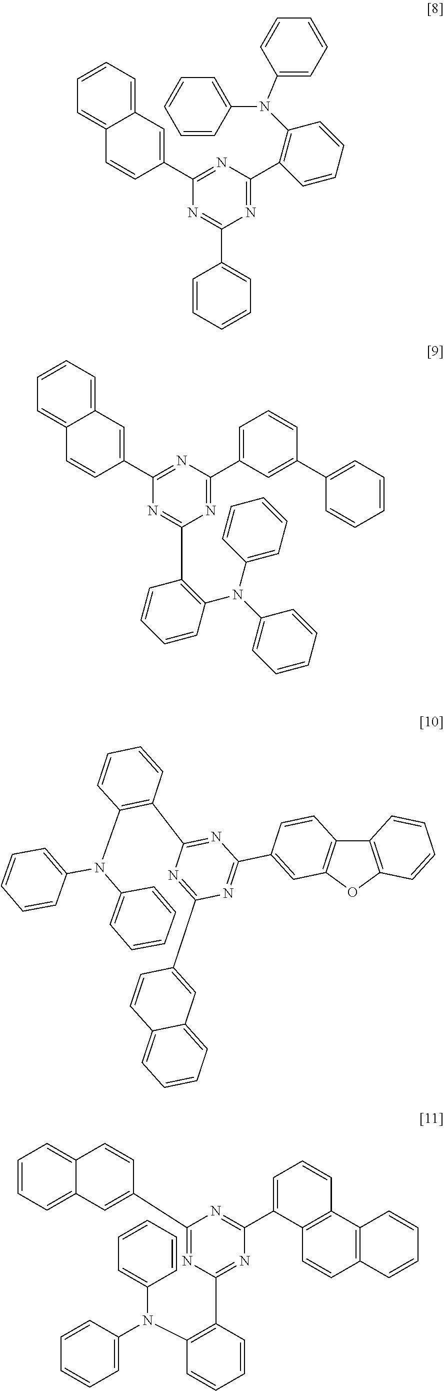 [SMILES: c1ccc(-c2nc(-c3ccc4ccccc4c3)nc(-c3ccccc3N(c3ccccc3)c3ccccc3)n2)cc1 ; c1ccc(-c2cccc(-c3nc(-c4ccc5ccccc5c4)nc(-c4ccccc4N(c4ccccc4)c4ccccc4)n3)c2)cc1 ; c1ccc(N(c2ccccc2)c2ccccc2-c2nc(-c3ccc4ccccc4c3)nc(-c3ccc4c(c3)oc3ccccc34)n2)cc1 ; c1ccc(N(c2ccccc2)c2ccccc2-c2nc(-c3ccc4ccccc4c3)nc(-c3cccc4c3ccc3ccccc34)n2)cc1]

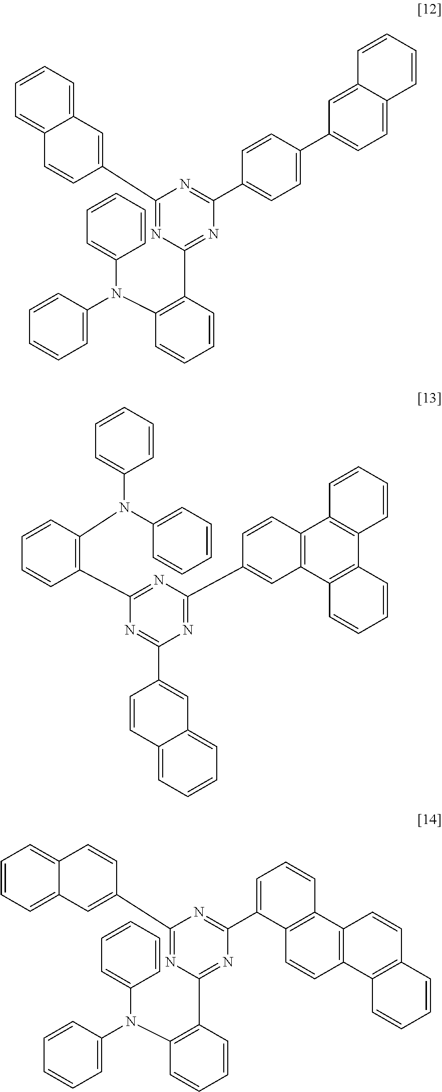 [SMILES: c1ccc(N(c2ccccc2)c2ccccc2-c2nc(-c3ccc(-c4ccc5ccccc5c4)cc3)nc(-c3ccc4ccccc4c3)n2)cc1 ; c1ccc(N(c2ccccc2)c2ccccc2-c2nc(-c3ccc4ccccc4c3)nc(-c3ccc4c5ccccc5c5ccccc5c4c3)n2)cc1 ; c1ccc(N(c2ccccc2)c2ccccc2-c2nc(-c3ccc4ccccc4c3)nc(-c3cccc4c3ccc3c5ccccc5ccc43)n2)cc1]

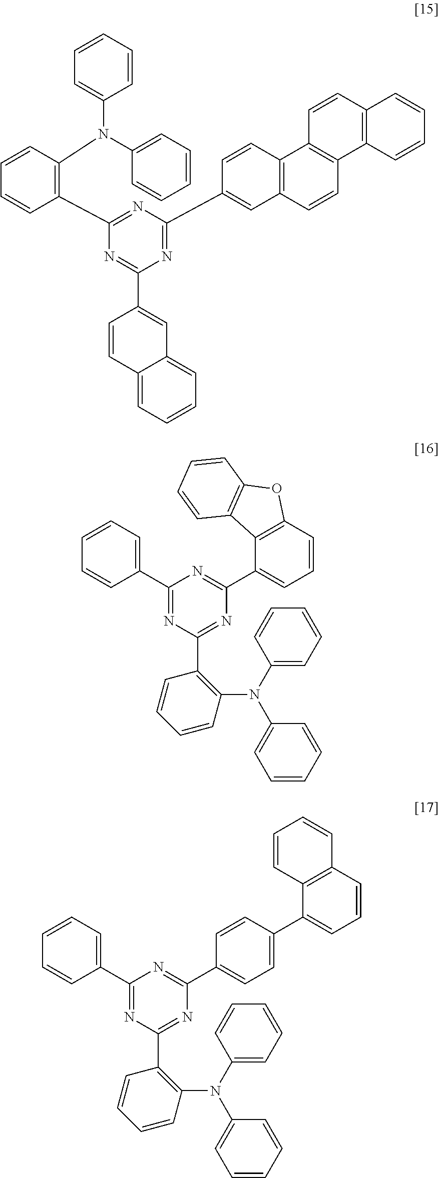

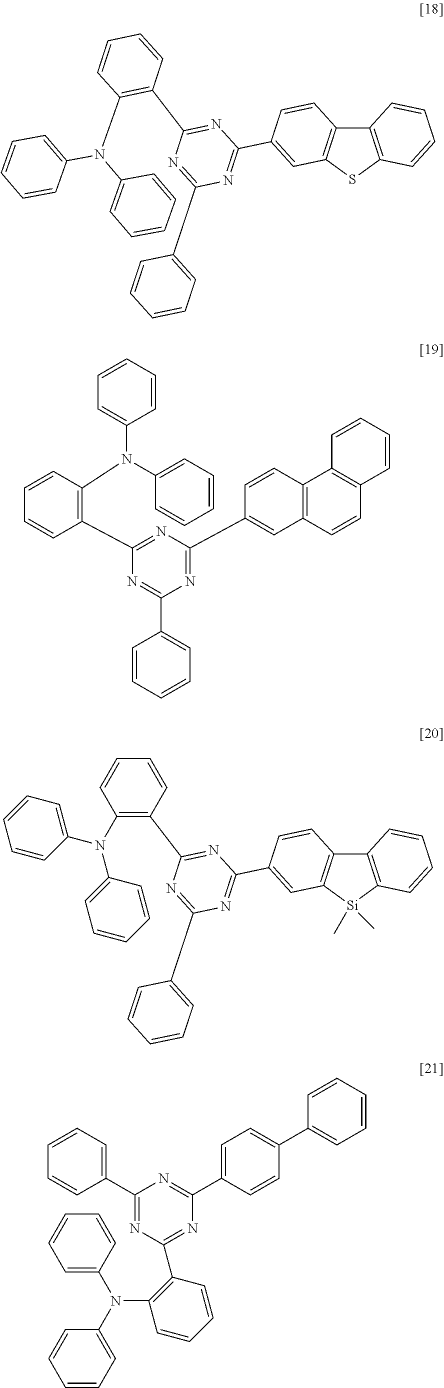 [SMILES: c1ccc(-c2nc(-c3ccc4c(c3)sc3ccccc34)nc(-c3ccccc3N(c3ccccc3)c3ccccc3)n2)cc1 ; c1ccc(-c2nc(-c3ccc4c(ccc5ccccc54)c3)nc(-c3ccccc3N(c3ccccc3)c3ccccc3)n2)cc1 ; C[Si]1(C)c2ccccc2-c2ccc(-c3nc(-c4ccccc4)nc(-c4ccccc4N(c4ccccc4)c4ccccc4)n3)cc21 ; c1ccc(-c2ccc(-c3nc(-c4ccccc4)nc(-c4ccccc4N(c4ccccc4)c4ccccc4)n3)cc2)cc1]

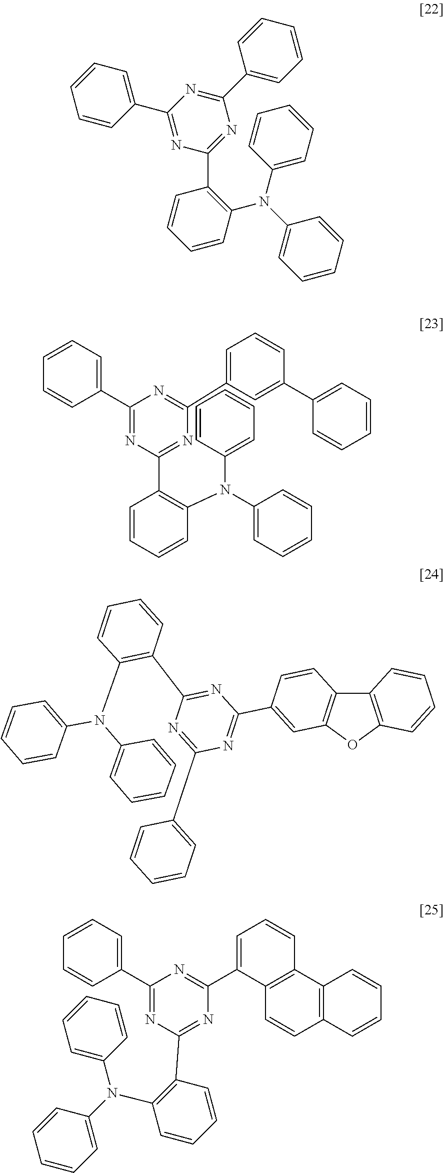

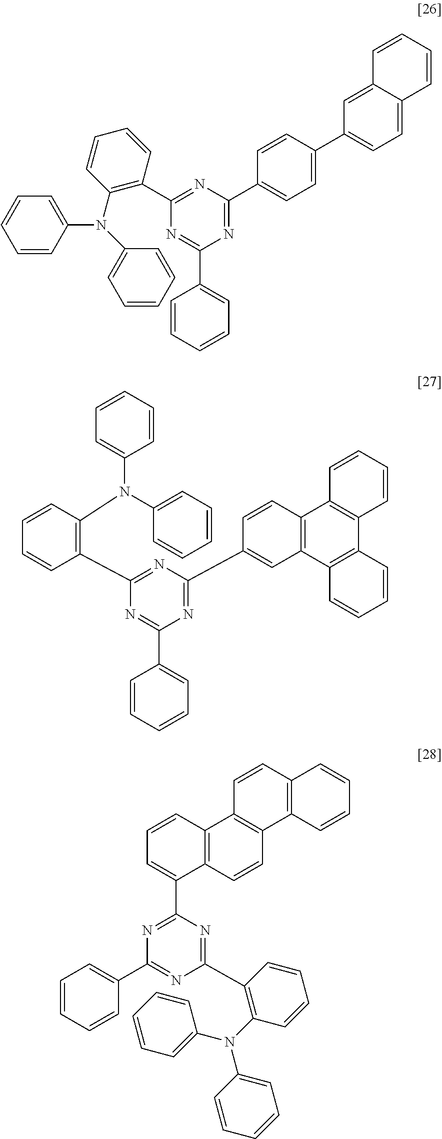 [SMILES: c1ccc(-c2nc(-c3ccc(-c4ccc5ccccc5c4)cc3)nc(-c3ccccc3N(c3ccccc3)c3ccccc3)n2)cc1 ; c1ccc(-c2nc(-c3ccc4c5ccccc5c5ccccc5c4c3)nc(-c3ccccc3N(c3ccccc3)c3ccccc3)n2)cc1 ; c1ccc(-c2nc(-c3ccccc3N(c3ccccc3)c3ccccc3)nc(-c3cccc4c3ccc3c5ccccc5ccc43)n2)cc1]

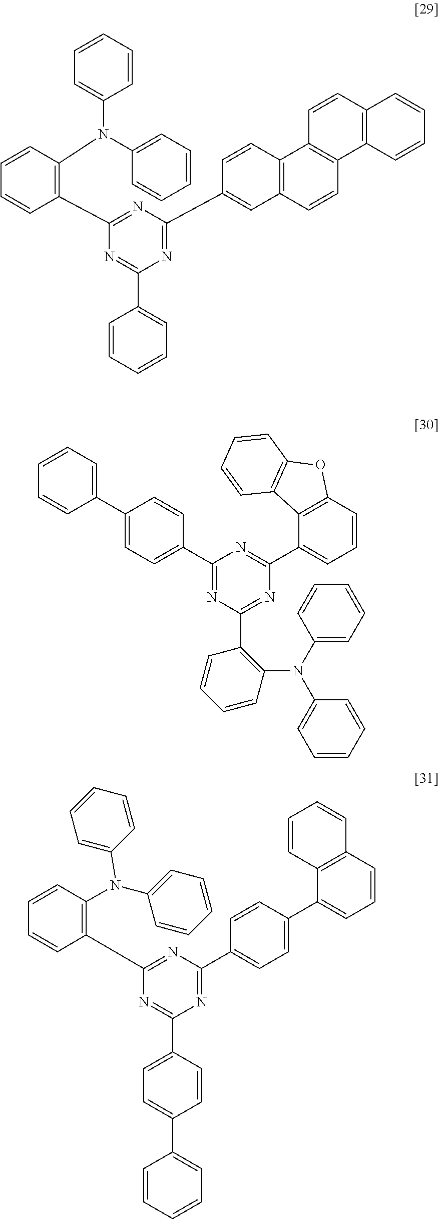

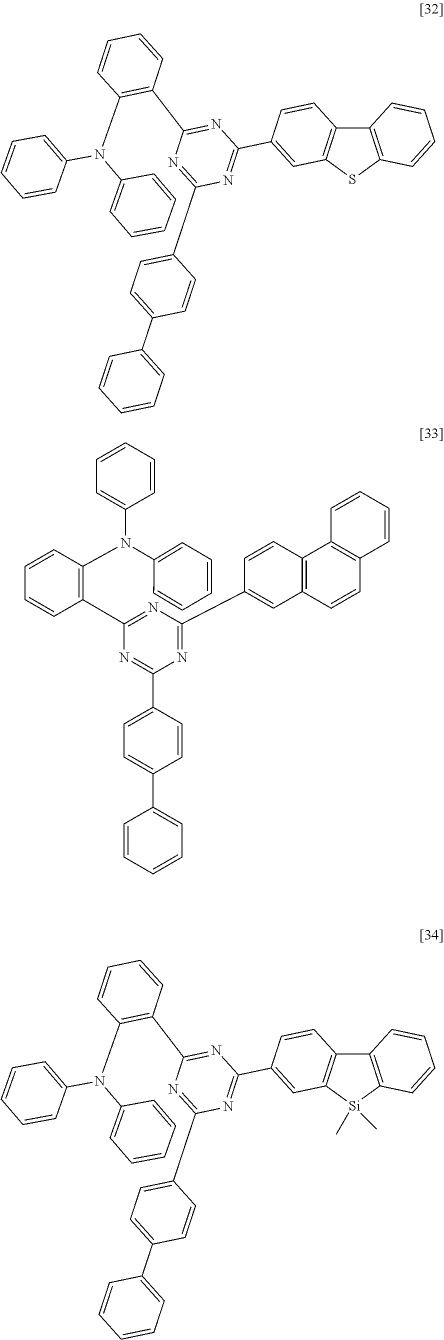

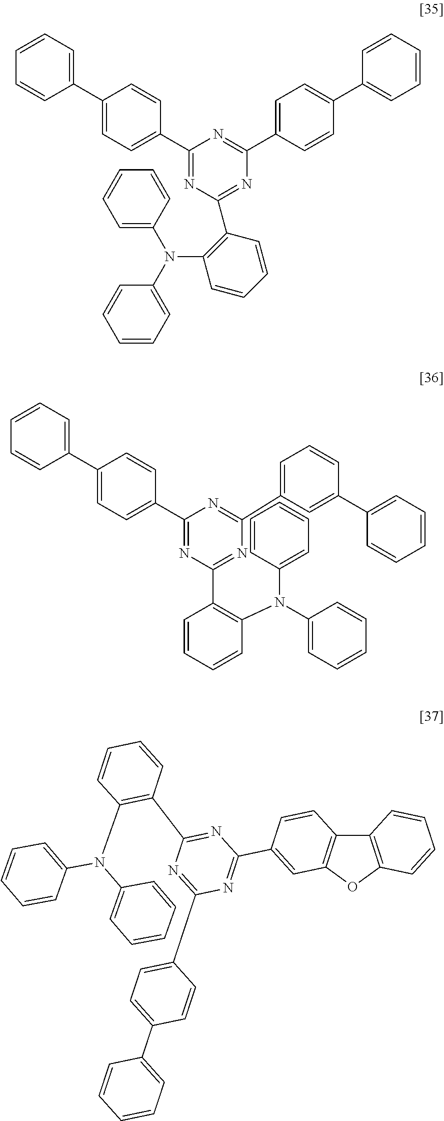 [SMILES: c1ccc(-c2ccc(-c3nc(-c4ccc(-c5ccccc5)cc4)nc(-c4ccccc4N(c4ccccc4)c4ccccc4)n3)cc2)cc1 ; c1ccc(-c2ccc(-c3nc(-c4cccc(-c5ccccc5)c4)nc(-c4ccccc4N(c4ccccc4)c4ccccc4)n3)cc2)cc1 ; c1ccc(-c2ccc(-c3nc(-c4ccc5c(c4)oc4ccccc45)nc(-c4ccccc4N(c4ccccc4)c4ccccc4)n3)cc2)cc1]

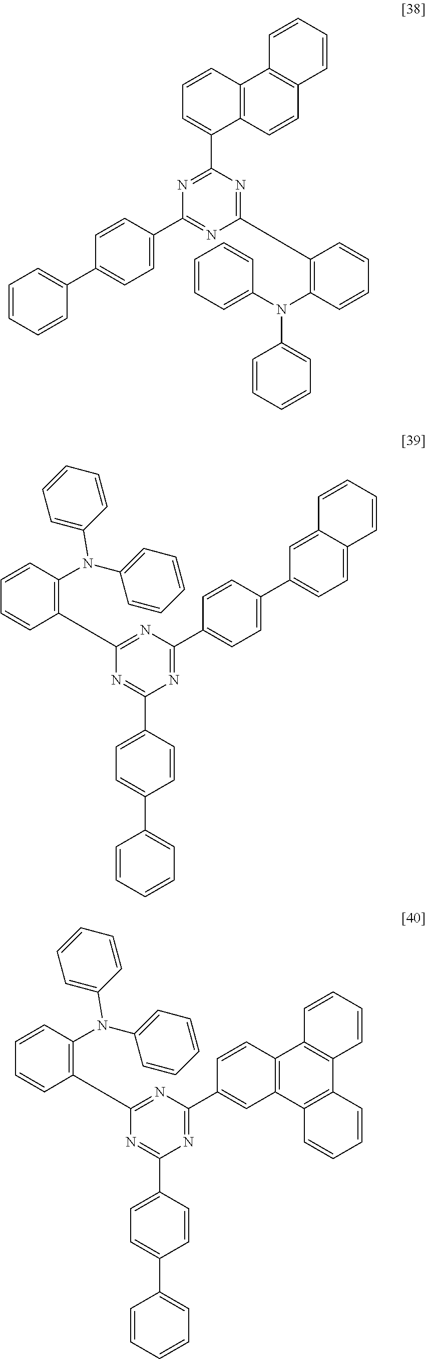 [SMILES: c1ccc(-c2ccc(-c3nc(-c4ccccc4N(c4ccccc4)c4ccccc4)nc(-c4cccc5c4ccc4ccccc45)n3)cc2)cc1 ; c1ccc(-c2ccc(-c3nc(-c4ccc(-c5ccc6ccccc6c5)cc4)nc(-c4ccccc4N(c4ccccc4)c4ccccc4)n3)cc2)cc1 ; c1ccc(-c2ccc(-c3nc(-c4ccc5c6ccccc6c6ccccc6c5c4)nc(-c4ccccc4N(c4ccccc4)c4ccccc4)n3)cc2)cc1]

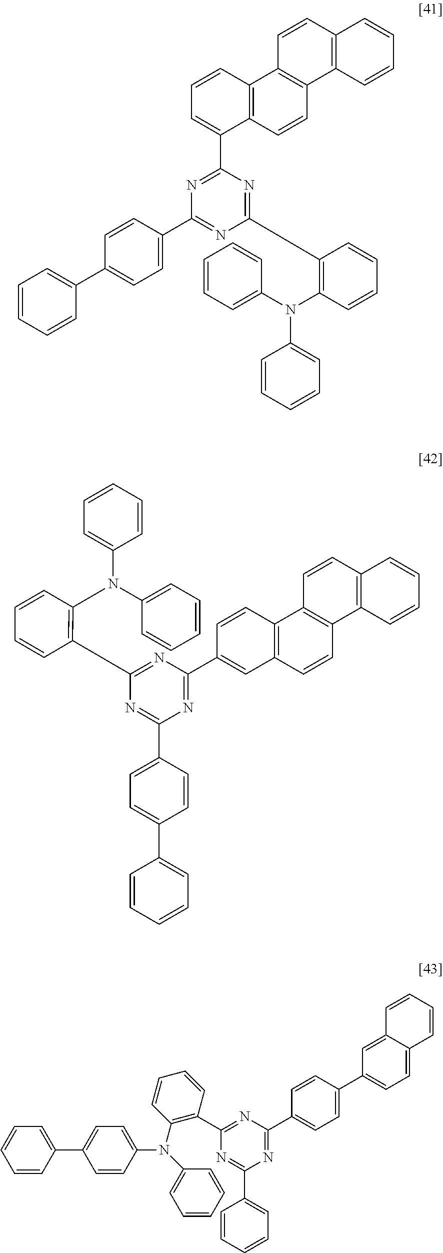 [SMILES: c1ccc(-c2ccc(-c3nc(-c4ccccc4N(c4ccccc4)c4ccccc4)nc(-c4cccc5c4ccc4c6ccccc6ccc54)n3)cc2)cc1 ; c1ccc(-c2ccc(-c3nc(-c4ccc5c(ccc6c7ccccc7ccc56)c4)nc(-c4ccccc4N(c4ccccc4)c4ccccc4)n3)cc2)cc1 ; c1ccc(-c2ccc(N(c3ccccc3)c3ccccc3-c3nc(-c4ccccc4)nc(-c4ccc(-c5ccc6ccccc6c5)cc4)n3)cc2)cc1]

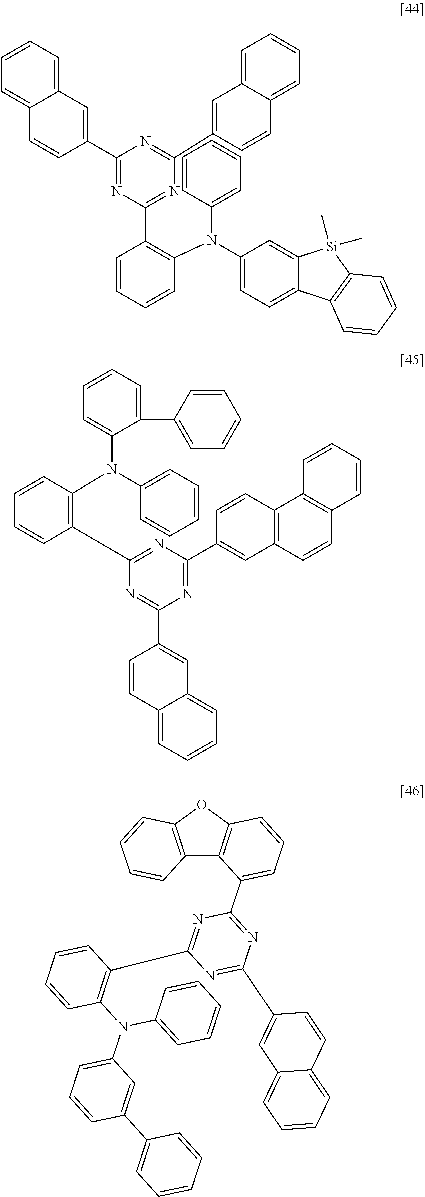

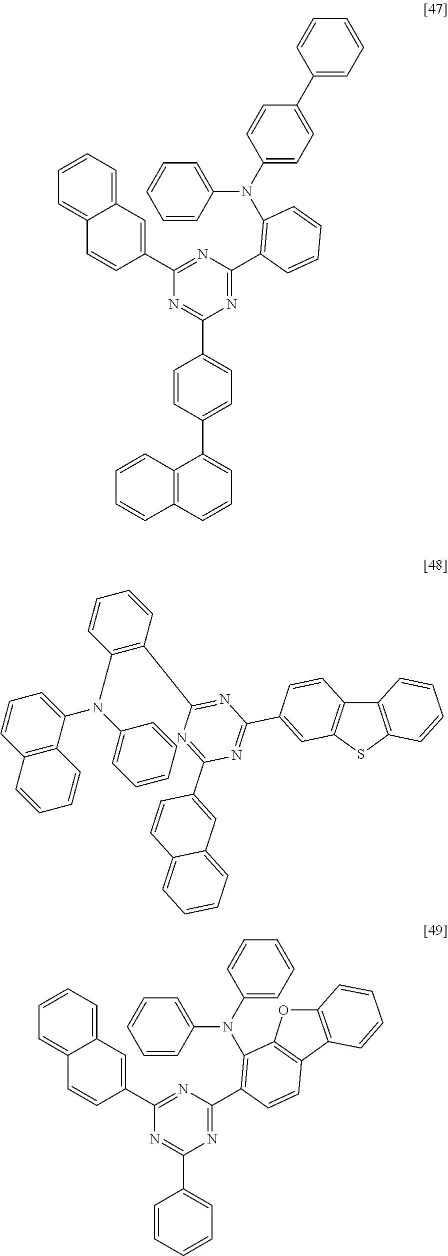

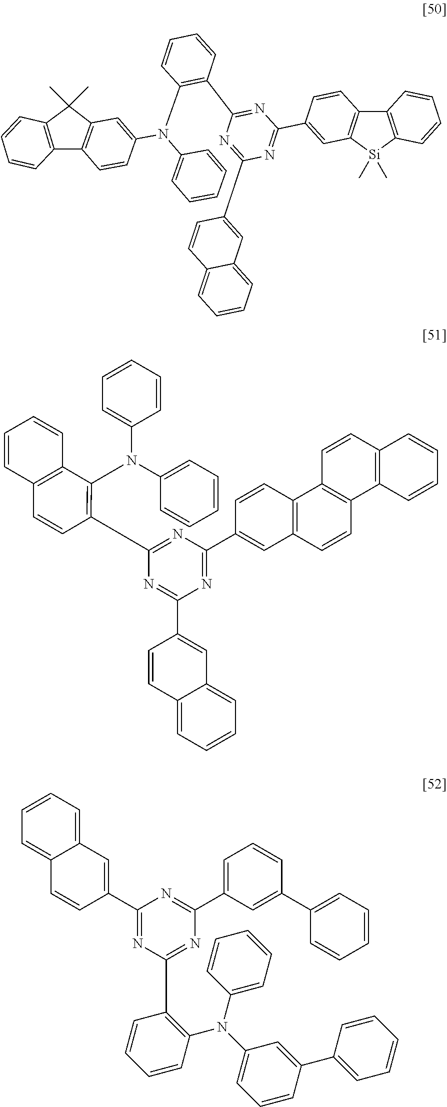 [SMILES: CC1(C)c2ccccc2-c2ccc(N(c3ccccc3)c3ccccc3-c3nc(-c4ccc5c(c4)[Si](C)(C)c4ccccc4-5)nc(-c4ccc5ccccc5c4)n3)cc21 ; c1ccc(N(c2ccccc2)c2c(-c3nc(-c4ccc5ccccc5c4)nc(-c4ccc5c(ccc6c7ccccc7ccc56)c4)n3)ccc3ccccc23)cc1 ; c1ccc(-c2cccc(-c3nc(-c4ccc5ccccc5c4)nc(-c4ccccc4N(c4ccccc4)c4cccc(-c5ccccc5)c4)n3)c2)cc1]

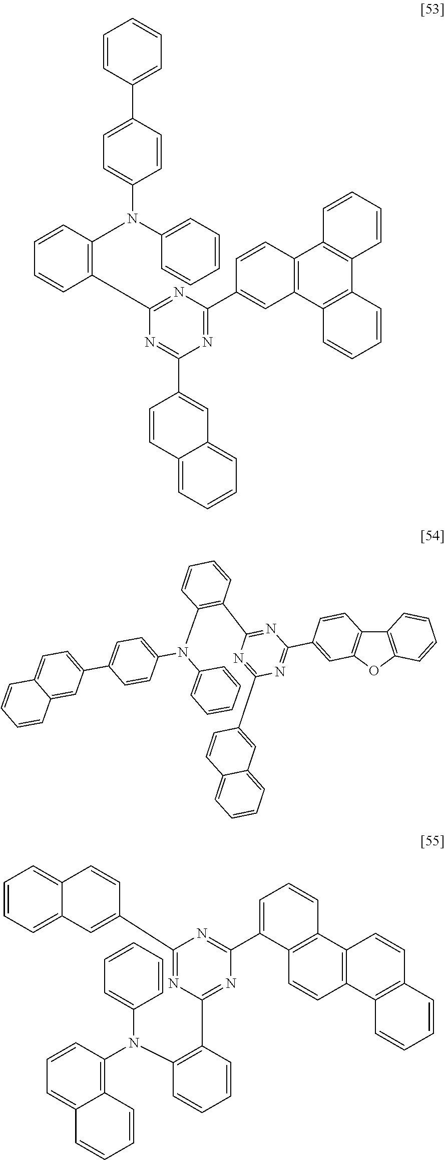 [SMILES: c1ccc(-c2ccc(N(c3ccccc3)c3ccccc3-c3nc(-c4ccc5ccccc5c4)nc(-c4ccc5c6ccccc6c6ccccc6c5c4)n3)cc2)cc1 ; c1ccc(N(c2ccc(-c3ccc4ccccc4c3)cc2)c2ccccc2-c2nc(-c3ccc4ccccc4c3)nc(-c3ccc4c(c3)oc3ccccc34)n2)cc1 ; c1ccc(N(c2ccccc2-c2nc(-c3ccc4ccccc4c3)nc(-c3cccc4c3ccc3c5ccccc5ccc43)n2)c2cccc3ccccc23)cc1]

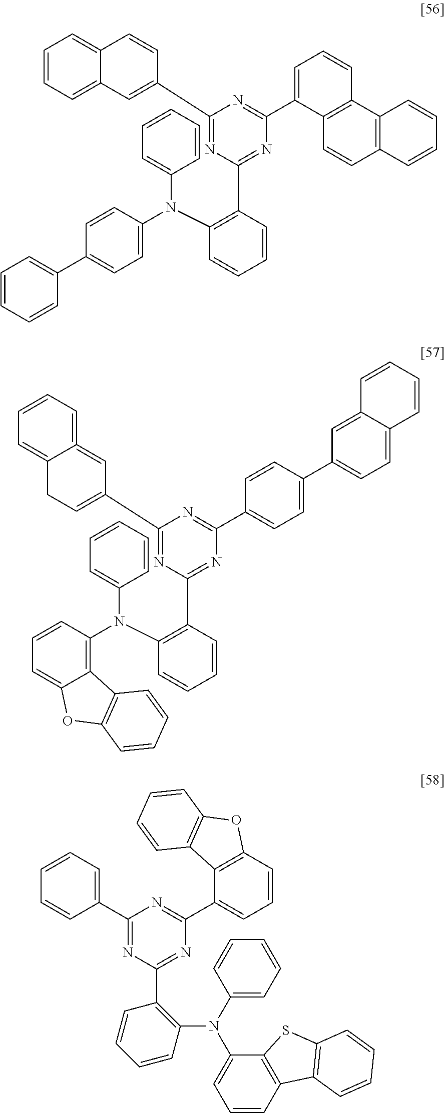

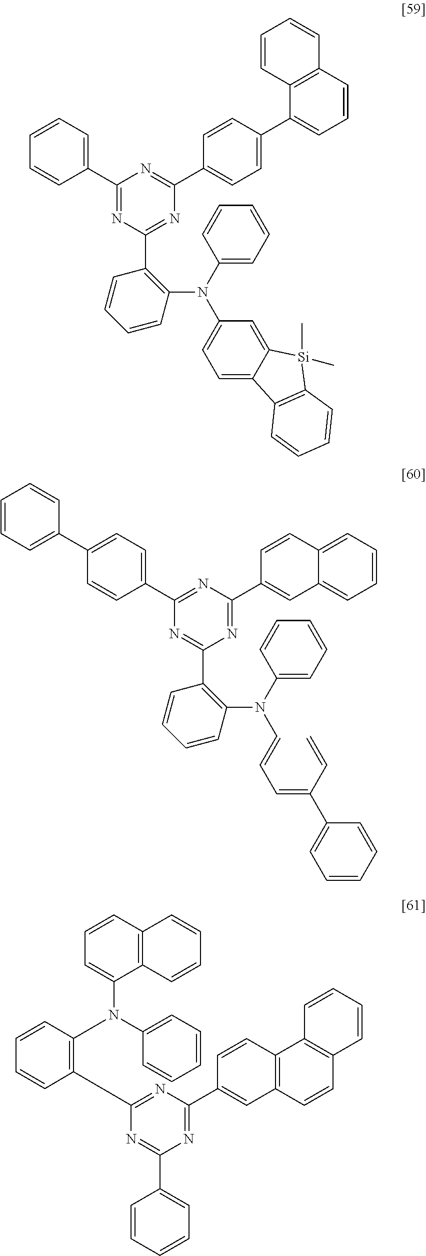 [SMILES: C[Si]1(C)c2ccccc2-c2ccc(N(c3ccccc3)c3ccccc3-c3nc(-c4ccccc4)nc(-c4ccc(-c5cccc6ccccc56)cc4)n3)cc21 ; C=C/C(=C\C=C\N(c1ccccc1)c1ccccc1-c1nc(-c2ccc(-c3ccccc3)cc2)nc(-c2ccc3ccccc3c2)n1)c1ccccc1 ; c1ccc(-c2nc(-c3ccc4c(ccc5ccccc54)c3)nc(-c3ccccc3N(c3ccccc3)c3cccc4ccccc34)n2)cc1]

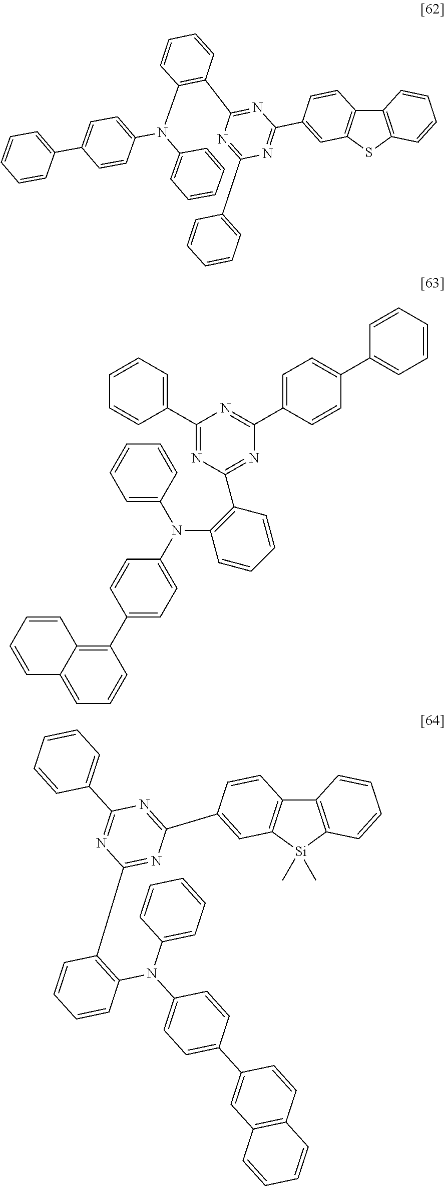

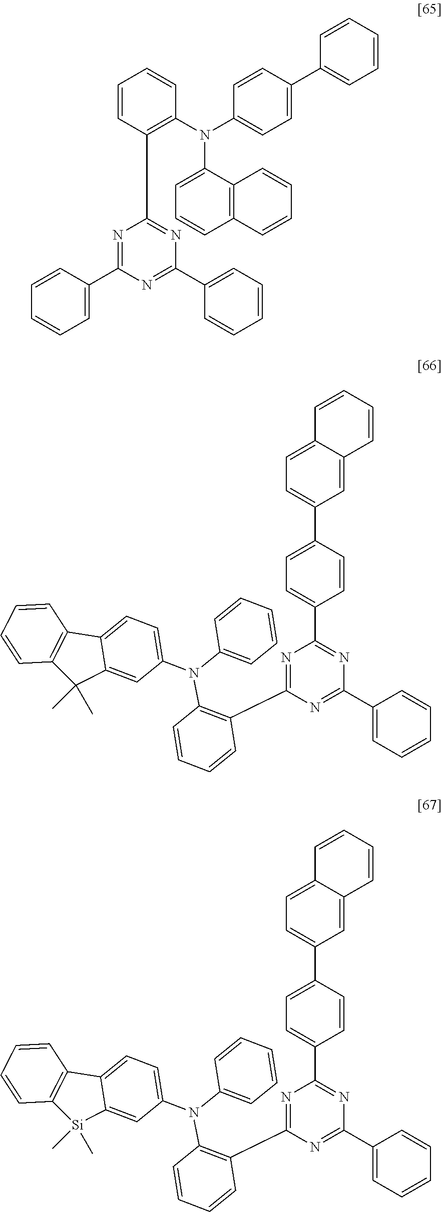 [SMILES: c1ccc(-c2ccc(N(c3ccccc3-c3nc(-c4ccccc4)nc(-c4ccccc4)n3)c3cccc4ccccc34)cc2)cc1 ; CC1(C)c2ccccc2-c2ccc(N(c3ccccc3)c3ccccc3-c3nc(-c4ccccc4)nc(-c4ccc(-c5ccc6ccccc6c5)cc4)n3)cc21 ; C[Si]1(C)c2ccccc2-c2ccc(N(c3ccccc3)c3ccccc3-c3nc(-c4ccccc4)nc(-c4ccc(-c5ccc6ccccc6c5)cc4)n3)cc21]

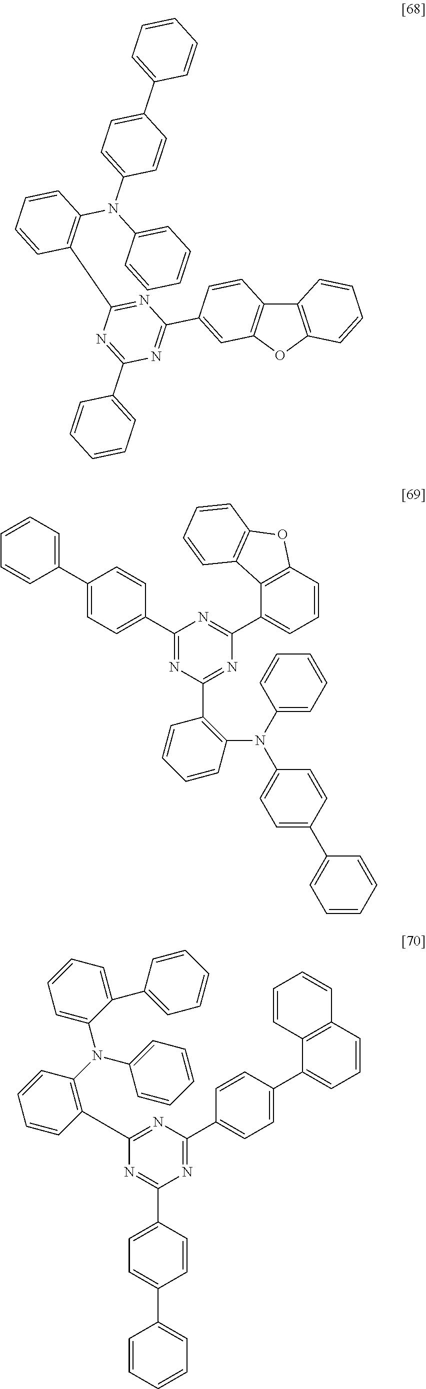 [SMILES: c1ccc(-c2ccc(N(c3ccccc3)c3ccccc3-c3nc(-c4ccccc4)nc(-c4ccc5c(c4)oc4ccccc45)n3)cc2)cc1 ; c1ccc(-c2ccc(-c3nc(-c4ccccc4N(c4ccccc4)c4ccc(-c5ccccc5)cc4)nc(-c4cccc5oc6ccccc6c45)n3)cc2)cc1 ; c1ccc(-c2ccc(-c3nc(-c4ccc(-c5cccc6ccccc56)cc4)nc(-c4ccccc4N(c4ccccc4)c4ccccc4-c4ccccc4)n3)cc2)cc1]

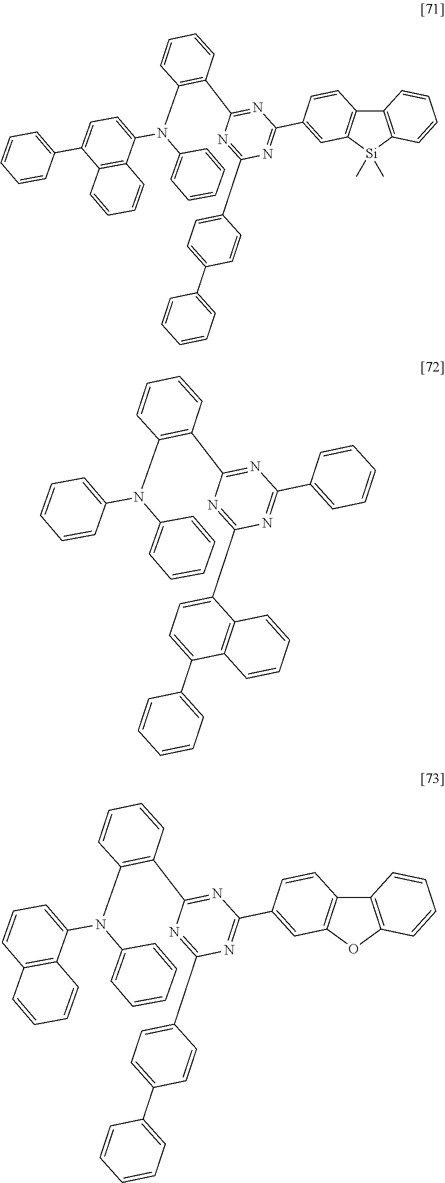

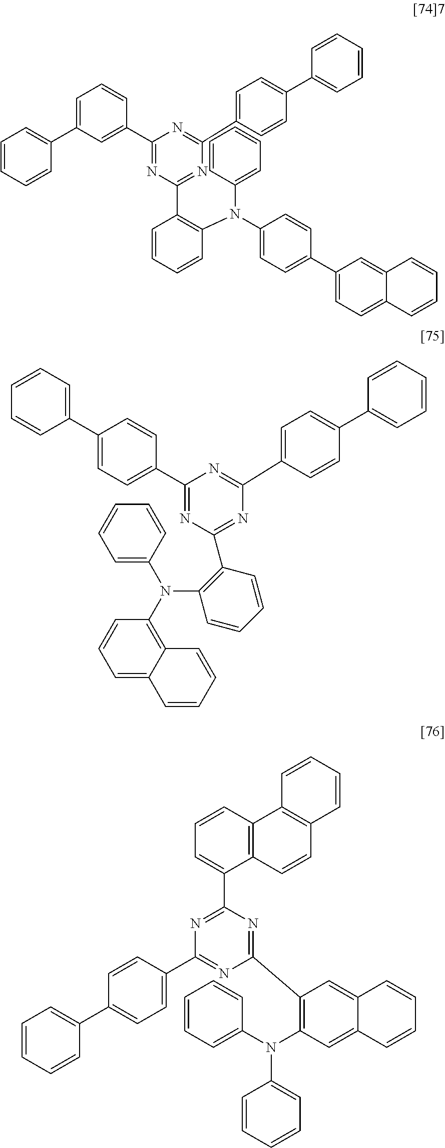 [SMILES: c1ccc(-c2ccc(-c3nc(-c4cccc(-c5ccccc5)c4)nc(-c4ccccc4N(c4ccccc4)c4ccc(-c5ccc6ccccc6c5)cc4)n3)cc2)cc1 ; c1ccc(-c2ccc(-c3nc(-c4ccc(-c5ccccc5)cc4)nc(-c4ccccc4N(c4ccccc4)c4cccc5ccccc45)n3)cc2)cc1 ; c1ccc(-c2ccc(-c3nc(-c4cc5ccccc5cc4N(c4ccccc4)c4ccccc4)nc(-c4cccc5c4ccc4ccccc45)n3)cc2)cc1]

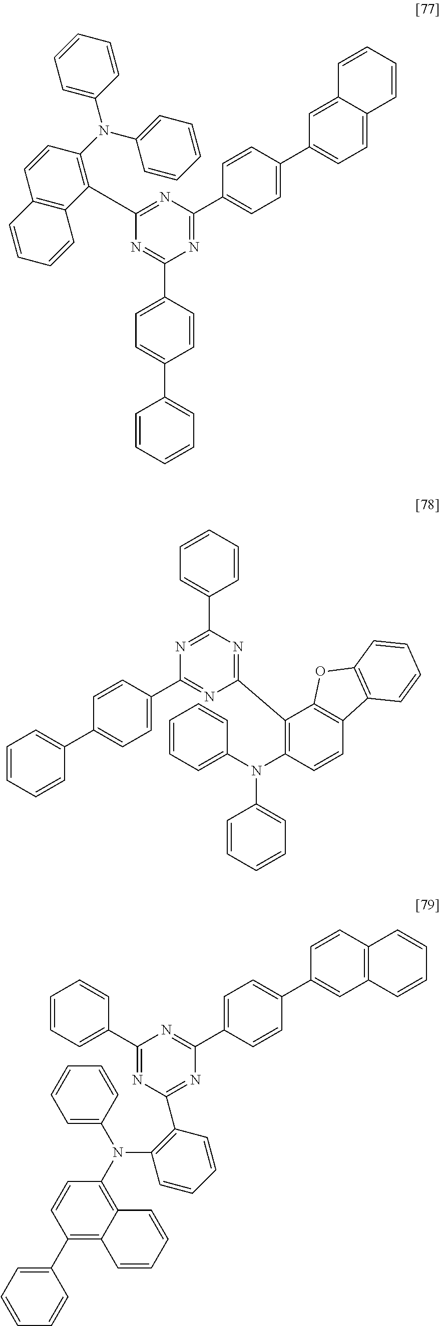 [SMILES: c1ccc(-c2ccc(-c3nc(-c4ccc(-c5ccc6ccccc6c5)cc4)nc(-c4c(N(c5ccccc5)c5ccccc5)ccc5ccccc45)n3)cc2)cc1 ; c1ccc(-c2ccc(-c3nc(-c4ccccc4)nc(-c4c(N(c5ccccc5)c5ccccc5)ccc5c4oc4ccccc45)n3)cc2)cc1 ; c1ccc(-c2nc(-c3ccc(-c4ccc5ccccc5c4)cc3)nc(-c3ccccc3N(c3ccccc3)c3ccc(-c4ccccc4)c4ccccc34)n2)cc1]

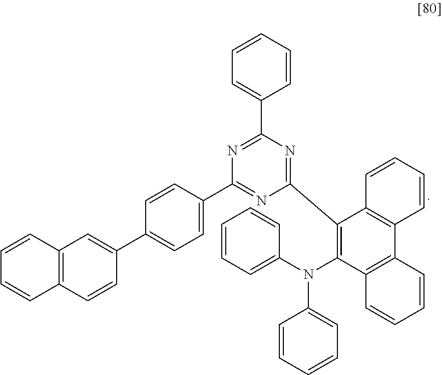 [SMILES: c1ccc(-c2nc(-c3ccc(-c4ccc5ccccc5c4)cc3)nc(-c3c(N(c4ccccc4)c4ccccc4)c4ccccc4c4ccccc34)n2)cc1]

8. The composition for an organic optoelectronic device of claim 1, wherein:
the composition includes the first compound represented by Chemical Formula 1 and the second compound represented by Chemical Formula 2,
the second compound is represented by one of Chemical Formula 2A-I to Chemical Formula 2D-I and Chemical Formula 2B-II to Chemical Formula 2D-II: 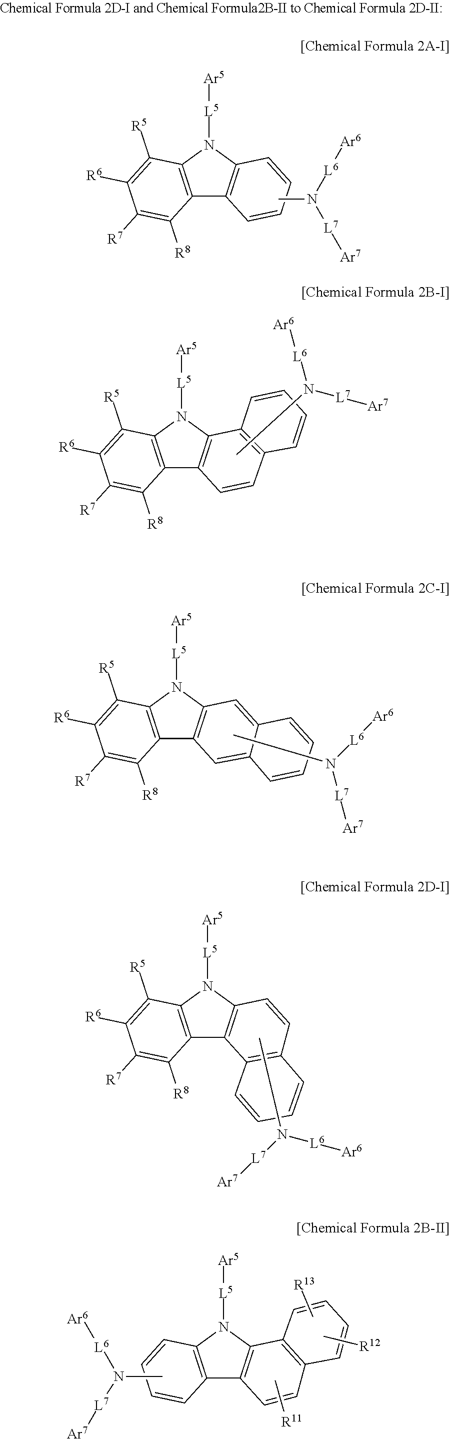 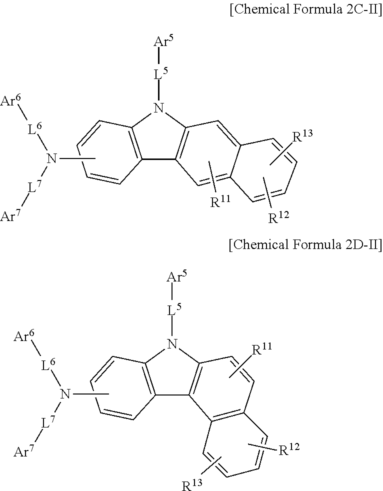
in Chemical Formula 2A-I to Chemical Formula 2D-I and Chemical Formula 2B-II to Chemical Formula 2D-II,
L⁵ to L⁷ and Ar⁵ to Ar⁷ are defined the same as those of Chemical Formula 2, and
R⁵ to R⁸ and R¹¹ to R¹³ are each independently hydrogen, deuterium, a substituted or unsubstituted C1 to C30 alkyl group, a substituted or unsubstituted C6 to C30 aryl group, or a substituted or unsubstituted C2 to C30 heterocyclic group.

9. The composition for an organic optoelectronic device of claim 8, wherein:
the second compound is represented by one of Chemical Formula 2A-I-2, Chemical Formula 2B-I-2, Chemical Formula 2C-I-2, and Chemical Formula 2D-I-2: 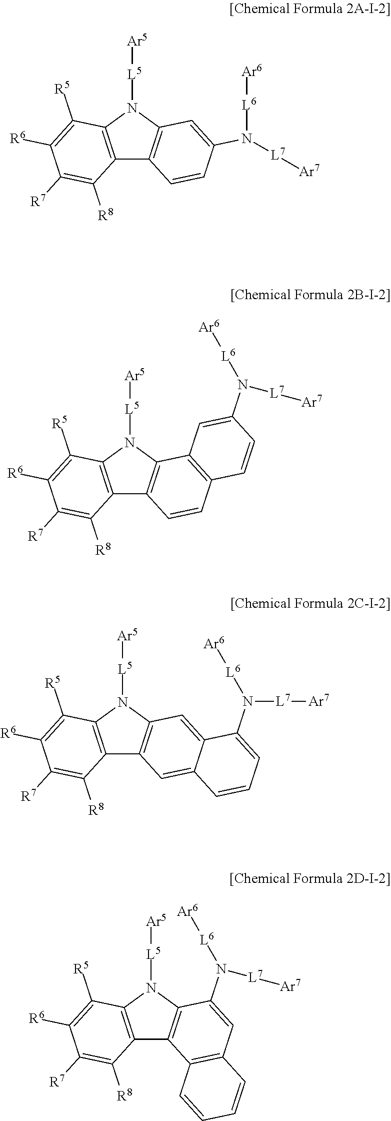
in Chemical Formula 2A-I-2, Chemical Formula 2B-I-2, Chemical Formula 2C-1-2, Chemical Formula 21D-I-2 and Chemical Formula 21D-I-2, L⁵ to L⁷, Ar⁵ to Ar⁷, and R⁵ to R⁸ are defined the same as those of Chemical Formula 2A-I to Chemical Formula 2D-I and Chemical Formula 2B-II to Chemical Formula 2D-II.

10. The composition for an organic optoelectronic device of claim 1, wherein:
the composition includes the first compound represented by Chemical Formula 1 and the second compound represented by Chemical Formula 2,
moieties *-L⁶-Ar⁶ and *-L⁷-Ar⁷ of Chemical Formula a are each independently a moiety of Group IV: 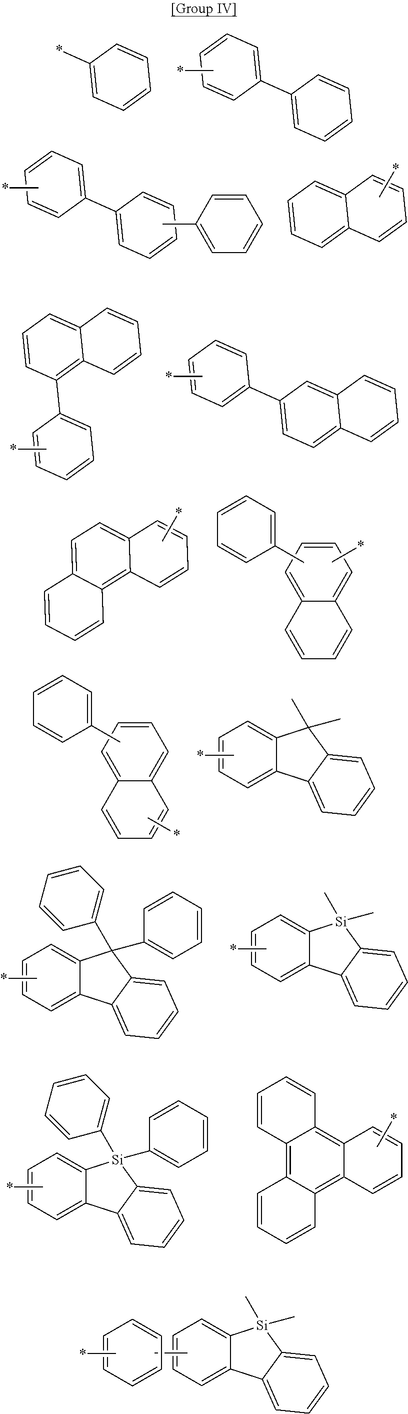 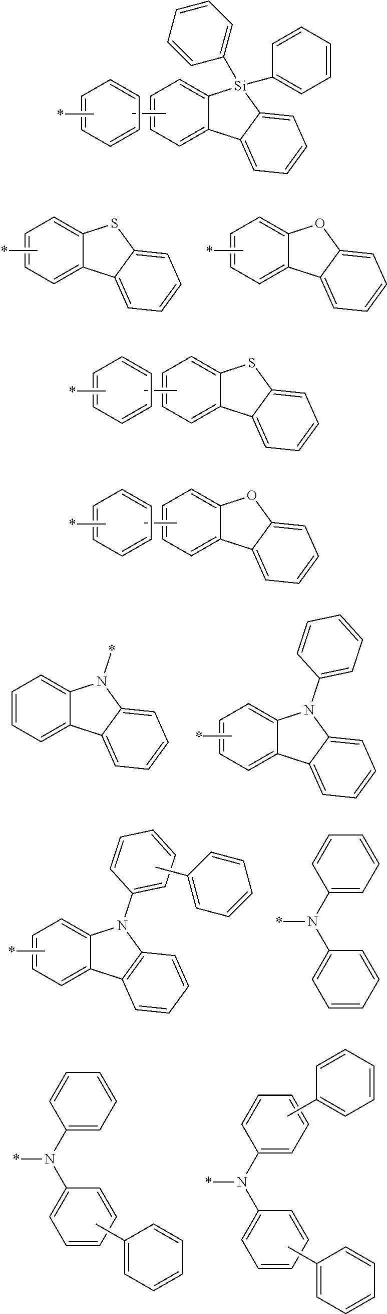
in Group IV, * is a linking point.

11. The composition for an organic optoelectronic device of claim 1, wherein:
the composition includes the first compound represented by Chemical Formula 1 and the second compound represented by Chemical Formula 2, and
the second compound is a compound of Group 2:

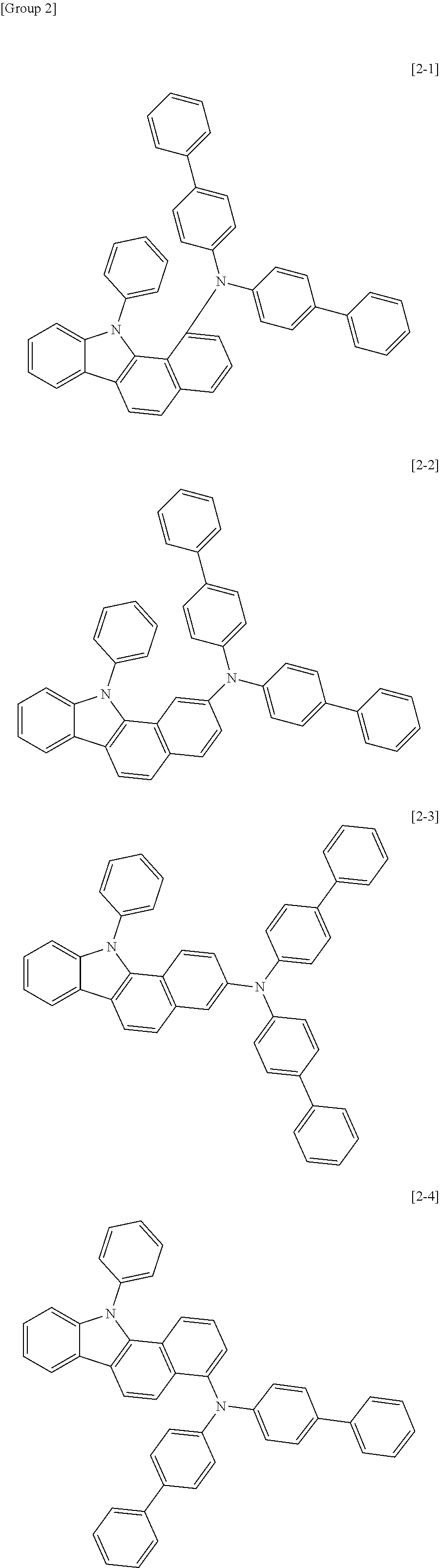 [SMILES: c1ccc(-c2ccc(N(c3ccc(-c4ccccc4)cc3)c3cccc4ccc5c6ccccc6n(-c6ccccc6)c5c34)cc2)cc1 ; c1ccc(-c2ccc(N(c3ccc(-c4ccccc4)cc3)c3ccc4ccc5c6ccccc6n(-c6ccccc6)c5c4c3)cc2)cc1 ; c1ccc(-c2ccc(N(c3ccc(-c4ccccc4)cc3)c3ccc4c(ccc5c6ccccc6n(-c6ccccc6)c45)c3)cc2)cc1 ; c1ccc(-c2ccc(N(c3ccc(-c4ccccc4)cc3)c3cccc4c3ccc3c5ccccc5n(-c5ccccc5)c43)cc2)cc1]

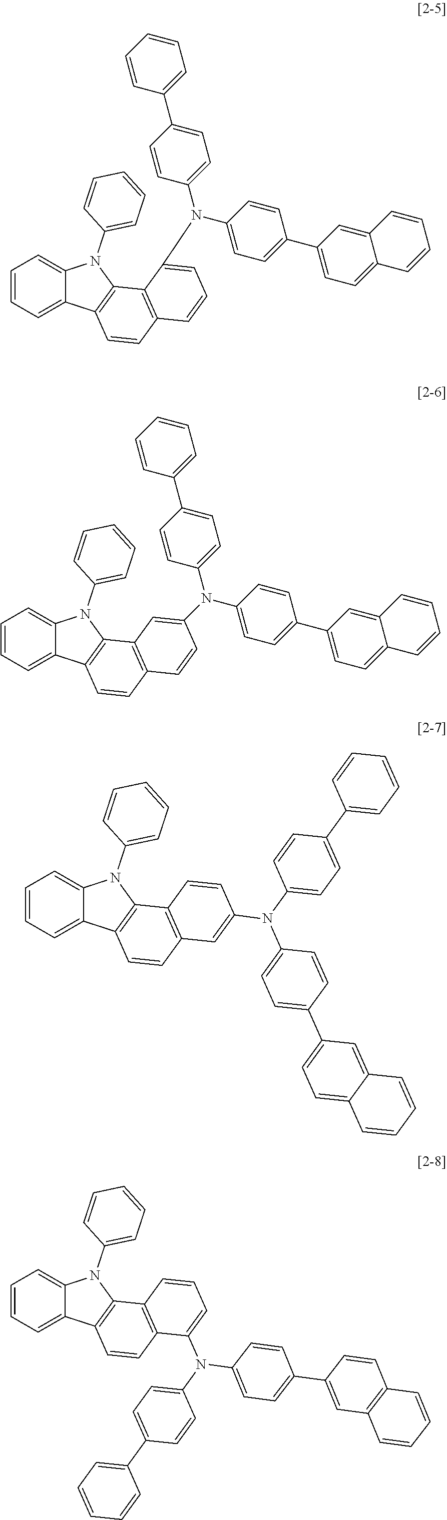 [SMILES: c1ccc(-c2ccc(N(c3ccc(-c4ccc5ccccc5c4)cc3)c3cccc4ccc5c6ccccc6n(-c6ccccc6)c5c34)cc2)cc1 ; c1ccc(-c2ccc(N(c3ccc(-c4ccc5ccccc5c4)cc3)c3ccc4ccc5c6ccccc6n(-c6ccccc6)c5c4c3)cc2)cc1 ; c1ccc(-c2ccc(N(c3ccc(-c4ccc5ccccc5c4)cc3)c3ccc4c(ccc5c6ccccc6n(-c6ccccc6)c45)c3)cc2)cc1 ; c1ccc(-c2ccc(N(c3ccc(-c4ccc5ccccc5c4)cc3)c3cccc4c3ccc3c5ccccc5n(-c5ccccc5)c43)cc2)cc1]

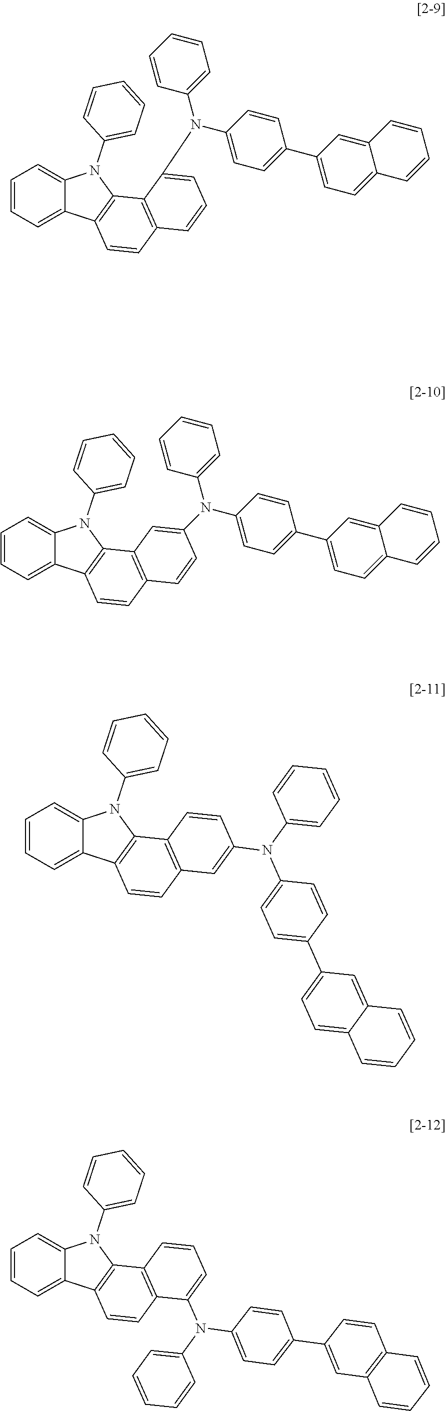 [SMILES: c1ccc(N(c2ccc(-c3ccc4ccccc4c3)cc2)c2cccc3ccc4c5ccccc5n(-c5ccccc5)c4c23)cc1 ; c1ccc(N(c2ccc(-c3ccc4ccccc4c3)cc2)c2ccc3ccc4c5ccccc5n(-c5ccccc5)c4c3c2)cc1 ; c1ccc(N(c2ccc(-c3ccc4ccccc4c3)cc2)c2ccc3c(ccc4c5ccccc5n(-c5ccccc5)c34)c2)cc1 ; c1ccc(N(c2ccc(-c3ccc4ccccc4c3)cc2)c2cccc3c2ccc2c4ccccc4n(-c4ccccc4)c32)cc1]

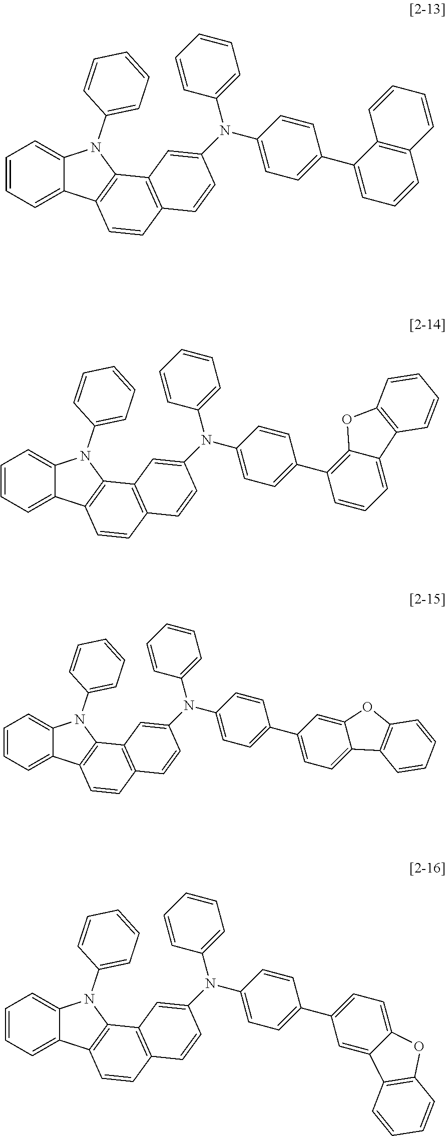 [SMILES: c1ccc(N(c2ccc(-c3cccc4ccccc34)cc2)c2ccc3ccc4c5ccccc5n(-c5ccccc5)c4c3c2)cc1 ; c1ccc(N(c2ccc(-c3cccc4c3oc3ccccc34)cc2)c2ccc3ccc4c5ccccc5n(-c5ccccc5)c4c3c2)cc1 ; c1ccc(N(c2ccc(-c3ccc4c(c3)oc3ccccc34)cc2)c2ccc3ccc4c5ccccc5n(-c5ccccc5)c4c3c2)cc1 ; c1ccc(N(c2ccc(-c3ccc4oc5ccccc5c4c3)cc2)c2ccc3ccc4c5ccccc5n(-c5ccccc5)c4c3c2)cc1]

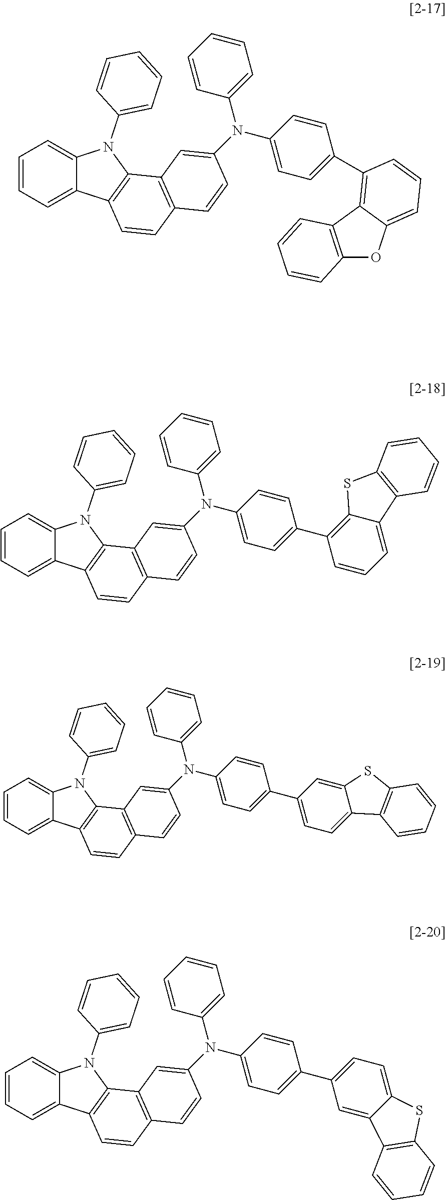 [SMILES: c1ccc(N(c2ccc(-c3cccc4oc5ccccc5c34)cc2)c2ccc3ccc4c5ccccc5n(-c5ccccc5)c4c3c2)cc1 ; c1ccc(N(c2ccc(-c3cccc4c3sc3ccccc34)cc2)c2ccc3ccc4c5ccccc5n(-c5ccccc5)c4c3c2)cc1 ; c1ccc(N(c2ccc(-c3ccc4c(c3)sc3ccccc34)cc2)c2ccc3ccc4c5ccccc5n(-c5ccccc5)c4c3c2)cc1 ; c1ccc(N(c2ccc(-c3ccc4sc5ccccc5c4c3)cc2)c2ccc3ccc4c5ccccc5n(-c5ccccc5)c4c3c2)cc1]

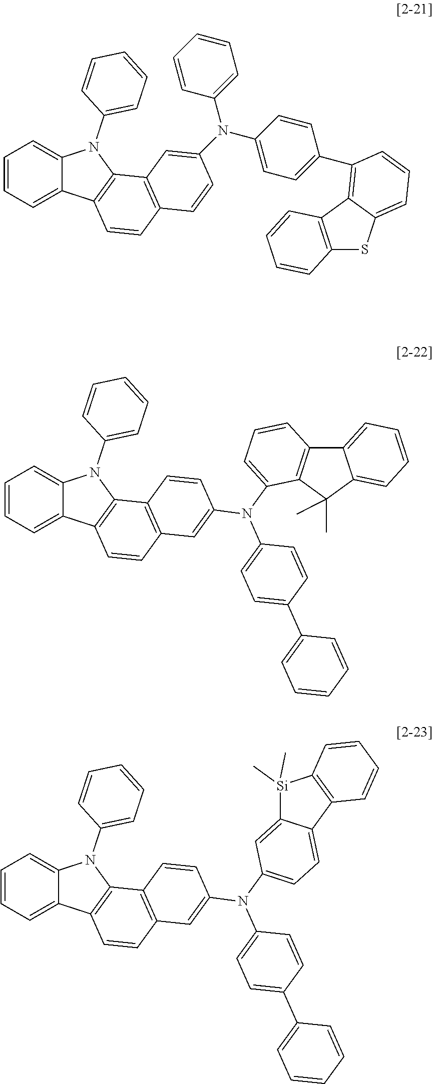 [SMILES: c1ccc(N(c2ccc(-c3cccc4sc5ccccc5c34)cc2)c2ccc3ccc4c5ccccc5n(-c5ccccc5)c4c3c2)cc1 ; CC1(C)c2ccccc2-c2cccc(N(c3ccc(-c4ccccc4)cc3)c3ccc4c(ccc5c6ccccc6n(-c6ccccc6)c45)c3)c21 ; C[Si]1(C)c2ccccc2-c2ccc(N(c3ccc(-c4ccccc4)cc3)c3ccc4c(ccc5c6ccccc6n(-c6ccccc6)c45)c3)cc21]

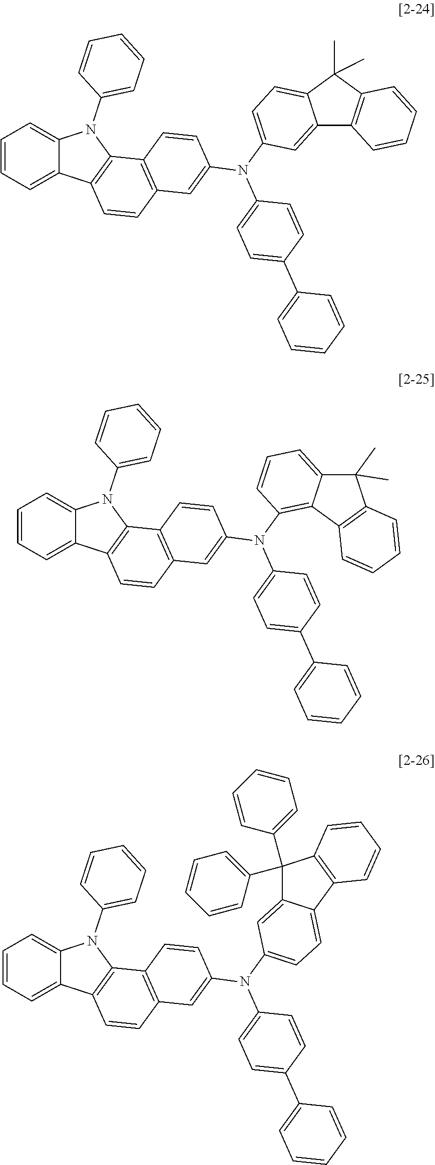 [SMILES: CC1(C)c2ccccc2-c2cc(N(c3ccc(-c4ccccc4)cc3)c3ccc4c(ccc5c6ccccc6n(-c6ccccc6)c45)c3)ccc21 ; CC1(C)c2ccccc2-c2c(N(c3ccc(-c4ccccc4)cc3)c3ccc4c(ccc5c6ccccc6n(-c6ccccc6)c45)c3)cccc21 ; c1ccc(-c2ccc(N(c3ccc4c(c3)C(c3ccccc3)(c3ccccc3)c3ccccc3-4)c3ccc4c(ccc5c6ccccc6n(-c6ccccc6)c45)c3)cc2)cc1]

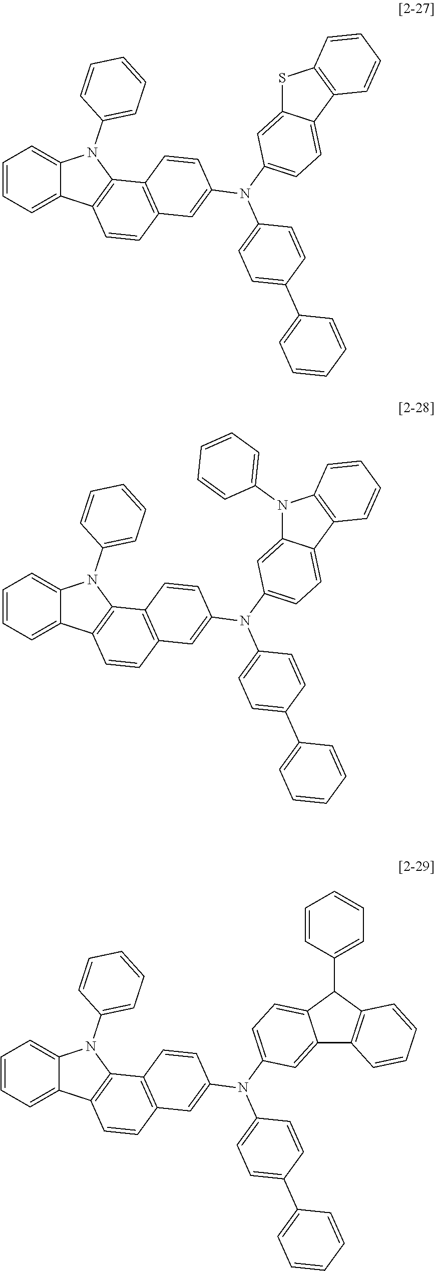 [SMILES: c1ccc(-c2ccc(N(c3ccc4c(ccc5c6ccccc6n(-c6ccccc6)c45)c3)c3ccc4c(c3)sc3ccccc34)cc2)cc1 ; c1ccc(-c2ccc(N(c3ccc4c(ccc5c6ccccc6n(-c6ccccc6)c45)c3)c3ccc4c5ccccc5n(-c5ccccc5)c4c3)cc2)cc1 ; c1ccc(-c2ccc(N(c3ccc4c(c3)-c3ccccc3C4c3ccccc3)c3ccc4c(ccc5c6ccccc6n(-c6ccccc6)c45)c3)cc2)cc1]

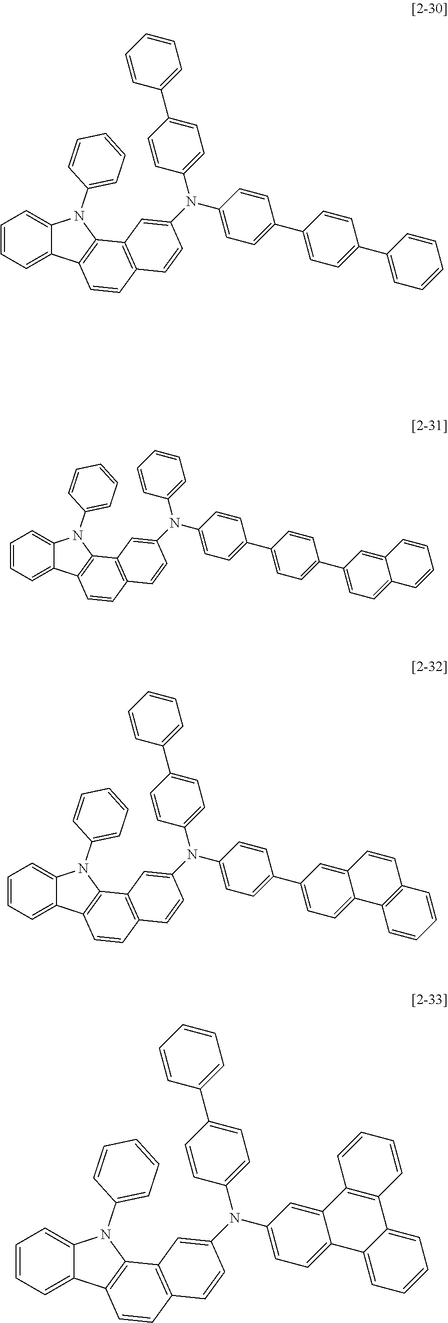 [SMILES: c1ccc(-c2ccc(-c3ccc(N(c4ccc(-c5ccccc5)cc4)c4ccc5ccc6c7ccccc7n(-c7ccccc7)c6c5c4)cc3)cc2)cc1 ; c1ccc(N(c2ccc(-c3ccc(-c4ccc5ccccc5c4)cc3)cc2)c2ccc3ccc4c5ccccc5n(-c5ccccc5)c4c3c2)cc1 ; c1ccc(-c2ccc(N(c3ccc(-c4ccc5c(ccc6ccccc65)c4)cc3)c3ccc4ccc5c6ccccc6n(-c6ccccc6)c5c4c3)cc2)cc1 ; c1ccc(-c2ccc(N(c3ccc4c5ccccc5c5ccccc5c4c3)c3ccc4ccc5c6ccccc6n(-c6ccccc6)c5c4c3)cc2)cc1]

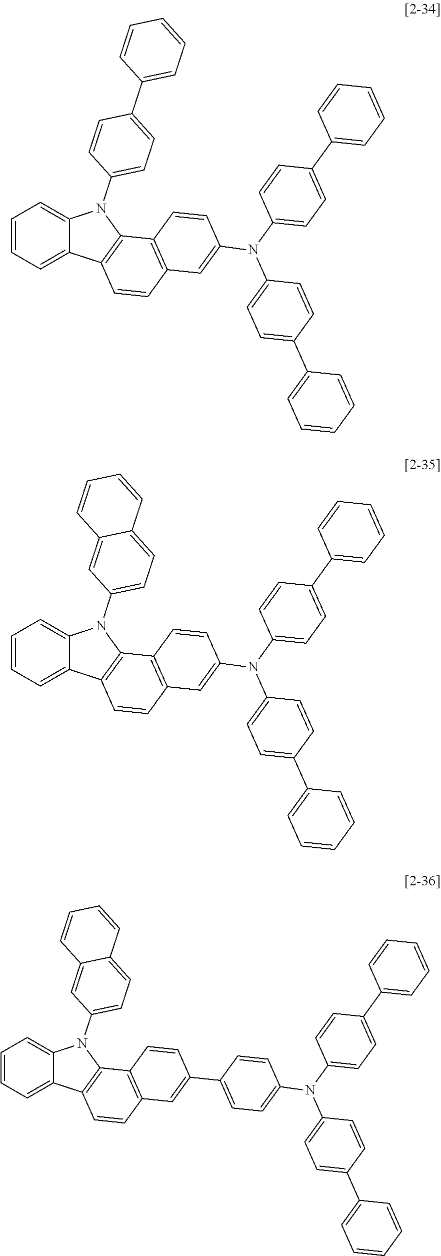

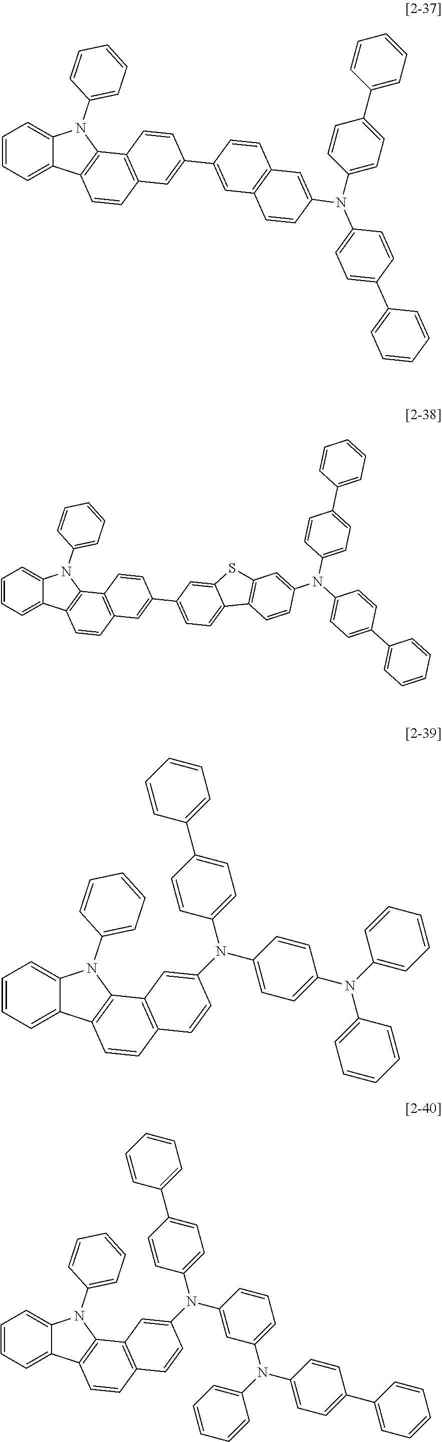 [SMILES: c1ccc(-c2ccc(N(c3ccc(-c4ccccc4)cc3)c3ccc4cc(-c5ccc6c(ccc7c8ccccc8n(-c8ccccc8)c67)c5)ccc4c3)cc2)cc1 ; c1ccc(-c2ccc(N(c3ccc(-c4ccccc4)cc3)c3ccc4c(c3)sc3cc(-c5ccc6c(ccc7c8ccccc8n(-c8ccccc8)c67)c5)ccc34)cc2)cc1 ; c1ccc(-c2ccc(N(c3ccc(N(c4ccccc4)c4ccccc4)cc3)c3ccc4ccc5c6ccccc6n(-c6ccccc6)c5c4c3)cc2)cc1 ; c1ccc(-c2ccc(N(c3ccccc3)c3cccc(N(c4ccc(-c5ccccc5)cc4)c4ccc5ccc6c7ccccc7n(-c7ccccc7)c6c5c4)c3)cc2)cc1]

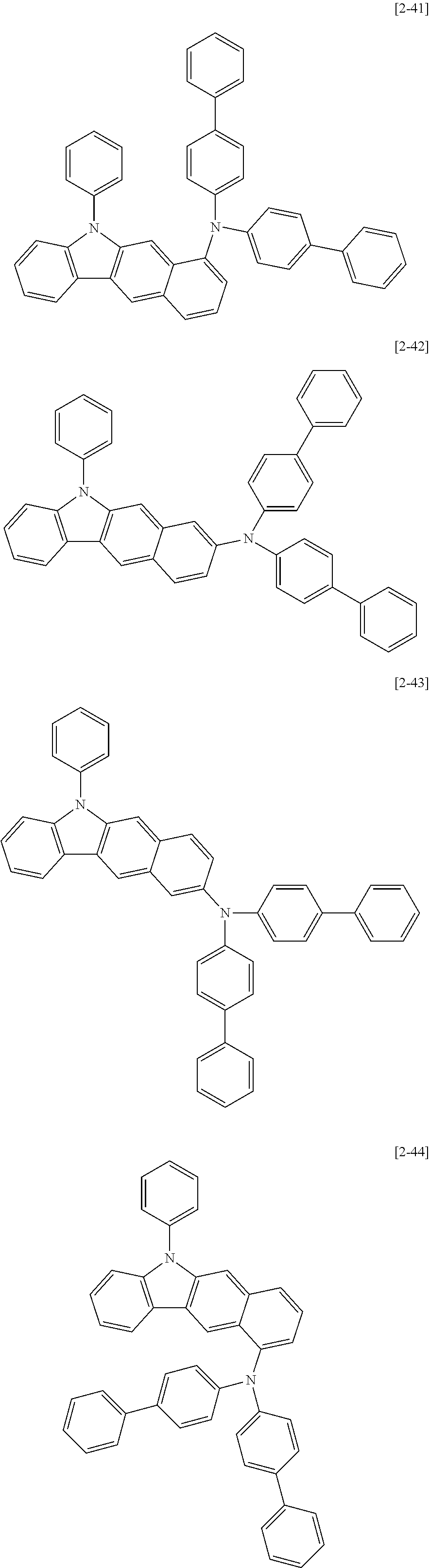 [SMILES: c1ccc(-c2ccc(N(c3ccc(-c4ccccc4)cc3)c3cccc4cc5c6ccccc6n(-c6ccccc6)c5cc34)cc2)cc1 ; c1ccc(-c2ccc(N(c3ccc(-c4ccccc4)cc3)c3ccc4cc5c6ccccc6n(-c6ccccc6)c5cc4c3)cc2)cc1 ; c1ccc(-c2ccc(N(c3ccc(-c4ccccc4)cc3)c3ccc4cc5c(cc4c3)c3ccccc3n5-c3ccccc3)cc2)cc1 ; c1ccc(-c2ccc(N(c3ccc(-c4ccccc4)cc3)c3cccc4cc5c(cc34)c3ccccc3n5-c3ccccc3)cc2)cc1]

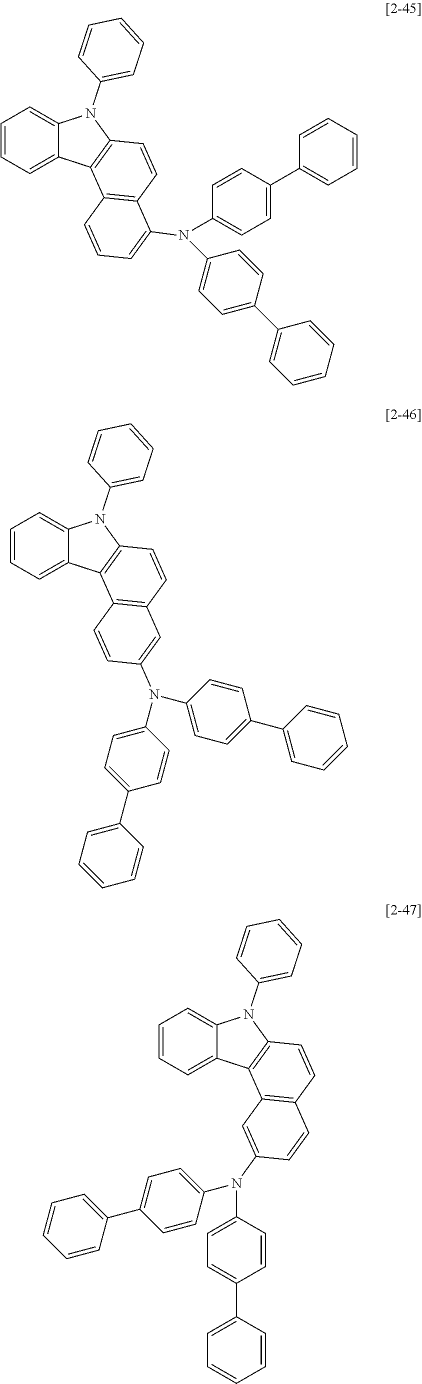 [SMILES: c1ccc(-c2ccc(N(c3ccc(-c4ccccc4)cc3)c3cccc4c3ccc3c4c4ccccc4n3-c3ccccc3)cc2)cc1 ; c1ccc(-c2ccc(N(c3ccc(-c4ccccc4)cc3)c3ccc4c(ccc5c4c4ccccc4n5-c4ccccc4)c3)cc2)cc1 ; c1ccc(-c2ccc(N(c3ccc(-c4ccccc4)cc3)c3ccc4ccc5c(c4c3)c3ccccc3n5-c3ccccc3)cc2)cc1]

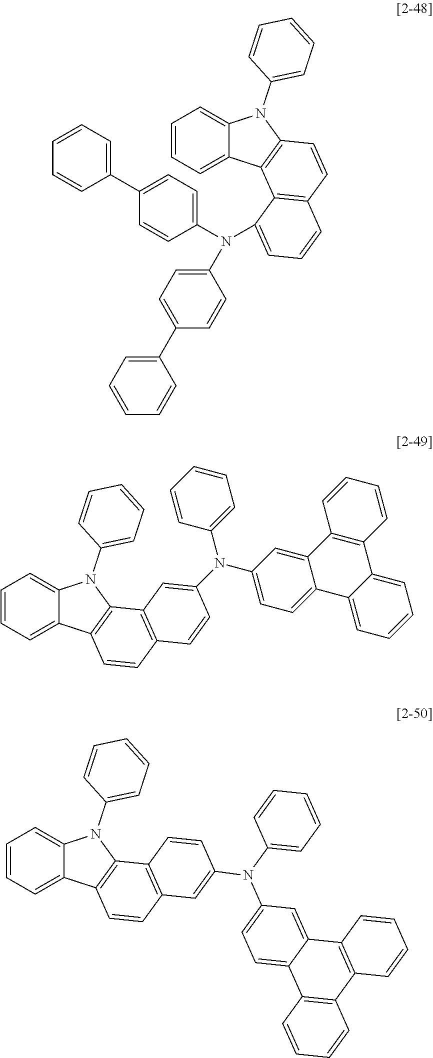 [SMILES: c1ccc(-c2ccc(N(c3ccc(-c4ccccc4)cc3)c3cccc4ccc5c(c6ccccc6n5-c5ccccc5)c34)cc2)cc1 ; c1ccc(N(c2ccc3c4ccccc4c4ccccc4c3c2)c2ccc3ccc4c5ccccc5n(-c5ccccc5)c4c3c2)cc1 ; c1ccc(N(c2ccc3c(ccc4c5ccccc5n(-c5ccccc5)c34)c2)c2ccc3c4ccccc4c4ccccc4c3c2)cc1]

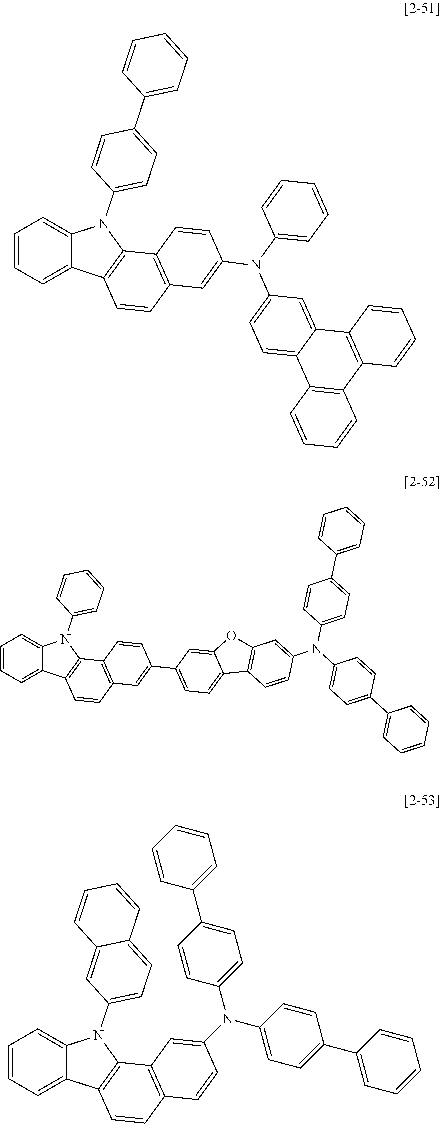 [SMILES: c1ccc(-c2ccc(-n3c4ccccc4c4ccc5cc(N(c6ccccc6)c6ccc7c8ccccc8c8ccccc8c7c6)ccc5c43)cc2)cc1 ; c1ccc(-c2ccc(N(c3ccc(-c4ccccc4)cc3)c3ccc4c(c3)oc3cc(-c5ccc6c(ccc7c8ccccc8n(-c8ccccc8)c67)c5)ccc34)cc2)cc1 ; c1ccc(-c2ccc(N(c3ccc(-c4ccccc4)cc3)c3ccc4ccc5c6ccccc6n(-c6ccc7ccccc7c6)c5c4c3)cc2)cc1]

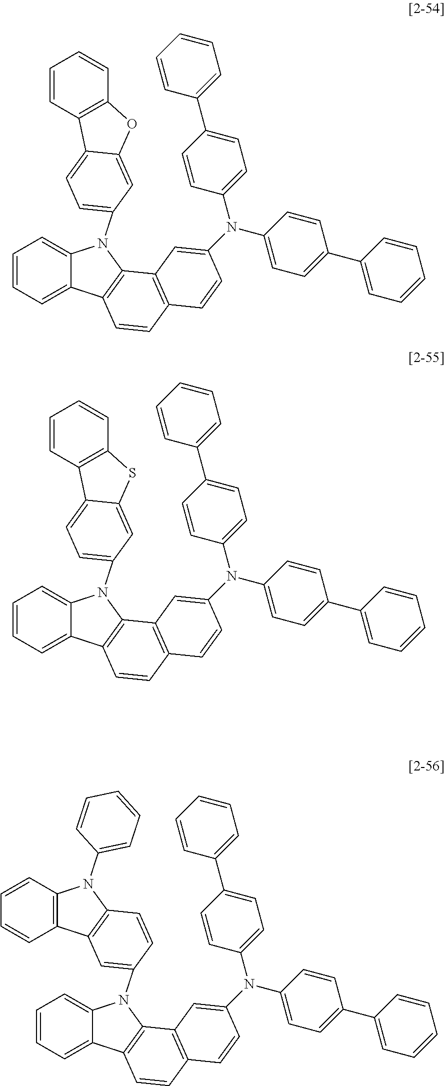 [SMILES: c1ccc(-c2ccc(N(c3ccc(-c4ccccc4)cc3)c3ccc4ccc5c6ccccc6n(-c6ccc7c(c6)oc6ccccc67)c5c4c3)cc2)cc1 ; c1ccc(-c2ccc(N(c3ccc(-c4ccccc4)cc3)c3ccc4ccc5c6ccccc6n(-c6ccc7c(c6)sc6ccccc67)c5c4c3)cc2)cc1 ; c1ccc(-c2ccc(N(c3ccc(-c4ccccc4)cc3)c3ccc4ccc5c6ccccc6n(-c6ccc7c(c6)c6ccccc6n7-c6ccccc6)c5c4c3)cc2)cc1]

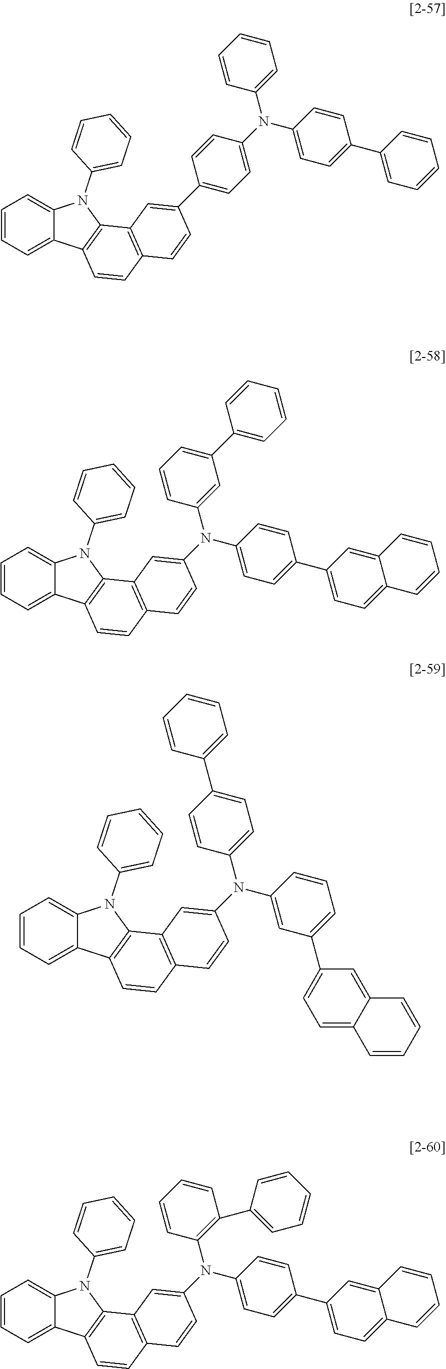 [SMILES: c1ccc(-c2ccc(N(c3ccccc3)c3ccc(-c4ccc5ccc6c7ccccc7n(-c7ccccc7)c6c5c4)cc3)cc2)cc1 ; c1ccc(-c2cccc(N(c3ccc(-c4ccc5ccccc5c4)cc3)c3ccc4ccc5c6ccccc6n(-c6ccccc6)c5c4c3)c2)cc1 ; c1ccc(-c2ccc(N(c3cccc(-c4ccc5ccccc5c4)c3)c3ccc4ccc5c6ccccc6n(-c6ccccc6)c5c4c3)cc2)cc1 ; c1ccc(-c2ccccc2N(c2ccc(-c3ccc4ccccc4c3)cc2)c2ccc3ccc4c5ccccc5n(-c5ccccc5)c4c3c2)cc1]

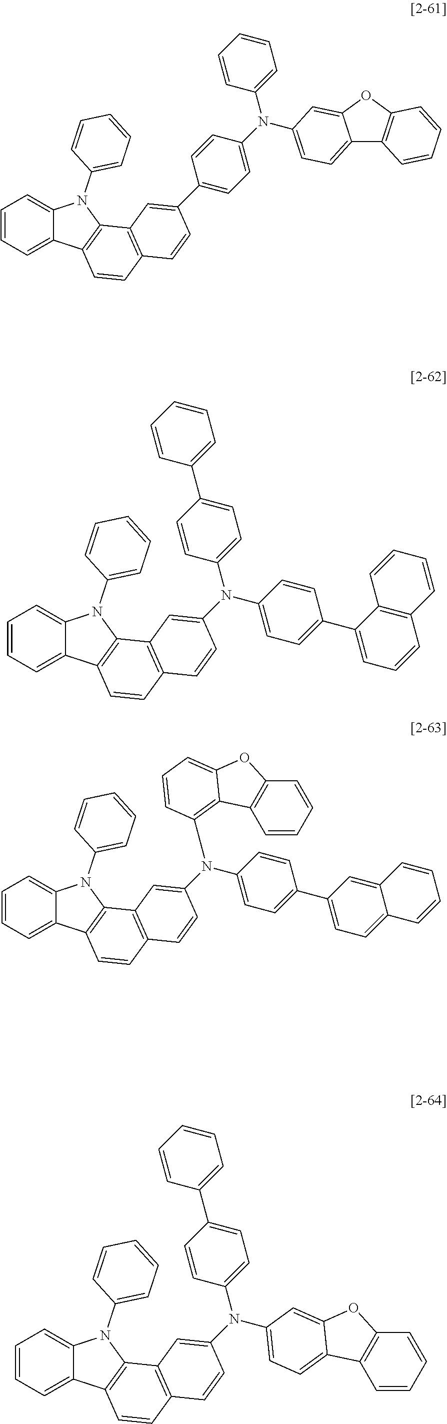 [SMILES: c1ccc(N(c2ccc(-c3ccc4ccc5c6ccccc6n(-c6ccccc6)c5c4c3)cc2)c2ccc3c(c2)oc2ccccc23)cc1 ; c1ccc(-c2ccc(N(c3ccc(-c4cccc5ccccc45)cc3)c3ccc4ccc5c6ccccc6n(-c6ccccc6)c5c4c3)cc2)cc1 ; c1ccc(-n2c3ccccc3c3ccc4ccc(N(c5ccc(-c6ccc7ccccc7c6)cc5)c5cccc6oc7ccccc7c56)cc4c32)cc1 ; c1ccc(-c2ccc(N(c3ccc4c(c3)oc3ccccc34)c3ccc4ccc5c6ccccc6n(-c6ccccc6)c5c4c3)cc2)cc1]

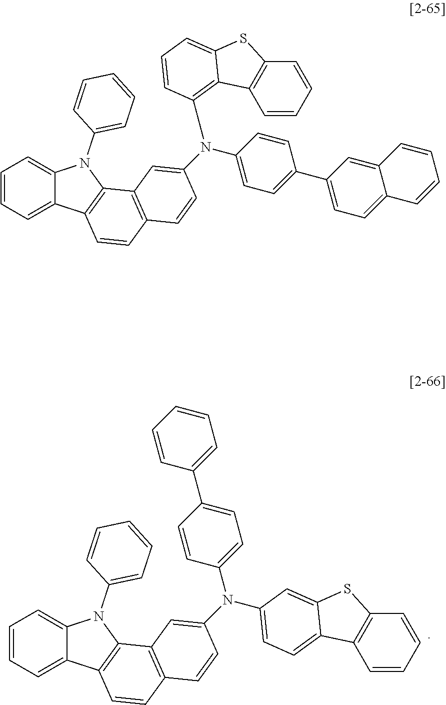 [SMILES: c1ccc(-n2c3ccccc3c3ccc4ccc(N(c5ccc(-c6ccc7ccccc7c6)cc5)c5cccc6sc7ccccc7c56)cc4c32)cc1 ; c1ccc(-c2ccc(N(c3ccc4c(c3)sc3ccccc34)c3ccc4ccc5c6ccccc6n(-c6ccccc6)c5c4c3)cc2)cc1]

12. The composition for an organic optoelectronic device of claim 1, wherein:
the composition includes the first compound represented by Chemical Formula 1 and the third compound represented by a combination of Chemical Formula 3 and Chemical Formula 4,
the third compound is represented by one of Chemical Formula 3A to Chemical Formula 3E: 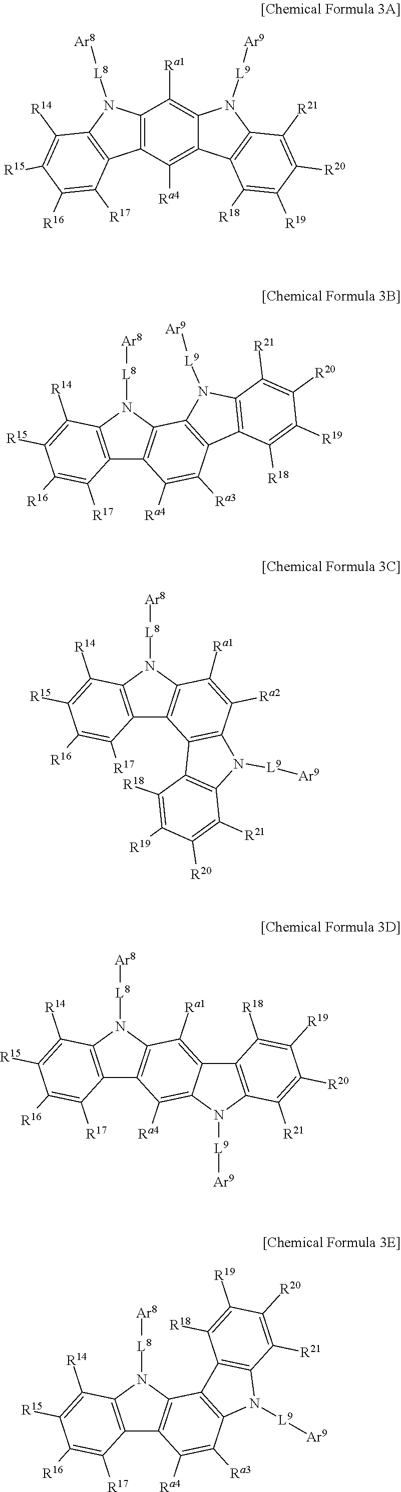
in Chemical Formula 3A to Chemical Formula 3E, L⁸, L⁹, Ar⁸, Ar⁹, R^{a1} to R^{a4}, and R¹⁴ to R²¹ are defined the same as those of Chemical Formula 3 and Chemical Formula 4.

13. The composition for an organic optoelectronic device of claim 12, wherein the third compound is represented by Chemical Formula 3 Å, Chemical Formula 3C, or Chemical Formula 3E.

14. The composition for an organic optoelectronic device of claim 1, wherein:
the composition includes the first compound represented by Chemical Formula 1 and the third compound represented by a combination of Chemical Formula 3 and Chemical Formula 4,
in Chemical Formulas 3 and 4,
L⁸ and L⁹ are each independently a single bond, a substituted or unsubstituted phenylene group or a substituted or unsubstituted pyridinylene group, and
Ar⁸ and Ar⁹ are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, a substituted or unsubstituted chrysenyl group, a substituted or unsubstituted carbazolyl group, a substituted or unsubstituted dibenzofuranyl group, a substituted or unsubstituted dibenzothiophenyl group, a substituted or unsubstituted pyridinyl group, a substituted or unsubstituted pyrimidinyl group, or a substituted or unsubstituted triazinyl group.

15. The composition for an organic optoelectronic device of claim 1, wherein:
the composition includes the first compound represented by Chemical Formula 1 and the third compound represented by a combination of Chemical Formula 3 and Chemical Formula 4,
moieties *-L⁸-Ar⁸ and *-L⁹-Ar⁹ of Chemical Formulas 3 and 4 are each independently a moiety of Group V: 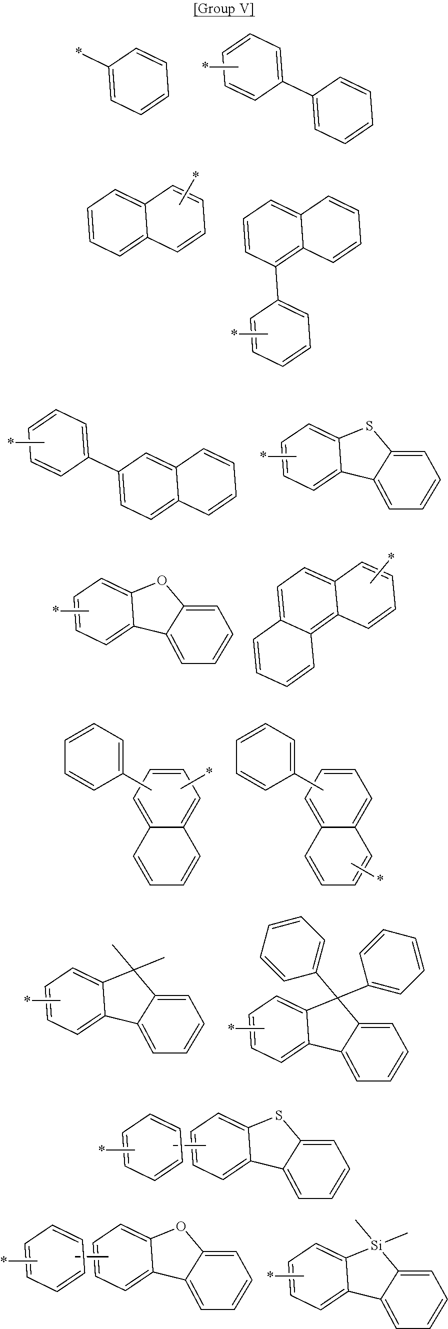 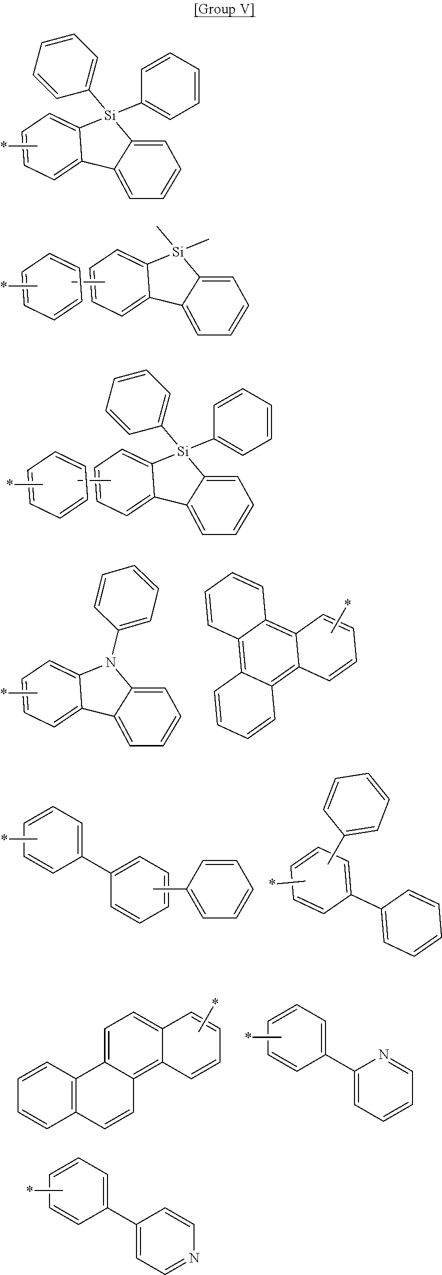
in Group V, * is a linking point.

16. The composition for an organic optoelectronic device of claim 1, wherein:
the composition includes the first compound represented by Chemical Formula 1 and the third compound represented by a combination of Chemical Formula 3 and Chemical Formula 4,
the third compound is a compound of Group 3:

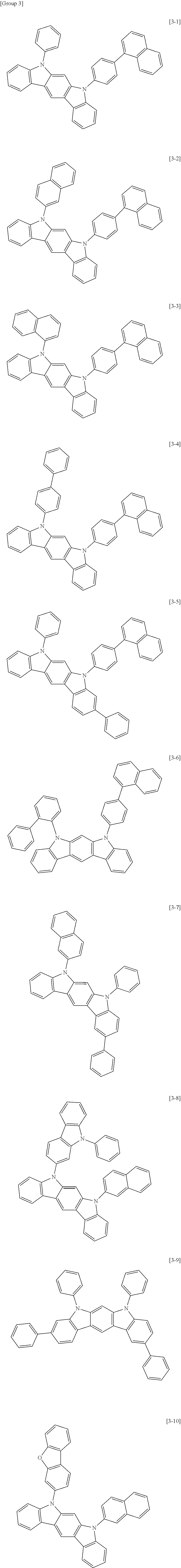

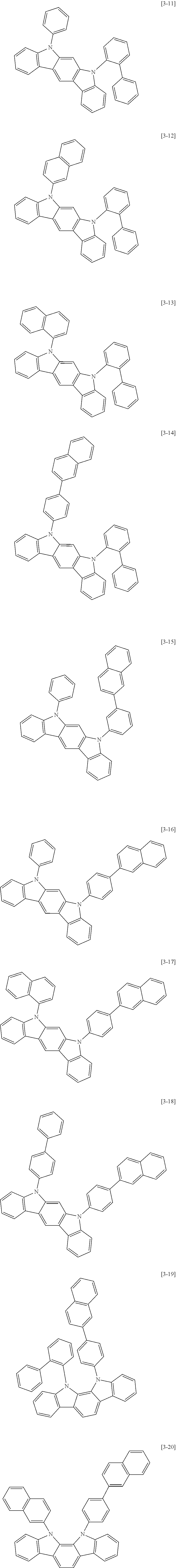

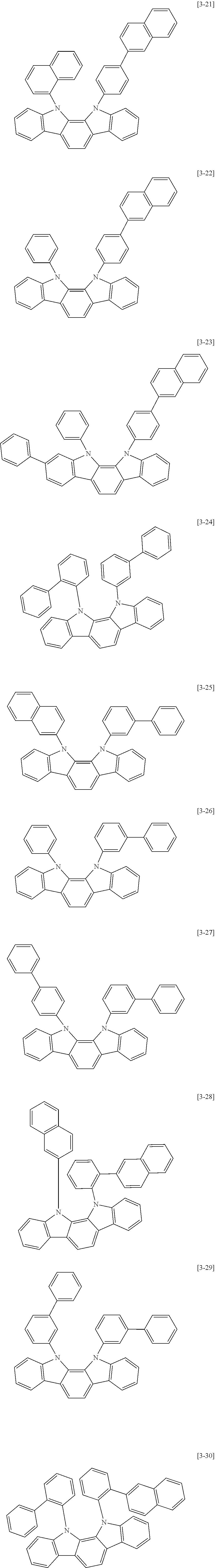

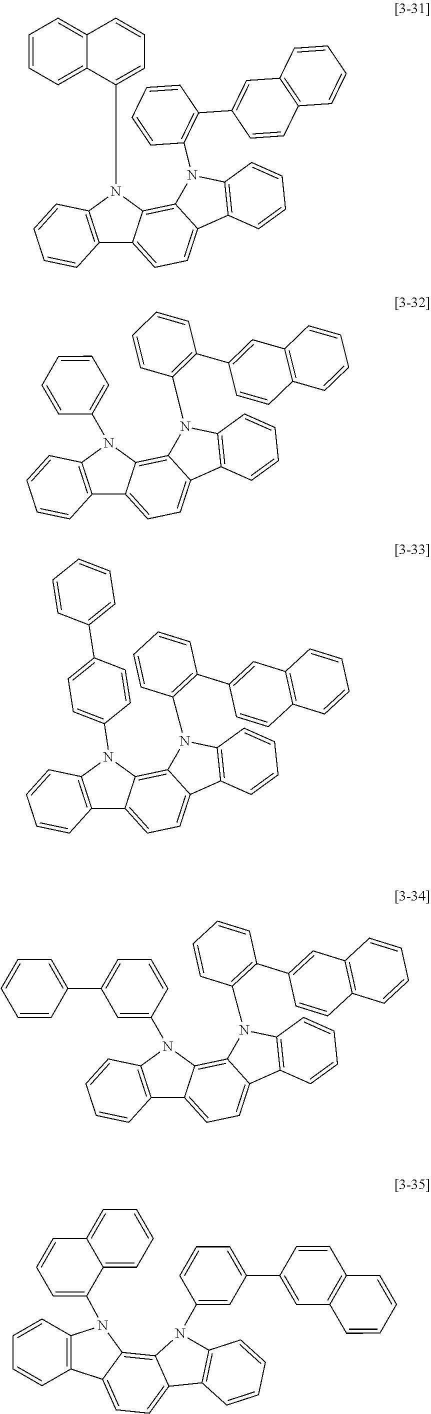

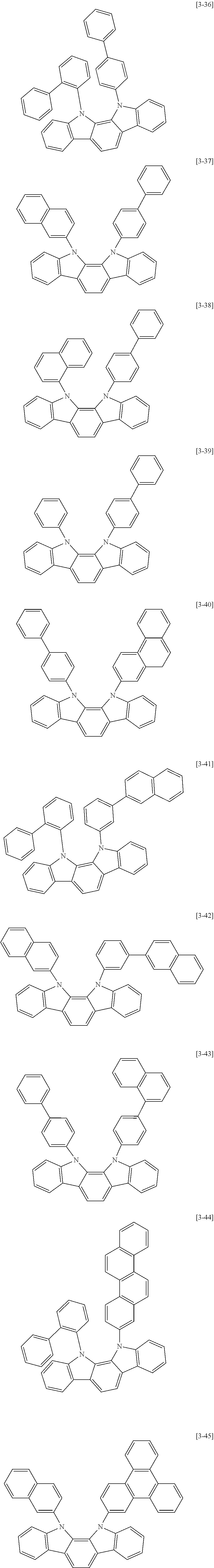

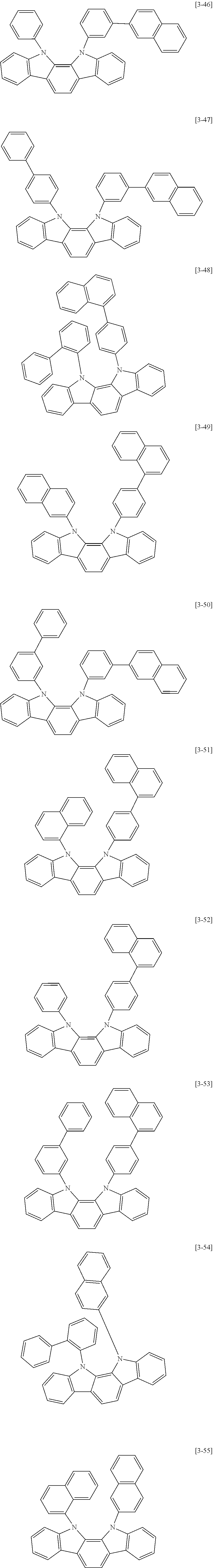

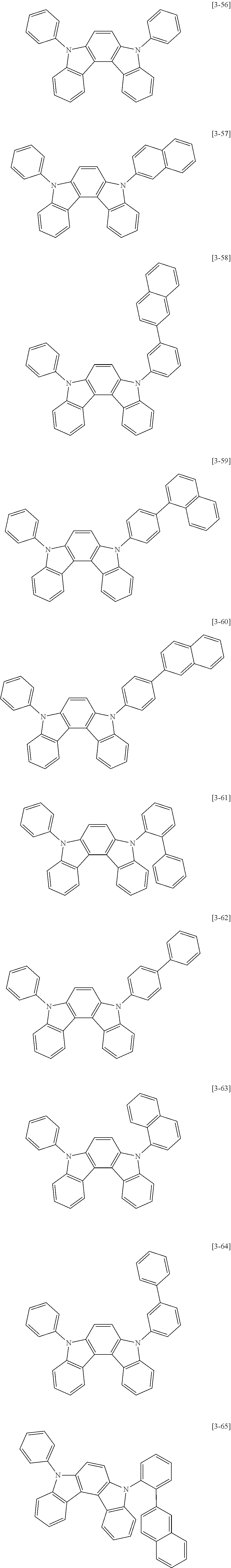

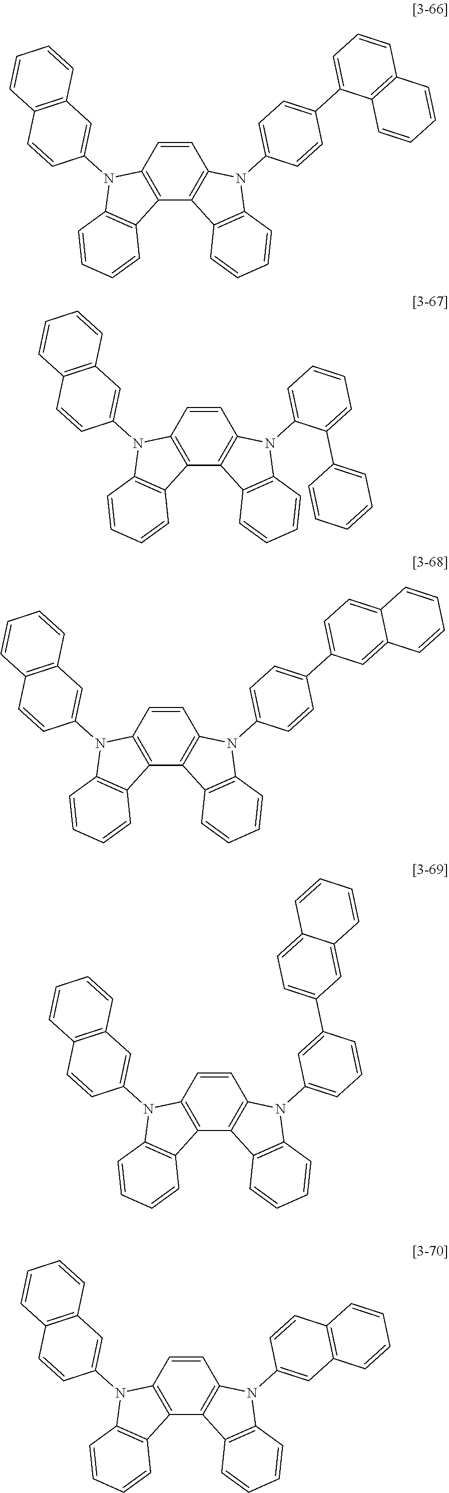 [SMILES: c1ccc2cc(-n3c4ccccc4c4c5c6ccccc6n(-c6ccc(-c7cccc8ccccc78)cc6)c5ccc43)ccc2c1 ; c1ccc(-c2ccccc2-n2c3ccccc3c3c4c5ccccc5n(-c5ccc6ccccc6c5)c4ccc32)cc1 ; c1ccc2cc(-c3ccc(-n4c5ccccc5c5c6c7ccccc7n(-c7ccc8ccccc8c7)c6ccc54)cc3)ccc2c1 ; c1cc(-c2ccc3ccccc3c2)cc(-n2c3ccccc3c3c4c5ccccc5n(-c5ccc6ccccc6c5)c4ccc32)c1 ; c1ccc2cc(-n3c4ccccc4c4c5c6ccccc6n(-c6ccc7ccccc7c6)c5ccc43)ccc2c1]

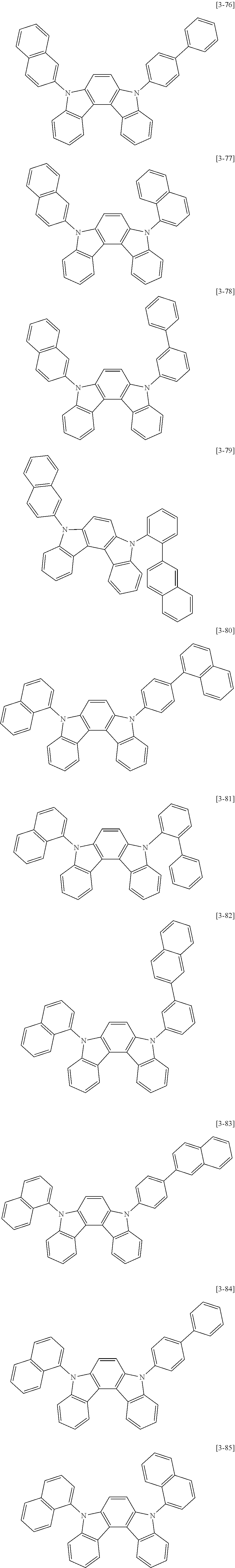

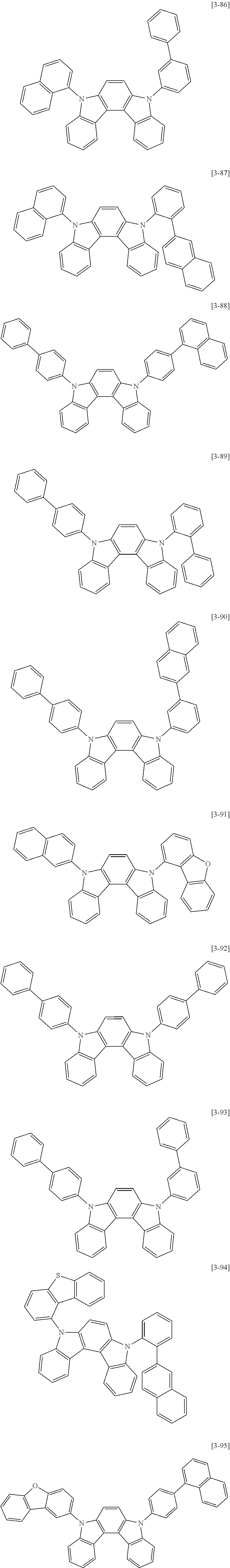

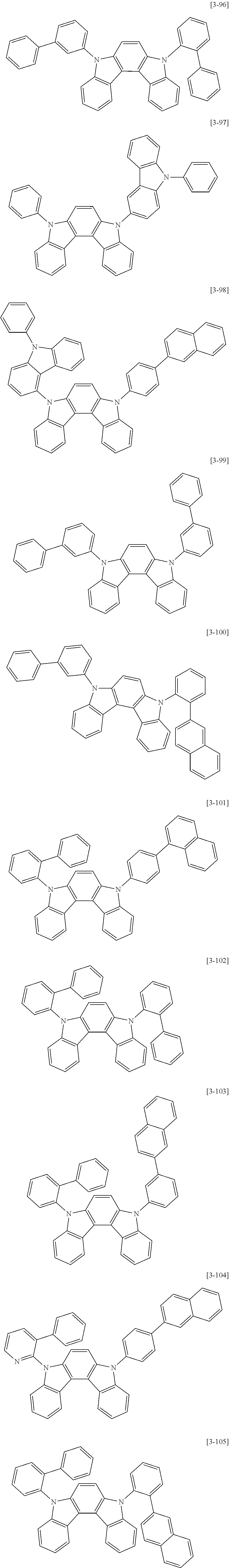

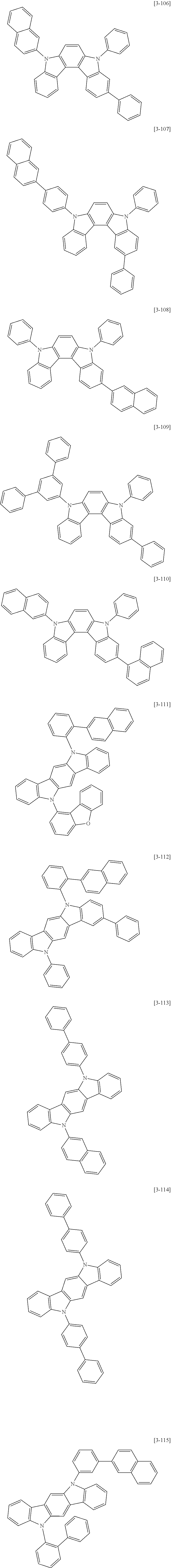

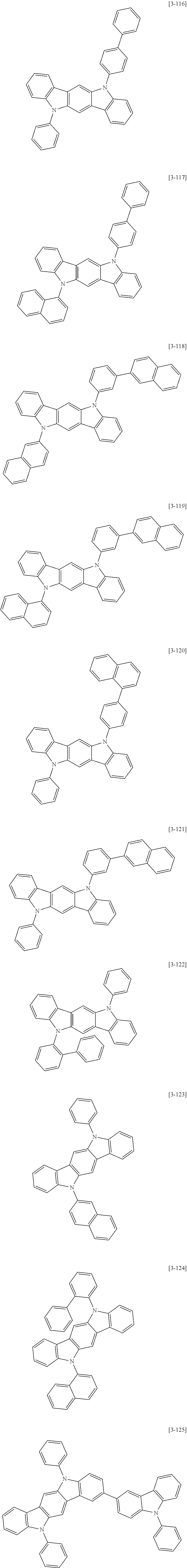

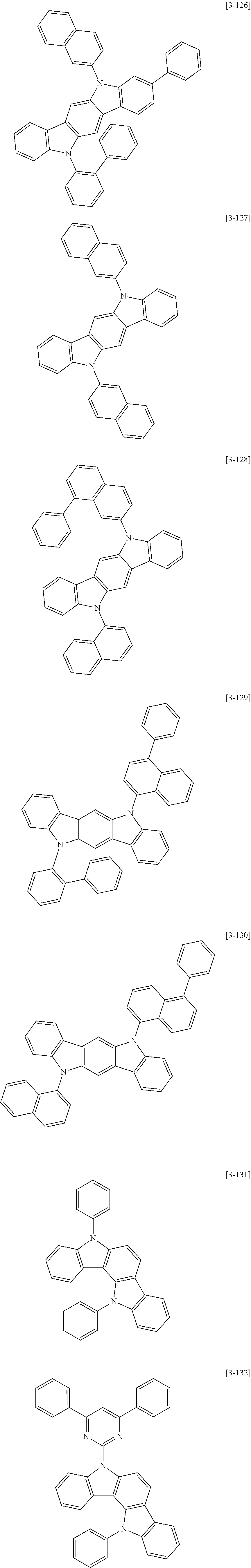

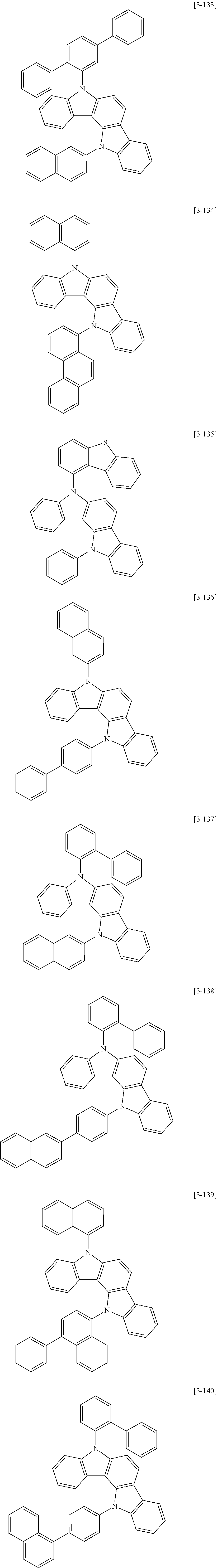

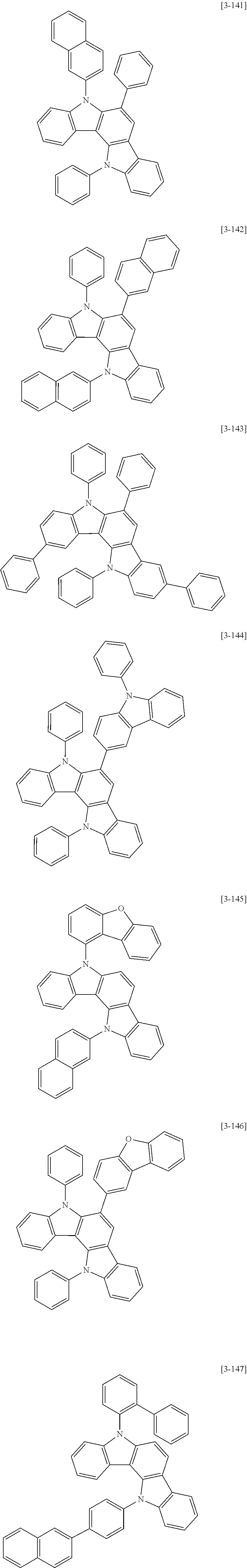

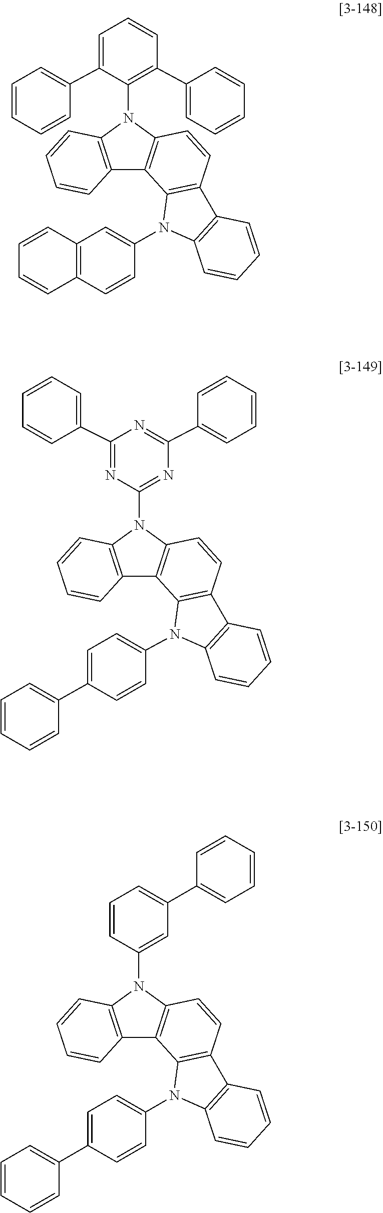 [SMILES: c1ccc(-c2cccc(-c3ccccc3)c2-n2c3ccccc3c3c2ccc2c4ccccc4n(-c4ccc5ccccc5c4)c23)cc1 ; c1ccc(-c2ccc(-n3c4ccccc4c4ccc5c(c6ccccc6n5-c5nc(-c6ccccc6)nc(-c6ccccc6)n5)c43)cc2)cc1 ; c1ccc(-c2ccc(-n3c4ccccc4c4ccc5c(c6ccccc6n5-c5cccc(-c6ccccc6)c5)c43)cc2)cc1]

17. An organic optoelectronic device, comprising:
an anode and a cathode facing each other, and
at least one organic layer between the anode and the cathode,
wherein the at least one organic layer includes a light emitting layer, and
the light emitting layer includes the composition for the organic optoelectronic device of claim 1.

18. The organic optoelectronic device of claim 17, wherein the composition for the organic optoelectronic device is a host of the light emitting layer.

19. A display device comprising the organic optoelectronic device of claim 17.
